(19) United States
(12) Patent Application Publication
(10) Pub. No.: US 2024/0294889 A1
(43) Pub. Date: Sep. 5, 2024

(54) DNASE FUSION POLYPEPTIDES AND RELATED COMPOSITIONS AND METHODS

(71) Applicant: Theripion, Inc., Shoreline, WA (US)

(72) Inventors: Jeffrey A. Ledbetter, Shoreline, WA (US); Martha S. Hayden-Ledbetter, Shoreline, WA (US)

(73) Assignee: Theripion, Inc., Shoreline, WA (US)

(21) Appl. No.: 18/646,706

(22) Filed: Apr. 25, 2024

Related U.S. Application Data
(62) Division of application No. 17/819,006, filed on Aug. 11, 2022.
Application No. 17/819,006 is a continuation-in-part of application No. PCT/US2022/016736, filed on Feb. 17, 2022.
(60) Provisional application No. 63/151,272, filed on Feb. 19, 2021.
(60) Provisional application No. 63/151,236, filed on Feb. 19, 2021.

(51) Int. Cl. C12N 9/22; A61K 38/00; A61P 11/00; C07K 16/24; C12N 9/16

(52) U.S. Cl. CPC C12N 9/22; A61P 11/00; C07K 16/241; C12N 9/16; C12Y 301/08001; A61K 38/00; C07K 2317/31; C07K 2317/622; C07K 2319/01; C07K 2319/30; C12Y 301/21001

(57) ABSTRACT

Compositions and methods relating to DNase fusion polypeptides are disclosed. The fusion polypeptides include a biologically active DNase joined to the amino-terminus of an immunoglobulin Fc region via a flexible polypeptide linker (e.g., a linker containing at least 26 amino acid residues). Typically, the DNase is a hyperactive and/or actin-resistant DNase1 variant (e.g., a variant of human DNase1 having one or more amino acid substitutions selected from substitutions at Asp-53, Tyr-65, Glu-69, Arg-74, Gly-105, and Ala-114 according to amino acid position numbering of mature wild-type human DNase1) or a DNase1L3 variant (e.g., a variant of human DNase1L3 in which the native nuclear localization signals are removed). In some embodiments, the fusion polypeptide includes a polypeptide segment located carboxyl-terminal to the Fc region and which may be, e.g., a biologically active paraoxonase. Also disclosed are dimeric proteins comprising first and second DNase fusion polypeptides as disclosed herein. The fusion polypeptides and dimeric proteins are useful in methods for therapy, including methods for treating diseases and disorders characterized by NETosis.

### CROSS-REFERENCE TO RELATED APPLICATIONS

This application is a division of U.S. application Ser. No. 17/819,006, filed Aug. 11, 2022, which is a continuation-in-part of International Application No. PCT/US2022/016736, filed Feb. 17, 2022, which claims the benefit of U.S. Provisional Application Nos. 63/151,272 and 63/151,236, filed Feb. 19, 2021. Each of the foregoing applications is incorporated by reference herein in its entirety.

### REFERENCE TO SEQUENCE LISTING

The instant application contains a Sequence Listing which has been submitted electronically in XML format and is hereby incorporated by reference in its entirety. Said XML Copy, created on Apr. 10, 2024, is named “4346-P3US-DIV_Seq_Listing_ST26” and is 299,468 bytes in size.

### BACKGROUND OF THE INVENTION

### DNase1

DNase1 is a calcium and magnesium-dependent endonuclease coded by the human DNASE] gene. DNase1 cleaves single-stranded and double-stranded DNA and chromatin. Cleavage of chromatin occurs in hypersensitive sites where chromatin is open and accessible, and is used in genomics to identify regions likely to contain active genes (Boyle et al., Cell 132:311-322, 2008). DNase1 helps degrade DNA during apoptosis (Samejima et al., Nat. Rev. Mol. Cell Biol. 6:677-688, 2008) and also degrades neutrophil extracellular traps (NETS) (Hakkim et al., Proc. Natl. Acad. Sci. USA 107:9813, 2010). DNase1 can digest biofilm DNA, resulting in a reduction in biofilm biomass, and increase antibiotic mediated biofilm killing (Tetz et al., Antimicrob. Agents Chemother. 53:1204-1209, 2009).

Accumulation of extracellular DNA from dead and dying cells was identified as a driver of disease in patients with systemic lupus erythematosus (SLE) long ago. DNase1 was administered to SLE patients at the Rockefeller starting in 1959. However, bovine DNase1 was used, and therapy was limited by its immunogenicity (see Valle et al., Autoimmunity Rev. 7:359, 2008). Decreased levels of DNase1 and DNase1 mutations with reduced activity have been reported in patients with SLE (see Valle et al., supra). Reduced DNase1 activity in urine was found in patients with lupus nephritis, and reduced DNase1 activity was a biomarker of disease progression (Pedersen et al., J. Pathol. Clin. Res. 4:193, 2018). In SLE patients, defective clearance of apoptotic debris and accumulation of chromatin contributes to breaking of tolerance and development of autoimmune disease.

Activated neutrophils extrude DNA bound with cytoplasmic and granule proteins, called neutrophil extracellular traps (NETs), to combat infectious agents, a process also known as “NETosis” (see Brinkmann, J. Innate Immun. 10:422-431, 2018). However, NETs are not targeted and often cause collateral tissue damage and inflammation if not effectively cleared. NETs have been implicated in driving disease progression in multiple uncurable and serious diseases. Evidence for the importance of NETs in psoriasis, cystic fibrosis, SLE, RA, type I diabetes, sepsis, small vessel vasculitis, IBD, type II diabetes, and obesity was recently reviewed (Mutua and Gershwin, Clin. Rev. Allergy Immunol, 2020, DOI 10.1007/s12016-020-08804-7). See also Michailidou, Front. Immunol. 11:619705, 2020 (reviewing involvement of neutrophils and NETs in vasculitis). NETs also play an important role in cardiovascular disease and thrombosis, and in cancer where NETs promote tumor progression and metastasis (Brinkmann, supra). In addition to their importance in cystic fibrosis, NETs also play a role in the clinicopathology of other respiratory diseases such as asthma and chronic obstructive pulmonary disease (COPD) (see Liu et al., Chinese Med. Journal 130:730-736, 2017; Twaddell et al., CHEST 156:774-782, 2019; Uddin et al., Front. Immunol. 10:47, 2019). Recently, NETs were found to contribute to immunothrombosis in COVID-19 acute respiratory distress syndrome (Middleton et al., Blood 136:1169, 2020; Zuo et al., JCI Insight 5:e138999, 2020; Barnes et al., J. Exp. Med. 217:e20200652, 2020).

DNase1 is capable of digesting NETs and works together with another extracellular DNase called DNase1L3 to clear NETs. Mice deficient in both DNase1 and DNase1L3 were unable to clear NETs and rapidly died of massive intravascular clot formation and organ damage after neutrophil activation (Brinkmann, supra). The contribution of NETs to thrombosis has already led to a clinical trial of wild-type DNase1 (Pulmozyme®) taken by inhalation therapy in COVID-19 patients (NCT04359654).

Recombinant wild-type human DNase1 (Pulmozyme®) was produced by Genentech and first approved for Cystic Fibrosis (CF) to digest DNA in the lungs and reduce congestion. High levels of DNA derived primarily from NETs are pathogenic in CF lung (Law et al., J. Inflammation, 14:1-8, 2017). Bacterial DNA from Pseudomonas aeruginosa biofilm formation is also present in many CF patients and may also be digested by DNase1 (Whitchurch et al., Science 295:1487, 2002).

Pulmozyme is delivered directly to the lungs by nebulization and is taken daily. Pulmozyme has been studied in patients with lung diseases other than CF, but has not been approved (Torbic et al., J. Pharmacy Practice 29:480, 2016). Pulmozyme was tested in a mouse lupus model (Verthelyi et al., Lupus 7:223, 1998) and in a phase I clinical trial in SLE patients (Davis et al., Lupus 8:68-76, 1999) without a decrease in disease measurements. Systemic administration of pulmozyme therapy may be limited because the enzyme is inhibited by globular actin that is abundant in blood and lungs (Ulmer et al., Proc. Natl. Acad. Sci. USA 93: 8225, 1996) and because of its short half-life.

Efforts have been made to enhance DNase1 for CF and SLE patients. A site-directed mutagenesis study of the DNA-binding interface identified 27 amino acid positions critical for enzyme activity and another 13 positions peripherally involved in DNA interactions with minimal impact on enzyme activity (see Pan et al., Prot. Sci. 7:628-636, 1998). This group also showed that increasing the local electrostatic attraction toward DNA by adding specific additional positively charged residues not only improved binding affinity to DNA but also improved functional activity and eliminated the inhibition by salt (Pan et al., Biochemistry 36:6624-6632, 1997; Pan and Lazarus, J. Biol. Chem. 273:11701-11708, 1998). Consistent with this observation, alanine replacement of two arginines (Arg-41 and Arg-111) that make critical contacts with DNA greatly reduced the DNA cleavage activity of human DNase1 (Pan et al., 1998, supra). Furthermore, the introduction of positively or negatively charged residues at sites distal to the DNA interface did not alter specific activity (Ulmer et al., Proc. Nati. Acad. Sci. USA 93:8225-8229, 1996).

The hyperactive DNase1 variants showed significant enhancement (up to 10,000-fold) under conditions of low DNA concentration and short DNA length (Pan and Lazarus, supra). Under optimal conditions for wild-type DNase1, however, the best variant showed activity only 7-fold higher and contained a single amino acid change (N74K). The N74K hyperactive variant showed reduced dependence on calcium (Pan and Lazarus, Prot. Sci. 8:1780-1788, 1999).

Ulmer et. al. (supra) studied amino acids important in actin binding and inhibition of DNase1 and showed that replacement of Ala-114 with glutamate, methionine, arginine, or tyrosine reduced inhibition of DNase1 by G-actin by greater than 1,000-fold. Elimination of G-actin-induced inhibition of DNase1 has also been shown by replacing Ala-114 with phenylalanine (A114F). See Pan et al., J. Biol. Chem. 273:18374-18381,1998. In addition to the A114 position, Asp-53, Tyr-65, and Val-67 were shown to be important residues for actin binding. See Ulmer et al., supra (showing about a 3-30-fold reduction in actin binding with alanine, methionine, arginine, or tyrosine replacement of Asp-53; about a 15—or 1,500-fold reduction with alanine or arginine replacement of Tyr-65; and about a 150—or 1,500-fold reduction with aspartate or lysine replacement of Val-67). Substitutions of Glu-69 with lysine or arginine did not reduce actin binding by more than two-fold, but these variants did exhibit increased DNase activity. See id.

Actin-resistant and hyperactive DNase1 mutant enzymes are further described in U.S. Pat. Nos. 6,348,343 and 6,391,607.

DNASE1 is polymorphic, and alleles that encode for enzyme with reduced activity are associated with increased risk for autoimmune disease. However, a naturally occurring allele, G105R, was found to encode a DNase1 variant with a three-fold increase in activity (Yasuda et al., Int. J. Biochem. Cell. Biol. 42:1216-1225, 2010). This is a minor allele found in African populations and could confer resistance to autoimmune disease (id.).

There is great potential for DNase1 therapy using actin/salt-resistant and hyperactive DNase1. However, it was not possible to obtain stable CHO cells that constitutively express an enhanced DNase1 despite screening of thousands of clones (see Lam et al., Biotech. Progress 32: 523-533, 2017). This was thought to be due to toxicity of the enzyme during high expression in CHO cells. Lam et al. (supra) describe an inducible expression system in CHO cells as one potential solution to the problem of manufacturing an enhanced DNase 1 enzyme.

Dwyer et al. (J. Biol. Chem. 274:9738-43, 1999) describe DNase1-Fc fusion proteins with impaired enzyme activity due to dimerization by the Fc domain. Dwyer et al. added short linkers (up to 7 aa) between the DNase1 and the Fc domain without improving activity (see id.). U.S. Pat. No. 8,841,416 to Ledbetter et al. describe a DNaseI-Fc fusion with a linker of 21 amino acids and having activity at least equivalent to recombinant DNase1 control.

Although wild-type DNase1 (Pulmozyme) therapy is FDA approved in CF, there is not a CF mouse model where DNase1 is active. Pulmozyme was approved for CF therapy without testing for activity in a relevant in vivo disease model (Greene, Hum. Exp. Toxicol., May:13 Suppl 1:S1-42, 1994). More recently, wild-type DNase1 therapy has been studied in several in vivo models with encouraging results. DNase1 was found to significantly reduce airway resistance but did not reduce inflammation in an acute asthma model (da Cunha et al., Exp. Lung Res. 42:66, 2016). In a silica-induced lung inflammation model, DNase1 therapy prevented DNA-mediated STING activation and blocked the downstream type I IFN response (Benmerzoug et al., Nat. Comm. 9:5226, 2018). In a model of acute lung injury, DNase1 therapy protected mice from lung edema and lung vascular permeability. DNase1 therapy also reduced NET formation and platelet sequestration in the lung (Caudrillier et al., J. Clin. Invest. 122:2661, 2012).

Studies in mouse tumor models have shown that NETs promote metastasis and that injection of DNase1 or DNase1-coated nanoparticles reduced tumor metastasis (Cools-Lartigue et al., J. Clin. Invest. 123:3446, 2013; Park et al., Sci. Translational Med. 8:361ra138, 2016). Another study showed that a combination of DNase1 and proteases significantly inhibited growth of human colon cancer cells in a nude mouse model (Trejo-Becirril et al., Integrative Cancer Therapies 15:NP35-NP43, 2016). In cancer patients, increased concentration of citrullinated histone H3, a marker of neutrophil NET formation, strongly predicted a poor clinical outcome (Thalin et al., PLoS ONE 13:e0191231, 2018).

In a murine DSS colitis model (Babicova et al., Folia Biolica (Praha) 64:10, 2018) DNase1 therapy caused a reduction in TNFα and myeloperoxidase in the colon. Another study (Li et al., J. Crohns Colitis 2020, 14:240-253, 2020) found that DNase1 therapy decreased cytokine production and attenuated accelerated thrombus formation and platelet activation after DSS-induced colitis.

DNase1 therapy in a hind-limb ischemia reperfusion model caused increased perfusion, decreased infiltrating inflammatory cells, and reduced a local marker of thrombosis (Albadawi et al., J. Vasc. Surg. 64:484, 2016). In a rat model of ischemia-reperfusion-induced acute kidney injury, DNase1 treatment showed significant renoprotective effects. Exogenous administration of DNase1 ameliorated both functional and histologic hallmarks of acute injury in kidneys of ischemic rats (Peer et al., Am. J. Nephrol. 43:195, 2016).

DNase1 treatment prevented organ damage and protected from death when given 4 or 6 hours after injury in a cecal ligation and puncture sepsis model in mice (Mai et al., Shock 44:166, 2015). Extracellular DNA from NETs has been recognized as a scaffold for thrombus formation and infusion of DNase1 prevented thrombosis in a model of inferior vena cava stenosis (Brill et al., J. Thrombosis Haemostasis 10:136, 2012).

### Paraoxonase 1 (PON1)

Paraoxonase 1 (PON1) is an antioxidant enzyme that inhibits oxidation of low-density lipoprotein (LDL) and preserves its function (Aviram et al., J. Clin. Invest. 101:1581-1590, 1998). PON1 has multiple substrates, including organophosphates and acyl homoserine lactones, the quorum sensing molecules made by Pseudomonas aeruginosa. PON1 activity is important in protection from arteriosclerosis and ischemic stroke (Litvinov et al., N. Am. J. Med. Sci. 4:523-532, 2012). In addition, PON1 activity is reduced in patients with intestinal inflammation including those with irritable bowel syndrome (IBS) or inflammatory bowel disease (IBD) such as Crohn's disease (Oran et al., J. Pak. Med. Assoc. 64:820-822, 2014; Szczelkik et al., Molecules 23:2603, 2018; Rothem et al., Free Rad. Biol. Med. 43:730-739, 2007). Studies in a mouse model for colitis have demonstrated that treatment with N-acetyl cysteine stimulates upregulation of PON1, and that increased PON1 activity is associated with amelioration of colon damage (You et al., Dig. Dis. Sci. 54:1643-1650, 2009).

PON1 is also deficient in multiple lung diseases. There is strong evidence that PON1 is important in protection from lung inflammation, and that low PON1 activity is associated with lung diseases. Oxidative stress is an important factor in the pathogenesis of asthma (see Sahiner et al., WAO Journal 4:151-158, 2011), and the antioxidant activity of PON1 is impaired in asthma patients. In children with asthma, PON1 levels were significantly lower than controls (p<0.001) (Emin et al., Allergol. Immunopathol. (Madr) 43:346-352, 2014). Additional studies have reported that PON1 activity is significantly lower in asthma patients (p<0.024) (Sarioglu et al., Iran J. Asthma Immunol. 14:60-66, 2015). Another study showed that PON1 activity is lower in asthmatic patients during active disease, but PONI activity increases during disease remission (Tolgyesi et al., Internat. Immunol. 21:967-975, 2009). This study also used gene profiling in an experimental mouse asthma model to identify PON1 downregulation as a potential target for monitoring disease activity (see id.). In addition, a mouse asthma model showed that PON1 overexpression reduced airway inflammation and remodeling, and reduced inflammatory cytokines (Chen et al., J. Cell Biochem. 119:793-805, 2018).

Compared to controls, patients with COPD were found to have significantly lower PON1 activity and increased oxidative stress as evidenced by lower levels of reduced thiol groups (Rumora et al., J. COPD 11:539-545, 2014). Another study found a polymorphism at −108 C>T in the PONI promoter was strongly associated with COPD (Rajkovic et al., J. Clin. Path. 71:963-970, 2018). Both the −108 TT genotype and T allele were strongly associated (p<0.001), and the −108T allele could be a factor in the reduced expression of PONI in COPD patients (id.).

PON1 activity was reported to be reduced in patients with sarcoidosis and interstitial lung disease, particularly during active disease (Ivanisevic et al., Eur. J. Clin. Invest. 46:418-424, 2016; Uzun et al., Curr. Med. Res. Opin. 24:1651-1657, 2008). Another study found lower PONI during hypoxia (Okur et al., Sleep Breath. 17:365-371, 2013). PONI activity was absent in patients with lung disease who had been exposed to sulfa mustard gas, even though the exposure was decades ago (Golmanesh et al., Immunopharmacol. and Immunotoxicol. 35:419-425, 2013).

PON1 and myeloperoxidase were reported to exert reciprocal control, in that an excess of PON1 inhibits myeloperoxidase, whereas an excess of myeloperoxidase inhibits PON1 (Huang et al., J. Clin. Invest. 123:3815-3828, 2013). Huang et al. found that both PONI and myeloperoxidase were tightly bound to apoA-I. This provides one mechanism for the decreased PON1 activity under conditions of inflammation, where neutrophils and eosinophils produce an excess of myeloperoxidase. However, under normal conditions, PON1 is in excess and represses the activity of myeloperoxidase. (See id.)

Recombinant PON1 (rPON1) was reported to inhibit PMA-induced differentiation of monocytic cell line THP-1 (Rosenblat et al., Atherosclerosis 219:49-56, 2011). Mice injected IP with rPON1 had reduced differentiation of monocytes to macrophages and reduced expression of CD11b and CD36. The total cellular peroxides of peritoneal macrophages decreased by 18%. (Id.)

PON1 therapy has been studied in experimental models. One study showed that recombinant human PON1 injection was able to protect guinea pigs from sarin and soman inhalation toxicity (Valiyaveettil et al., Biochem. Pharmacol. 81:800-809, 2011; Valiyaveettil et al., Toxicol. Letters 202:203-208, 2011). Other studies showed therapy with PON1 protected from organophosphate poisoning in mice (Bajaj et al., Appl. Biochem. Biotechnol. 180:165, 2016; Stevens et al., Proc. Natl. Acad. Sci. USA 105:12780-12784, 2008). These studies used recombinant PON1 purified from bacterial inclusion bodies or made in T. ni larvae with a baculovirus vector. Both groups also expressed PON1 192K, a variant derived from the rabbit sequence, that helps stabilize and increase PON1 enzymatic activity. (See Bajaj et al., supra; Stevens et al., supra.)

Expression of PON1 in bacteria has also been used to select mutants that have enhanced enzymatic activity towards organophosphates and nerve agents (see Aharoni et al., Proc. Nati. Acad. Sci. USA 101:482-487, 2004; Goldsmith et al., Chemistry and Biology 19:456, 2012). Engineering of PON1 was done by directed evolution to find a variant with improved, soluble expression in bacteria. One evolved PON1 was crystallized and the PON1 structure described (see Harel et al., Nature Struct. Mol. Biol. 11:412-419, 2004). Another PON1 variant widely used is G3C9, a hybrid PON1 formed from shuffling mouse, rat, rabbit, and human PON1 sequences (see Aharoni et al., Proc. Natl. Acad. Sci. USA 101:482-487, 2004). Other PON1 variants with increased organophosphatase activity but reduced esterase activity have been described (see Aharoni et al., supra). Further evolution of G3C9 to increase activity towards G-type nerve agents tabun, sarin, soman, and cyclosarin has also been reported (Gupta et al., Nat. Chem. Biol. 7:120-125, 2011; Goldsmith et al., Chemistry and Biology 19:456-466, 2012). When compared to human PON1, G3C9 has amino acid differences at 55 positions, and the further evolved PON1 variants have even more amino acid differences (Goldsmith et al., supra).

PON1 and the PON1 variant G3C9 have been tested in mouse models of experimental colitis (Yamashita et al., J. Immunol. 191:949-960, 2013). PON1 therapy was effective in TNBS-induced colitis and was equivalent to anti-TNFα therapy, currently the most effective clinically used therapeutic agent in man. However, in a chronic colitis model with CD4⁺CD45RBhi^{gh} cell transfer, PON1 was not effective but G3C9 showed strong efficacy. Efficacy of G3C9 was equivalent to anti-TNFα in the chronic colitis model. (Id.)_In another study, PON1 activity was found to correlate with severity of Crohn's disease (Sczceklik et al., Molecules 23:2603, 2018).

Another approach towards therapy with PON1 has been to inject HDL-like particles, formed from reconstituted apo A-I plus PON1, a therapy termed BL-3050 (Gaidukov et al., BMC Clinical Pharmacol. 9:18, 2009). In these studies, both G3C9 expressed in bacteria and BL-3050 complexes were protective in a mouse model of organophosphate poisoning (id.).

PON1 has been expressed as a fusion protein attached to the C-terminus of a mAb heavy chain specific for insulin receptor (see, e.g., Boado et al., Mol. Pharm. 5:1037, 2008). Expression of this hybrid heavy chain with an insulin receptor-specific light chain resulted in secretion of a bifunctional antibody that bound insulin receptor and had PON1 enzyme activity (id.). This hybrid molecule was expressed in mammalian cells (COS and CHO) (see id.; Boado et al., Biotechnol. and Bioengineering, 108:186, 2011). The hybrid molecule was also tested in nonhuman primates and was found to penetrate the blood/brain barrier; however, the molecule was rapidly cleared from the peripheral blood into the peripheral tissues, primarily the liver (Boado et al. (2011), supra). There are no reports of clinical testing of this molecule. Expression levels of the fusion protein in CHO cells were low (5-10 mg/liter) even after multiple transfections and subcloning (see id.).

PONI has also been fused with a membrane transduction domain, termed PEP-1-PON1 (Kim et al., Biomaterials 64:45-56, 2015). This molecule inhibited the inflammatory response of a microglial cell line and protected against dopaminergic cell death in vivo in a mouse model of Parkinson's disease (id.).

Paraoxonase enzymes are able to hydrolyse QS molecules that are acyl homoserine lactones (AHL), including C12HSL (N-3-oxo-dodecanoyl-L-Homoresine Lactone), the primary QS molecule from P. aeruginosa. Studies of human PON1 indicate that it has pleiotropic enzyme activities, hydrolyzing a variety of substrates, including lactones (Billecke et al., Drug Metabolism and Disposition 28:1335-1342, 2000; Khersonsky and Tawfik, Biochemistry 44:6371-6382, 2005; Gaidukov and Tawfik, J. Lipid Res. 48:1637-1646, 2007; Bajaj et al., Protein Science 22:1799-1807, 2013). One study in Drosophila melanogaster demonstrated protection from Pseudomonas aeruginosa lethality by expression of a human PONI transgene capable of hydrolyzing a critical quorum sensing molecule N-3-oxodecanoyl homoserine lactone (30C12-HSL) (Stoltz et al., J. Clin. Invest. 118:3123-3131, 2008).

### SUMMARY OF THE INVENTION

In one aspect, the present invention provides a fusion polypeptide comprising, from an amino-terminal position to a carboxyl-terminal position, D-L1-X, wherein D is a biologically active DNase having at least 90% or at least 95% identity with the amino acid sequence shown in (i) residues 21-280 of SEQ ID NO:4, (ii) residues 21-280 of SEQ ID NO:52, (iii) residues 21-280 of SEQ ID NO:74, (iv) residues 21-280 of SEQ ID NO:76, (v) residues 21-280 of SEQ ID NO:78, or (vi) residues 21-290 of SEQ ID NO:34, LI is a first polypeptide linker, and X is an immunoglobulin Fc region. Preferably, L1 comprises at least 26 amino acid residues. For example, in some embodiments, Li consists of from 26 to 60 amino acid residues or from 26 to 36 amino acid residues. In particular variations, LI comprises four or more tandem repeats of the amino acid sequence of SEQ ID NO:29; in some such embodiments, LI has the amino acid sequence shown in SEQ ID NO:8 or SEQ ID NO:10.

In some embodiments of a fusion polypeptide as above wherein the DNase has at least 90% or at least 95% identity with the amino acid sequence shown in residues 21-280 of SEQ ID NO:4 or residues 21-280 of SEQ ID NO:52, the DNase contains at least one amino acid substitution at a position corresponding to an amino acid of human DNase1 (SEQ ID NO:30) selected from N74, G105, and A114, wherein (1) the amino acid substitution at the position corresponding to N74 of human DNase1 (N74 substitution), if present, increases DNA binding of the DNase relative to human DNase1, (2) the amino acid substitution at the position corresponding to G105 of human DNase1 (G105 substitution), if present, increases DNA binding of the DNase relative to human DNase1, and (3) the amino acid substitution at the position corresponding to A114 of human DNase1 (A114 substitution), if present, decreases G-actin-induced inhibition of endonuclease activity of the DNase relative to human DNase1. In some such variations, the fusion polypeptide contains both the N74 and G105 substitutions, both the G105 and A114 substitutions, or each of the N74, G105, and A114 substitutions. Particularly suitable amino acid substitutions at these positions are lysine at the position corresponding to N74 of human DNase1, arginine at the position corresponding to G105 of human DNase1, and/or phenylalanine at the position corresponding to A114 of human DNase1. In some embodiments wherein the fusion polypeptide contains both the N74 and G105 substitutions, the fusion polypeptide does not contain the A114 substitution. In more specific variations, the DNase has the amino acid sequence shown in (i) residues 21-280 of SEQ ID NO:4, (ii) residues 21-280 of SEQ ID NO:6, or (iii) residues 21-280 of SEQ ID NO:52.

In some embodiments of a fusion polypeptide as above wherein the DNase has at least 90% or at least 95% identity with the amino acid sequence shown in residues 21-280 of SEQ ID NO:74, residues 21-280 of SEQ ID NO:76, or residues 21-280 of SEQ ID NO:78, the DNase contains at least one amino acid substitution at a position corresponding to an amino acid of human DNase1 (SEQ ID NO:30) selected from D53, Y65, and E69, and optionally contains at least one amino acid substitution at a position corresponding to an amino acid of human DNase1 selected from N74 and G105, wherein (1) the amino acid substitution at the position corresponding to D53 of human DNase1 (D53 substitution), if present, decreases G-actin-induced inhibition of endonuclease activity of the DNase relative to human DNase1, (2) the amino acid substitution at the position corresponding to Y65 of human DNase1 (Y65 substitution), if present, decreases G-actin-induced inhibition of endonuclease activity of the DNase relative to human DNase1, (3) the amino acid substitution at the position corresponding to E69 of human DNase1 (E69 substitution), if present, decreases G-actin-induced inhibition of endonuclease activity of the DNase relative to human DNase1, (4) the amino acid substitution at the position corresponding to N74 of human DNase1 (N74 substitution), if present, increases DNA binding of the DNase relative to human DNase1, and (5) the amino acid substitution at the position corresponding to G105 of human DNase1 (G105 substitution), if present, increases DNA binding of the DNase relative to human DNase1. In some such variations, the fusion polypeptide contains (i) the D53 substitution, (ii) the Y65 substitution, (iii) the E69 substitution, (iv) each of the D53, N74, and G105 substitutions, (v) each of the Y65, N74, and G105 substitutions, (vi) each of the E69, N74, and G105 substitutions, (vii) each of the D53, Y65, N74, and G105 substitutions, (viii) each of the D53, E69, N74, and G105 substitutions, (ix) each of the Y65, E69, N74, and G105 substitutions, or (x) each of the D53, Y65, E69, N74, and G105 substitutions. Particularly suitable amino acid substitutions at these positions are arginine at the position corresponding to D53 of human DNase1, alanine at the position corresponding to Y65 of human DNase1, lysine at the position corresponding to E69 of human DNase1, lysine at the position corresponding to N74 of human DNase1, and/or arginine at the position corresponding to G105 of human DNase1. In more specific variations, the DNase has the amino acid sequence shown in (i) residues 21-280 of SEQ ID NO:74, (ii) residues 21-280 of SEQ ID NO:76, (iii) residues 21-280 of SEQ ID NO:78, (iv) residues 21-280 of SEQ ID NO:80, (v) residues 21-280 of SEQ ID NO:82, or (vi) residues 21-280 of SEQ ID NO:84.

In some embodiments of a fusion polypeptide as above wherein the DNase has at least 90% or at least 95% identity with the amino acid sequence shown in residues 21-290 of SEQ ID NO:34, each of the amino acids at positions corresponding to R80, R95, and N96 of SEQ ID NO:34 is alanine or serine (e.g., each of R80, R95, and N96 is alanine, or each of R80, R95, and N96 is serine). In more specific variations, the DNase has the amino acid sequence shown in (i) amino acid residues 21-290 of SEQ ID NO:36 or (ii) amino acid residues 21-290 of SEQ ID NO:38.

In some embodiments of a fusion polypeptide as above, the immunoglobulin Fc region is a human Fc region such as, e.g., a human Fe variant comprising one or more (e.g., from one to 10) amino acid substitutions relative to the wild-type human sequence. Particularly suitable Fc regions include human γ1 and γ4 Fc regions. In some variations, the Fc region is a human γ1 Fe variant in which Eu residue C220 is replaced by serine; in some such embodiments Eu residues C226 and C229 are each replaced by serine, and/or Eu residue P238 is replaced by serine. In further variations comprising an Fc region as above, the Fc region is a human γ1 Fe variant in which Eu residue P331 is replaced by serine. In some embodiments, the Fc region has at least 90% or at least 95% identity with the amino acid sequence shown in (i) residues 1-232 or 1-231 of SEQ ID NO:12, (ii) residues 1-232 or 1-231 of SEQ ID NO:14, or (iii) residues 1-232 or 1-231 of SEQ ID NO:16. In more specific variations, the Fc region has the amino acid sequence shown in (i) residues 1-232 or 1-231 of SEQ ID NO:12, (ii) residues 1-232 or 1-231 of SEQ ID NO:14, or (iii) residues 1-232 or 1-231 of SEQ ID NO:16.

In certain embodiments of a fusion polypeptide as above, the fusion polypeptide comprises an amino acid sequence having at least 90% or at least 95% identity with the amino acid sequence shown in (i) residues 21-538 or 21-537 of SEQ ID NO:18, (ii) residues 21-548 or 21-547 of SEQ ID NO:20, (iii) residues 21-538 or 21-537 of SEQ ID NO:55, (iv) residues 21-548 or 21-547 of SEQ ID NO:56, (v) residues 21-538 or 21-537 of SEQ ID NO:86, (vi) residues 21-538 or 21-537 of SEQ ID NO:88, (vii) residues 21-538 or 21-537 of SEQ ID NO:90, (viii) residues 21-538 or 21-537 of SEQ ID NO:92, (ix) residues 21-538 or 21-537 of SEQ ID NO:94, (x) residues 21-538 or 21-537 of SEQ ID NO:96, (xi) residues 21-548 or 21-547 of SEQ ID NO:36, or (xii) residues 21-548 or 21-547 of SEQ ID NO:38. In some embodiments, the fusion polypeptide comprises the amino acid sequence shown in SEQ ID NO:49, SEQ ID NO:50, SEQ ID NO:53, or SEQ ID NO:54. In other embodiments, the fusion polypeptide comprises the amino acid sequence shown in SEQ ID NO:97, SEQ ID NO:98, SEQ ID NO:99, SEQ ID NO:100, SEQ ID NO:101, SEQ ID NO:102, SEQ ID NO:103, or SEQ ID NO:104. In more specific variations, the fusion polypeptide comprises the amino acid sequence shown in (i) residues 21-538 or 21-537 of SEQ ID NO:18, (ii) residues 21-548 or 21-547 of SEQ ID NO:20, (iii) residues 21-538 or 21-537 of SEQ ID NO:55, (iv) residues 21-548 or 21-547 of SEQ ID NO:56, (v) residues 21-538 or 21-537 of SEQ ID NO:86, (vi) residues 21-538 or 21-537 of SEQ ID NO:88, (vii) residues 21-538 or 21-537 of SEQ ID NO:90, (viii) residues 21-538 or 21-537 of SEQ ID NO:92, (ix) residues 21-538 or 21-537 of SEQ ID NO:94, (x) residues 21-538 or 21-537 of SEQ ID NO:96, (xi) residues 21-548 or 21-547 of SEQ ID NO:36, or (xii) residues 21-548 or 21-547 of SEQ ID NO:38.

In some embodiments of a fusion polypeptide as above, the fusion polypeptide further includes a polypeptide segment located carboxyl-terminal to the Fc region (also referred to herein as a “third polypeptide segment”). In particular variations, the third polypeptide segment is a biologically active paraoxonase such as, e.g., human paraoxonase 1 (PON1) or a functional variant thereof. A fusion polypeptide comprising a third polypeptide segment as above may be represented by the formula D-L1-X-L2-P (from an amino-terminal position to a carboxyl-terminal position), wherein D, L1, and X are each defined as above, wherein L2 is a second polypeptide linker and is optionally present, and wherein P is the third polypeptide segment. In some embodiments of a fusion polypeptide in which L2 is present, L2 comprises at least eight amino acid residues (e.g., L2 consists of from 12 to 25 amino acid residues); in more specific variations, L2 has the amino acid sequence shown in SEQ ID NO:32.

In some embodiments of a fusion polypeptide as above comprising a biologically active paraoxonase, the paraoxonase has at least 80% or at least 90% identity with the amino acid sequence shown in residues 16-355 or 26-355 of SEQ ID NO:24 and does not contain an amino-terminal leader sequence corresponding to residues 1-15 of SEQ ID NO:24. In some embodiments, the biologically active paraoxonase has at least 95% identity with the amino acid sequence shown in (i) residues 16-355 or 26-355 of SEQ ID NO:24, (ii) residues 16-355 or 26-355 of SEQ ID NO:40, or (iii) residues 16-355 or 26-355 of SEQ ID NO:42. In some variations, the amino acid at a position corresponding to Q192 of the human paraoxonase 1 Q192 isoform (hPON1-Q192; SEQ ID NO:22) is lysine or arginine. In other, non-mutually exclusive variations, the amino acid at the position corresponding to H115 of hPON1-Q192 is tryptophan. In specific variations, the paraoxonase has an amino acid sequence selected from (i) residues n-355 of SEQ ID NO:24, (ii) residues n-355 of SEQ ID NO:40, and (iii) residues n-355 of SEQ ID NO:42, wherein n is an integer from 16 to 26, inclusive. In particular embodiments of a fusion polypeptide comprising a paraoxonase and having the formula D-L1-X-L2-P as above, the fusion polypeptide comprises an amino acid sequence having at least 90% or at least 95% identity with the amino acid sequence shown in (i) residues 21-896 of SEQ ID NO:26, (ii) residues 21-896 of SEQ ID NO:44, (iii) residues 21-896 of SEQ ID NO:46, (iv) residues 21-906 of SEQ ID NO:48, (v) residues 21-896 of SEQ ID NO:58, (vi) residues 21-906 of SEQ ID NO:60, (vii) residues 21-906 of SEQ ID NO:68, or (viii) residues 21-906 of SEQ ID NO:70; in some embodiments, the fusion polypeptide comprises the amino acid sequence shown in (i) residues 21-896 of SEQ ID NO:26, (ii) residues 21-896 of SEQ ID NO:44, (iii) residues 21-896 of SEQ ID NO:46, (iv) residues 21-906 of SEQ ID NO:48, (v) residues 21-896 of SEQ ID NO:58, (vi) residues 21-906 of SEQ ID NO:60, (vii) residues 21-906 of SEQ ID NO:68, or (viii) residues 21-906 of SEQ ID NO:70.

In another aspect, the present invention provides a dimeric protein comprising a first fusion polypeptide and a second fusion polypeptide, wherein each of said first and second fusion polypeptides is a fusion polypeptide as described above.

In another aspect, the present invention provides a polynucleotide encoding a fusion polypeptide as described above.

In still another aspect, the present invention provides an expression cassette comprising a DNA segment encoding a fusion polypeptide as described above and which is operably linked to a promoter. In particular variations, the biologically active DNase of the encoded fusion polypeptide (a) includes at least one of the N74, G105, and A114 substitutions (e.g., a DNase having one, two, or all three substitutions and having at least 90% or at least 95% identity with amino acid residues 21-280 of SEQ ID NO:4) or (b) includes at least one of the D53, Y65, and E69 substitutions and optionally includes at least one of the N74 and G105 substitutions (e.g., a DNase having one, two, or all three of the D53, Y65, and E69 substitutions and having at least 90% or at least 95% identity with residues 21-280 of SEQ ID NO:74, residues 21-280 of SEQ ID NO:76, or residues 21-280 of SEQ ID NO:78). Also provided is a cultured cell into which has been introduced an expression cassette as described above, wherein the cell expresses the DNA segment. In a related aspect, the present invention provides a stable cell line comprising, within its genomic DNA, an expression cassette as described above, wherein the stable cell line constitutively expresses the DNA segment. In some embodiments, the stable cell line is a Chinese hamster ovary (CHO) cell line.

In another aspect, the present invention provide a vector comprising an expression cassette as described above.

In another aspect, the present invention provides a method of making a fusion polypeptide. The method generally includes (i) culturing a cell into which has been introduced an expression cassette as described above, wherein the cell expresses the DNA segment and the encoded fusion polypeptide is produced, and (ii) recovering the fusion polypeptide. In some variations, the cultured cell is a stable cell line as described above.

In yet another aspect, the present invention provides a method of making a dimeric protein. The method generally includes (i) culturing a cell into which has been introduced an expression cassette as described above, wherein the cell expresses the DNA segment and the encoded fusion polypeptide is produced as a dimeric protein, and (ii) recovering the dimeric protein. In some variations, the cultured cell is a stable cell line as described above.

In another aspect, the present invention provides a composition comprising a fusion polypeptide as described above and a pharmaceutically acceptable carrier.

In another aspect, the present invention provides a composition comprising a dimeric protein as described above and a pharmaceutically acceptable carrier.

In some embodiments of a composition as described above, the composition is formulated for delivery to the lung by nebulization.

In still another aspect, the present invention provides a method for treating a disease or disorder characterized by NETosis. The method generally includes administering to a subject having the disease or disorder characterized by NETosis an effective amount of a fusion polypeptide or dimeric protein as described above. In some embodiments, the disease or disorder is an inflammatory lung disease such as, for example, chronic obstructive pulmonary disease (COPD), asthma, cystic fibrosis (CF), bronchiectasis, hypoxia, acute respiratory distress syndrome (ARDS) (e.g., COVID-19-associated ARDS), or interstitial lung disease (e.g., idiopathic pulmonary fibrosis (IPF) or sarcoidosis). In other embodiments, the disease or disorder is an inflammatory skin disease such as, for example, psoriasis or atopic dermatitis. In other embodiments, the disease or disorder is an autoimmune disease such as, e.g., systemic lupus erythematosus (SLE) (e.g., SLE with lupus nephritis), rheumatoid arthritis (RA), psoriasis, type 1 diabetes mellitus, antiphospholipid syndrome, vasculitis, or systemic sclerosis. In other embodiments, the disease or disorder is an autoinflammatory disease such as, for example, an inflammatory bowel disease (IBD) (e.g., Crohn's disease or ulcerative colitis) or gout. In other embodiments, the disease or disorder is a neurological disease or disorder such as, for example, a chronic neurodenerative disease (e.g., Alzheimer's disease or multiple sclerosis), a central nervous system infection (e.g., meningitis, encephalitis, or cerebral malaria), or ischemic stroke. In other embodiments, the disease or disorder is a metabolic disease such as, e.g., type 2 diabetes or obesity. In other embodiments, the disease or disorder is a cardiovascular disease such as, for example, a cardiovascular disease characterized by atherosclerosis (e.g., coronary heart disease or ischemic stroke). In still other embodiments, the disease or disorder is selected from thrombosis, sepsis, and ischemia reperfusion. In other embodiments, the disease or disorder is a chronic liver disease such as, e.g., nonalcoholic steatohepatitis (NASH). In yet other embodiments, the disease or disorder is a fibrotic disease such as, e.g., systemic sclerosis, systemic lupus erythematosus (SLE), an inflammatory lung disease, a chronic liver disease, or a chronic kidney disease (e.g., lupus nephritis, IgA nephropathy, or membranous glomerulonephritis).

In another aspect, the present invention provides a method for reducing NETosis in a subject. The method generally includes administering to a subject having NETosis an effective amount of a fusion polypeptide or dimeric protein as described above.

In another aspect, the present invention provides a method for protecting a subject from aging. The method generally includes administering to the subject an effective amount of a fusion polypeptide or dimeric protein as described above.

In certain embodiments of a method for treating a disease or disorder characterized by NETosis, reducing NETosis, or protecting from aging as above, the method is a combination therapy further comprising administering an effective amount of a biologically active paraoxonase. In some variations, the paraoxonase has at least 80% or at least 90% identity with the amino acid sequence shown in residues 16-355 or 26-355 of SEQ ID NO:24 and does not contain an amino-terminal leader sequence corresponding to residues 1-15 of SEQ ID NO:24. In some such embodiments, the biologically active paraoxonase has at least 95% identity with the amino acid sequence shown in (i) residues 16-355 or 26-355 of SEQ ID NO:24, (ii) residues 16-355 or 26-355 of SEQ ID NO:40, or (iii) residues 16-355 or 26-355 of SEQ ID NO:42. In other, non-mutually exclusive variations, the paraoxonase is contained within a paraoxonase fusion polypeptide comprising, from an amino terminal position to a carboxyl terminal position, Xₚ-L2-P, wherein Xₚ is an immunoglobulin heavy chain constant region (e.g., an immunoglobulin Fc region), L2 is a polypeptide linker (e.g., a linker comprising at least eight amino acid residues) and is optionally present, and P is the paraoxonase (or contained with a dimeric protein formed by dimerization of the fusion polypeptide). In some such embodiments, the paraoxonase fusion polypeptide comprises an amino acid sequence having at least 90% or at least 95% identity with the amino acid sequence shown in (i) residues 21-613 of SEQ ID NO:61, (ii) residues 21-613 of SEQ ID NO:62, (iii) residues 21-613 of SEQ ID NO:63, (iv) residues 21-610 of SEQ ID NO:64, (v) residues 21-610 of SEQ ID NO:65, or (vi) residues 21-610 of SEQ ID NO:66; in more specific variations, the paraoxonase fusion polypeptide comprises the amino acid sequence shown in (i) residues 21-613 of SEQ ID NO:61, (ii) residues 21-613 of SEQ ID NO:62, (iii) residues 21-613 of SEQ ID NO:63, (iv) residues 21-610 of SEQ ID NO:64, (v) residues 21-610 of SEQ ID NO:65, or (vi) residues 21-610 of SEQ ID NO:66.

In some embodiments of a method for treating a disease or disorder characterized by NETosis as above, the disease or disorder is a cancer. In certain variations, the cancer treatment is a combination therapy. In some combination therapy embodiments, the combination therapy includes an immunomodulatory therapy comprising an anti-PD-1/PD-Li therapy, an anti-CTLA-4 therapy, a CAR T-cell therapy, or a combination thereof (e.g., both an anti-PD-1/PD-L1 therapy and an anti-CTLA-4 therapy). In other combination therapy embodiments, the combination therapy includes radiation therapy or chemotherapy. In some combination therapy embodiments, the combination therapy includes a targeted therapy; in some such embodiments, the targeted therapy includes (i) a therapeutic monoclonal antibody targeting a specific cell-surface or extracellular antigen (e.g., VEGF, EGFR, CTLA-4, PD-1, or PD-L1) or (ii) a small molecule targeting an intracellular protein such as, for example, an intracellular enzyme (e.g., a proteasome, a tyrosine kinase, a cyclin-dependent kinase, serine/threonine-protein kinase B-Raf (BRAF), or a MEK kinase).

These and other aspects of the invention will become evident upon reference to the following detailed description of the invention.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

A “polypeptide” is a polymer of amino acid residues joined by peptide bonds, whether produced naturally or synthetically. Polypeptides of less than about 50 amino acid residues may also be referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The terms “amino-terminal” (or “N-terminal”) and “carboxyl-terminal” (or “C-terminal”) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The terms “polynucleotide” and “nucleic acid” are used synonymously herein and refer to a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment that, when read from the 5′ to the 3′ direction, encodes the sequence of amino acids of the specified polypeptide. Also, for example, in the context of a fusion polypeptide as described herein, different components of the fusion polypeptide (e.g., a DNase, linker(s), an immunoglobulin Fc region, a paraoxonase) may each be referred to as a polypeptide segment.

The term “biologically active,” when used in reference to a polypeptide segment of a fusion molecule as described herein, means a polypeptide that causes a measurable or detectable physiological, biochemical, or molecular effect in a biological system. When used in specific reference to a polypeptide segment that is a DNase or paraoxonase (PON), “biologically active” means that the polypeptide exhibits the same type of enzymatic activity as a corresponding, naturally occurring enzyme (e.g., the same type of enzymatic activity as a full-length, wild-type human DNase1/DNase1L3 or PON1, respectively), allowing for differences in degree of activity, enzyme kinetics, and the like. An immunoglobulin Fc region, as referenced herein, is understood to be “biologically active” at least by virtue of its dimerizing and FcRn-binding activities.

Unless the context clearly indicates otherwise, reference herein to “DNase” (e.g., “DNase1” or “DNase1L3”) and “paraoxonase” (e.g., “paraoxonase 1” or “PON1”) is understood to include naturally occurring polypeptides of the foregoing, as well as functional variants and functional fragments thereof.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “enhanced DNase1” as used herein denotes a hyperactive and/or actin-resistant variant of a naturally occurring DNase1 (e.g., human DNase1), the variant having (a) at least one amino acid substitution that decreases G-actin-induced inhibition of endonuclease activity relative to the naturally occurring DNase1, and/or (b) at least one amino acid substitution that increases DNA binding relative to the naturally occurring DNase1. In some embodiments, the enhanced DNase1 comprises the at least one amino acid substitution that decreases G-actin-induced inhibition of endonuclease activity. In some embodiments, the enhanced DNase1 comprises both the at least one amino acid substitution that decreases G-actin-induced inhibition of endonuclease activity and the at least one amino acid substitution that increases DNA binding. In some preferred embodiments, an enhanced DNase1 is a hyperactive variant of wild-type human DNase1 (mature amino acid sequence shown in SEQ ID NO:30).

The term “linker” or “polypeptide linker” is used herein to indicate two or more amino acids joined by peptide bond(s) and linking two discrete, separate polypeptide regions. The linker is typically designed to allow the separate polypeptide regions (such as, e.g., a DNase polypeptide linked to an Fc region) to perform their separate functions. The linker can be a portion of a native sequence, a variant thereof, or a synthetic sequence. Linkers are also referred to herein using the abbreviation “L.” The use of a numerical identifier (“1” or “2”) with “L” is used herein to differentiate among linkers joining different fusion components: “L1” refers to a linker joining a biologically active DNase to the N-terminus of an Fc region, and “L2” refers to a linker joining the C-terminus of an Fc region to a second polypeptide segment such as, e.g., a biologically active paraoxonase. In the context of a polypeptide chain containing both L1 and L2 linkers, the linkers may be the same or different with respect to amino acid sequence.

The term “expression cassette” is used to denote a DNA construct that comprises a segment encoding a polypeptide of interest operably linked to additional segments that provide for its transcription in an appropriate host cell. Such additional sequences include a promoter and, typically, a transcription terminator, and may also include one or more selectable markers, an enhancer, a polyadenylation signal, etc.

The term “vector” is used to denote a polynucleotide produced by recombinant DNA techniques for delivering genetic material into a cell, where it can be replicated. As is well-known in the art, it may refer, e.g., to a plasmid, a cosmid, a viral vector, an artificial chromosome, a cloning vector, or an expression vector. The term “expression vector” is used to denote a vector comprising an expression cassette.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “secretory signal sequence” is a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

The term “recombinant” when used with reference, e.g., to a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all. By the term “recombinant nucleic acid” herein is meant nucleic acid, originally formed in vitro, in general, by the manipulation of nucleic acid using, e.g., polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes disclosed herein. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes disclosed herein. Similarly, a “recombinant protein” is a protein made using recombinant techniques, i.e., through the expression of a recombinant nucleic acid as depicted above.

The term “heterologous,” when used with reference to portions of a nucleic acid, indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, “heterologous,” when used in reference to portions of a protein, indicates that the protein comprises two or more subsequences that are not found in the same relationship to each other in nature (e.g., two or more segments of a fusion polypeptide).

As used herein, the term “immunoglobulin” refers to a protein consisting of one or more polypeptides substantially encoded by immunoglobulin gene(s). One form of immunoglobulin constitutes the basic structural unit of an intact, native antibody. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a γ1 heavy chain constant region. Each immunoglobulin heavy chain possesses a constant region that consists of constant region protein domains (CH1, hinge, CH2, and CH3; IgG3 also contains a CH4 domain) that are essentially invariant for a given subclass in a species. DNA sequences encoding human and non-human immunoglobulin chains are known in the art. (See, e.g., Ellison et al., DNA 1:11-18, 1981; Ellison et al., Nucleic Acids Res. 10:4071-4079, 1982; Kenten et al., Proc. Nati. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31:169-217, 1994.

An “immunoglobulin hinge” is that portion of an immunoglobulin heavy chain connecting the CHI and CH2 domains. The hinge region of human γ1 corresponds approximately to Eu residues 216-230.

The terms “Fe fragment,” “Fe region,” or “Fc domain,” as used herein, are synonymous and refer to the portion of an immunoglobulin that is responsible for binding to antibody receptors on cells and the Clq component of complement (in the absence of any amino acid changes, relative to the naturally occurring sequence, to remove such binding activity). Fe stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, CH2, and CH3 domains. As used herein, the term also refers to a single chain consisting of CH3, CH2, and at least a portion of the hinge sufficient to form a disulfide-linked dimer with a second such chain. As used herein, the term Fc region further includes variants of naturally occurring hinge-CH2-CH3 sequences, wherein the variants are capable of forming dimers at least through dimerization of the CH3 domain and including such variants that have increased or decreased Fc receptor-binding or complement-binding activity while retaining at least sufficient binding to the neonatal Fc receptor (FcRn) to confer improved half-life to a fusion partner in vivo (relative to the fusion partner in the absence of the Fc region). The abbreviated term “Fc” may also be used herein to denote “Fe region” when referring to a fusion polypeptide by its general amino- to carboxyl-terminal structure (e.g., “DNase1-Li-Fc-L2-PON1”).

The term “dimer” or “dimeric protein,” as used herein, refers to a multimer of two (“first” and “second”) fusion polypeptides as disclosed herein linked together via dimerization of the Fc region.

Fusion polypeptides of the present disclosure may be referred to herein by formulae such as, for example, “DNase1-Li—Fe,” “DNase1L3-L1-Fc,” “DNase1-Li-Fc-L2-PONI,” or “DNaseiL3-Li-Fc-L2-PONI.” In each such case, unless the context clearly dictates otherwise, a term referring to a particular segment of a fusion polypeptide (e.g., “DNase1,” “DNaseiL3,” “Li” or “L2” (for first or second polypeptide linkers, respectively), “Fc” (for “Fe region”), etc.) is understood to have the meaning ascribed to such term herein and is inclusive of the various embodiments as described herein.

The term “disease or disorder characterized by NETosis,” as used herein, means a disease or disorder in which NETosis—the process wherein activated neutrophils extrude DNA bound with cytoplasmic and granule proteins, called neutrophil extracellular traps (NETs)-plays at least some part of the clinicopathology.

The term “effective amount,” in the context of treatment of a disease or disorder by administration of a soluble fusion polypeptide or dimeric protein to a subject as described herein, refers to an amount of such molecule that is sufficient to inhibit the occurrence or ameliorate one or more symptoms of the disease or disorder. For example, in the specific context of treatment of an inflammatory lung disease by administration of a fusion protein to a subject as described herein, the term “effective amount” refers to an amount of such molecule that is sufficient to modulate an inflammatory response in the subject so as to inhibit the occurrence or ameliorate one or more symptoms of the inflammatory lung disease. An effective amount of an agent is administered according to the methods of the present invention in an “effective regime.” The term “effective regime” refers to a combination of amount of the agent being administered and dosage frequency adequate to accomplish treatment or prevention of the disease.

The term “patient” or “subject,” in the context of treating a disease or disorder as described herein, includes mammals such as, for example, humans and other primates. The term also includes domesticated animals such as, e.g., cows, hogs, sheep, horses, dogs, and cats.

The term “combination therapy” refers to a therapeutic regimen that involves the provision of at least two distinct therapies to achieve an indicated therapeutic effect. For example, a combination therapy may involve the administration of two or more chemically distinct active ingredients, or agents, for example, a soluble DNase1 fusion polypeptide or dimeric protein according to the present invention and another agent such as, e.g., another anti-inflammatory or immunomodulatory agent. Alternatively, a combination therapy may involve the administration of a soluble DNase1 fusion polypeptide or dimeric protein according to the present invention, alone or in conjunction with another agent, as well as the delivery of another therapy (e.g., radiation therapy). The distinct therapies constituting a combination therapy may be delivered, e.g., as simultaneous, overlapping, or sequential dosing regimens. In the context of the administration of two or more chemically distinct agents, it is understood that the active ingredients may be administered as part of the same composition or as different compositions. When administered as separate compositions, the compositions comprising the different active ingredients may be administered at the same or different times, by the same or different routes, using the same or different dosing regimens, all as the particular context requires and as determined by the attending physician.

The term “targeted therapy,” in the context of treating cancer, refers to a type of treatment that uses a therapeutic agent to identify and attack a specific type of cancer cell, typically with less harm to normal cells. In some embodiments, a targeted therapy blocks the action of an enzyme or other molecule involved in the growth and spread of cancer cells. In other embodiments, a targeted therapy either helps the immune system to attack cancer cells or delivers a toxic substance directly to cancer cells. In certain variations, a targeted therapy uses a small molecule drug or a monoclonal antibody as a therapeutic agent.

The phrase “protect from aging,” as used herein, refers to inhibition or mitigation of any of broad aspects of aging, including, for example, age-related changes in systemic inflammation or disease risk, as indicated by accepted biomarkers. Protection from aging may also include treatment of an age-related disease where the disease is present in a subject, such as, for example, a chronic inflammatory, autoimmune, neurodegenerative, cardiovascular, or fibrotic disease.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wisconsin). Other methods for comparing amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least 80%, at least 90%, or at least 95% sequence identity relative to each other.

Percent sequence identity is determined by conventional methods. (See, e.g., Altschul et al., Bull. Math. Bio. 48:603, 1986, and Henikoff and Henikoff, Proc. Natl. Acad. Sci. USA 89:10915, 1992.) For example, two amino acid sequences can be aligned to optimize the alignment scores using a gap opening penalty of 10, a gap extension penalty of 1, and the “BLOSUM62” scoring matrix of Henikoff and Henikoff, supra. The percent identity is then calculated as: ([Total number of identical matches]/ [length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and a second amino acid sequence. The FASTA algorithm is described by Pearson and Lipman, Proc. Nat'l Acad. Sci. USA 85:2444, 1988, and by Pearson, Meth. Enzymol. 183:63, 1990. Briefly, FastA first characterizes sequence similarity by identifying regions shared by the query sequence (e.g., residues 21-280 of SEQ ID NO:4) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Illustrative parameters for FASTA analysis are: ktup=1, gap opening penalty=10, gap extension penalty=1, and substitution matrix=blosum62. These parameters can be introduced into a FASTA program by modifying the scoring matrix file (“SMATRIX”), as explained in Appendix 2 of Pearson, Meth. Enzymol. 183:63, 1990.

The term “corresponding to,” when applied to positions of amino acid residues in a reference sequence to describe positions within a subject sequence, means corresponding positions in the subject sequence when the reference and subject sequences are optimally aligned.

When such a value is expressed as “about” X or “approximately” X, the stated value of X will be understood to be accurate to ±10%.

Where aspects or embodiments of the present invention are described in terms of a Markush group or other grouping of alternatives, the present invention encompasses not only the entire group listed as a whole, but each member of the group individually and all possible subgroups of the main group. The present invention also envisages the explicit exclusion of one or more of any of the group members as embodiments.

### BRIEF DESCRIPTION OF THE DRAWINGS

FIG. 1 shows a Western blot of culture supernatants (serum free) from transiently transfected HEK293 cells expressing DNase1-Fc and DNase1-Fc-PON1 fusion proteins. Transfections and Western blot analysis were performed as described in Example 1, infra. From left to right: Lane 1—LICOR Chameleon™ molecular weight markers; Lane 2, Lane 3, Lane 4 - transfection supernatants from three different individual clones (#1, #2, #4) of hDNaseTM-(g4s)4-Fc-PON1-Q192K; Lane 5 -THER4-RNase (control); Lane 6 - LICOR Chameleon MW Markers; Lane 7 - hDNaseTM-(g4s)4-Fc #4; Lane 8 - hDNaseTM-(g4s)6-Fc #2; Lane 9 - THER4 (control); Lane 10 - mock transfection.

FIG. 2 shows reducing SDS-PAGE analysis of DNase1-Fc and DNase1-Fc-PON1 fusion proteins purified from CHO clone spent culture supernatants. Stable CHO cell generation, Protein A purification of fusion proteins, and SDS-PAGE were performed as described in Example 1, infra. From left to right: Lane 1 - THER4PON1-K (control); Lane2 - hDNaseTM-(g4s)4-Fc-PON1-K; Lane 3 - hDNaseTM-(g4s)4-Fc; Lane 4 - hDNaseTM-(g4s)6-Fc; Lane5 - LI-COR Chameleon® MW markers; Lane 6 - BioRad Precision Plus Kaleidoscope MW markers.

FIGS. 3A and 3B show raw data from a modified SYTOX™ Green fluorescence assay assessing nuclease activity of DNase1 fusion proteins at two different substrate concentrations, 220 μM or 66 μM salmon sperm DNA (see Example 2, infra).

FIGS. 4A and 4B show Michaelis-Menten plots of data from two different DNase activity experiments using a modified SYTOX™ Green fluorescence assay (see Example 2, infra).

FIGS. 5A and 5B show results from a SYTOX™ Green fluorescence assay comparing relative actin-induced inhibition of DNase activity of recombinant human DNase1 and a DNase1-Fc fusion protein (see Example 2, infra).

FIGS. 6A and 6B show Michaelis-Menten and Lineweaver-Burk plots, respectively, for a representative experiment measuring the arylesterase activity of a DNase1-Fc-PON1 fusion protein (see Example 3, infra).

FIG. 7 shows the results of a fluorometric assay measuring the organophosphatase activity of a DNase1-Fc-PON1 fusion protein and an organophosphatase positive control enzyme (see Example 3, infra).

FIGS. 8 and 9 show the results of a pharmacokinetic study of a DNase1-Fc fusion protein in BALB/c wild-type mice (see Example 7, infra). FIG. 8 shows the concentration (g/ml) of DNase1-Fc in plasma over time following IV injection, and FIG. 9 shows the nuclease activity of DNase1-Fc present in the mouse plasma (see id.)

FIGS. 10 and 11 show therapeutic treatment of a 4T1 breast tumor mouse model with DNase1-Fc fusion protein (see Example 8, infra). FIG. 10 shows the effect of DNase1-Fc on 4T1 tumor volume on day 18, and FIG. 11 shows survival data through day 20 (see id.).

### DESCRIPTION OF THE INVENTION

### I. Overview

The present invention provides compositions and methods relating to fusion polypeptides comprising a biologically active DNase linked to the N-terminus of an immunoglobulin Fc region. In some embodiments, the DNase is an enhanced DNase1, typically a hyperactive variant of wild-type human DNase1 (mature amino acid sequence shown in SEQ ID NO:30). Some preferred enhanced DNase1 polypeptides contain amino acid substitutions at two or all three of the positions corresponding to N74, G105, and A114 of human DNase1. Other preferred enhanced DNase1 polypeptides contain amino acid substitutions at one, two, or all three of the positions corresponding to D53, Y65, and E69 of human DNase1 and optionally contain amino acid substitutions at one or both of the positions corresponding to N74 and G105 of human DNase1. In other embodiments, the DNase is a truncated variant of human wild-type DNase1L3 in which the carboxyl-terminal nuclear localization signal (referred to herein as NLS2, corresponding to amino acid residues 291-305 of SEQ ID NO:34) is deleted. The DNase is linked to the N-terminus of the Fc region via a flexible polypeptide linker, which preferably contains at least 26 amino acid residues. In some aspects, the fusion polypeptide is a bispecific construct further comprising a polypeptide segment located carboxyl-terminal to the Fc region (also referred to herein as a “third polypeptide segment”). An exemplary third polypeptide segment is a biologically active paraoxonase such as, e.g., a human paraoxonase 1 (PON1) or functional variant thereof.

The fusion molecules of the present invention are useful, for example, for treating diseases or disorders characterized by NETosis, the process wherein activated neutrophils extrude DNA bound with cytoplasmic and granule proteins, called neutrophil extracellular traps (NETs). NETs, which cause tissue damage by themselves or by increasing the pro-inflammatory response (see Mutua and Gershwin, Clin. Rev. Allergy Immunol., 2020), are implicated in a variety of serious diseases and conditions. For example, NETs can play a role in enhancement of inflammation seen in autoimmune disease, including, e.g., systemic lupus erythematosus (SLE) (e.g., SLE with lupus nephritis), rheumatoid arthritis, and psoriasis, and are also associated with autoinflammatory diseases such as, for example, inflammatory bowel disease (IBD) (see id.). NETs also play a role in sepsis, metabolic diseases (e.g., type 2 diabetes and obesity), neurological diseases (e.g., Alzheimer's disease, multiple sclerosis, meningitis, cerebral malaria, and ischemic stroke), infectious disease, cardiovascular disease and thrombosis, tumor progression and metastasis, liver disease (e.g., alcohol-associated liver disease (ALD), portal hypertension, nonalcoholic fatty liver disease (NAFLD) such as nonalcoholic steatohepatitis (NASH), and complications following liver transplantation), and various inflammatory lung diseases, including, e.g., cystic fibrosis, asthma, chronic obstructive pulmonary disease (COPD), and COVID-19-associated acute respiratory distress syndrome (ARDS) (see, e.g., id.; Brinkmann, J. Innate Immun. 10:422-431, 2018; Liu et al., Chinese Med. Journal 130:730-736, 2017; Twaddell et al., CHEST 156:774-782, 2019; Uddin et al., Front. Immunol. 10:47, 2019; Middleton et al., Blood 136:1169, 2020; Zuo et al., JCI Insight 5:e138999, 2020; Barnes et al., J. Exp. Med. 217:e20200652, 2020; Hilscher and Shah, Semin. Liver Dis. 40:171-179, 2020). The use of DNase for the treatment of diseases or disorders characterized by NETosis is further supported, for example, by the use of recombinant wild-type DNase (Pulmozyme®) for treatment of cystic fibrosis, a current clinical trial of Pulmozyme in COVID-19 patients (NCT04359654), and several animal model studies, including, e.g., models of acute asthma, silica-induced lung inflammation, acute lung injury, cancer, colitis, ischemia reperfusion, sepsis, and thrombosis (see, e.g., da Cunha et al., Exp. Lung Res. 42:66, 2016; Benmerzoug et al., Nat. Comm. 9:5226, 2018; Caudrillier et al., J. Clin. Invest. 122:2661, 2012; Cools-Lartigue et al., J. Clin. Invest. 123:3446, 2013; Park et al., Sci. Translational Med. 8:361ra138, 2016; Trejo-Becirril et al., Integrative Cancer Therapies 15:NP35-NP43, 2016; Thalin et al., PLoS ONE 13:e0191231, 2018; Babicova et al., Folia Biolica (Praha) 64:10, 2018; Li et al., J. Crohns Colitis 2020, 14:240-253, 2020; Albadawi et al., J. Vasc. Surg. 64:484, 2016; Peer et al., Am. J. Nephrol. 43:195, 2016; Mai et al., Shock 44:166, 2015; Brill et al., J. Thrombosis Haemostasis 10:136, 2012; Manda-Handzlik and Demkow, Cells 8:1477, 2019; Delgado-Rizo et al., Front. Immunol. 8:81, 2017). The molecules of the present disclosure, which provide stable, active DNase-Fc fusion proteins, including DNase fusions with increased DNA binding and/or elimination of actin inhibition, have improved therapeutic potential in these and other diseases relative to wild-type DNase1.

DNase fusion molecules as described herein are also useful in treatment of antiphospholipid syndrome (APLS). Antiphospholipid syndrome is an autoimmune disease characterized by antibodies to phospholipids that cause a hypercoagulable state. Thrombosis occurs in both arteries and veins and causes pregnancy complications including miscarriage and stillbirths. Antiphospholipid syndrome can be primary (occurring in the absence of any other related disease) or secondary (occurring with another autoimmune disease such as, e.g., SLE). In rare cases, APLS leads to rapid organ failure due to generalized thrombosis; this is termed catastrophic antiphospholipid syndrome (CAPS or Asherson syndrome) and is associated with a high risk of death. Patients with acute respiratory distress associated with COVID-19 have antiphospholipid antibodies that promote thrombosis in vitro and in mouse thrombosis models (Zuo et al., JCI Insight 5:e138999, 2020; Zuo et al., Sci. Trans. Med. 12:eabd3876, 2020). The antiphospholipid antibodies stimulate neutrophil NET formation, providing a scaffold for thrombus formation. Digestion of NETs by DNase fusions as described herein can prevent thrombus formation and can help dissolve existing clots to restore blood flow. DNase fusion molecules of the present invention may also be used together with thrombolytics including, e.g., tPA, urokinase, and streptokinase.

Bispecific DNase-Fc fusions further comprising a biologically active paraoxonase provide additional therapeutic benefit for the treatment of diseases amenable to DNase-mediated therapy, including, for example, through its antioxidant, anti-inflammatory, atheroprotective, and/or neuroprotective properties. A bispecific DNase-Fc fusion molecule comprising a paraoxonase as described herein may be used, e.g., for treatment of an autoimmune disease or an inflammatory disease. For example, studies support use of a paraoxonase for treatment of autoimmune disease such as systemic lupus erythematosus (SLE). The autoantibody titer in many patients with systemic lupus erythematosus (SLE) is correlated with loss of activity of PON1 (see Batukla et al., Ann. NYAcad. Sci. 1108:137-146, 2007), and SLE-disease activity assessed by SLEDAI and SLE disease related organ damage assessed by SLICC/ACR damage index are negatively correlated with PON1 activity (see Ahmed et al., EXCLI Journal 12:719-732, 2013). Other studies support use of a paraoxonase for treatment of inflammatory disease such as inflammatory lung diseases. Studies strongly suggest, for example, that PON1 is important in protection from lung inflammation, and further show that low PON1 activity is associated with lung diseases such as, e.g., asthma, chronic obstructive pulmonary disease (COPD), and interstitial lung disease (e.g., idiopathic pulmonary fibrosis (IPF) or sarcoidosis) (see, e.g., Sahiner et al., WAO Journal 4:151-158, 2011; Emin et al., Allergol. Immunopathol. (Madr) 43:346-352, 2014; Sarioglu et al., Iran J. Asthma Immunol. 14:60-66, 2015; Tolgyesi et al., Internat. Immunol. 21:967-975, 2009; Chen et al., J. Cell Biochem. 119:793-805, 2018; Rumora et al., J. COPD 11:539-545, 2014; Rajkovic et al., J. Clin. Path. 71:963-970, 2018; Ivanisevic et al., Eur. J. Clin. Invest. 46:418-424, 2016; Uzun et al., Curr. Med. Res. Opin. 24:1651-1657, 2008; Okur et al., Sleep Breath. 17:365-371, 2013; Golmanesh et al., Immunopharmacol. and Immunotoxicol. 35:419-425, 2013). Another study showed that low PON1 activity is correlated with the severity of Crohn's disease (Sczceklik et al., Molecules 23:2603, 2018). In addition, recombinant PON1 therapy has shown efficacy in animal models of organophosphate poisoning and colitis (see, e.g., Valiyaveettil et al., Biochem. Pharmacol. 81:800-809, 2011; Valiyaveettil et al., Toxicol. Letters 202:203-208, 2011; Bajaj et al., Appl. Biochem. Biotechnol. 180:165, 2016; Stevens et al., Proc. Natl. Acad. Sci. USA 105:12780-12784, 2008; Yamashita et al., J. Immunol. 191:949-960, 2013). Bispecific DNase-Fc-PON1 molecules of the present disclosure provide a means to retarget PON1 from HDL to DNA and the sites of inflammatory NETs and provide additional therapeutic benefit in these and other diseases.

Bispecific DNase fusion molecules comprising a paraoxonase as described herein may also be used, e.g., for treatment of a neurological disease. Such bispecific molecules are transported to the brain where they deliver a protective paraoxonase enzyme. For example, PON1 is protective in the brain because of its anti-oxidant properties. Exemplary neurological diseases amenable to treatment using an DNase-Fc-PON bispecific molecule of the present invention include multiple sclerosis and Alzheimer's disease.

DNase fusion molecules as described herein, including embodiments further comprising a paraoxonase, may also be used for treatment of an inflammatory skin disease. Many studies have reported the involvement of reactive oxygen species and lipid peroxidation in the pathogenesis of inflammatory skin diseases (see, e.g., Simonetti et al., Antioxidants 10:697, 2021). For example, significantly lower levels of paraoxonase 1 (PON1) and a significantly higher levels of myeloperoxidase (MPO) and lipid peroxides have been found in atopic dermatitis (see id.), and increased MPO and decreased PON1 activity have also been described in psoriatic children (see Bacchetti et al., Archives of Dermatological Research 312:33-39). In psoriasis and atopic dermatitis, neutrophil activation during inflammation leads to extruded NETs with attached MPO, causing the oxidation of lipids and damaging PON1 enzyme, which normally protects lipids from oxidation by digesting lipid peroxides. The digestion of NETs with DNase1 releases the attached MPO, allowing its clearance and removing a primary driver of oxidation, and therapy with PON1 restores protection from oxidative damage and rebalances the redox potential in patients. Thus, PON1 and DNase therapies are particularly effective as a combination.

DNase fusion molcecules as described herein, including embodiments further comprising a paraoxonase, may also be used to protect from aging. DNase-Fc is useful for slowing aging because of its ability to digest NETs. NETs are a primary driver of inflammation and oxidative stress because they provide the scaffold for myeloperoxidase (MPO) and elastase produced by neutrophils and other cells. Digestion of the NETs by DNase-Fc allows the release and clearance of the inflammatory enzymes. DNase-Fc also eliminates inflammatory activation through other DNA sensing pathways (e.g., STING, TLR9). In addition, embodiments further comprising a paraoxonase are particularly useful because oxidative stress is known to be a primary cause of aging. In particular, oxidized lipids are a key target for aging because of their promotion of inflammation through uptake by macrophage scavenger receptors and their damage to vascular endothelial cells. See, e.g., Moldogazieva et al. (Oxidative Medicine and Cellular Longevity, Volume 2019, Article ID 3085756) and Barrera et al. (Antioxidants 7:102, 2018) for discussions on the link between lipid oxidation and aging. See also Hajri (Frontiers In Bioscience, Landmark 23:1822-1847, 2018), which describes the toxic effects of oxidized lipids on inflammation and cardiac function. PON1 is known to protect lipids from oxidation by digestion of lipid peroxides. Thus, there is a direct link between increasing PON1 enzyme levels and slowing of aging. In addition, PON1 enzyme activity is known to be deficient in multiple age-related diseases including cardiovascular disease, autoimmune disease, and pulmonary disease, showing that there is a clinical need for increased PON1.

In some aspects, the present invention provides compositions and methods for producing fusion polypeptides and dimeric proteins as described herein, including such compositions and methods for the constitutive expression of DNase-Fc fusions in stable cell lines such as CHO. Previous efforts to obtain stable CHO cells that constitutively express an actin/salt-resistant and hyperactive DNase1 were unsuccessful despite screening thousands of clones (see Lam et al., Biotech. Progress 32: 523-533, 2017). In certain embodiments, DNase-Fc fusions comprising an enhanced DNase1 as described herein are readily expressed in stable lines with minimal screening, thereby overcoming this problem in the art. The ability to readily generate stable cell lines expressing certain enhanced DNase1-Fc molecules of the present disclosure indicate that these molecules have advantages in feasibility and cost of industrial scale biologic drug manufacturing.

### II. Fusion Polypeptides and Dimeric Proteins

In one aspect, the present invention provides a fusion polypeptide comprising, from an amino-terminal position to a carboxyl-terminal position, D-L1-X, wherein D is a biologically active DNase, L1 is a first polypeptide linker, and X is an immunoglobulin Fc region. In some embodiments, the biologically active DNase is a naturally occurring DNase1 polypeptide or a functional variant or fragment thereof, more typically an enhanced DNase1 as described further herein. In certain alternative variations, the DNase is a truncated form of a naturally occurring DNase1L3 in which the carboxyl terminal nuclear location signal is deleted, or a functional variant or fragment thereof. In some embodiments, the fusion polypeptide further includes a polypeptide segment located carboxyl-terminal to the Fc region (also referred to herein as a “third polypeptide segment”). Typically, the third polypeptide segment is a biologically active polypeptide such as, for example, a biologically active paraoxonase (e.g., a naturally occurring paraoxonase 1 (PON1) polypeptide or a functional variant or fragment thereof). Such a fusion polypeptide comprising a third polypeptide segment may be represented by the formula D-L1-X-L2-P (from an amino-terminal position to a carboxyl-terminal position), wherein D, L1, and X are each as previously defined, wherein L2 is a second polypeptide linker and is optionally present, and wherein P is the third polypeptide segment.

Functional variants of a naturally occurring DNase or paraoxonase can be readily identified using routine assays for assessing the variant for enzyme activity. For example, DNase1 and DNase1L3 variants, including hyperactive DNase1 variants, may be assayed for nuclease activity using (i) a DNA-methyl green assay, which measures the decrease of A26o as the methyl green dye is released from hydrolyzed DNA (see, e.g., Sinicropi et al., Anal. Biochem. 222:351-358; Pan and Lazarus, J. Biol. Chem. 273:11701-11708, 1998), (ii) a Kunitz hyperchromicity assay, in which the A260, due to the DNA absorption, increases as a function of degradation (see, e.g., Kunitz, J. Gen. Physiol. 33:349-362, 1950; Pan and Lazarus, supra); (iii) a plasmid digestion assay, which uses either supercoiled or linear plasmid DNA as a substrate and measures the disappearance of substrate and/or appearance of digestion products (e.g., appearance of linear or relaxed products from supercoiled DNA) (see, e.g., Pan and Lazarus, supra), or (iv) a SYTOX™ Green fluorescence assay, which measures the decrease in fluorescence as DNA labeled with SYTOX Green dye is hydrolyzed (see Example 2, infra). DNase1 variants may also be assayed for G-actin-induced inhibition of nuclease activity using DNase preincubated with G-actin prior to addition of substrate DNA (see, e.g., Pan et al., J. Biol. Chem. 273:18374-18381, 1998). In addition, paraoxonase 1 (PON1) variants may be assayed for phosphotriesterase activity using diethyl p-nitrophenol phosphate (paraoxon) as a substrate, or for arylesterase activity using phenyl acetate as a substrate (see, e.g., Graves and Scott, Curr Chem Genomics 2:51-61, 2008).

Naturally occurring polypeptide segments for use in accordance with the present disclosure (e.g., a naturally occurring DNase or paraoxonase) include naturally occurring variants such as, for example, allelic variants and interspecies homologs consistent with the disclosure.

Functional variants of a particular reference polypeptide (e.g., a wild-type human DNase1 as shown in residues 23-282 of SEQ ID NO:2 or an enhanced DNase1 as shown in residues 21-280 of SEQ ID NO:4, residues 21-280 of SEQ ID NO:74, residue 21-280 of SEQ ID NO:76, or residues 21-280 of SEQ ID NO:78) are generally characterized as having one or more amino acid substitutions, deletions, or additions relative to the reference polypeptide. These changes are preferably of a minor nature, that is conservative amino acid substitutions (see, e.g., Table 1, infra, which lists some exemplary conservative amino acid substitutions) and other substitutions that do not significantly affect the folding or activity of the protein or polypeptide; small deletions, typically of one to about 30 amino acids; and small amino- or carboxyl-terminal extensions. Conservative substitutions may also be selected from the following: 1) Alanine, Glycine; 2) Aspartate, Glutamate; 3) Asparagine, Glutamine; 4) Arginine, Lysine; 5) Isoleucine, Leucine, Methionine, Valine; 6) Phenylalanine, Tyrosine, Tryptophan; 7) Serine, Threonine; and 8) Cysteine, Methionine (see, e.g., Creighton, Proteins (1984)).

TABLE 1
Conservative amino acid substitutions

| Basic | Acidic | Polar | Hydrophobic | Aromatic | Small |
|---|---|---|---|---|---|
| Arginine | Glutamate | Glutamine | Leucine | Phenylalanine | Glycine |
| Lysine | Aspartate | Asparagine | Isoleucine | Tryptophan | Alanine |
| Histidine | | | Valine | Tyrosine | Serine |
| | | | Methionine | | Threonine |
| | | | | | Methionine |

Essential amino acids in a naturally occurring polypeptide can be identified according to procedures known in the art, such as site-directed mutagenesis or alanine-scanning mutagenesis (Cunningham and Wells, Science 244:1081-1085, 1989; Bass et al., Proc. Natl. Acad. Sci. USA 88:4498-4502, 1991). In the latter technique, single alanine mutations are introduced at every residue in the molecule, and the resultant mutant molecules are tested for biological activity (e.g., nuclease activity for DNase1 variants) to identify amino acid residues that are critical to the activity of the molecule. In addition, sites of relevant protein interactions can be determined by analysis of crystal structure as determined by such techniques as nuclear magnetic resonance, crystallography or photoaffinity labeling. The identities of essential amino acids can also be inferred from analysis of homologies with related proteins (e.g., species orthologs retaining the same protein function).

Multiple amino acid substitutions can be made and tested using known methods of mutagenesis and screening, such as those disclosed by Reidhaar-Olson and Sauer Science 241:53-57, 1988 or Bowie and Sauer Proc. Natl. Acad. Sci. USA 86:2152-2156, 1989. Briefly, these authors disclose methods for simultaneously randomizing two or more positions in a polypeptide, selecting for functional polypeptide, and then sequencing the mutagenized polypeptides to determine the spectrum of allowable substitutions at each position. Another method that can be used is region-directed mutagenesis (Derbyshire et al., Gene 46:145, 1986; Ner et al., DNA 7:127, 1988).

Variant nucleotide and polypeptide sequences can also be generated through DNA shuffling. (See, e.g., Stemmer, Nature 370:389, 1994; Stemmer, Proc. Nat'l Acad. Sci. USA 91:10747, 1994; International PCT Publication No. WO 97/20078.) Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

As previously discussed, a polypeptide fusion in accordance with the present invention can include a polypeptide segment corresponding to a “functional fragment” of a particular polypeptide. Routine deletion analyses of nucleic acid molecules can be performed to obtain functional fragments of a nucleic acid molecule encoding a given polypeptide. As an illustration, DNase1-encoding DNA molecules having the nucleotide sequence of residues 67-846 of SEQ ID NO:1 or residues 61-840 of SEQ ID NO:3 can be digested with Bal31 nuclease to obtain a series of nested deletions. The fragments are then inserted into expression vectors in proper reading frame, and the expressed polypeptides are isolated and tested for nuclease activity. One alternative to exonuclease digestion is to use oligonucleotide-directed mutagenesis to introduce deletions or stop codons to specify production of a desired fragment. Alternatively, particular fragments of a gene encoding a polypeptide can be synthesized using the polymerase chain reaction.

Accordingly, using methods such as discussed above, one of ordinary skill in the art can prepare a variety of polypeptides that (i) are substantially identical to a reference polypeptide (e.g., a human wild-type DNase1 as shown in residues 23-282 of SEQ ID NO:2 or an enhanced DNase1 as shown in residues 21-280 of SEQ ID NO:4, residues 21-280 of SEQ ID NO:74, residues 21-280 of SEQ ID NO:76, or residues 21-280 of SEQ ID NO:78) and (ii) retains the desired functional properties of the reference polypeptide.

Polypeptide segments used within the present invention (e.g., polypeptide segments corresponding to a DNase, paraoxonase, or Fc fragment) may be obtained from a variety of species. If the protein is to be used therapeutically in humans, it is preferred that human polypeptide sequences be employed. However, non-human sequences can be used, as can variant sequences. For other uses, including in vitro diagnostic uses and veterinary uses, polypeptide sequences from humans or non-human animals can be employed, although sequences from the same species as the patient may be preferred for in vivo veterinary use or for in vitro uses where species specificity of intermolecular reactions is present. Thus, polypeptide segments for use within the present invention can be, without limitation, human, non-human primate, rodent, canine, feline, equine, bovine, ovine, porcine, lagomorph, and avian polypeptides, as well as variants thereof.

In some embodiments, the DNase segment is a human wild-type DNase1 or a functional variant or fragment thereof. For example, in some embodiments, the DNase comprises an amino acid sequence having at least 80% identity with (i) amino acid residues 23-282 of SEQ ID NO:2, (ii) amino acid residues 21-280 of SEQ ID NO:4, (iii) amino acid residues 21-280 of SEQ ID NO:6, (iv) amino acid residues 21-280 of SEQ ID NO:52, (v) residues 21-280 of SEQ ID NO:74, (vi) residues 21-280 of SEQ ID NO:76, (vii) residues 21-280 of SEQ ID NO:78, (viii) residues 21-280 of SEQ ID NO:80, (ix) residues 21-280 of SEQ ID NO:82, or (x) residues 21-280 of SEQ ID NO:84. In more particular embodiments, the DNase comprises an amino acid sequence having at least 85%, at least 90%, or at least 95% identity with (i) amino acid residues 23-282 of SEQ ID NO:2, (ii) amino acid residues 21-280 of SEQ ID NO:4, (iii) amino acid residues 21-280 of SEQ ID NO:6, (iv) amino acid residues 21-280 of SEQ ID NO:52, (v) residues 21-280 of SEQ ID NO:74, (vi) residues 21-280 of SEQ ID NO:76, (vii) residues 21-280 of SEQ ID NO:78, (viii) residues 21-280 of SEQ ID NO:80, (ix) residues 21-280 of SEQ ID NO:82, or (x) residues 21-280 of SEQ ID NO:84. In yet other embodiments, the DNase comprises an amino acid sequence having at least 96%, at least 97%, at least 98%, at least 99%, or 100% sequence identity with (i) amino acid residues 23-282 of SEQ ID NO:2, (ii) amino acid residues 21-280 of SEQ ID NO:4, (iii) amino acid residues 21-280 of SEQ ID NO:6, (iv) amino acid residues 21-280 of SEQ ID NO:52, (v) residues 21-280 of SEQ ID NO:74, (vi) residues 21-280 of SEQ ID NO:76, (vii) residues 21-280 of SEQ ID NO:78, (viii) residues 21-280 of SEQ ID NO:80, (ix) residues 21-280 of SEQ ID NO:82, or (x) residues 21-280 of SEQ ID NO:84.

In certain preferred embodiments, the DNase segment is a hyperactive and/or actin-resistant variant of a naturally occurring DNase1 (an “enhanced DNase1”) such as, e.g., an enhanced human DNase1 variant. For example, in some variations, the DNase contains at least one amino acid substitution at a position corresponding to an amino acid of mature wild-type human DNase1 (SEQ ID NO:30) selected from N74, G105, and A114, wherein (1) the amino acid substitution at the position corresponding to N74 of human DNase1 (N74 substitution), if present, increases DNA binding of the DNase relative to human DNase1, (2) the amino acid substitution at the position corresponding to G105 of human DNase1 (G105 substitution), if present, increases DNA binding of the DNase relative to human DNase1, and (3) the amino acid substitution at the position corresponding to A114 of human DNase1 (A114 substitution), if present, decreases G-actin-induced inhibition of endonuclease activity of the DNase relative to human DNase1. Suitable amino acids for substitution at these positions include lysine, arginine, or histidine at the position corresponding to N74 of human DNase1, lysine, arginine, or histidine at the position corresponding to G105 of human DNase1, and/or phenylalanine, glutamate, methionine, arginine, or tyrosine at the position corresponding to A114 of human DNase1. In some variations, an enhanced human DNase1 variant as above contains at least two of the N74, G105, and A114 substitutions (e.g., substitutions at at least the positions corresponding to both N74 and G105 or both G105 and A114 of human DNase1). In some embodiments, the enhanced human DNase1 contains each of the N74, G105, and A114 substitutions. In certain embodiments, the enhanced DNase1 comprises an amino acid sequence having at least 80%, at least 85%, at least 90%, at least 95%, at least 96%, at least 97%, at least 98%, at least 99%, or 100% identity with (i) amino acid residues 21-280 of SEQ ID NO:4 or (ii) amino acid residues 21-280 of SEQ ID NO:52. In some variations, the enhanced DNase1 comprises an amino acid sequence having at least 90%, at least 95%, at least 96%, at least 97%, at least 98%, or at least 99% identity with amino acid residues 21-280 of SEQ ID NO:4, wherein the enhanced DNase1 contains each of the N74, G105, and A114 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In other variations, the enhanced DNase1 comprises an amino acid sequence having at least 90%, at least 95%, at least 96%, at least 97%, at least 98%, or at least 99% identity with amino acid residues 21-280 of SEQ ID NO:52, wherein the enhanced DNase1 contains each of the N74 and G105 substitutions but does not contain any other amino acid substitution that increases DNA binding of the DNase relative to human DNase1, and/or does not contain the A114 substitution or any other amino acid substitution that decreases G-actin-induced inhibition of endonuclease activity of the DNase relative to human DNase1. Enhanced DNase1-Fc fusions that lack actin resistance are particularly beneficial for decreasing toxicity to host cells (e.g., CHO) to enable higher expression of the fusion molecule.

In other preferred embodiments, the DNase segment is an enhanced human DNase1 variant containing at least one amino acid substitution at a position corresponding to an amino acid of mature wild-type human DNase1 (SEQ ID NO:30) selected from D53, Y65, and E69, and optionally containing at least one amino acid substitution corresponding to an amino acid of mature wild-type human DNase 1 selected from N74 and G105, wherein (1) the amino acid substitution at the position corresponding to D53 of human DNase1 (D53 substitution), if present, decreases G-actin-induced inhibition of endonuclease activity of the DNase relative to human DNase1, (2) the amino acid substitution at the position corresponding to Y65 of human DNase1 (Y65 substitution), if present, decreases G-actin-induced inhibition of endonuclease activity of the DNase relative to human DNase1, (3) the amino acid substitution at the position corresponding to E69 of human DNase1 (E69 substitution), if present, decreases G-actin-induced inhibition of endonuclease activity of the DNase relative to human DNase1, (4) the amino acid substitution at the position corresponding to N74 of human DNase1 (N74 substitution), if present, increases DNA binding of the DNase relative to human DNase1, and (5) the amino acid substitution at the position corresponding to G105 of human DNase1 (G105 substitution), if present, increases DNA binding of the DNase relative to human DNase1. Suitable amino acids for substitution at these positions include alanine, methionine, arginine, or tyrosine at the position corresponding to D53 of human DNase1, alanine or arginine at the position corresponding to Y65 of human DNase1, lysine or arginine at the position corresponding to E69 of human DNase1, lysine, arginine, or histidine at the position corresponding to N74 of human DNase1, and/or lysine, arginine, or histidine at the position corresponding to G105 of human DNase1. In some variations, an enhanced human DNase1 variant as above contains only one of the D53, Y65, and E69 substitutions; in other variations, an enhanced DNase1 variant as above contains at least two of the D53, Y65, and E69 substitutions (e.g., both the D53 and Y65 substitutions, or each of the D53, Y65, and E69 substitutions). In other, non-mutually exclusive variations, an enhanced DNase1 variant as above contains both the N74 and G105 substitutions. For example, an enhanced DNase1 variant as above may contain (i) each of the D53, N74, and G105 substitutions, (ii) each of the Y65, N74, and G105 substitutions, (iii) each of the E69, N74, and G105 substitutions, (iv) each of the D53, Y65, N74, and G105 substitutions, or (v) each of the D53, Y65, E69, N74, and G105 substitutions. In certain embodiments, the enhanced DNase1 comprises an amino acid sequence having at least 80%, at least 85%, at least 90%, at least 95%, at least 96%, at least 97%, at least 98%, at least 99%, or 100% identity with (i) residues 21-280 of SEQ ID NO:74, (ii) residues 21-280 of SEQ ID NO:76, (iii) residues 21-280 of SEQ ID NO:78, (iv) residues 21-280 of SEQ ID NO:80, (v) residues 21-280 of SEQ ID NO:82, or (vi) residues 21-280 of SEQ ID NO:84.

For example, in some variations, an enhanced DNase1 variant as above comprises an amino acid sequence having at least 90%, at least 95%, at least 96%, at least 97%, at least 98%, or at least 99% identity with amino acid residues 21-280 of SEQ ID NO:74, wherein the enhanced DNase1 contains each of the D53, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In other variations, the enhanced DNase1 comprises an amino acid sequence having at least 90%, at least 95%, at least 96%, at least 97%, at least 98%, or at least 99% identity with amino acid residues 21-280 of SEQ ID NO:76, wherein the enhanced DNase1 contains each of the Y65, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In other variations, the enhanced DNase1 comprises an amino acid sequence having at least 90%, at least 95%, at least 96%, at least 97%, at least 98%, or at least 99% identity with amino acid residues 21-280 of SEQ ID NO:78, wherein the enhanced DNase1 contains each of the E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In yet other variations, the enhanced DNase1 comprises an amino acid sequence having at least 90%, at least 95%, at least 96%, at least 97%, at least 98%, or at least 99% identity with amino acid residues 21-280 of SEQ ID NO:74 or amino acid residues 21-280 of SEQ ID NO:76, wherein the enhanced DNase1 contains each of the D53, Y65, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In yet other variations, the enhanced DNase1 comprises an amino acid sequence having at least 90%, at least 95%, at least 96%, at least 97%, at least 98%, or at least 99% identity with amino acid residues 21-280 of SEQ ID NO:74 or amino acid residues 21-280 of SEQ ID NO:78, wherein the enhanced DNase1 contains each of the D53, E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In yet other variations, the enhanced DNase1 comprises an amino acid sequence having at least 90%, at least 95%, at least 96%, at least 97%, at least 98%, or at least 99% identity with amino acid residues 21-280 of SEQ ID NO:76 or amino acid residues 21-280 of SEQ ID NO:78, wherein the enhanced DNase1 contains each of the Y65, E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In still other variations, the enhanced DNase1 comprises an amino acid sequence having at least 90%, at least 95%, at least 96%, at least 97%, at least 98%, or at least 99% identity with amino acid residues 21-280 of SEQ ID NO:74, amino acid residues 21-280 of SEQ ID NO:76, or amino acid residues 21-280 of SEQ ID NO:78, wherein the enhanced DNase1 contains each of the D53, Y65, E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

In some embodiments, the DNase segment is an enhanced human DNase1 variant with intermediate sensitivity to inhibition by actin. For example, in some variations, the enhanced human DNase1 contains amino acid substitutions, relative to wild-type human DNase1, that cause about 50% to about 90% or about 70% to about 90% reduction in actin inhibition (i.e., the enhanced DNase1 retains about 10% to about 50% or about 10% to about 30% of the actin inhibition of wild-type human DNase1). An exemplary nuclease assay for measuring actin inhibition is described in Example 11, infra. To analyze actin inhibition using such an assay, RFU (Relative Fluorescence Units) measured in the presence of actin is divided by the RFU measured in the absence of actin to obtain a fraction that is plotted as a function of actin (inhibitor) concentration, and nonlinear regression analysis of the curves is performed to measure the Ki (defined as the concentration of inhibitor required to reach 50% inhibition of the enzyme activity) as described in more detail in Example 11 and in Ulmer et al. (Proc. Natd. Acad. Sci. USA 93:8225-8229, 1996). For wild-type DNase1, the Ki is expected to be lower than the Ki for a DNase1 variant with intermediate sensitivity to actin inhibition. For example, a variant with 90% reduction in inhibition requires 10 times (lOX) more actin to inhibit the enzyme activity by 50%, so the Ki for this variant would be 1OX greater than that observed for the wild-type DNase1; a variant with 50% reduction in inhibition requires 2X more actin to reach 50% inhibition, so the Ki for this variant would be 2X greater than that observed for wild-type. Enhanced DNase1-Fc fusion molecules with intermediate sensitivity to inhibition by actin are particularly advantageous because (a) relative to enhanced DNase1-Fc fusion molecules with complete or near complete resistance to inhibition by actin, these molecules can be expressed at higher levels due to reduced toxicity to host cells such as, e.g., CHO, and (b) these molecules are still able to overcome actin inhibition in vivo, thereby providing improved therapeutic efficacy relative to non-actin-resistant DNase1-Fc fusions.

Exemplary DNase1 variants with intermediate sensitivity to inhibition by actin include enhanced DNase1 variants containing one or both of the D53 and Y65 substitutions as described above (e.g., an enhanced DNase1 variant wherein (i) the amino acid at the position corresponding to D53 of human DNase1 is alanine, methionine, arginine, or tyrosine, and/or (ii) the amino acid at the position corresponding to Y65 of human DNase1 is alanine). In some embodiments, an enhanced DNase1 with intermediate sensitivity to inhibition by actin comprises an amino acid sequence having at least 90%, at least 95%, at least 96%, at least 97%, at least 98%, at least 99%, or 100% identity with (i) amino acid residues 21-280 of SEQ ID NO:74, (ii) amino acid residues 21-280 of SEQ ID NO:76, (iii) amino acid residues 21-280 of SEQ ID NO:78, (iv) amino acid residues 21-280 of SEQ ID NO:80, (v) amino acid residues 21-280 of SEQ ID NO:82, or (vi) amino acid residues 21-280 of SEQ ID NO:84. In certain variations, an enhanced DNase1 having intermediate sensitivity to inhibition by actin is an enhanced DNase1 variant containing both of the D53 and Y65 substitutions as described above; for example, in some embodiments, an enhanced DNase1 with intermediate sensitivity to inhibition by actin comprises an amino acid sequence having at least 90%, at least 95%, at least 96%, at least 97%, at least 98%, at least 99%, or 100% identity with SEQ ID NO:101, wherein (i) the amino acid at the position corresponding to D53 of human DNase1 is alanine, methionine, arginine, or tyrosine, and (ii) the amino acid at the position corresponding to Y65 of human DNase1 is alanine.

In other embodiments, the DNase is a truncated form of human wild-type DNase1L3 in which the carboxyl-terminal nuclear localization signal (referred to herein as NLS2, corresponding to amino acid residues 291-305 of SEQ ID NO:34) is deleted, or a functional variant or fragment of such truncated form of human DNase1L3. For example, in some embodiments, the DNase comprises an amino acid sequence having at least 80%, at least 85%, at least 90%, or at least 95% identity with (i) amino acid residues 21-290 of SEQ ID NO:34, (ii) amino acid residues 21-290 of SEQ ID NO:36, or (iii) amino acid residues 21-290 of SEQ ID NO:38. In more particular embodiments, the DNase comprises an amino acid sequence having at least 96%, at least 97%, at least 98%, at least 99%, or 100% sequence identity with (i) amino acid residues 21-290 of SEQ ID NO:34, (ii) amino acid residues 21-290 of SEQ ID NO:36, or (iii) amino acid residues 21-290 of SEQ ID NO:38. In certain preferred embodiments comprising a truncated form of human DNase1L3 or functional variant thereof, the DNase is a variant DNase1L3 containing one or more amino acid substitutions relative to wild-type human DNase1L3 (SEQ ID NO:34) that inactivate the nuclear localization signal in the N-terminal half of the molecule (referred to herein as NLS1, corresponding to amino acid residues 80-96 of SEQ ID NO:34); in some such embodiments, each of the amino acids at positions corresponding to R80, R95, and N96 of SEQ ID NO:34 is alanine or serine (e.g., each of R80, R95, and N96 is alanine, or each of R80, R95, and N96 is serine). In more specific embodiments of a variant, truncated form of DNase1L3 as above in which the wild-type NLS1 consensus sequence is removed, the DNase has the amino acid sequence shown in (i) amino acid residues 21-290 of SEQ ID NO:36 or (ii) amino acid residues 21-290 of SEQ ID NO:38.

In certain embodiments comprising a third polypeptide segment carboxyl-terminal to the Fc region, the third polypeptide segment is a biologically active polypeptide. In some preferred embodiments, the biologically active polypeptide carboxyl-terminal to the Fc region is a biologically active paraoxonase such as, e.g., a human paraoxonase 1 (PON1) or a functional variant or fragment thereof. For example, in some embodiments, the paraoxonase (a) has at least 80%, at least 85%, at least 90%, or at least 95% identity with amino acid residues 16-355 or 26-355 of SEQ ID NO:24 or SEQ ID NO:22 and (b) does not contain an amino-terminal leader sequence corresponding to residues 1-15 of SEQ ID NO:24 (also shown as residues 1-15 of SEQ ID NO:22); in some such embodiments, the biologically active paraoxonase has at least 96%, at least 97%, at least 98%, or at least 99% identity with amino acid residues 16-355, 17-355, 18-355, 19-355, 20-355, 21-355, 22-355, 23-355, 24-355, 25-355, or 26-355 of SEQ ID NO:24 or SEQ ID NO:22.

In some variations of a DNase fusion polypeptide comprising a human PON1 or functional variant or fragment thereof, the amino acid at a position corresponding to Q192 of the human paraoxonase 1 Q192 isoform (PON1-Q192; SEQ ID NO:22) is lysine or arginine. In other, non-mutually exclusive variations, the paraoxonase contains at least one of the following amino acid substitutions relative to hPON1-Q192: (i) aspartate at the position corresponding to 18R; (ii) arginine or glycine at the position corresponding to N19; (iii) glutamine at the position corresponding to H20; (iv) lysine at the position corresponding to Q21; (v) glutamate or phenylalanine at the position corresponding to Y24; (vi) phenylalanine at the amino acid corresponding to L28; (vii) valine at the position corresponding to A30; (viii) histidine at the position corresponding to L31; (ix) threonine at the position corresponding to Q35; (x) valine at the position corresponding to 148; (xi) aspartate at the position corresponding to E49; (xii) asparagine at the position corresponding to T50; (xiii) leucine or isoleucine at the position corresponding to M55; (xiv) valine at the position corresponding to L69; (xv) methionine at the position corresponding to K75; (xvi) aspartate at the position corresponding to N78; (xvii) aspartate at the position corresponding to N80; (xviii) lysine at the position corresponding to S81; (xix) serine at the position corresponding to P82; (xx) valine at the position corresponding to T96; (xxi) serine at the position corresponding to L98; (xxii) glutamate at the position corresponding to G101; (xxiii) asparagine at the position corresponding to S105; (xxiv) threonine at the position corresponding to K106; (xxv) leucine at the position corresponding to F107; (xxvi) isoleucine at the position corresponding to V109; (xxvii) threonine at the position corresponding to SI11; (xxviii) tryptophan or alanine at the position corresponding to H115; (xxix) threonine at the position corresponding to A126; (xxx) valine at the position corresponding to M127; (xxxi) arginine at the position corresponding to H134; (xxxii) glutamine at the position corresponding to D136; (xxxiii) serine at the position corresponding to A137; (xxxiv) serine at the position corresponding to K138; (xxxv) valine at the position corresponding to L143; (xxxvi) serine at the position corresponding to N166; (xxxvii) valine at the position corresponding to L167; (xxxviii) alanine at the position corresponding to G180; (xxxix) glutamate at the position corresponding to Y185; (xl) glutamine at the position corresponding to F186; (xli) lysine or alanine at the position corresponding to L187; (xlii) lysine at the position corresponding to Yi90; (xxliii) glutamine at the position corresponding to L191; (xliv) lysine at the position corresponding to Q192; (xlv) lysine at the position corresponding to W194; (xlvi) histidine at the position corresponding to Y197; (xlvii) glutamate at the position corresponding to L198; (xlviii) glutamine at the position corresponding to L200; (xlix) lysine at the position corresponding to W202; (1) phenylalanine at the position corresponding to Y204; (Ii) threonine at the position corresponding to V206; (lii) asparagine at the position corresponding to S211; (liii) aspartate at the position corresponding to E212; (liv) serine or methionine at the position corresponding to F222; (lv) aspartate at the position corresponding to N265; (lvi) valine at the position corresponding to E276; (lvii) glutamine at the position corresponding to M289; (lviii) leucine at the position corresponding to 1291; (lix) glutamate or tyrosine at the position corresponding to F293; (lx) proline at the position corresponding to S296; (lxi) lysine at the position corresponding to E297; (lxii) glycine at the position corresponding to A301; (lxiii) aspartate at the position corresponding to N309; (lxiv) serine at the position corresponding to T312; (lxv) valine at the position corresponding to Q319; (lxvi) serine at the position corresponding to T332; and alanine at the position corresponding to S335.

In certain embodiments comprising a paraoxonase, the paraoxonase is a variant of human PON1 identified as G3C9 (Aharoni et al., Proc. Nati. Acad. Sci. USA 101:482-487, 2004; Harel et al., Nat. Struct. Mol. Biol. 11:412-419, 2004; Mukherjee and Gupta, J. Toxicol. 2020:1-16, 2020; Goldsmit et al., Chemistry and Biology 19:456-466, 2012), or a functional variant or fragment thereof. G3C9 includes 55 amino acid substitutions from the human isoform, improving the catalytic activity against nerve agents and improving soluble expression in bacteria. The full length form of this paraoxonase sequence variant is listed in SEQ ID NO:39 (nucleotide) and SEQ ID NO:40 (amino acid). In some embodiments, a GC39 paraoxonase or functional variant thereof for use in accordance with the present invention has at least 90% or at least 95% identity with amino acid residues 16-355 or 26-355 of SEQ ID NO:40; in some such embodiments, the GC39 paraoxonase or functional variant thereof has at least 96%, at least 97%, at least 98%, or at least 99% identity with amino acid residues 16-355, 17-355, 18-355, 19-355, 20-355, 21-355, 22-355, 23-355, 24-355, 25-355, or 26-355 of SEQ ID NO:40.

In some embodiments, a paraoxonase is a modified or further variant form of GC39 identified as IIG1 (also referred to as M-IIG1 herein; see Goldsmith et al., Chemistry & Biology 19, 456-466, 2012), or a functional variant or fragment thereof. The full length sequence of M-IIG1 is shown in SEQ ID NO:41 (nucleotide) and SEQ ID NO:42 (amino acid). In some embodiments, a M-IIG1 paraoxonase or functional variant thereof for use in accordance with the present invention has at least 90% or at least 95% identity with amino acid residues 16-355 or 26-355 of SEQ ID NO:42; in some such embodiments, the M-IIG1 paraoxonase or functional variant thereof has at least 96%, at least 97%, at least 98%, or at least 99% identity with amino acid residues 16-355, 17-355, 18-355, 19-355, 20-355, 21-355, 22-355, 23-355, 24-355, 25-355, or 26-355 of SEQ ID NO:42.

In more specific variations, the paraoxonase has an amino acid sequence selected from (i) residues 16-355 of SEQ ID NO:24, (ii) residues 17-355 of SEQ ID NO:24, (iii) residues 18-355 of SEQ ID NO:24, (iv) residues 19-355 of SEQ ID NO:24, (v) residues 20-355 of SEQ ID NO:24, (vi) residues 21-355 of SEQ ID NO:24, (vii) residues 22-355 of SEQ ID NO:24, (viii) residues 23-355 of SEQ ID NO:24, (ix) residues 24-355 of SEQ ID NO:24, (x) residues 25-355 of SEQ ID NO:24, and (xi) residues 26-355 of SEQ ID NO:24 (i.e., an amino acid sequence selected from residues n-355 of SEQ ID NO:24, wherein n is an integer from 16 to 26, inclusive). In other variations, the paraoxonase has an amino acid sequence selected from (i) residues 16-355 of SEQ ID NO:22, (ii) residues 17-355 of SEQ ID NO:22, (iii) residues 18-355 of SEQ ID NO:22, (iv) residues 19-355 of SEQ ID NO:22, (v) residues 20-355 of SEQ ID NO:22, (vi) residues 21-355 of SEQ ID NO:22, (vii) residues 22-355 of SEQ ID NO:22, (viii) residues 23-355 of SEQ ID NO:22, (ix) residues 24-355 of SEQ ID NO:22, (x) residues 25-355 of SEQ ID NO:22, and (xi) residues 26-355 of SEQ ID NO:22 (i.e., an amino acid sequence selected from residues n-355 of SEQ ID NO:22, wherein n is an integer from 16 to 26, inclusive). In still other variations, the paraoxonase has an amino acid sequence selected from (i) residues 16-355 of SEQ ID NO:40, (ii) residues 17-355 of SEQ ID NO:40, (iii) residues 18-355 of SEQ ID NO:40, (iv) residues 19-355 of SEQ ID NO:40, (v) residues 20-355 of SEQ ID NO:40, (vi) residues 21-355 of SEQ ID NO:40, (vii) residues 22-355 of SEQ ID NO:40, (viii) residues 23-355 of SEQ ID NO:40, (ix) residues 24-355 of SEQ ID NO:40, (x) residues 25-355 of SEQ ID NO:40, and (xi) residues 26-355 of SEQ ID NO:40 (i.e., an amino acid sequence selected from residues n-355 of SEQ ID NO:40, wherein n is an integer from 16 to 26, inclusive). In yet other variations, the paraoxonase has an amino acid sequence selected from (i) residues 16-355 of SEQ ID NO:42, (ii) residues 17-355 of SEQ ID NO:42, (iii) residues 18-355 of SEQ ID NO:42, (iv) residues 19-355 of SEQ ID NO:42, (v) residues 20-355 of SEQ ID NO:42, (vi) residues 21-355 of SEQ ID NO:42, (vii) residues 22-355 of SEQ ID NO:42, (viii) residues 23-355 of SEQ ID NO:42 (ix) residues 24-355 of SEQ ID NO:42, (x) residues 25-355 of SEQ ID NO:42, and (xi) residues 26-355 of SEQ ID NO:42 (i.e., an amino acid sequence selected from residues n-355 of SEQ ID NO:42, wherein n is an integer from 16 to 26, inclusive).

Polypeptide linkers for use in accordance with the present invention can be naturally occurring, synthetic, or a combination of both. The linker joins two separate polypeptide regions (e.g., an Fc region and a DNase) and maintains the linked polypeptide regions as separate and discrete domains of a longer polypeptide. The linker can allow the separate, discrete domains to cooperate yet maintain separate properties (e.g., in the case of an Fc region linked to a DNase, Fc receptor (e.g., FcRn) binding may be maintained for the Fc region, while functional properties of the DNase (e.g., DNA binding and nuclease activity) will be maintained). For examples of the use of naturally occurring as well as artificial peptide linkers to connect heterologous polypeptides, see, e.g., Hallewell et al., J. Biol. Chem. 264, 5260-5268, 1989; Alfthan et al., Protein Eng. 8, 725-731, 1995; Robinson and Sauer, Biochemistry 35, 109-116, 1996; Khandekar et al., J. Biol. Chem. 272, 32190-32197, 1997; Fares et al., Endocrinology 139, 2459-2464, 1998; Smallshaw et al., Protein Eng. 12, 623-630, 1999; U.S. Pat. No. 5,856,456.

Typically, residues within the linker polypeptide are selected to provide an overall hydrophilic character and to be non-immunogenic and flexible. As used herein, a “flexible” linker is one that lacks a substantially stable higher-order conformation in solution, although regions of local stability are permissible. In general, small, polar, and hydrophilic residues are preferred, and bulky and hydrophobic residues are undesirable. Areas of local charge are to be avoided; if the linker polypeptide includes charged residues, they will ordinarily be positioned so as to provide a net neutral charge within a small region of the polypeptide. It is therefore preferred to place a charged residue adjacent to a residue of opposite charge. In general, preferred residues for inclusion within the linker polypeptide include Gly, Ser, Ala, Thr, Asn, and Gln; more preferred residues include Gly, Ser, Ala, and Thr; and the most preferred residues are Gly and Ser. In general, Phe, Tyr, Trp, Pro, Leu, Ile, Lys, and Arg residues will be avoided (unless present within an immunoglobulin hinge region of the linker), Pro residues due to their hydrophobicity and lack of flexibility, and Lys and Arg residues due to potential immunogenicity. The sequence of the linker will also be designed to avoid unwanted proteolysis.

Typically, linker L1-joining the DNase to the N-terminus of the Fc region-comprises at least 16 amino acid residues (e.g., at least 17, at least 18, at least 19, at least 20, at least 21, at least 22, at least 23, at least 24, or at least 25 amino acid residues). More typically, linker L1 comprises at least 26 amino acid residues. In more particular variations, L1 consists of from 16 to 60 amino acid residues, from 21 to 60 amino acid residues, from 26 to 60 amino acid residues, from 16 to 51 amino acid residues, from 21 to 51 amino acid residues, from 26 to 51 amino acid residues, from 16 to 50 amino acid residues, from 21 to 50 amino acid residues, from 26 to 50 amino acid residues, from 16 to 46 amino acid residues, from 21 to 46 amino acid residues, from 26 to 46 amino acid residues, from 16 to 45 amino acid residues, from 21 to 45 amino acid residues, from 26 to 45 amino acid residues, from 16 to 41 amino acid residues, from21 to 41 amino acid residues, from 26 to 41 amino acid residues, from 16 to 40 amino acid residues, from 21 to 40 amino acid residues, from 26 to 40 amino acid residues, from 16 to 36 amino acid residues, from 21 to 36 amino acid residues, or from 26 to 36 amino acid residues. In still more specific variations, Li consists of 26 amino acid residues, 27 amino acid residues, 28 amino acid residues, 29 amino acid residues, 30 amino acid residues, 31 amino acid residues, 32 amino acid residues, 33 amino acid residues, 34 amino acid residues, 35 amino acid residues, or 36 amino acid residues. In some embodiments, L1 comprises or consists of the amino acid sequence shown in SEQ ID NO:8 or SEQ ID NO:10.

Exemplary L2 linkers comprise at least three amino acid residues and are typically up to 60 amino acid residues. In certain variations, L2 linkers comprise at least four, at least five, at least six, at least seven, at least eight, at least 9, or at least 10 amino acid residues. In more specific variations, L2 consists of from six to 30, from six to 25, from six to 20, from seven to 30, from seven to 25, from seven to 20, from eight to 30, from eight to 25, from eight to 20, from nine to 30, from nine to 25, from nine to 20, from 10 to 30, from 10 to 25, from 10 to 20, from 11 to 30, from 11 to 25, from 11 to 20, from 12 to 30, from 12 to 25, or from 12 to 20 amino acid residues. In some embodiments, L2 comprises or consists of the amino acid sequence shown in SEQ ID NO:32.

In certain embodiments, a polypeptide linker comprises a plurality of glycine residues. For example, in some embodiments, a polypeptide linker (e.g., LI) comprises a plurality of glycine residues and optionally at least one serine residue (e.g., a plurality of serine residues). In particular variations, a polypeptide linker (e.g., LI) comprises the sequence Gly-Gly-Gly-Gly-Ser (SEQ ID NO:29), such as, e.g., two or more tandem repeats of the amino acid sequence of SEQ ID NO:29. In some embodiments, a linker comprises the sequence [Gly-Gly-Gly-Gly-Ser]n ([SEQ ID NO:29]n), where n is a positive integer such as, for example, an integer from 4 to 8, from 4 to 7, or from 4 to 6.

In a specific variation of a polypeptide linker comprising the formula [Gly-Gly-Gly-Gly-Ser]n, n is 4. In another specific variation of a polypeptide linker comprising the formula [Gly-Gly-Gly-Gly-Ser]n, n is 6. In yet another specific variation of a polypeptide linker comprising the formula [Gly-Gly-Gly-Gly-Ser]n, n is 5. In certain embodiments, a polypeptide linker comprises a series of glycine and serine residues (e.g., [Gly-Gly-Gly-Gly-Ser]n, where n is defined as above) inserted between two other sequences of the polypeptide linker (e.g., inserted between Asp-Leu-Ser at the N-terminal end of the linker and Thr-Gly-Leu at the C-terminal end of the linker). In other embodiments, a polypeptide linker includes glycine and serine residues (e.g., [Gly-Gly-Gly-Gly-Ser]n, where n is defined as above) attached at one or both ends of another sequence of the polypeptide linker.

In some embodiments, the immunoglobulin Fc region is a human IgG Fc region having, relative to the wild-type human IgG sequence, an amino acid substitution in the CH2 region so that the molecule is not glycosylated, including but not limited to an amino acid substitution at N297 (EU numbering for human IgG heavy chain constant region) (corresponding to amino acid position 82 of SEQ ID NO:12 or SEQ ID NO:14). In another embodiment, the Fc region is human IgG1 (yl) with the three cysteines of the hinge region (C220, C226, C229) each changed to a non-cysteine residue (e.g., serine) and, optionally, the proline at position 238 of the CH2 domain changed to a non-proline residue (e.g., serine or aspartate). In another embodiment, the Fc region is human γ1 with cysteine C220 changed to a non-cysteine residue (e.g., serine) and, optionally, the proline at position 238 of the CH2 domain changed to a non-proline residue (e.g., serine or aspartate). In another embodiment, the Fc region is human γ1 with N297 changed to a non-asparagine residue (e.g., alanine, glutamine, or glycine). In another embodiment, the Fc region is human γ1 with one or more amino acid substitutions between Eu positions 292 and 300. In another embodiment, the Fc region is human γ1 with one or more amino acid additions or deletions at any position between residues 292 and 300. In another embodiment, the Fc region is human γ1 with an SCC hinge (i.e., with cysteine C220 changed to serine and with a cysteine at each of Eu positions 226 and 229) or an SSS hinge (i.e., each of the three cysteines at Eu positions 220, 226, and 229 changed to serine). In further embodiments, the Fc region is human γ1 with an SCC hinge and an amino acid substitution at P238. In another embodiment, the Fc domain is human γ1 with amino acid substitutions that alter binding by Fe gamma receptors (I, II, III) without affecting FcRn binding important for half-life. In further embodiments, an Fc region is as disclosed in Ehrhardt and Cooper, Curr. Top. Microbiol. Immunol. 2010 Aug. 3 (Immunoregulatory Roles for Fe Receptor-Like Molecules); Davis et al., Ann. Rev. Immunol. 25:525-60, 2007 (Fc receptor-like molecules); or Swainson et al., J. Immunol. 184:3639-47, 2010.

In some embodiments, the Fc region comprises an amino acid substitution that alters the antigen-independent effector functions of the fusion protein. In some such embodiments, the Fc region includes an amino acid substitution that alters the circulating half-life of the resulting molecule. Such Fe variants exhibit either increased or decreased binding to FcRn when compared to an Fc region lacking these substitutions and, therefore, confer increased or decreased half-life, respectively, of the resulting molecule in serum. Fe variants with improved affinity for FcRn are anticipated to have longer serum half-lives, and such Fe variants have useful applications in methods of treating mammals where long half-life of the administered Fc fusion is desired and where increased transport through the lungs to the circulation is desired. In contrast, Fe variants with decreased FcRn binding affinity are expected to have shorter half-lives, and such variants are also useful, for example, for administration to a mammal where a shortened circulation time may be advantageous (e.g., where the fusion protein has toxic side effects when present in the circulation for prolonged periods). For treatment of conditions characterized by NETosis, fine tuning of the half-life of a DNase-Fc fusion protein through altering FcRn binding may be useful since NETs, while contributing to pathogenesis, are also beneficial in certain settings; digestion of pathogenic NETs may not require as long a half life, so more rapid clearance of the DNase may allow neutrophils to form NETs in their protective anti-microbial function. Fe variants with decreased FcRn binding affinity are also less likely to cross the placenta and, thus, are also useful in the treatment of diseases or disorders in pregnant women. In addition, other applications in which reduced FcRn binding affinity may be desired include those applications in which localization to the brain, kidney, and/or liver is desired. In one exemplary embodiment, the fusion molecules of the invention exhibit reduced transport across the epithelium of kidney glomeruli from the vasculature. In another embodiment, the fusion molecules of the invention exhibit reduced transport across the blood brain barrier (BBB) from the brain, into the vascular space. In one embodiment, a fusion molecule with altered FcRn binding comprises an Fc region having one or more amino acid substitutions within the “FcRn binding loop” of the Fc domain. Exemplary amino acid substitutions that alter FcRn binding activity are disclosed in International PCT Publication No. WO 05/047327, which is incorporated by reference herein. Exemplary amino acid substitutions that increase FcRn binding activity are also described, e.g., by Wang et al., Protein Cell 9:63-73, 2018 (see, e.g., Table 1). In some embodiments, an Fe variant with increased FcRn binding activity has an amino acid substitution at each of Eu positions 252, 254, and 256 (e.g., M252Y, S254T, and T256E). In other variations, an Fe variant with increased FcRn binding activity has an amino acid substitution at each of Eu positions 428 and 434 (e.g., M428L and N434S).

In other embodiments, a fusion polypeptide of the present invention comprises an Fe variant comprising an amino acid substitution that alters one or more antigen-dependent effector functions of the polypeptide, in particular antibody-dependent cellular cytotoxicity (ADCC) or complement activation, e.g., as compared to a wild type Fc region. In an exemplary embodiment, such fusion polypeptides exhibit altered binding to an Fe gamma receptor (FcγR, e.g., CD16). Such fusion polypeptides exhibit either increased or decreased binding to FcγR when compared to wild-type polypeptides and, therefore, mediate enhanced or reduced effector function, respectively. Fe variants with improved affinity for FcγRs are anticipated to enhance effector function, and such variants have useful applications in methods of treating mammals where target molecule destruction is desired. In contrast, Fe variants with decreased FcγR binding affinity are expected to reduce effector function, and such fusion proteins are also useful, for example, for treatment of conditions in which target cell destruction is undesirable, e.g., where normal cells may express target molecules, or where chronic administration of the fusion molecule might result in unwanted immune system activation. In one embodiment, the fusion polypeptide comprising an Fc region exhibits at least one altered antigen-dependent effector function selected from the group consisting of opsonization, phagocytosis, complement dependent cytotoxicity (CDC), antibody-dependent cellular cytotoxicity (ADCC), or effector cell modulation as compared to a polypeptide comprising a wild-type Fe region. In typical embodiments of a DNase fusion molecule comprising an Fe variant with altered antigen-dependent effector function, the Fe variant has one or more reduced effector functions relative to the corresponding wild-type Fc region.

In one embodiment, a fusion polypeptide comprising an Fc region exhibits altered binding to an activating FcγR (e.g., FcγI, FcγIIa, or FcγRIIIa). In another embodiment, the fusion protein exhibits altered binding affinity to an inhibitory FcγR (e.g., FcγRIIb). Exemplary amino acid substitutions which alter FcR or complement binding activity are disclosed in International PCT Publication No. WO 05/063815, which is incorporated by reference herein. Exemplary Fe variants with reduced effector function are also described, e.g., by Tam et al., Antibodies 6:12, 2017 (describing variants of human IgG1 (yl) and IgG4(γ4)); Wang et al., Protein Cell 9:63-73, 2018 (see, e.g., Table 1); Lo et al., J. Biol. Chem. 292:3900-3908, 2017; Idusogie et al., J. Immunol. 164:4178-4184, 2000 (each of which is incorporated by reference herein). Suitable Fe variants that reduce antigen-dependent effector function include, for example, variants having an amino acid substitution at Eu position 238 and/or position 331 (e.g., P238S and/or P331S or P331A). In addition, amino acid substitutions at Eu positions 234 and 235 of human Fe (e.g., L234A/L235A in IgG1, or F234A/L235A in IgG4) reduce FcγR binding and have been shown to reduce cytokine storm when introduced into anti-CD3 mAb (see, e.g., Wang et al., supra), and an amino acid substitution at Eu position 329 (e.g., P329A) is highly effective at reducing Clq binding (see, e.g., Lo et al., supra). Other exemplary approaches to removing ADCC and CDC effector functions is to make hybrid Fe domains derived from human IgG2 (Eu positions 118-260) and IgG4 (Eu positions 261-447), or to modify human IgG2 to contain selected amino acid substitutions from IgG4. See Wang et al., supra.

A fusion polypeptide comprising an Fc region may also comprise an amino acid substitution that alters the glycosylation of the Fc region. For example, the Fc domain of the fusion protein may have a mutation leading to reduced glycosylation (e.g., N- or O-linked glycosylation) or may comprise an altered glycoform of the wild-type Fc domain (e.g., a low fucose or fucose-free glycan). In another embodiment, the molecule has an amino acid substitution near or within a glycosylation motif, for example, an N-linked glycosylation motif that contains the amino acid sequence NXT or NXS. Exemplary amino acid substitutions which reduce or alter glycosylation are disclosed in International PCT Publication No. WO 05/018572 and US Patent Application Publication No. 2007/0111281, which are incorporated by reference herein.

Particularly suitable amino acid substitutions to reduce glycosylation and which also reduce ADCC and CDC effector functions of Fe include amino acid substitutions at Eu position 297 (e.g., N297A, N297Q, or N297G). See, e.g., Wang et al., supra. N297 substitutions may also be paired with substitutions at position 265 (e.g., D265A) to further reduce CDC. See, e.g., Lo et al., supra.

It will be understood by those of skill in the art that various embodiments of Fe variants as described herein can be combined in the fusion polypeptides of the present invention, unless the context clearly indicates otherwise.

In some embodiments, the immunoglobulin Fc region comprises an amino acid sequence having at least 80%, at least 85%, at least 90%, or at least 95% identity with an amino acid sequence selected from sequence shown in (i) residues 1-232 or 1-231 of SEQ ID NO:12, (ii) residues 1-232 or 1-231 of SEQ ID NO:14, or (iii) residues 1-232 or 1-231 of SEQ ID NO:16. In yet other embodiments, the Fc region comprises an amino acid sequence having at least 96%, at least 97%, at least 98%, at least 99%, or 100% identity with the amino acid sequence shown in (i) residues 1-232 or 1-231 of SEQ ID NO:12, (ii) residues 1-232 or 1-231 of SEQ ID NO:14, or (iii) residues 1-232 or 1-231 of SEQ ID NO:16.

In some embodiments of a fusion polypeptide comprising D-L1-X as described above, the fusion polypeptide comprises an amino acid sequence having at least 80%, at least 85%, at least 90%, or at least 95% identity with an amino acid sequence selected from sequence shown in (i) residues 21-538, 21-537, 1-538, or 1-537 of SEQ ID NO:18, (ii) residues 21-548, 21-547, 1-548, or 1-547 of SEQ ID NO:20, (iii) residues 21-538, 21-537, 1-538, or 1-537 of SEQ ID NO:55, (iv) residues 21-548, 21-547, 1-548, or 1-547 of SEQ ID NO:56, (v) residues 21-538, 21-537, 1-538, or 1-537 of SEQ ID NO:86, (vi) residues 21-538, 21-537, 1-538, or 1-537 of SEQ ID NO:88, (vii) residues 21-538, 21-537, 1-538, or 1-537 of SEQ ID NO:90, (viii) residues 21-538, 21-537, 1-538, or 1-537 of SEQ ID NO:92, (ix) residues 21-538, 21-537, 1-538, or 1-537 of SEQ ID NO:94, (x) residues 21-538, 21-537, 1-538, or 1-537 of SEQ ID NO:96, (xi) residues 21-548, 21-547, 1-548, or 1-547 of SEQ ID NO:36, or (xii) residues 21-548, 21-547, 1-548, or 1-547 of SEQ ID NO:38. In yet other embodiments, the fusion polypeptide comprises an amino acid sequence having at least 96%, at least 97%, at least 98%, at least 99%, or 100% identity with the amino acid sequence shown in (i) residues 21-538, 21-537, 1-538, or 1-537 of SEQ ID NO:18, (ii) residues 21-548, 21-547, 1-548, or 1-547 of SEQ ID NO:20, (iii) residues 21-538, 21-537, 1-538, or 1-537 of SEQ ID NO:55, (iv) residues 21-548, 21-547, 1-548, or 1-547 of SEQ ID NO:56, (v) residues 21-538, 21-537, 1-538, or 1-537 of SEQ ID NO:86, (vi) residues 21-538, 21-537, 1-538, or 1-537 of SEQ ID NO:88, (vii) residues 21-538, 21-537, 1-538, or 1-537 of SEQ ID NO:90, (viii) residues 21-538, 21-537, 1-538, or 1-537 of SEQ ID NO:92, (ix) residues 21-538, 21-537, 1-538, or 1-537 of SEQ ID NO:94, (x) residues 21-538, 21-537, 1-538, or 1-537 of SEQ ID NO:96, (xi) residues 21-548, 21-547, 1-548, or 1-547 of SEQ ID NO:36, or (xii) residues 21-548, 21-547, 1-548, or 1-547 of SEQ ID NO:38.

In some embodiments of a fusion polypeptide comprising D-L1-X as described above, the fusion polypeptide comprises the amino acid sequence shown in SEQ ID NO:49, SEQ ID NO:50, SEQ ID NO:53, SEQ ID NO:54, SEQ ID NO:97, SEQ ID NO:98, SEQ ID NO:99, SEQ ID NO:100, SEQ ID NO:101, SEQ ID NO:102, SEQ ID NO:103, or SEQ ID NO:104. In certain variations of a fusion polypeptide comprising the amino acid sequence shown in SEQ ID NO:49, SEQ ID NO:50, SEQ ID NO:53, SEQ ID NO:54, SEQ ID NO:97, SEQ ID NO:98, SEQ ID NO:99, SEQ ID NO:100, SEQ ID NO:101, SEQ ID NO:102, SEQ ID NO:103, or SEQ ID NO:104, (i) the amino acid at each of positions 315 and 316 is alanine; (ii) the amino acid at position 319 is serine; (iii) the amino acid at position 378 is alanine, glutamine, or glycine, and the amino acid at position 346 is optionally alanine; (iv) the amino acid at position 410 is alanine, glycine, or serine; (v) the amino acid at position 412 is serine or alanine; (vi) the amino acid at position 333 is tyrosine, the amino acid at position 335 is threonine, and the amino acid at position 337 is glutamate; and/or (vii) the amino acid at position 509 is leucine, and the amino acid at position 515 is serine. In other, non-mutually exclusive variations of a fusion polypeptide comprising the amino acid sequence shown in SEQ ID NO:49, SEQ ID NO:50, SEQ ID NO:53, SEQ ID NO:54, SEQ ID NO:97, SEQ ID NO:98, SEQ ID NO:99, SEQ ID NO:100, SEQ ID NO:101, SEQ ID NO:102, SEQ ID NO:103, or SEQ ID NO:104, the amino acid sequence corresponding to positions 261-296 includes a Gly-Ser tandem repeat sequence having a formula selected from the group consisting of (i) [Gly-Gly-Gly-Gly-Ser]n, wherein n is an integer from 4 to 7, (ii) [Gly-Gly-Gly-Ser]n, wherein n is an integer from 5 to 9, and (iii) [Gly-Gly-Ser]n, wherein n is an integer from 6 to 12; in some such embodiments, the amino acid sequence corresponding to positions 261-296 includes a Gly-Ser tandem repeat sequence having a formula selected from the group consisting of (i) [Gly-Gly-Gly-Gly-Ser]n, wherein n is an integer from 4 to 6, (ii) [Gly-Gly-Gly-Ser]n, wherein n is an integer from 5 to 7, and (iii) [Gly-Gly-Ser]n, wherein n is an integer from 6 to 10. In yet other, non-mutually exclusive variations of a fusion polypeptide comprising the amino acid sequence shown in SEQ ID NO:49, SEQ ID NO:50, SEQ ID NO:53, SEQ ID NO:54, SEQ ID NO:97, SEQ ID NO:98, SEQ ID NO:99, SEQ ID NO:100, SEQ ID NO:101, SEQ ID NO:102, SEQ ID NO:103, or SEQ ID NO:104, the amino acid at position 261 is aspartate, the amino acid at position 262 is leucine, the amino acid at position 263 is serine, the amino acid at position 294 is threonine, the amino acid at position is 295 is glycine, and/or the amino acid at position 296 is leucine. In other, non-mutually exclusive variations of a fusion polypeptide comprising the amino acid sequence shown in SEQ ID NO:49, SEQ ID NO:53, SEQ ID NO:97, SEQ ID NO:98, SEQ ID NO:99, SEQ ID NO:100, or SEQ ID NO:101, the amino acid at each of positions 74 and 105 is independently lysine or arginine. In other, non-mutually exclusive variations of a fusion polypeptide comprising the amino acid sequence shown in SEQ ID NO:49, the amino acid at position 114 is phenylalanine. In other, non-mutually exclusive variations of a fusion polypeptide comprising the amino acid sequence shown in SEQ ID NO:97, (i) the amino acid at position 53 is arginine, alanine, methionine, or tyrosine, (ii) the amino acid at position 65 is alanine or arginine, and/or (iii) the amino acid at position 69 is lysine or arginine. In other, non-mutually exclusive variations of a fusion polypeptide comprising the amino acid sequence shown in SEQ ID NO:97, SEQ ID NO:98, or SEQ ID NO:101, the amino acid position 53 is arginine. In other, non-mutually exclusive variations of a fusion polypeptide comprising the amino acid sequence shown in SEQ ID NO:97, SEQ ID NO:99, or SEQ ID NO:101, the amino acid at position 65 is alanine. In other, non-mutually exclusive variations of a fusion polypeptide comprising the amino acid sequence shown in SEQ ID NO:97 or SEQ ID NO:100, the amino acid at position 69 is lysine.

In some embodiments of a fusion polypeptide comprising D-L1-X-L2-P as described above and where P is a paraoxonase, the fusion polypeptide comprises an amino acid sequence having at least 80%, at least 85%, at least 90%, or at least 95% identity with the amino acid sequence shown in (i) residues 21-896 or 1-896 of SEQ ID NO:26, (ii) residues 21-896 or 1-896 of SEQ ID NO:44, (iii) residues 21-896 or 1-896 of SEQ ID NO:46, (iv) residues 21-906 or 1-906 of SEQ ID NO:48, (v) residues 21-896 or 1-896 of SEQ ID NO:58, (vi) residues 21-906 or 1-906 of SEQ ID NO:60, (vii) residues 21-906 or 1-906 of SEQ ID NO:68, or (viii) residues 21-906 or 1-906 of SEQ ID NO:70. In yet other embodiments, the fusion polypeptide comprises an amino acid sequence having at least 96%, at least 97%, at least 98%, at least 99%, or 100% identity with the amino acid sequence shown in (i) residues 21-896 or 1-896 of SEQ ID NO:26, (ii) residues 21-896 or 1-896 of SEQ ID NO:44, (iii) residues 21-896 or 1-896 of SEQ ID NO:46, (iv) residues 21-906 or 1-906 of SEQ ID NO:48, (v) residues 21-896 or 1-896 of SEQ ID NO:58, (vi) residues 21-906 or 1-906 of SEQ ID NO:60, (vii) residues 21-906 or 1-906 of SEQ ID NO:68, or (viii) residues 21-906 or 1-906 of SEQ ID NO:70.

The present invention also provides dimeric proteins comprising first and second polypeptide fusions as described above. Accordingly, in another aspect, the present invention provides a dimeric protein comprising a first fusion polypeptide and a second fusion polypeptide, wherein each of the first and second fusion polypeptides comprises, from an amino-terminal position to a carboxyl-terminal position, D-L1-X, wherein D is a biologically active DNase, L1 is a first polypeptide linker comprising at least 26 amino acid residues, and X is an immunoglobulin Fc region. In some embodiments, each of the first and second fusion polypeptides further includes a polypeptide segment located carboxyl-terminal to the immunoglobulin Fc region (a “third polypeptide segment”). In particular variations, the third polypeptide segment is a biologically active paraoxonase. Such a fusion polypeptide comprising a third polypeptide segment may be represented by the formula D-L1-X-L2-P (from an amino-terminal position to a carboxyl-terminal position), wherein D, LI, and X are each as previously defined, wherein L2 is a second polypeptide linker and is optionally present, and wherein P is the third polypeptide segment.

### III. Materials and Methods for Making Polypeptide Fusions and Dimeric Proteins

The present invention also provides polynucleotide molecules, including DNA and RNA molecules, that encode the fusion polypeptides disclosed above. The polynucleotides of the present invention include both single-stranded and double-stranded molecules. Polynucleotides encoding various segments of a fusion polypeptide (e.g., an Fe fragment; DNase and P polypeptide segments) can be generated and linked together to form a polynucleotide encoding a fusion polypeptide as described herein using known methods for recombinant manipulation of nucleic acids.

DNA sequences encoding DNases (e.g., DNase 1 or DNase1L3), paraoxonases (e.g., PON1), and immunoglobulin Fc regions are generally known in the art. Exemplary DNA sequences are disclosed herein (see Sequence Listing). Additional DNA sequences encoding any of these polypeptides can be readily generated by those of ordinary skill in the art based on the genetic code. Counterpart RNA sequences can be generated by substitution of U for T. Those skilled in the art will readily recognize that, in view of the degeneracy of the genetic code, considerable sequence variation is possible among polynucleotide molecules encoding a given polypeptide. DNA and RNA encoding functional variants and fragments of such polypeptides can also be obtained using known recombinant methods to introduce variation into a polynucleotide sequence, followed by expression of the encoded polypeptide and determination of functional activity (e.g., nuclease activity) using an appropriate screening assay.

Methods for preparing DNA and RNA are well known in the art. For example, complementary DNA (cDNA) clones can be prepared from RNA that is isolated from a tissue or cell that produces large amounts of RNA encoding a polypeptide of interest. Total RNA can be prepared using guanidine HCl extraction followed by isolation by centrifugation in a CsCl gradient (Chirgwin et al., Biochemistry 18:52-94, 1979). Poly (A)′ RNA is prepared from total RNA using the method of Aviv and Leder (Proc. Nati. Acad. Sci. USA 69:1408-1412, 1972). Complementary DNA is prepared from poly(A)+RNA using known methods. In the alternative, genomic DNA can be isolated. Methods for identifying and isolating cDNA and genomic clones are well known and within the level of ordinary skill in the art, and include the use of the sequences disclosed herein, or parts thereof, for probing or priming a library. Polynucleotides encoding polypeptides of interest are identified and isolated by, for example, hybridization or polymerase chain reaction (“PCR,” Mullis, U.S. Pat. No. 4,683,202). Expression libraries can be probed with antibodies to the polypeptide of interest, receptor fragments, or other specific binding partners.

The polynucleotides of the present invention can also be prepared by automated synthesis. The production of short, double-stranded segments (60 to 80 bp) is technically straightforward and can be accomplished by synthesizing the complementary strands and then annealing them. Longer segments (typically >300 bp) are assembled in modular form from single-stranded fragments that are from 20 to 100 nucleotides in length. Automated synthesis of polynucleotides is within the level of ordinary skill in the art, and suitable equipment and reagents are available from commercial suppliers. See generally Glick and Pasternak, Molecular Biotechnology, Principles & Applications of Recombinant DNA, ASM Press, Washington, D.C. , 1994; Itakura et al., Ann. Rev. Biochem. 53:323-356, 1984; and Climie et al., Proc. Natl. Acad. Sci. USA 87:633-637, 1990.

In another aspect, materials and methods are provided for producing the polypeptide fusions of the present invention, including dimeric proteins comprising the fusion polypeptides. The fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N Y, 1989, and Ausubel et al., eds., Current Protocols in Molecular Biology, Green and Wiley and Sons, N Y, 1993.

In general, for production of a fusion polypeptide in a host cell, a DNA sequence encoding the fusion polypeptide is operably linked to other genetic elements required for its expression, typically including a transcription promoter and terminator, within an expression cassette. Typically, the expression cassette is contained within an expression vector for delivery into a host cell. The vector will also commonly contain one or more selectable markers and one or more origins of replication, although those skilled in the art will recognize that within certain systems selectable markers may be provided on separate vectors, and replication of the exogenous DNA may be provided by integration of an expression cassette into the host cell genome such as, e.g., in the generation of stable cell lines. Selection of promoters, terminators, selectable markers, vectors and other elements is a matter of routine design within the level of ordinary skill in the art. Many such elements are described in the literature and are available through commercial suppliers.

To direct a DNase fusion polypeptide into the secretory pathway of a host cell, a secretory signal sequence is provided in the expression cassette. The encoded secretory peptide may be that of the native DNase (e.g., amino acid residues 1-22 of SEQ ID NO:2), or may be derived from another secreted protein (e.g., t-PA; see U.S. Pat. No. 5,641,655) or synthesized de novo. An engineered cleavage site may be included at the junction between the secretory peptide and the remainder of the polypeptide fusion to optimize proteolytic processing in the host cell. The secretory signal sequence is operably linked to the DNA sequence encoding the polypeptide fusion, i.e., the two sequences are joined in the correct reading frame and positioned to direct the newly synthesized polypeptide fusion into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). Secretory signal sequences suitable for use in accordance with the present invention include, for example, polynucleotides encoding the human VK3 leader peptide (SEQ ID NO:28).

Expression of fusion polypeptides as described herein, via a host cell secretory pathway, is expected to result in the production of dimeric proteins via dimerization of the Fc region. Accordingly, in another aspect, the present invention provides dimeric proteins comprising first and second fusion polypeptides as described above (e.g., a dimeric protein comprising a first fusion polypeptide and a second fusion polypeptide, wherein each of the first and second fusion polypeptides comprises, from an amino-terminal position to a carboxyl-terminal position, D-L1-X or D-L1-X-L2-P as described herein). Dimers may also be assembled in vitro upon incubation of component polypeptides under suitable conditions. In general, in vitro assembly will include incubating the protein mixture under denaturing and reducing conditions followed by refolding and reoxidation of the polypeptides to form dimers. Recovery and assembly of proteins expressed in bacterial cells is disclosed below.

Cultured mammalian cells are suitable hosts for use within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g., CHO-K1, ATCC No. CCL 61; CHO-DG44, Urlaub et al., Proc. Natl. Acad. Sci. USA 77:4216-4220, 1980) cell lines. Additional suitable cell lines are known in the art and available from public depositories such as the American Type Culture Collection, Manassas, Virginia. Strong transcription promoters can be used, such as promoters from SV-40, cytomegalovirus, or myeloproliferative sarcoma virus. See, e.g., U.S. Pat. No. 4,956,288 and U.S. Patent Application Publication No. 20030103986. Other suitable promoters include those from metallothionein genes (U.S. Pat. Nos. 4,579,821 and 4,601,978) and the adenovirus major late promoter. Expression vectors for use in mammalian cells include pZP-1, pZP-9, and pZMP21, which have been deposited with the American Type Culture Collection, 10801 University Blvd., Manassas, VA USA under accession numbers 98669, 98668, and PTA-5266, respectively, and derivatives of these vectors.

Drug selection is generally used to select for cultured mammalian cells into which foreign DNA has been inserted. Such cells are commonly referred to as “transfectants. “Cells that have been cultured in the presence of the selective agent and are able to pass the gene of interest to their progeny—by virtue of integration of the expression cassette into its genomic DNA—are referred to as “stable transfectants.” An exemplary selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g., hygromycin resistance, multi-drug resistance, puromycin acetyltransferase) can also be used. Cell-surface markers and other phenotypic selection markers can be used to facilitate identification of transfected cells (e.g., by fluorescence-activated cell sorting), and include, for example, CD8, CD4, nerve growth factor receptor, green fluorescent protein, and the like.

In some aspects, the present invention provides a stable cell line containing, within its genomic DNA, an expression cassette encoding a DNase-Fc fusion polypeptide as described herein, wherein the stable cell line constitutively expresses the encoded DNase-Fc fusion. Stable cell lines can be generated by methods generally known in the art, which generally include the identification of single stable cell clones from a polyclonal colony of stable transfectants by limited dilution and expansion. Protein expression of selected clones can then be assessed to identify high-expressing clones for expansion. In some embodiments, the stable cell line is a mammalian cell line such as, e.g., a Chinese hamster ovary (CHO) cell line. Preferably, the stable cell line constitutively expresses an enhanced DNase1-Fc fusion as described herein (e.g., an enhanced DNase1-Fc fusion polypeptide comprising an amino acid sequence having at least 90%, at least 95%, at least 96%, at least 97%, at least 98%, at least 99%, or 100% identity with the amino acid sequence shown in (i) residues 21-538, 21-537, 1-538, or 1-537 of SEQ ID NO:18, (ii) residues 21-548, 21-547, 1-548, or 1-547 of SEQ ID NO:20, (iii) residues 21-538, 21-537, 1-538, or 1-537 of SEQ ID NO:55, (iv) residues 21-548, 21-547, 1-548, or 1-547 of SEQ ID NO:56, (v) residues 21-538, 21-537, 1-538, or 1-537 of SEQ ID NO:86, (vi) residues 21-538, 21-537, 1-538, or 1-537 of SEQ ID NO:88, (vii) residues 21-538, 21-537, 1-538, or 1-537 of SEQ ID NO:90, (viii) residues 21-538, 21-537, 1-538, or 1-537 of SEQ ID NO:92, (ix) residues 21-538, 21-537, 1-538, or 1-537 of SEQ ID NO:94, (x) residues 21-538, 21-537, 1-538, or 1-537 of SEQ ID NO:96, (xi) residues 21-896 or 1-896 of SEQ ID NO:26, (xii) residues 21-906 or 1-906 of SEQ ID NO:48, (xiii) residues 21-896 or 1-896 of SEQ ID NO:58, or (xiv) residues 21-906 or 1-906 of SEQ ID NO:60. In some embodiments, the expressed DNase1-Fc fusion polypeptide comprises the amino acid sequence as shown in SEQ ID NO:49, SEQ ID NO:50, SEQ ID NO:53, SEQ ID NO:54, SEQ ID NO:97, SEQ ID NO:98, SEQ ID NO:99, SEQ ID NO:100, SEQ ID NO:101, SEQ ID NO:102, SEQ ID NO:103, or SEQ ID NO:104. In some embodiments, the expressed DNase1-Fe fusion polypeptide is an enhanced DNase1-Fc fusion polypeptide with intermediate sensitivity to inhibition by actin (e.g., a DNase1-Fc fusion polypeptide wherein the DNase is an enhanced DNase1 containing one or more of the D53, Y65, and E69 substitutions as described herein and having intermediate sensitivity to inhibition by actin). In some variations, the stable cell line is a mammalian cell line (e.g., CHO) capable of producing the DNase1-Fc fusion polypeptide at a concentration of at least 25 mg/L, at least 50 mg/L, at least 100 mg/L, at least 200 mg/L, or at least 500 mg/L of the cell culture. Typically, a DNase1-Fc fusion polypeptide as described herein can be expressed at levels of approximately 25-100 mg/L from initial isolated clones. Recloning of the initial cultures can often stabilize and increase expression by 2-3 fold. Amplification of the expression level can also be achieved by further plating of cells at low density in increasing levels of methotrexate from an initial concentration of 50 nM up to as much as 1 μM. Once cells have adapted, further rounds of limiting dilution cloning are required to maintain high expression levels.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and International PCT Publication No. WO 94/06463.

Insect cells can be infected with recombinant baculovirus, commonly derived from Autographa californica nuclear polyhedrosis virus (AcNPV) (see King and Possee, The Baculovirus Expression System: A Laboratory Guide, Chapman & Hall, London; O'Reilly et al., Baculovirus Expression Vectors: A Laboratory Manual, Oxford University Press., New York, 1994; and Richardson, Ed., Baculovirus Expression Protocols. Methods in Molecular Biology, Humana Press, Totowa, N J, 1995). Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, MD). The transfer vector (e.g., PFASTBAC1; Life Technologies) contains a Tn7 transposon to move the DNA encoding the protein of interest into a baculovirus genome maintained in E. coli as a large plasmid called a “bacmid” (see Hill-Perkins and Possee, J. Gen. Virol. 71:971-976, 1990; Bonning et al., J. Gen. Virol. 75:1551-1556, 1994; Chazenbalk and Rapoport, J. Biol. Chem. 270:1543-1549, 1995). Using techniques known in the art, a transfer vector encoding a polypeptide fusion is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodopterafrugiperda cells, such as Sf9 cells. Recombinant virus that expresses the polypeptide fusion is subsequently produced. Recombinant viral stocks are made by methods commonly used in the art.

For protein production, the recombinant virus is used to infect host cells, typically a cell line derived from the fall armyworm, Spodopterafrugiperda (e.g., Sf9 or Sf21 cells) or Trichoplusia ni (e.g., HIGH FIVE cells; Invitrogen, Carlsbad, CA) (see generally Glick and Pasternak, supra; see also U.S. Pat. No. 5,300,435). Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (e.g., King and Possee, supra; O'Reilly et al., supra.; Richardson, supra).

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT] vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii, and Candida maltosa are known in the art. See, e.g., Gleeson et al., J. Gen. Microbiol. 132:3459-3465, 1986; Cregg, U.S. Pat. No. 4,882,279; and Raymond et al., Yeast 14:11-23, 1998. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well-known in the art (see, e.g., Sambrook et al., supra). When expressing a fusion polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine HCl or urea. The denatured polypeptide can then be refolded and dimerized by diluting the denaturant, such as by dialysis against a solution of urea and a combination of reduced and oxidized glutathione, followed by dialysis against a buffered saline solution. In the alternative, the protein may be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted polypeptides can be recovered from the periplasmic space in a soluble and functional form by disrupting the cells (by, for example, sonication or osmotic shock) and recovering the protein, thereby obviating the need for denaturation and refolding. See, e.g., Lu et al., J. Immunol. Meth. 267:213-226, 2002.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

In some variations, for production of an actin-resistant DNase-Fc fusion polypeptide as described herein (e.g., an actin-resistant DNase1-Fc fusion polypeptide comprising an amino acid sequence having at least 90%, at least 95%, at least 96%, at least 97%, at least 98%, at least 99%, or 100% identity with the amino acid sequence shown in (i) residues 21-538, 21-537, 1-538, or 1-537 of SEQ ID NO:18, (ii) residues 21-548, 21-547, 1-548, or 1-547 of SEQ ID NO:20, (iii) residues 21-538, 21-537, 1-538, or 1-537 of SEQ ID NO:86, (iv) residues 21-538, 21-537, 1-538, or 1-537 of SEQ ID NO:88, (v) residues 21-538, 21-537, 1-538, or 1-537 of SEQ ID NO:90, (vi) residues 21-538, 21-537, 1-538, or 1-537 of SEQ ID NO:92, (vii) residues 21-538, 21-537, 1-538, or 1-537 of SEQ ID NO:94, (viii) residues 21-538, 21-537, 1-538, or 1-537 of SEQ ID NO:96, (ix) residues 21-896 or 1-896 of SEQ ID NO:26, or (x) residues 21-906 or 1-906 of SEQ ID NO:48), host cells are cultured in the presence of a DNase1 inhibitor to reduce toxicity of the enhanced DNase during its expression. DNase1 inhibitors are generally known in the art and include, for example, the inhibitor rutin (see, e.g., Kolarevic et al., Chem. Biodiversity 16:e1900060, 2019). In certain embodiments, the host cells cultured in the presence of a DNase1 inhibitor are mammalian cells such as, e.g., CHO.

Proteins of the present invention are purified by conventional protein purification methods, typically by a combination of chromatographic techniques. See generally Affinity Chromatography: Principles &Methods, Pharmacia LKB Biotechnology, Uppsala, Sweden, 1988; and Scopes, Protein Purification: Principles and Practice, Springer-Verlag, New York, 1994. Proteins comprising an immunoglobulin Fc region can be purified by affinity chromatography on immobilized protein A. Additional purification steps, such as gel filtration, can be used to obtain the desired level of purity or to provide for desalting, buffer exchange, and the like.

For example, fractionation and/or conventional purification methods can be used to obtain fusion polypeptides and dimeric proteins of the present invention purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are suitable. Exemplary chromatographic media include those media derivatized with phenyl, butyl, or octyl groups, such as Phenyl-Sepharose FF (Pharmacia), Toyopearl butyl 650 (Toso Haas, Montgomeryville, PA), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well-known and widely used in the art, and are available from commercial suppliers. Selection of a particular method for polypeptide isolation and purification is a matter of routine design and is determined in part by the properties of the chosen support. See, e.g., Affinity Chromatography: Principles & Methods (Pharmacia LKB Biotechnology 1988); Doonan, Protein Purification Protocols (The Humana Press 1996).

Additional variations in protein isolation and purification can be devised by those of skill in the art. For example, antibodies that specifically bind a fusion polypeptide or dimeric protein as described herein (e.g., an antibody that specifically binds a polypeptide segment corresponding to a DNase) can be used to isolate large quantities of protein by immunoaffinity purification.

The proteins of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (see, e.g., M. Deutscher, (ed.), Meth. Enzymol. 182:529, 1990). Within additional embodiments of the invention, a fusion of the polypeptide of interest and an affinity tag (e.g., maltose-binding protein) may be constructed to facilitate purification. Moreover, receptor- or ligand-binding properties of a fusion polypeptide or dimer thereof can be exploited for purification.

In some variations, an enhanced but non-actin-resistant DNase1-Fc fusion molecule as described herein (i.e., a fusion in which the DNase does not contain any amino acid substitution, relative to human DNase1, that decreases G-actin-induced inhibition of endonuclease activity of the DNase relative to human DNase1) is treated with ethylenediamine (EDA) following purification in order to reduce the molecule's actin binding, thereby providing a DNase-Fc fusion molecule that is resistant to actin. The EDA-treated DNase-Fc is then further treated to remove EDA using methods generally known in the art (see, e.g., U.S. Pat. No. 11,225,648). Such embodiments are advantageous for achieving higher expression of the DNase-Fc for pharmaceutical use, since expression of the non-actin-resistant form of DNase-Fc would be less toxic to host cells (e.g., CHO). In particular embodiments, the purified DNase-Fc to be treated with EDA comprises an amino acid sequence having at least 90%, at least 95%, at least 96%, at least 97%, at least 98%, at least 99%, or 100% identity with the amino acid sequence shown in (i) residues 21-538 or 21-537 of SEQ ID NO:55, (ii) residues 21-548 or 21-547 of SEQ ID NO:56, (iii) residues 21-896 of SEQ ID NO:58, or (iv) residues 21-906 of SEQ ID NO:60.

The polypeptides of the present invention are typically purified to at least about 80% purity, more typically to at least about 90% purity and preferably to at least about 95%, at least about 96%, at least about 97%, at least about 98%, or at least about 99% purity with respect to contaminating macromolecules, particularly other proteins and nucleic acids, and free of infectious and pyrogenic agents. The polypeptides of the present invention may also be purified to a pharmaceutically pure state, which is greater than 99.9% pure. In certain preparations, purified polypeptide is substantially free of other polypeptides, particularly other polypeptides of animal origin.

### IV. Methods of Use and Pharmaceutical Compositions

The fusion polypeptides and dimeric proteins of the present invention can be used to provide DNase-mediated therapy for the treatment of various diseases or disorders. The DNase fusion polypeptides are particularly useful, e.g., for treatment of diseases and disorders characterized by NETosis. In some aspects relating to bispecific fusions further comprising a third polypeptide segment as described herein (e.g., a paraoxonase), the fusion polypeptides and dimeric proteins may further provide one or more additional biological activities for such treatment.

In particular aspects, the present invention provides methods for treating a disease or disorder selected from an inflammatory disease, an autoimmune disease, a neurological disease, an infectious disease, a metabolic disease, a cardiovascular disease, a liver disease, a fibrotic disease, thrombosis, sepsis, ischemia reperfusion, biofilm formation by a gram-negative bacteria (e.g., Pseudomonas aeruginosa), exposure to sulfur mustard gas, exposure to an organophosphate, and cancer. The methods generally include administering to a subject having the disease or disorder an effective amount of a fusion polypeptide or dimeric protein as described herein.

Inflammatory diseases amenable to treatment in accordance with the present invention include, for example, inflammatory lung diseases such as, for example, asthma, cystic fibrosis (CF), chronic obstructive pulmonary disease (COPD), bronchiectasis, hypoxia, interstitial lung disease (e.g., idiopathic pulmonary fibrosis (IPF) or sarcoidosis), and acute respiratory distress syndrome (ARDS). In particular embodiments comprising treatment of ARDS, the ARDS is associated with COVID-19. In some variations, a patient having the inflammatory lung disease is a patient that has been exposed to sulfur mustard gas (SM). In other variations, a patient having the inflammatory lung disease is a patient that has been exposed to an organophosphate, such as an insecticide (e.g., parathion, malathion, chlorpyrifos, diazinon, dichlorvos, phosmet, fenitrothion, terbufos, tetrachlorvinphos, azamethiphos, or azinphos-methyl) or other neurotoxin (e.g., tabun, sarin, soman, or cyclosarin).

Other inflammatory diseases amenable to treatment in accordance with the present invention include autoinflammatory diseases (i.e., innate immune system activation disorders characterized by seemingly unprovoked episodes of inflammation and a relative lack of obvious autoimmune pathology). Exemplary autoinflammatory diseases include inflammatory bowel disease (IBD) (e.g., Crohn's disease, ulcerative colitis), Behcet's disease, systemic onset juvenile idiopathic arthritis (JIA), gout, pseudogout, storage (Gaucher's) disorders, hereditary angioedema (HAE), atypical hemolytic uremic syndrome, familial Mediterranean fever (FMF), TNF-receptor associated periodic fever syndrome (TRAPS), cryopyrin-associated periodic syndromes (CAPS)), NOD2-associated autoinflammatory disease (NAID), and Blau syndrome.

In yet other embodiments, an inflammatory disease or disorder for treatment in accordance with the present invention is selected from rheumatoid arthritis, systemic lupus erythematosus, multiple sclerosis, type 1 diabetes, type 2 diabetes, hepatitis (e.g., non-alcoholic steatohepatitis (NASH)), ankylosing spondylitis, psoriasis, psoriatic arthritis, dermatitis (e.g., atopic dermatitis), diverticulitis, irritable bowel syndrome, and nephritis. In still other embodiments, the inflammatory disease or disorder is an inflammatory skin disease (e.g., psoriasis or atopic dermatitis).

Autoimmune diseases amenable to treatment in accordance with the present invention include, for example, rheumatoid arthritis, systemic lupus erythematosus, psoriasis, multiple sclerosis, type 1 diabetes, antiphospholipid syndrome, vasculitis, and systemic sclerosis (also known as scleroderma). In other embodiments, the autoimmune disease is selected from coeliac disease, neuritis, polymyositis, juvenile rheumatoid arthritis, psoriatic arthritis, vitiligo, Sjogren's syndrome, autoimmune pancreatitis, autoimmune hepatitis, glomerulonephritis, lupus nephritis, autoimmune vasculitis, sarcoidosis, autoimmune thyroid diseases, Hashimoto's thyroiditis, Graves disease, Wegener's granulomatosis, myasthenia gravis, Addison's disease, autoimmune uveoretinitis, pemphigus vulgaris, primary biliary cirrhosis, pernicious anemia, sympathetic opthalmia, uveitis, autoimmune hemolytic anemia, pulmonary fibrosis, chronic beryllium disease, and idiopathic pulmonary fibrosis. In some variations comprising treatment of vasculitis, the vasculitis is selected from small vessel vasculitis and medium vessel vasculitis; in other variations, the vasculitis is large vessel vasculitis.

In some embodiments of a method for treating antiphospholipid syndrome, thrombosis, or ischemic stroke, a DNase fusion molecule as described herein is administered to a patient as one of the distinct therapies of a combination therapy comprising a thrombolytic agent. In some such embodiments, the thrombolytic agent is selected from anistreplase, reteplase, streptokinase, t-PA, urokinase, tenecteplase, and rokinase.

Neurological diseases amenable to treatment in accordance with the present invention include, for example, neurodegenerative diseases characterized by inflammation in the CNS such as, e.g., multiple sclerosis, Alzheimer's disease, Parkinson's disease, or amylotrophic lateral sclerosis (ALS). In more specific variations of a method for treating multiple sclerosis (MS), the MS is spino-optical MS, primary progressive MS (PPMS), or relapsing remitting MS (RRMS). In other embodiments, a neurological disease for treatment in accordance with the present invention is a CNS infection such as, e.g., meningitis, encephalitis, or cerebral malaria. In more particular variations of a method for treating meningitis, the meningitis is a bacterial meningitis; in some such embodiments, the CNS infection is an S. pneumoniae, N. meningitis, S. aureus, E. coli, A. baumanii, S. oralis, S. capitis, or S. epidermidis infection. Other neurological diseases or disorders amenable to treatment with DNase-Fc fusion molecules as described herein include, for example, acute brain injury such as, e.g., ischemic stroke. In still other embodiments, the neurological disease is a brain cancer such as, e.g., an intracranial tumor selected from astrocytoma, anaplastic astrocytoma, glioblastoma, oligodendroglioma, anaplastic oligodendroglioma, ependymoma, primary CNS lymphoma, medulloblastoma, germ cell tumor, pineal gland neoplasm, meningioma, pituitary tumor, tumor of the nerve sheath (e.g., schwannoma), chordoma, craniopharyngioma, and a choroid plexus tumor (e.g., choroid plexus carcinoma).

Infectious diseases amenable to treatment in accordance with the present invention include, for example, bacterial infections, viral infections, fungal infections, and parasitic infections. In some embodiments comprising treatment of a parasitic infection, the infection is a Trypanosoma brucei, Leishmania, Plasmodium falciparum, or Toxoplasma gondii infection. In some embodiments comprising treatment of a bacterial infection, the infection is a Staphylococcus aureus, Streptococcus pneumoniae, or Mycobacterium tuberculosis infection. In other embodiments, the bacterial infection is a Pseudomonas aeruginosa infection. In yet other embodiments, the bacterial infection is a Borrelia burgdorferi infection (Lyme disease). In some embodiments comprising treatment of a viral infection, the infection is an influenza virus (e.g., influenza A virus) or respiratory syncytial virus (RSV) infection. In still other embodiments, the infection is a CNS infection such as, for example, meningitis (e.g., a bacterial meningitis), encephalitis, or cerebral malaria.

Metabolic diseases that may be treated in accordance with the present invention include, for example, type 2 diabetes and obesity.

Cardiovascular diseases that may be treated in accordance with the present invention include, for example, cardiovascular diseases characterized by atherosclerosis. In some embodiments, the cardiovascular disease characterized by atherosclerosis is coronary heart disease or ischemic stroke. In more particular variations comprising treatment of coronary heart disease, the coronary heart disease is characterized by acute coronary syndrome.

Liver diseases or disorders amenable to treatment in accordance with the present invention include chronic liver diseases such as, e.g., nonalcoholic fatty liver disease (NAFLD), alcohol-associated liver disease (ALD), portal hypertension, and complications following liver transplantation. In some variations comprising treatment of nonalcoholic fatty liver disease (NAFLD), the NAFLD is nonalcoholic steatohepatitis (NASH).

Fibrotic diseases or disorders amenable to treatment in accordance with the present invention include systemic sclerosis, systemic lupus erythematosus, inflammatory lung diseases, chronic liver diseases, and chronic kidney diseases. In some variations comprising treatment of an inflammatory lung disease, the fibrotic disease is cystic fibrosis, chronic obstructive pulmonary disease, interstitial lung disease (e.g., idiopathic pulmonary fibrosis or sarcoidosis), acute respiratory distress syndrome, or asthma. In some variations comprising treatment of a chronic liver disease, the fibrotic disease is nonalcoholic steatohepatitis, alcohol-associated liver disease, portal hypertension, or a complication following liver transplantation. In some variations comprising treatment of a chronic kidney disease, the fibrotic disease is lupus nephritis, IgA nephropathy, or membranous glomerulonephritis.

Cancers that may be treated in accordance with the present invention include, for example, the following: a cancer of the head and neck (e.g., a cancer of the oral cavity, orophyarynx, nasopharynx, hypopharynx, nasal cavity or paranasal sinuses, larynx, lip, or salivary gland); a lung cancer (e.g., non-small cell lung cancer, small cell carcinoma, or mesothelimia); a gastrointestinal tract cancer (e.g., colorectal cancer, gastric cancer, esophageal cancer, or anal cancer); gastrointestinal stromal tumor (GIST); pancreatic adenocarcinoma; pancreatic acinar cell carcinoma; a cancer of the small intestine; a cancer of the liver or biliary tree (e.g., liver cell adenoma, hepatocellular carcinoma, hemangiosarcoma, extrahepatic or intrahepatic cholangiosarcoma, cancer of the ampulla of vater, or gallbladder cancer); a breast cancer (e.g., metastatic breast cancer or inflammatory breast cancer); a gynecologic cancer (e.g., cervical cancer, ovarian cancer, fallopian tube cancer, peritoneal carcinoma, vaginal cancer, vulvar cancer, gestational trophoblastic neoplasia, or uterine cancer, including endometrial cancer or uterine sarcoma); a cancer of the urinary tract (e.g., prostate cancer; bladder cancer; penile cancer; urethral cancer, or kidney cancer such as, for example, renal cell carcinoma or transitional cell carcinoma, including renal pelvis and ureter); testicular cancer; a cancer of the central nervous system (CNS) such as an intracranial tumor (e.g., astrocytoma, anaplastic astrocytoma, glioblastoma, oligodendroglioma, anaplastic oligodendroglioma, ependymoma, primary CNS lymphoma, medulloblastoma, germ cell tumor, pineal gland neoplasm, meningioma, pituitary tumor, tumor of the nerve sheath (e.g., schwannoma), chordoma, craniopharyngioma, a chloroid plexus tumor (e.g., chloroid plexus carcinoma), or other intracranial tumor of neuronal or glial origin) or a tumor of the spinal cord (e.g., schwannoma, meningioma); an endocrine neoplasm (e.g., thyroid cancer such as, for example, thyroid carcinoma, medullary cancer, or thyroid lymphoma; a pancreatic endocrine tumor such as, for example, an insulinoma or glucagonoma; an adrenal carcinoma such as, for example, pheochromocytoma; a carcinoid tumor; or a parathyroid carcinoma); a skin cancer (e.g., squamous cell carcinoma; basal cell carcinoma; Kaposi's sarcoma; or a malignant melanoma such as, for example, an intraocular melanoma); a bone cancer (e.g., a bone sarcoma such as, for example, osteosarcoma, osteochondroma, or Ewing's sarcoma); multiple myeloma; a chloroma; a soft tissue sarcoma (e.g., a fibrous tumor or fibrohistiocytic tumor); a tumor of the smooth muscle or skeletal muscle; a blood or lymph vessel perivascular tumor (e.g., Kaposi's sarcoma); a synovial tumor; a mesothelial tumor; a neural tumor; a paraganglionic tumor; an extraskeletal cartilaginous or osseous tumor; and a pluripotential mesenchymal tumor. In some such embodiments, a DNase fusion molecule as described herein is administered to a cancer patient as one of the distinct therapies of a combination therapy such as, for example, a combination therapy comprising an immunomodulatory therapy (e.g., a CAR T-cell therapy (see, e.g., June et al., Science 359:1361-1365, 2018) or a therapy comprising an immune checkpoint inhibitor), a radiation therapy, or a chemotherapy.

In certain embodiments, a combination cancer therapy comprises a DNase fusion molecule as described herein and a targeted therapy such as, e.g., a therapeutic monoclonal antibody targeting a specific cell-surface or extracellular antigen, or a small molecule targeting an intracellular protein (e.g., an intracellular enzyme). Exemplary antibody targeted therapies include anti-VEGF (e.g., bevacizumab), anti-EGFR (e.g., cetuximab), anti-CTLA-4 (e.g., ipilimumab), anti-PD-1 (e.g., nivolumab), anti-PD-L1 (e.g., pembrolizumab), and anti-LAG-3 (e.g., relatlimab). Exemplary small molecule targeted therapies include proteasome inhibitors (e.g., bortezomib), tyrosine kinase inhibitors (e.g., imatinib), cyclin-dependent kinase inhibitors (e.g., seliciclib); BRAF inhibitors (e.g., vemurafenib or dabrafenib); and MEK kinase inhibitors (e.g., trametinib).

Table 2 is a non-exclusive list of approved antibodies and antibody-drug conjugates for which combination therapy with a DNase fusion molecule is possible. These targeted therapies rely at least partially on antibody-mediated cellular killing and, in some aspects, benefit from combination with DNase-Fc therapy through improved contact between cytotoxic lymphocytes and tumor cells. In other aspects, removal of extracellular DNA using DNase-Fc molecules after tumor cell killing reduces inflammation and improves the generation of anti-tumor immunity.

TABLE 2
Antibody Cancer Therapy Compounds for Use in Combination with DNase Fusion
Proteins

| Generic Name | Brand Name | Target | Company |
|---|---|---|---|
| Brentuximab Vedotin | Adcetris | CD30 drug conjugate | Seagen |
| Ofatumumab | Arzerra | CD20 | Novartis |
| Bevacizumab | Avastin | VEGF | Genentech/Roche |
| Tositumomab | Bexxar | CD20 | GalaxoSmithKline |
| Belantamab Mafodotin | Blenrep | BCMA drug conjugate | GalaxoSmithKline |
| Blinatumomab | Blincyto | CD19 X CD3 bispecific | Amgen |
| Alemtuzumab | Campath | CD52 | Genzyme/Sanofi |
| Ramucirumab | Cyramza | VEGFR2 | Imclone/Eli Lilly |
| Naxitamab | Danyelza | Ganglioside GD2 | Y-mAbs |
| Daratumumab | Darzalex | CD38 | Janssen |
| Elotuzumab | Empliciti | SLAMF7 | Bristol-Myers |
| | | | Squibb/Abbvie |
| F am-trastuzumab | Enhertu | HER2 drug conjugate | AstraZeneca |
| Cetuximab | Erbitux | EGFR | Eli Lilly/Merck KG/A |
| Trastuzumab | Herceptin | HER2 | Genentech |
| Kadcyla | Ado-trastuzumab | HER2 | Genentech |
| Olaratumab | Lartruvo | PDGFR | Eli Lilly |
| Gemtuzumab | Mylotarg | CD33 drug conjugate | Pfizer |
| Ozogamicin | | | |
| Enfortumab | Padcev | Nectin-4 | Seagen |
| Pertuzumab | Perjeta | HER2 | Genentech |
| Polatuzumab | Polivy | CD79b | Genentech |
| Necitumumab | Portrazza | EGFR | Eli Lilly |
| Rituximab | Rituxan | CD20 | Genentech/IDEC |
| Tisotumab vedotin | Tivdak | Tissue Factor drug conjugate | Seagen |
| Dinutuximab | Unituxin | Ganglioside GD2 | United Therapies |
| Panitumumab | Vectibix | EGFR | Amgen |
| Ibritumomab tiuxetan | Zevalin | CD20 isotope conjugate | Acrotech |
| Generic Name | Brand Name | Target | Company |
| Loncastuximab | Zynlonta | CD19 drug conjugate | ADC Therapeutics |
| Tesirine | | | |
| Obinutuzumab | Gazyva | CD20 | Roche |

In some cancer combination therapy variations comprising an immune checkpoint inhibitor, the combination therapy includes an anti-PD-1/PD-L1 therapy, an anti-CTLA-4 therapy, an anti-LAG3 therapy, or a combination thereof (e.g., a combination of both anti-CLTA-4 therapy and anti-PD-1/PD-Li therapy or both anti-LAG-3 therapy and anti-PD-1/PD-Li therapy). In certain aspects, DNase fusion molecules as described herein can increase the response rate to anti-CTLA-4, anti-PD-1/PD-L1, or anti-LAG-3 therapy, as well as the response rate to a combination thereof. Fusion molecules of the invention may also be useful for reducing the toxicity associated with anti-CTLA-4, anti-PD-1/PD-L1, anti-LAG-3, or a combination thereof. Table 3 is a non-exclusive list of approved checkpoint inhibitors for which combination therapy with a DNase fusion molecule is possible.

TABLE 3
Checkpoint Inhibitors for Cancer Therapy for Use in
Combination with DNase Fusion Proteins

| Generic Name | Brand Name | Target | Company |
|---|---|---|---|
| Ipilimumab | Yervoy | CTLA-4 | Bristol-Myers Squibb |
| Nivolumab | Opdivo | PD-1 | Bristol-Myers Squibb/Ono |
| Pembrolizumab | Keytruda | PD-1 | Merck Sharpe & Dohme |
| Atezolizumab | Tecentriq | PD-L1 | Genentech/Roche |
| Avelumab | Bavencio | PD-L1 | Merck KG/A and Pfizer |
| Durvalumab | Imfinzi | PD-L1 | Medimmune/Astra Zeneca |
| Cemiplimab | Libayo | PD-1 | Regeneron |
| Relatlimab | Opdualag | LAG-3 | Bristol-Myers Squibb |
| | (combination | | |
| | with Nivolumab) | | |

In certain variations, a cancer treated in accordance with the present invention is selected from malignant melanoma, renal cell carcinoma, non-small cell lung cancer, bladder cancer, and head and neck cancer. These cancers have shown responses to immune checkpoint inhibitors anti-PD-1/PD-Li and anti-CTLA-4. See Grimaldi et al., Expert Opin. Biol. Ther. 16:433-41, 2016; Gunturi et al., Curr. Treat. Options Oncol. 15:137-46, 2014; Topalian et al., Nat. Rev. Cancer 16:275-87, 2016. Thus, in some more specific variations, any of these cancers is treated with a DNase fusion molecule as described herein in combination with an anti-PD-1/PD-L1 therapy, an anti-CTLA-4 therapy, or both.

In some embodiments, a combination cancer therapy comprises a DNase fusion molecule as described herein and a chemotherapy. Chemotherapy drugs cause tumor cell death and release of DNA, resulting in inflammation. Extracellular DNA will also reduce the T cell response to tumor antigens released or exposed during tumor cell death. The ability of T cells to respond to tumor antigens depends on cellular contact of T cells with antigen presenting cells, a process that may be prevented by extracellular DNA. Therapy with DNase-Fc fusion molecules can improve clinical responses when given in combination with chemotherapy agents. Common chemotherapy compounds include, for example, methotrexate, adriamycin, 5-flurouracil, paclitaxel, busulfan, bleomycin, chlorambucil, idarubucin, hydroxyurea, gemcitabine, thalidomide, etoposide, arsenic trioxide, vinblastine, daunorubicin, vincristine, doxorubicin, procarbazine, tamoxifen, fludarabine, and prednisolone. Chemotherapy compounds that are kinase inhibitors include, for example, alectinib, brigatinib, avapritinib, erdafitinib, bosutinib, encorafenib, zanubrutinib, vandetanib, cabozantinib, cobimetinib, mobocertinib, trametinib, binimetinib, neratinib, sorafenib, pemigatinib, erlotinib, sunitinib, regorafenib, dasatinib, asciminib, dabrafenib, upadacitinib, entrectinib, ripretinib, selpercatinib, ivosidenib, infigratinib, tucatinib, pexidartinib, iapatinib, crizotinib, gilteritinib, dacomitinib, larotrectinib, gefitinib, fedratinib, ruxolitinib, selumetinib, lenvatinib, lorlatinib, ibrutinib, afatinib, pralsetinib, enasidenib, ceritinib, niraparib, and vemurafenib.

Chimeric antigen receptor (CAR) T-cell therapy can also benefit from combination with DNase-Fc fusion molecules as described herein. Two exemplary CAR T-cell therapies that are FDA approved are YESCARTA® (axicabtagene ciloleucel) by Gilead and ABECMA® (idecabtagene vicleucel) by Bristol-Myers Squibb.

In other aspects, the present invention provides methods for reducing NETosis in a subject. The method generally includes administering to a subject having NETosis an effective amount of a fusion polypeptide or dimeric protein as described herein. In some embodiments, the NETosis is associated with the presence of a disease or disorder in the subject (e.g., a disease or disorder discussed above). In other embodiments, the NETosis is associated with a risk of developing such a disease or disorder; in particular variations, treatment with the fusion molecule reduces the risk of developing the disease or disorder in the subject.

In another aspect, the present invention provides a method for protecting a subject from aging. The method generally includes administering to the subject an effective amount of a fusion polypeptide of a fusion polypeptide or dimeric protein as described herein. In some embodiments, the subject has an age-related disease or disorder (e.g., an inflammatory disease, an autoimmune disease, a neurodegenerative disease, a cardiovascular disease, or a fibrotic disease). In other embodiments, the subject is at risk of developing such an age-related disease or disorder, and treatment with the fusion molecule reduces the risk of the disease or disorder in the subject.

In certain embodiments of a method for treating a disease or disorder, reducing NETosis, or protecting from aging as above, the method is a combination therapy further comprising administering to the patient (a) an effective amount of a DNase fusion polypeptide having the formula D-L1-X as described above, or a dimeric protein formed by dimerization of the fusion polypeptide, and (b) an effective amount of a biologically active paraoxonase. In some variations, the paraoxonase has at least 80%, at least 85%, at least 90%, or at least 95% identity with the amino acid sequence shown in residues 16-355 or 26-355 of SEQ ID NO:24 and does not contain an amino-terminal leader sequence corresponding to residues 1-15 of SEQ ID NO:24. In some such embodiments, the biologically active paraoxonase has at least 90%, at least 95%, at least 96%, at least 97%, at least 98%, at least 99%, or 100% identity with the amino acid sequence shown in (i) residues 16-355 or 26-355 of SEQ ID NO:24, (ii) residues 16-355 or 26-355 of SEQ ID NO:40, or (iii) residues 16-355 or 26-355 of SEQ ID NO:42. In other, non-mutually exclusive variations, the paraoxonase is contained within a paraoxonase fusion polypeptide comprising, from an amino terminal position to a carboxyl terminal position, Xₚ-L2-P, wherein Xₚ is an immunoglobulin heavy chain constant region (e.g., an immunoglobulin Fc region as described herein for a DNase-Fc fusion), L2 is a polypeptide linker (e.g., an L2 linker as described herein for a bispecific DNase-PON fusion), wherein L2 is optionally present, and P is the paraoxonase. In some such embodiments, the paraoxonase fusion polypeptide comprises an amino acid sequence having at least 90%, at least 95%, at least 96%, at least 97%, at least 98%, at least 99%, or 100% identity with the amino acid sequence shown in (i) residues 21-613 of SEQ ID NO:61, (ii) residues 21-613 of SEQ ID NO:62, (iii) residues 21-613 of SEQ ID NO:63, (iv) residues 21-610 of SEQ ID NO:64, (v) residues 21-610 of SEQ ID NO:65, or (vi) residues 21-610 of SEQ ID NO:66.

In particular variations comprising treatment with either a bispecific DNase-Fc-PON fusion or a combination of a DNase-Fc fusion and a paraoxonase as described herein, the disease or disorder to be treated is selected from an inflammatory lung disease (e.g., cystis fibrosis, interstitial lung disease (e.g., idiopathic pulmonary fibrosis or sarcoidosis), acute respiratory distress syndrome, chronic obstructive pulmonary disease, asthma, exposure to sulfur mustard gas, or exposure to an organophosphate), biofilm formation by a gram-negative bacteria (e.g., Pseudomonas aeruginosa), sepsis, an autoimmune disease (e.g., rheumatoid arthritis, vasculitis, systemic sclerosis, or systemic lupus erythematosus (e.g., systemic lupus erythematosus with lupus nephritis)), an autoinflammatory disease (e.g., inflammatory bowel disease), a cardiovascular disease (e.g., coronary heart disease or ischemic stroke), a chronic liver disease (e.g., nonalchoholic steatohepatitis), an inflammatory skin disease (e.g., psoriasis or atopic dermatitis), and a fibrotic disease (e.g., systemic sclerosis, lupus nephritis, an inflammatory lung disease, or a chronic liver disease). The combination of DNase and paraoxonase (using either a bispecific fusion or a combination therapy with monospecific molecules) is particularly effective in these exemplary disease conditions. For example, DNase and PON1 each contributes to inhibition of biofilm formation by Pseudomonas aeruginosa in inflammatory lung disease such as, e.g., cystic fibrosis. Also, in addition to the targeting of NETs by DNase-Fc, therapy with a paraoxonase (e.g., Fc-PON1) will counteract the oxidative stress from myeloperoxidase (MPO) release, an important pathological process in many inflammatory, autoimmune, and fibrotic disease conditions. The combination may also be beneficial in patients recovering from cancer therapy, including radiation and chemotherapy.

For therapeutic use, a fusion polypeptide or dimeric protein as described herein is delivered in a manner consistent with conventional methodologies associated with management of the disease or disorder for which treatment is sought. In accordance with the disclosure herein, an effective amount of the fusion polypeptide or dimeric protein is administered to a subject in need of such treatment for a time and under conditions sufficient to prevent or treat the disease or disorder.

Subjects for administration of fusion polypeptides or dimeric proteins as described herein include patients at high risk for developing a particular disease or disorder as well as patients presenting with an existing disease or disorder. In certain embodiments, the subject has been diagnosed as having the disease or disorder for which treatment is sought. Further, subjects can be monitored during the course of treatment for any change in the disease or disorder (e.g., for an increase or decrease in clinical symptoms of the disease or disorder). Also, in some variations, the subject does not suffer from another disease or disorder requiring treatment that involves administration of an DNase protein.

In prophylactic applications, pharmaceutical compositions or medicants are administered to a patient susceptible to, or otherwise at risk of, a particular disease in an amount sufficient to eliminate or reduce the risk or delay the onset of the disease. In therapeutic applications, compositions or medicants are administered to a patient suspected of, or already suffering from such a disease in an amount sufficient to cure, or at least partially arrest, the symptoms of the disease and its complications. An amount adequate to accomplish this is referred to as a therapeutically or pharmaceutically effective dose or amount. In both prophylactic and therapeutic regimes, agents are usually administered in several dosages until a sufficient response (e.g., enhanced alveolar fluid clearance in inflammatory lung disease) has been achieved. Typically, the response is monitored and repeated dosages are given if the desired response starts to fade.

To identify subject patients for treatment according to the methods of the invention, accepted screening methods may be employed to determine risk factors associated with a specific disease or to determine the status of an existing disease identified in a subject. Such methods can include, for example, determining whether an individual has relatives who have been diagnosed with a particular disease. Screening methods can also include, for example, conventional work-ups to determine familial status for a particular disease known to have a heritable component. Toward this end, nucleotide probes can be routinely employed to identify individuals carrying genetic markers associated with a particular disease of interest. In addition, a wide variety of immunological methods are known in the art that are useful to identify markers for specific diseases. Screening may be implemented as indicated by known patient symptomology, age factors, related risk factors, etc. These methods allow the clinician to routinely select patients in need of the methods described herein for treatment. In accordance with these methods, treatment using a fusion polypeptide or dimeric protein of the present invention may be implemented as an independent treatment program or as a follow-up, adjunct, or coordinate treatment regimen to other treatments.

For treatment of a disease or disorder characterized by NETosis, subject patients likely to benefit from NET-targeted therapy may be identified, for example, by measuring NETs in candidate patients. There are multiple ways to measure NETs in vivo (for a review, see, e.g., Masuda et al., Clin. Chim. Acta 459:89-93, 2016). One method uses flow cytometry to measure cell-associated DNA by staining with a non-cell permeable DNA dye (Zharkova et al., Cytometry A 95:268-278, 2019). Other methods include measuring myeloperoxidase activity associated with DNA or measuring citrullinated histone H3 (see, e.g., Matsuda et al., supra). Yet another method measures NETosis by incubating blood neutrophils with autologous plasma (Abrams et al., Am. J. Resp. Crit. Care Med. 200:869, 2019). This method was able to predict disseminated intravascular coagulation in critically ill patients (id.).

For administration, a fusion polypeptide or dimeric protein in accordance with the present invention is formulated as a pharmaceutical composition. A pharmaceutical composition comprising a fusion polypeptide or dimeric protein as described herein can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, e.g., Gennaro (ed.), Remington's Pharmaceutical Sciences (Mack Publishing Company, 19^{th} ed. 1995). Formulations may further include one or more excipients, preservatives, solubilizers, buffering agents, albumin to prevent protein loss on vial surfaces, etc.

A pharmaceutical composition comprising a fusion polypeptide or dimeric protein of the present invention is administered to a subject in an effective amount. The fusion polypeptide or dimeric protein may be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, the fusion polypeptide or dimeric protein may be administered to a subject in a single bolus delivery, via continuous delivery (e.g., continuous transdermal delivery) over an extended time period, or in a repeated administration protocol (e.g., on an hourly, daily, or weekly basis).

In some embodiments, a pharmaceutical composition comprising a fusion molecule or dimeric protein of the present invention is formulated for delivery to the lung by nebulization. Previous studies of pulmonary delivery of Fc fusion proteins, including erythropoietin-Fe, interferon Betala-Fe, and FSH-Fc, have shown that the immunoglobulin transport FcRn pathway is active in the lungs and provides 20% to 50% bioavilability (see Bitonti et al., Proc. Natl. Acad. Sci. USA 101:9763-9768, 2004; Bitonti and Dumont, Adv. Drug Deliv. Rev. 58:1106, 2006; Valle et al., J. Interferon Cytokine Res. 32:178-184, 2012). Pulmonary delivery of DNase-Fc fusion molecules as described herein could act both locally and in circulation and peripheral organs. In certain embodiments wherein the treatment is a combination therapy with a paraoxonase as described herein, both the DNase-Fc fusion molecule and the paraoxonase (e.g., an Fc-PON1 fusion as described herein) are delivered by a nebulizer. In some variations comprising delivery to the lung by a nebulizer, the disease or disorder to be treated is selected from an inflammatory lung disease (e.g., cystis fibrosis, interstitial lung disease (e.g., idiopathic pulmonary fibrosis or sarcoidosis), acute respiratory distress syndrome (ARDS), chronic obstructive pulmonary disease (COPD), asthma, exposure to sulfur mustard gas, or exposure to an organophosphate), biofilm formation by a gram-negative bacteria (e.g., Pseudomonas aeruginosa), and sepsis. In other, non-mutually exclusive variations, an FcRn-binding fusion molecule for delivery to the lung by nebulization (e.g., a DNase1-Fc fusion as described herein) comprises an Fe variant with increased FcRn-binding affinity relative to the corresponding wild-type Fe, thereby increasing fusion molecule half-life and increasing concentration in the blood after inhalation with a nebulizer.

Multiple biologics have been successfully formulated to retain biologic activity after nebulization (see, e.g., Hertel et al., Adv. Drug Deliv. Rev. 93:79-94, 2015). Formulations for delivery of a DNase-Fc fusion molecule of the present invention may include an excipient suitable for pulmonary delivery such as, e.g., Polysorbate 80 (PS80) or Polysorbate 20 (PS20), surfactants that are included in many biopharmaceutical formulations. Such stabilizing excipients protect proteins from degradation at the air-liquid interface when applied above their critical micelle concentration (for example, PS80 above 0.01% was effective in stabilizing G-CSF, LDH, rhConIFN, and t-Pa; and PS20 applied at 0.04% was effective for protection of Fc-gamma RIIb). Another suitable stabilizing excipient is HP-beta-cyclodextrin (HP-beta-CD) applied at, e.g., 0.35% or above (see Hertel et al., supra). In some variations, a stabilizing excipient is not required for nebulized delivery of a DNase-Fc fusion molecule as described herein (for example, wild-type human DNase 1 (Pulmozyme®) has been shown to not aggregate and remain stable after nebulization with either jet or vibrating mesh (VM) nebulizers without requiring excipients, see Cipolla et al., Pharm. Res. 11:491-498, 1994; Scherer et al., J. Pharm. Sci. 100:98-109, 2011).

Determination of effective dosages is typically based on animal model studies followed up by human clinical trials and is guided by determining effective dosages and administration protocols that significantly reduce the occurrence or severity of the subject disease or disorder in model subjects. Effective doses of the compositions of the present invention vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy. Accordingly, a therapeutically or prophylactically effective amount is also one in which any undesired collateral effects are outweighed by beneficial effects (e.g., in the case of treatment of inflammatory lung disease, where any undesired collateral effects are outweighed by any beneficial effects such as, for example, improved alveolar fluid clearance, improved lung physiology and function, etc.). For administration of a fusion polypeptide or dimeric protein of the present invention, a dosage typically ranges from about 0.1 μg to 100 mg/kg or 1 μg/kg to about 50 mg/kg, and more usually 10 μg to 5 mg/kg of the subject's body weight. In more specific embodiments, an effective amount of the agent is between about 1 μg/kg and about 20 mg/kg, between about 10 μg/kg and about 10 mg/kg, or between about 0.1 mg/kg and about 5 mg/kg. Dosages within this range can be achieved by single or multiple administrations, including, e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bi-monthly intervals. Alternatively, administrations can be on an irregular basis as indicated by monitoring of clinical symptoms of the disease or disorder and/or monitoring of disease biomarkers or other disease correlates.

In some specific embodiments comprising delivery by inhalation with a nebulizer (for example, for treatment of an inflammatory lung disease such as, e.g., cystic fibrosis or interstitial lung disease), a fusion polypeptide or dimeric protein of the present invention is administered to the lung by a nebulizer at a dose of from about 1 mg to about 5 mg or from about 2 mg to about 4 mg per day. In other specific embodiments comprising systemic administration (for example, for treatment of systemic lupus erythematosus, lupus nephritis, rheumatoid arthritis, or inflammatory bowel disease), a fusion polypeptide or dimeric protein of the present invention is administered (e.g., by intravenous or subcutaneous injection) at a dose of from about 50 mg to about 1,000 mg, from about 50 mg to about 800 mg, from about 50 mg to about 500 mg, from about 100 mg to about 500 mg, from about 100 mg to about 400 mg, from about 100 mg to about 300 mg, from about 150 mg to about 250 mg, or about 200 mg every month to six weeks. For combination therapy with a paraoxonase as described herein, each of the DNase-Fc fusion molecule and the paraoxonase (e.g., an Fc-PON1 fusion as described herein) may be administered at these doses.

Particularly suitable animal models for evaluating efficacy of a DNase composition of the present invention for treatment of inflammatory lung disease include, for example, a murine ovalbumin-induced acute asthma model as described by da Cunha et al. (Exp. Lung Res. 42:66, 2016) (showing significantly reduced airway resistance with wild-type (wt) DNase1 treatment), a murine silica-induced lung inflammation model as described by Benmerzoug et al. (Nat. Comm. 9:5226, 2018) (showing prevention of DNA-mediated STING activation and blockade of the downstream type I IFN response with wt DNase1 treatment), and a murine model of transfusion-related acute lung injury as described by Caudrillier et al. (J. Clin. Invest. 122:2661, 2012) (showing protection from lung edema and lung vascular permeability as well as reduced NET formation and platelet sequestration in the lung with wt DNase1 treatment).

Suitable animal models for evaluating efficacy of a fusion composition (e.g., a DNase-Fc-PON1 fusion) as described herein for treatment of exposure to sulfur mustard gas or an organophosphate include, for example, a guinea pig model as describe by Valiyaveettil et al. (Biochem. Pharmocol. 81:800-809, 2011; Toxicol. Letters 202:203-208, 2011) (showing protection from sarin and soman inhalation toxicity with recombinant human PON1 injection) and mouse models as described by Bajaj et al. (Appl. Biochem. Biotechnol. 180:165-176, 2016) and Stevens et al. (Proc. Natl. Acad. Sci. USA 105:12780-12784, 2008) (showing protection from organophosphate poisoning using a recombinant Q192K variant of PON1).

A particularly suitable animal model for evaluating efficacy of a DNase composition as described herein for treatment of an inflammatory bowel disease is a dextran sulfate sodium (DSS)-induced colitis model in mice. See, e.g., Babicova et al., Folia Biolica (Praha) 64:10, 2018 (showing reduction in TNFα and myeloperoxidase in the colon with wt DNase1 treatment of DSS-induced colitis); Li et al., J. Crohns Colitis 2020, 14:240-253, 2020 (showing decreased cytokine production and attenuated accelerated thrombus formation and platelet activation with DNase1 treatment of DSS-induced colitis). Another suitable model is a TNBS-induced colitis model and a chronic colitis model with CD4⁺CD45RBhi^{gh} cell transfer in mice. See, e.g., Yamashita et al., J. Immunol. 191:949-960, 2013 (showing efficacy of PON1 therapy in the TNBS-induced colitis model and a PON1 variant (G3C9) in the chronic colitis model).

Also known is the collagen-induced arthritis (CIA) model for rheumatoid arthritis (RA) (see, e.g., Brand et al., Nat. Protoc. 2:1269-1275, 2007). CIA shares similar immunological and pathological features with RA, making it an ideal model for evaluating efficacy of DNase compositions. Another suitable model for RA is PG-PS (proteoglycan-polysaccharide)-induced arthritis in Lewis rats (see, e.g., Esser et al., Arthritis and Rheumatism 28:1402-1411, 1985; Brooks et al., Proc. Intl. Soc. Mag. Reason. Med. 11:1526, 2003).

Suitable animal models for multiple sclerosis (MS) include, for example, experimental allergic encephalomyelitis (EAE) models that rely on the induction of an autoimmune response in the CNS by immunization with a CNS antigen (also referred to as an “encephalitogen” in the context of EAE), which leads to inflammation, demyelination, and weakness (see, e.g., Constantinescu et al., British Journal of Pharmacology 164:1079-1106, 2011).

Other known animal models for evaluating efficacy of DNase1 fusion molecules of the present invention include, e.g., a hind-limb ischemia reperfusion model as described by Albadawi et al. (J. Vasc. Surg. 64:484, 2016) (showing increased perfusion, decreased infiltrating inflammatory cells, and reduction of a local thrombosis marker with wt DNase1 therapy), a rat model of ischemia-reperfusion-induced acute kidney injury as described by Peer et al. (Am. J. Nephrol. 43:195, 2016) (showing renoprotective effects with wt DNase1 therapy), a cecal ligation and puncture sepsis model in mice as described by Mai et al. (Shock 44:166, 2015) (showing prevention of organ damage and protection from death with wt DNase l therapy), and a model of inferior vena cava stenosis as described by Brill et al. (J. Thrombosis Haemostasis 10:136, 2012) (showing prevention of thrombosis with wt DNase1 therapy).

Fusion molecules of the present invention can also be evaluated for anti-tumor activity in animal tumor models. For example, efficacy of DNase-Fc treatment in reducing tumor metastasis associated with NET formation can be evaluated in mouse models as described by, e.g., Cools-Lartigue et al. (J. Clin. Invest. 123:3446, 2013) (showing reduction in metastasis of injected tumor (lung carcinoma) cells with systemic administration of wt DNase1 in a model of severe postoperative infection) and Park et al. (Sci. Translational Med. 8:361ra138, 2016) (showing reduction in metastasis of breast cancer cells to the lung with systemic administration of wt DNase1-coated nanoparticles). Also known is a model utilizing nude mice subcutaneously grafted with the human colon cancer cell line SW480 as described, e.g., by Trejo-Becirril et al. (Integrative Cancer Therapies 15:NP35-NP43, 2016) (showing inhibition of tumor growth with a combination of DNase1 and proteases).

Another known animal tumor model is B16 melanoma, a poorly immunogenic tumor. Multiple models of tumor immunotherapy have been studied. See Ngiow et al., Adv. Immunol. 130:1-24, 2016. The B16 melanoma model has been studied extensively with checkpoint inhibitors anti-CTLA-4, anti-PD-1, and the combination thereof. Anti-CTLA-4 alone has a potent therapeutic effect in this model only when combined with GM-CSF transduced tumor vaccine, or combined with anti-PD-1. See Weber, Semin. Oncol. 37:430-439, 2010; Ai et al., Cancer Immunol. Immunother. 64:885-92, 2015; Haanen et al., Prog. Tumor Res. 42:55-66, 2015. Efficacy of a DNase fusion molecule for treatment of malignant melanoma is shown, for example, by slowed tumor growth following administration to B16 melanoma mice that have formed palpable subcutaneous tumor nodules. Efficacy of a DNase fusion molecule can be evaluated in B16 melanoma mice either alone or, alternatively, in combination with another anti-cancer therapy (e.g., anti-CTLA-4, with or without tumor vaccine or with or without anti-PD-1/PD-L1). For example, tumor rejection in B16 melanoma mice using a combination of a DNase fusion molecule as described herein and anti-CTLA-4, in the absence of tumor vaccine, demonstrates an enhanced response to anti-CTLA-4 using the DNase therapy. In exemplary studies to evaluate DNase fusion molecules comprising human protein sequences, which are functionally active in mice but are expected to be immunogenic in these models (and thereby likely to result in formation of neutralizing antibodies after 7-10 days), mice may be administered a fusion molecule of the present invention for a short period (for example, one week, administered in, e.g., two doses of about 40 mg/kg three days apart), and tumor growth then monitored, typically for two to three weeks after injection with the fusion molecule.

Dosage of the pharmaceutical composition may be varied by the attending clinician to maintain a desired concentration at a target site. For example, if an intravenous mode of delivery is selected, local concentration of the agent in the bloodstream at the target tissue may be between about 1-50 nanomoles of the composition per liter, sometimes between about 1.0 nanomole per liter and 10, 15, or 25 nanomoles per liter depending on the subject's status and projected measured response. Higher or lower concentrations may be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar. Dosing may also vary, e.g., depending on the activity of the DNase fusion molecule being administered. For example, if the DNase-Fc fusion is not actin-resistant (i.e., a fusion in which the DNase does not contain any amino acid substitution, relative to human DNase1, that decreases G-actin-induced inhibition of endonuclease activity of the DNase relative to human DNase1), inhibition by actin may be overcome by increasing the dose relative to a dose that is effective for a similar but actin-resistant DNase-Fc fusion molecule.

A pharmaceutical composition comprising a fusion polypeptide or dimeric protein as described herein can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants. See, e.g., Bremer et al., Pharm. Biotechnol. 10:239, 1997; Ranade, “Implants in Drug Delivery,” in Drug Delivery Systems 95-123 (Ranade and Hollinger, eds., CRC Press 1995); Bremer et al., “Protein Delivery with Infusion Pumps,” in Protein Delivery: Physical Systems 239-254 (Sanders and Hendren, eds., Plenum Press 1997); Yewey et al., “Delivery of Proteins from a Controlled Release Injectable Implant,” in Protein Delivery: Physical Systems 93-117 (Sanders and Hendren, eds., Plenum Press 1997). Other solid forms include creams, pastes, other topological applications, and the like.

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer. See, e.g., Gombotz and Pettit, Bioconjugate Chem. 6:332, 1995; Ranade, “Role of Polymers in Drug Delivery,” in Drug Delivery Systems 51-93 (Ranade and Hollinger, eds., CRC Press 1995); Roskos and Maskiewicz, “Degradable Controlled Release Systems Useful for Protein Delivery,” in Protein Delivery: Physical Systems 45-92 (Sanders and Hendren, eds., Plenum Press 1997); Bartus et al., Science 281:1161, 1998; Putney and Burke, Nature Biotechnology 16:153, 1998; Putney, Curr. Opin. Chem. Biol. 2:548, 1998. Polyethylene glycol (PEG)-coated nanospheres can also provide carriers for intravenous administration of therapeutic proteins. See, e.g., Gref et al., Pharm. Biotechnol. 10:167, 1997.

Other dosage forms can be devised by those skilled in the art, as shown by, e.g., Ansel and Popovich, Pharmaceutical Dosage Forms and Drug Delivery Systems (Lea & Febiger, 5th ed. 1990); Gennaro (ed.), Remington's Pharmaceutical Sciences (Mack Publishing Company, 19th ed. 1995), and Ranade and Hollinger, Drug Delivery Systems (CRC Press 1996).

In some embodiments comprising systemic administration of a fusion polypeptide or dimeric protein as described herein, the fusion molecule is formulated in a buffered saline (for example, saline buffered with 2 mM carbonate, pH 7.5, plus 1 mM calcium chloride).

Pharmaceutical compositions as described herein may also be used in the context of combination therapy. For example, for combination therapy with a paraoxonase as described herein, each of the DNase-Fc fusion molecule and the paraoxonase (e.g., an Fc-PON1 fusion as described herein) may be formulated in a buffered saline (for example, saline buffered with 2 mM carbonate, pH 7.5, plus 1 mM calcium chloride), either separately or as a mixture.

Pharmaceutical compositions may be supplied as a kit comprising a container that comprises a fusion polypeptide or dimeric protein as described herein. A therapeutic molecule can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a therapeutic protein. Such a kit may further comprise written information on indications and usage of the pharmaceutical composition.

The invention is further illustrated by the following non-limiting examples.

### Example 1: Construction and Expression of DNase1-Fc and DNase1-Fc-PON1 Fusion Proteins

Constructs were designed and synthesized and cassettes with the correct sequences were combined to generate synthetic fusion genes encoding unique DNase1 fusion molecules. Each construct included a variant of wild-type human DNase1 (nucleotide and encoded amino acid sequences as shown in SEQ ID NOs:1 and 2; see also GenBank accession number NM_005223; the amino acid sequence of the mature wild-type human DNase1, corresponding to residues 23-282 of SEQ ID NO:2, is also shown in SEQ ID NO:30). The DNase1 variant contained amino acid substitutions, relative to the mature wild-type human sequence, at positions N74 (N74K), G105 (G105R), and A114 (A114F); the variant DNase1 nucleotide and encoded amino acid sequences corresponding to the mature protein are shown in residues 61-840 of SEQ ID NO:3 and residues 21-280 of SEQ ID NO:4, respectively). The signal peptide sequence utilized was the human VK3 leader peptide (nucleotide and amino acid sequences as shown in SEQ ID NO:27 and SEQ ID NO:28), an efficient heterologous leader peptide that drives high level expression and secretion of fusion proteins. The DNase1 cassette was fused to the N-terminus of a human IgG1 Fe variant (SSS hinge, P238S, P331S) using a peptide linker, either a (Gly4Ser)4 linker ((g4s)4; nucleotide and amino acid sequences as shown in SEQ ID NO:7 and SEQ ID NO:8) or a (Gly4Ser)6 linker ((g4s)6; nucleotide and amino acid sequences as shown in SEQ ID NO:9 and SEQ ID NO:10). The Fe variant nucleotide and amino acid sequences are shown in SEQ ID NO:13 and SEQ ID NO:14. The completed DNase1-Fc constructs generated are as follows:
[hVK3LP]-[hDNase1 N74K-G105R-A114F]-(g4s)4-[SSShinge-P238S-P331S Fc] (also referred to hereinbelow as hDNaseTM-(g4s)4-Fc or DNase™-(g4s)4-Fc; nucleotide and amino acid sequences as shown in SEQ ID NO:17 and SEQ ID NO:18); and
[hVK3LP]-[hDNase1 N74K-G105R-A114F]-(g4s)6-[SSShinge-P238S-P331S Fc] (also referred to hereinbelow as hDNaseTM-(g4s)6-Fc or DNase™-(g4s)6-Fc; nucleotide and amino acid sequences as shown in SEQ ID NO:19 and SEQ ID NO:20).

Bispecific constructs further comprising a functional PON1 enzyme were also generated by fusing the carboxyl end of hDNaseTM-(g4s)4-Fc or hDNaseTM-(g4s)6-Fc to a peptide linker containing an N-linked glycosylation site (NGS; nucleotide and amino acid sequences as shown in SEQ ID NO:31 and SEQ ID NO:32) followed by a variant of human PON1 (PON1 Q192K; nucleotide and amino acid sequences as shown in SEQ ID NO:23 and SEQ ID NO:24) in which the first 15 amino acids of the uncleaved leader peptide were deleted. The completed DNase-Fc-PON1 constructs are as follows:
[Human VK3LP]-[hDNase1 N74K-G105R-A114F]-(g4s)4-[SSShinge-P238S-P331S Fe]-NGS-[PON1 Q192K], (nucleotide and amino acid sequences as shown in SEQ ID NO:25 and SEQ ID NO:26) (also referred to hereinbelow as hDNaseTM-(g4s)4-Fc-PON1-Q192K, hDNaseTM-(g4s)4-Fc-PON1-K, or DNase™-(g4s)4-Fc-P1-K); and
[Human VK3LP]-[hDNase1 N974K-G105R-A114F]-(g4s)6-[SSShinge-P238S-P331S Fe]-NGS-[PON1 Q192K], (nucleotide and amino acid sequences as shown in SEQ ID NO:47 and SEQ ID NO:48).

### Transient Expression of DNase1-Fc and DNase1 -Fc-PON] Fusion Proteins

All fusion genes were assembled in a pUC based vector, then inserted into a multiple cloning site of the mammalian expression vector pDG, a pcDNA3 plasmid derivative containing a CMV promoter to drive expression of the fusion gene. Plasmid DNA was prepared using QIAGEN (Germantown, MD) mini or maxiprep plasmid DNA kits. Purified plasmid DNA was transfected into HEK293 cells plated at approximately 50-75% confluence, using Polyfect (QIAGEN, Germantown, MD) transfection reagent according to the manufacturer's instructions. Culture media was changed to DMEM Fluorobrite™ (Life Technologies, Carlsbad, CA) serum free media on the day after transfections, and transfected cells incubated for an additional 48 hours prior to harvest of culture supernatants.

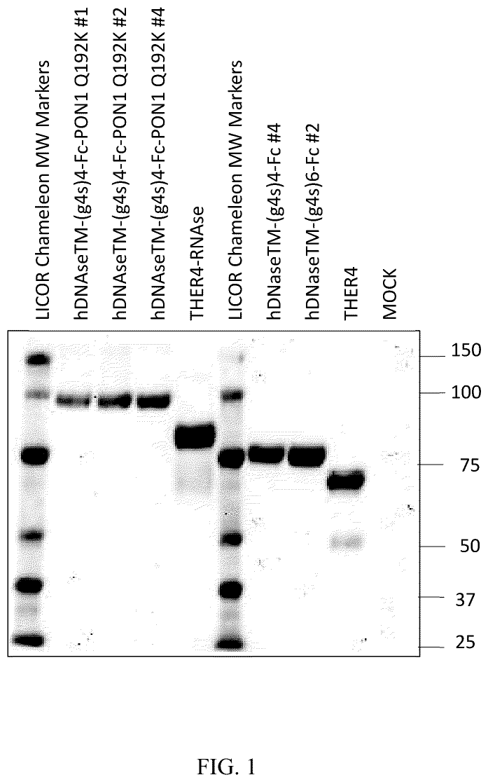

FIG. 1 shows the results of Western blot analysis of fusion protein expression from a representative set of HEK293 transient transfections. Culture supernatants were loaded directly for gel electrophoresis as follows: supernatants were collected, sample volume 4X reducing LDS sample buffer added (Life Technologies, Carlsbad, CA), samples heated for 10 minutes at 72° C., the samples centrifuged for 1 minute at 3000 rpm in a microfuge, and 30 μl sample in loading buffer (one half) from each sample was loaded onto 4-12% Bis-Tris NuPAGE gels (Life Technologies, Carlsbad, CA). Protein molecular weight markers were included on each gel (Chameleon® DUO molecular weight markers; LI-COR Biosciences, Lincoln, NE). SDS-PAGE gels were run in NuPAGE MOPS running buffer (Life Technologies, Carlsbad, CA) under reducing conditions at 185 volts for approximately 1-1.5 hours. Gels were blotted to nitrocellulose membranes using the XCell blot module (Life Technologies, Carlsbad, CA) and NUPAGE-MOPS (Life Technologies, Carlsbad, CA) transfer buffer containing 10% methanol. Blots were blocked in LI-COR Odyssey Intercept™ blocking buffer, followed by incubation with diluted goat anti-human IgG (Fc specific) secondary antibodies (Jackson Immunoresearch, West Grove, PA). Secondary antibody was conjugated with Alexafluor 790 near IR dye (LI-COR Odyssey detection) and diluted in blocking buffer 1:25,000 at 4° C. and rocked overnight. Blots were washed four times with Tris buffered saline (TBS) containing 0.1% Tween 20. Blots were then rinsed in TBS buffer and scanned with a LICOR Odyssey™ scanner. Transfection samples were loaded as follows: Lane 1 - LICOR Chameleon™ molecular weight markers; Lane 2, Lane 3, Lane 4 -transfection supernatants from three different individual clones (#1, #2, #4) of hDNaseTM-(g4s)4-Fc-PON1-Ql92K; Lane 5 - THER4-RNase (control; apoA1-(g4s)4-SSShinge-P238S-P331S-Fc-NGS-RNase; see US Patent Application Publication No. 2018/0201664); Lane 6 - LICOR Chameleon MW Markers; Lane 7 - hDNaseTM-(g4s)4-Fc #4; Lane 8 - hDNaseTM-(g4s)6-Fc #2; Lane 9 - THER4 (control; apoA1-(g4s)4-SSShinge-P238S-P331S-Fc; see International PCT Publication No. WO 2017/044424); Lane 10 - mock transfection. The approximate molecular weight (kDa) of each marker band is indicated on the right side of the Western blot.

### Stable Expression of DNase1-Fc and DNase1-Fc-PON] Fusion Proteins

Stable production of the DNase1 fusion proteins was achieved by electroporation of a selectable, amplifiable plasmid, pDG, containing the DNase1 fusion molecule cDNAs under the control of the CMV promoter, into Chinese Hamster Ovary (CHO) DG44 cells. The pDG vector is a modified version of pcDNA3 encoding the DHFR selectable marker with an attenuated promoter to increase selection pressure for the plasmid. Plasmid DNA (200 μg) was prepared using QIAGEN HISPEED maxiprep kits. Purified plasmid was linearized at a unique AscI site (New England Biolabs, Ipswich, MA Catalog #R0558), purified by phenol extraction (Sigma-Aldrich, St Louis, MO), ethanol precipitated, washed, and resuspended in tissue culture media, Excell 302 (Catalog #14324, SAFC/Sigma Aldrich, St Louis, MO). Salmon sperm DNA (Sigma-Aldrich, St. Louis, MO) was added as carrier DNA just prior to phenol extraction and ethanol precipitation. Plasmid and carrier DNA were coprecipitated, and the 400 μg was used to transfect 2×10⁷ CHO DG44 cells by electroporation.

For electroporation, cells were grown to logarithmic phase in Excell 302 media (Catalog #13424C, SAFC Biosciences/Sigma-Aldrich, St. Louis, MO) containing glutamine (4 mM), pyruvate, recombinant insulin (1 μg/ml), penicillin-streptomycin, and 2xDMEM nonessential amino acids (all from Life Technologies, Grand Island, NY), hereinafter referred to as “Excell 302 complete” media. Media for untransfected cells and cells to be transfected also contained HT (diluted from a 100x solution of hypoxanthine and thymidine) (Invitrogen/Life Technologies, Grand Island, NY). Electroporations were performed at 280 volts, 950 microFarads, using a BioRad (Hercules, CA) GenePulser electroporation unit with capacitance extender. Electroporation was performed in 0.4 cm gap sterile, disposable cuvettes. Electroporated cells were incubated for 5 minutes after electroporation prior to transfer of the treated cells to non-selective Excell 302 complete media in T75 flasks. Transfected cells were incubated overnight at 37° C., 5% CO2 in non-selective media to permit recovery, prior to selective plating in 96-well flat-bottom plates (Costar) at varying serial dilutions ranging from 250 cells/well (2500 cells/ml) to 2000 cells/well (20,000 cells/ml). Culture media for cell cloning was Excell 302 complete containing 50 nM methotrexate. Transfection plates were fed at five-day intervals with 80 μl fresh media. After the first couple of feedings, 100 μl media was removed and replaced with fresh media. Plates were monitored and individual wells with clones were fed until clonal outgrowth became close to confluent, after which clones were expanded into 24-well dishes containing 1 ml media/well. Aliquots of the culture supernatants from the original 96-well plate were harvested to a second 96-well plate prior to transfer and expansion of the cells in the 24-well plates. This second plate was frozen until performing dilutions for ELISA analysis to estimate IgG concentrations.

Screening Culture Supernatants for Production Levels of Recombinant Fusion Proteins: Once clonal outgrowth of initial transfectants was sufficient, serial dilutions of culture supernatants from master wells were thawed and the dilutions screened for expression of DNase1 fusion protein by use of an -IgG sandwich ELISA. Briefly, NUNC Maxisorp plates were coated overnight at 4° C. with 2 microgram/ml F(ab′2) goat anti-human IgG (Jackson Immunoresearch, West Grove, PA; Catalog #109-006-098) in PBS. Plates were blocked in PBS/3% BSA, and serial dilutions of culture supernatants incubated at room temperature for 2-3 hours or overnight at 4° C. Plates were washed three times in PBS/0.05% Tween 20, and incubated with horseradish-peroxidase-conjugated F(ab′2) goat anti-human IgG (Jackson Immunoresearch, West Grove, PA, Catalog #109-036-098) at 1:7500-1:10,000 in PBS/0.5% BSA, for 1-2 hours at room temperature. Plates were washed five times in PBS/0.05% Tween 20, and binding detected with SureBlue Reserve™ TMB substrate (KPL Labs/SeraCare, Gaithersburg, MD; catalog #53-00-02). Reactions were stopped by addition of equal volume of 1N HCl, and absorbance per well on each plate was read at 450 nM on a VarioSkan LUX plate reader (ThermoFisher Scientific, Waltham, MA). Concentrations were estimated by comparing the OD450 of the dilutions of culture supernatants to a standard curve generated using serial dilutions of a known standard, a protein-A-purified human -IgG fusion protein with the identical -Ig tail to clones described in this disclosure, typically THER4 (apoAl-(g4s)4-SSShinge-P238S-P331S Fc; see International PCT Publication No. WO 2017/044424) or an scFv-Fc targeted to human CD180 (G28-8). Data was collected and analyzed using platereader software and Microsoft Office Excel or GraphPad Prism 8.4.3 (San Diego, CA). The best expressing DNase-lnk-Fc clones expressed in the range of 25-55 μg/ml, while the best expressing DNase-lnk-Fc-PON1-K clones expressed at approximately 10-30 μg/ml.

Culture supernatants were collected from spent CHO cell cultures expressing the fusion proteins, filtered through 0.2 m PES express filters (Nalgene, Rochester, NY) and subjected to affinity chromatography using slow rotation of culture supernatants with Protein A-agarose (IPA 300 crosslinked agarose) slurry in 50 ml sterile, conical centrifuge tubes at 4° C. (Repligen, Waltham, MA). Fusion protein bound to protein A agarose was recovered by centrifugation, and culture supernatants removed, replaced, and the incubation process repeated until the desired volume of supernatant was processed. The final protein A agarose slurry was then loaded into sterile, acid-washed econocolumns (BioRad, Hercules, CA) to wash the resin. Columns were then washed with several column volumes of column wash buffer (Gentle Ag-Ab binding buffer, Pierce/ThermoFisher, Waltham, MA) to remove any residual culture supernatant, prior to elution. Bound protein was then eluted from the resin using gentle Ag/Ab elution buffer (Pierce/ThermoFisher, Waltham, MA). Fractions (0.8-1.0 ml) were collected and protein concentration of aliquots (2 μl) from each fraction were determined at 280 nM using a Nanodrop (Wilmington DE) microsample spectrophotometer, with blank determination using elution buffer alone. Fractions containing fusion protein were pooled, and buffer exchange was performed by dialysis using Spectrum Laboratories G2 (Ranch Dominguez, CA, Catalog #G235057, Fisher Scientific catalog #08-607-007) float-a-lyzer units (MWCO 20 kDa) against [0.9% sodium chloride, 5 mM sodium bicarbonate, 1 mM HEPES buffer, 1 mM calcium chloride, pH 7.5]. Dialysis was performed in sterile, 2.2-liter Corning roller bottles at 4° C. overnight. After dialysis, protein was filtered using 0.2 μM filter units, and aliquots tested for endotoxin contamination using Pyrotell LAL gel clot system single test vials (STV) (Catalog #G2006, Associates of Cape Cod, East Falmouth, MA).

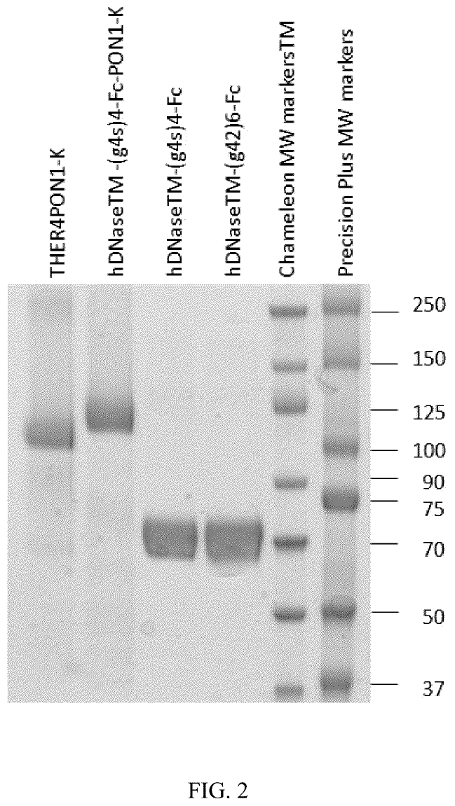

FIG. 2 shows purified proteins from stable CHO transfections after SDS-PAGE electrophoresis and staining with Imperial Protein Stain (Pierce/ThermoFisher, Waltham, MA/Rockville, MD; Catalog #24615). Five micrograms each protein-A-purified fusion protein was incubated in 1X LDS reducing sample buffer at 72° C. for 10 minutes followed by loading onto 4-12% Bis-Tris NuPAGE gels (Life Technologies, Carlsbad, CA). Protein molecular weight markers were included on each gel (Chameleon® DUO molecular weight markers (LI-COR Biosciences, Lincoln, NE) and Precision Plus molecular weight markers (BioRad, Hercules, CA)). SDS-PAGE gels were run in NuPAGE MOPS running buffer (Life Technologies, Carlsbad, CA) under reducing conditions at 175 volts for approximately 2 hours. Gels were washed three times in 100 ml distilled water, then stained in 30 ml Imperial Stain (Pierce/ThermoFisher, Waltham, MA) at room temperature with rocking for 2.5 hours. Gels were destained by washing 4 times in 100-200 ml distilled water and placed in a plastic sleeve prior to photographing/scanning of the stained gel. Lanes were loaded as follows: Lane 1 -THER4PON1-K (control; apoA1-(g4s)4-SSShinge-P238S-P331S-Fc-NGS-PON1-K; see International PCT Publication No. WO 2017/044424); Lane2 - hDNaseTM-(g4s)4-Fc-PON1-K; Lane 3 -hDNaseTM-(g4s)4-Fc; Lane 4 - hDNaseTM-(g4s)6-Fc; Lane5 - LI-COR Chameleon® MW markers; Lane 6 - BioRad Precision Plus Kaleidoscope MW markers. The approximate molecular weight (kDa) of each marker band is indicated on the right side of the stained gel.

### Example 2: DNase Activity of DNase1-Fc and DNase1-Fc-PON1 Fusion Proteins

Nuclease activity of DNase1 fusion proteins was assessed using a modified SYTOX™ Green fluorescence assay. SYTOX Green dye (5 mM solution in DMSO, Catalog #S7020) was obtained from Molecular Probes/Invitrogen (ThermoFisher Scientific, Waltham, MA). Salmon sperm DNA was also obtained from ThermoFisher Scientific (UltraPure, sheared, phenol purified, Catalog #15632011). Enzyme activity assays were performed either by titrating the substrate concentration with a fixed enzyme concentration, or by titrating the enzyme concentration with a fixed substrate concentration. For substrate titration experiments, salmon sperm DNA was mixed with SYTOX Green stain at a ratio of 14 μM sytox green to 400 μM (140 ug/ml) salmon sperm DNA in 1X DNase reaction buffer (10 mM Tris, pH 7.5, 5 mM MgCl₂, 1 mM CaCl₂)), and the mixture was incubated at 37° C. for approximately 2 hours to label and equilibrate. Appropriate dilutions of the stock labeled DNA were made for enzyme activity assays. One-third fold serial dilutions of SYTOX Green labeled DNA were prepared in a separate plate and 50 μl of each dilution was transferred to the assay plate to generate the following substrate concentrations in the final reactions: 1) 220 μM salmon sperm DNA (SS DNA) and 10 μM SYTOX Green; 2) 147 μM SS DNA and 6.7 μM SYTOX Green; 3) 98.78 μM SS DNA and 4.4 μM SYTOX Green; 4) 66 μM SS DNA and 3.01 μM SYTOX Green; and 5) 44 μM SS DNA and 2.05 μM SYTOX Green.

Each fusion protein or recombinant DNase 1 enzyme was prepared at two times the desired final concentration (9 nM initial, or 4.5 nM final concentration) to be assessed in 1X DNase reaction buffer. Fifty microliters of each fusion protein or recombinant enzyme were added to each well of the assay plate containing the labeled substrate. Plates were immediately transferred to the plate reader, and fluorescence was measured using 485 nm excitation and 528 nm emission wavelengths. Sample fluorescence was monitored using a kinetic assay on a VarioSkan LUX fluorescent plate reader (Thermo Scientific, Waltham, MA) with readings every 45 seconds for a total of 40 minutes. RFU (Relative Fluorescence Units) were plotted as a function of time for each well. The fluorescence signal in each well decreased more rapidly with increasing nuclease activity.

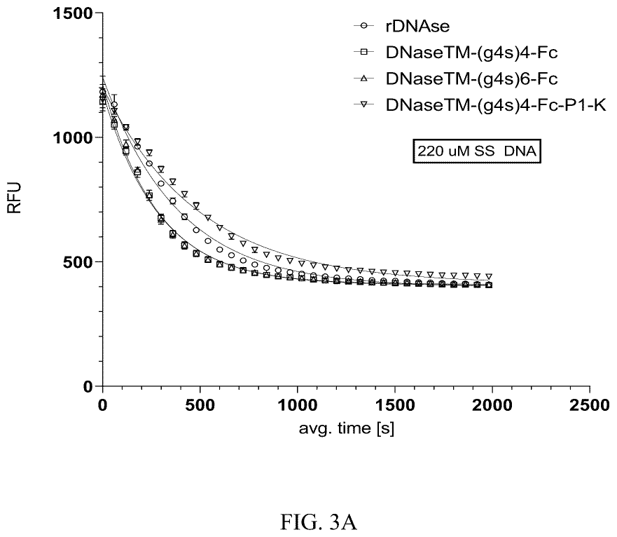

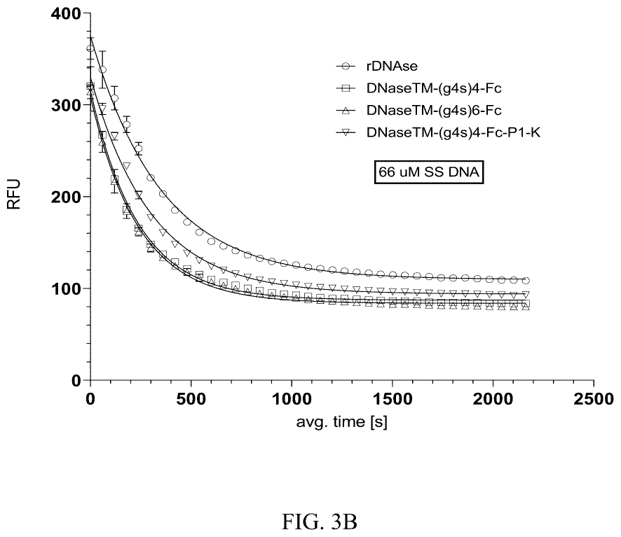

FIGS. 3A and 3B show a graphical representation of the raw data analyzing the relative fluorescence units (RFU) as a function of time at two different substrate concentrations, 220 μM or 66 M salmon sperm DNA. For these experiments, the enzyme concentration used was fixed at 4.5 nM. The specific activity of the DNase1TM-(g4s)4-Fc and DNase1TM-(g4s)6-Fc fusion proteins was higher than the recombinant wild-type human DNase1 at all substrate concentrations. At lower concentrations of substrate (the more physiologically relevant levels), the bispecific fusion protein (DNase1™-(g4s)4-Fc-P1-K) showed higher activity than the recombinant wild-type DNase1, while at higher concentrations of DNA substrate, the wild-type DNase1 had higher activity than the bispecific fusion protein.

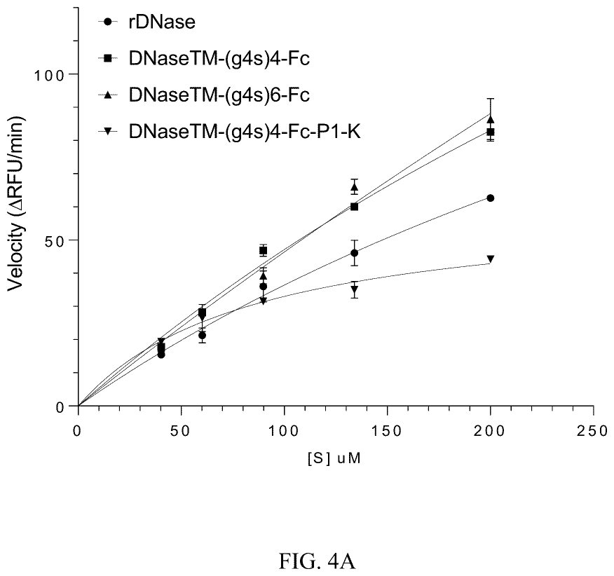

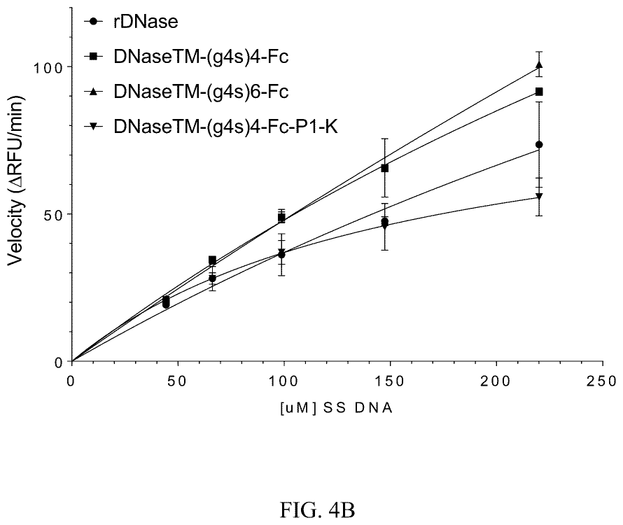

FIGS. 4A and 4B show Michaelis-Menten plots of the data from two different DNase activity experiments (“Experiment #1” and “Experiment #2,” respectively) using these conditions. The estimated Km and Vmax for each fusion protein in Experiment #1 and Experiment #2 are shown below in Table 4, with the tabulated data for each experiment listed. The units of the Vmax or maximal velocity are in moles SYTOX-labeled salmon DNA digested/min, and the units for the Km are in moles labeled salmon sperm DNA. Raw data was analyzed using the SkanIt™ software for the VarioSkan LUX plate reader (Thermo Scientific, Waltham, MA), then the data further analyzed using Microsoft Excel (Redmond, WA) and GraphPad Prism 8.4.3 (San Diego CA).

TABLE 4
Tabulated Michaelis Menten Data for DNase Fusion Proteins at 4.5 nM

| | | DNaseTM- | DNaseTM- | DNaseTM- |
|---|---|---|---|---|
| | rDNase | (g4s)4-Fc | (g4s)6-Fc | (g4s)4Fc-Pl-K |
| Experiment #1 | | | | |
| Vmax | 332.5 | 374.7 | 968.9 | 95.91 |
| Km | 533.6 | 681.9 | 1919 | 159.7 |
| Experiment #2 | | | | |
| Vmax | 230.8 | 349.4 | 874.3 | 61.13 |
| Km | 533.6 | 641.6 | 1785 | 85.29 |

Vmax: μmol salmon sperm DNA digested/min
Km: μmol salmon sperm DNA

Enzyme activity assays were also performed in the presence or absence of actin to assess whether the DNase fusion proteins were sensitive to actin inhibition. These assays were performed either by titrating the substrate concentration with a fixed enzyme concentration, or by titrating the enzyme concentration with a fixed substrate concentration. For substrate titration experiments, salmon sperm DNA was mixed with SYTOX Green stain at a ratio of 14 μM sytox green to 400 μM (140 μg/ml) salmon sperm DNA in 1X DNase reaction buffer (10 mM Tris, pH 7.5, 5 mM MgCl₂, 1 mM CaCl₂)), and the mixture was incubated at 37° C. for approximately 2 hours to label and equilibrate.

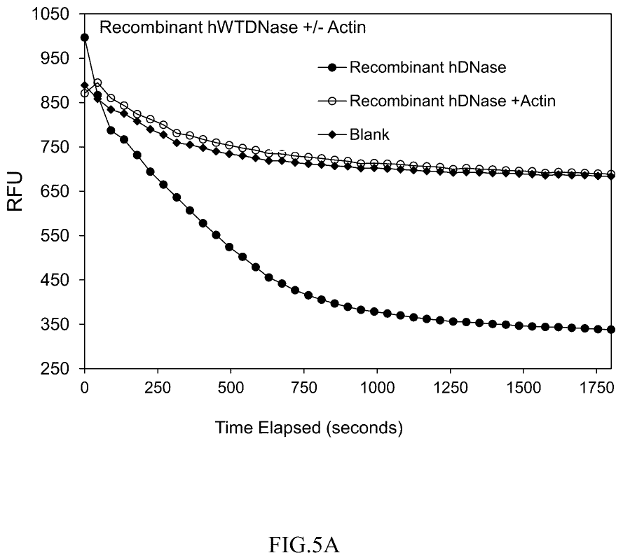

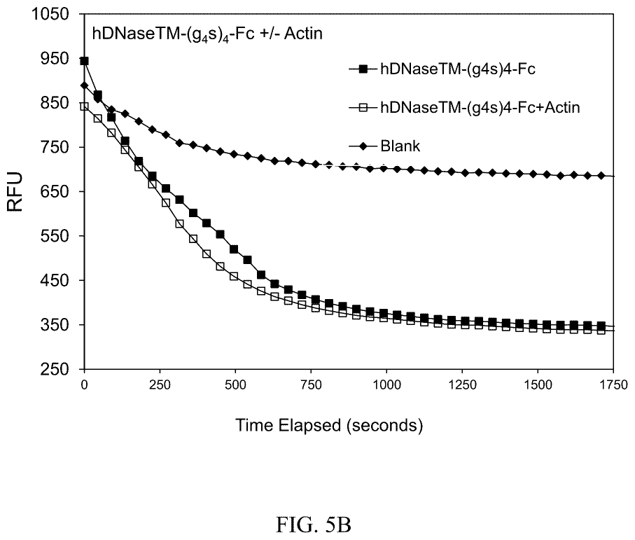

Each fusion protein or recombinant DNase1 enzyme was prepared at four times the desired final concentration (18 nM initial, or 4.5 nM final concentration) to be assessed in 1X DNase reaction buffer. Each 4X sample was then mixed 1:1 with a solution containing rabbit skeletal muscle actin protein (Cytoskeleton, Denver, CO., Catalog #AKL99-A) at 4X=160 μg/ml, or a final concentration in the nuclease reaction of 40 μg/ml; or reaction buffer containing no actin. Samples containing 9 nM fusion protein with 80 μg/ml actin were incubated at 37° C. for 45-60 minutes prior to addition of the labeled SYTOX green substrate. Sytox green substrate was prepared by adding 160 g/ml salmon sperm DNA to 14 μM Sytox green dye in 1X DNase reaction buffer and equilibrating at 37° C. for 60-120 minutes. Fifty microliters of each actin-treated fusion protein or recombinant enzyme were added to each well of the assay plate containing 50 μl of the labeled substrate. Plates were immediately transferred to the plate reader, and fluorescence was measured using 485 nm excitation and 528 nm emission wavelengths. Sample fluorescence was monitored using a kinetic assay on a VarioSkan LUX fluorescent plate reader (Thermo Scientific, Waltham, MA) with readings every 45 seconds for a total of 40 minutes. RFU (Relative Fluorescence Units) were plotted as a function of time for each well. The fluorescence signal in each well decreased more rapidly with increasing nuclease activity. FIGS. 5A and 5B show graphs plotting the RFU as a function of time for the DNase™-(g4s)4-Fc molecule and for the recombinant human DNase. The nuclease activity of the recombinant human DNase1 was inhibited by actin, while the fusion protein variants were not inhibited under these conditions.

### Example 3: Paraoxonase Activity of DNase1-Fc-PON1 Fusion Protein

### Arvlesterase Activity

Phenyl acetate was used as a substrate to assess arylesterase activity of the DNase1TM-(g4s)4-Fc-PON1-Q192K fusion protein. Enzyme activity assays were performed either by titrating the substrate concentration with a fixed enzyme concentration, or by titrating the enzyme concentration with a fixed substrate concentration. For the phenyl acetate activity assays, reaction buffer contained 20 mM Tris-HCl, pH 8.0, 1 mM CaCl₂). Substrate solution contained reaction butter and 10 mM phenyl acetate, while enzyme solution contained reaction buffer and enzyme(s) at the appropriate concentrations. Substrate solution was diluted 1:1 with enzyme solutions for a final concentration of 5 mM phenyl acetate substrate in the reactions. Fusion protein (enzyme) concentration was present in the assays titrated in ⅓ serial dilutions from 40 μg/ml (40, 26.8, 17.96, 12.05, 8.06, 5.4, 3.62, and 2.4 g/ml). Sample absorption was monitored using a kinetic assay on a VarioSkan LUX fluorescent plate reader, with readings every 45 seconds for a total of 10 minutes. Molar absorptivity at 270 nm serves as a measure of phenol formed as a function of time. The change in absorption was converted into a reaction rate using the molar extinction coefficient of 1310 M⁻¹cm⁻¹ for phenol.

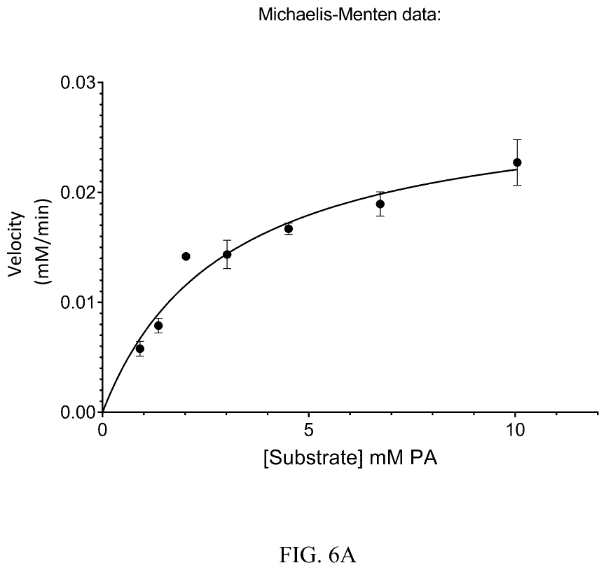

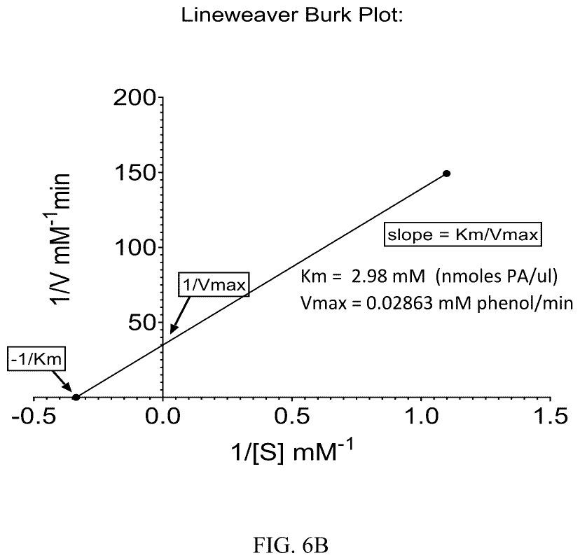

In addition, arylesterase activity assays were performed titrating the amount of phenyl acetate substrate present in ⅓-fold serial increments as follows: 15 mM, 10.05 mM, 6.73 mM, 4.51 mM, 3.02 mM, 2.03 mM, 1.36 mM, and 0.91 mM substrate. In each case, the amount of fusion protein (enzyme) present was 7.5 μg/ml. For the DNase1TM-(g4s)4-Fc-PON1-Q192K molecules, this concentration corresponds to 75 nM. FIGS. 6A and 6B show Michaelis-Menten and Lineweaver-Burk plots, respectively, for a representative experiment measuring the arylesterase activity of the DNase™-(g4s)4-Fc-PON1-Q192K fusion protein. When multiple experiments were compared, the Vmax average was approximately 0.027 mM phenol/min (mmoles/liter=nmoles/ul) and the Km was approximately 2.7 mM phenyl acetate (mmoles/liter=nmoles phenyl acetate/ul).

### Organophosphatase Activity

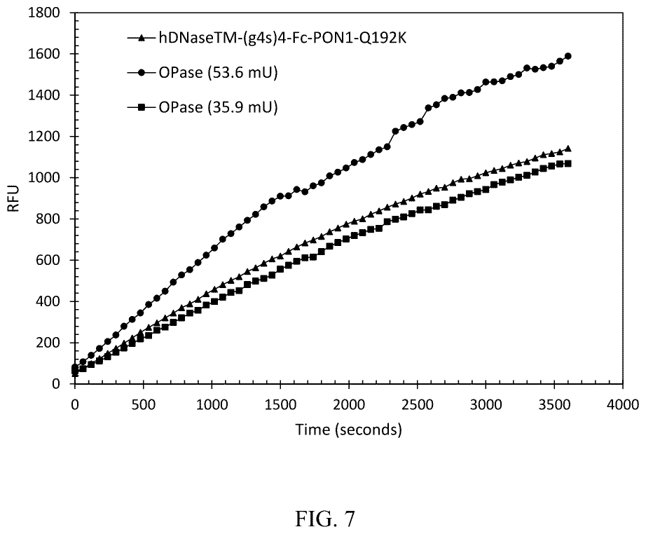

The Enzcheck paraoxonase activity assay (Catalog #E33702, ThermoFisher Scientific, Waltham, MA) was used to assess organophosphatase activity of the DNase1TM-(g4s)4-Fc-PON1-Q192K fusion protein. This assay is a very sensitive fluorometric assay for the organophosphatase activity of paraoxonase that uses excitation/emission maxima of 360/450 nm to measure the conversion of a fluorogenic organophosphate analog provided with the kit. The assay can either be set up as a kinetic assay or terminated after a particular period of time for an endpoint assay. The change in relative fluorescence units (RFU) per unit time is converted to the units of paraoxonase in the sample using the standard curve generated from the fluorescent standard and the conversion factor that 1 U unit of paraoxonase generates 1 nmol of fluorescent product per minute at 37° C. The amount of paraoxonase present in the fusion protein samples can be compared to the paraoxonase positive control provided with the kit. FIG. 7 shows a graphical representation of the organophosphatase activity of the DNase1TM-(g4s)4-Fc-PON1-Q192K fusion protein compared to the RFU curves generated by two different dilutions of the organophosphate positive control in the average to above average activity range for human serum sample comparisons. The fusion protein tested had been kept at 4° C. for 11 months, but still had activity above average in the PON1 assay.

Similar activity assays to the arylesterase and organophosphatase can be performed for the PON1 enzyme activities present in fusion proteins as described herein, such as, e.g., peroxidase activity assays (Amplex Red peroxide/peroxidase assay, ThermoFisher Scientific, Waltham MA, Catalog number: A22188), a general phosphatase assay (ENzChek phosphatase assay, ThermoFisher Scientific, Waltham MA, Catalog number: 12020), or lactonase activity assays using DHC or other substrates similar to those described by Billecke et al. (Drug Metabol. Disposit. 28:1335-1342, 2000) or Murillo-Gonzalez et al. (Clin. Chim. Acta 500:47-53, 2020).

### Example 4: Assays to Assess Inhibition of Biofilm Formation by DNase-Fc and DNase-Fc-PON1 Fusion Proteins

DNase-Fc and DNase-Fc-PON1 fusion proteins are characterized in biofilm formation assays to evaluate inhibition of Pseudomonas aeruginosa biofilm formation. Pseudomonas aeruginosa cultures are grown from an isolated bacterial colony by inoculation into rich media such as 2xYT or LB media. Overnight cultures are diluted 1:100 in tryptic soy broth/0.5% glucose (Teknova, Hollister, CA), and the diluted cultures used to inoculate 100 l/well into a 96 well tissue culture plate. Wells are precoated with each fusion protein of interest at different concentrations, prior to inoculation with the diluted bacterial culture. Alternatively, fusion proteins are added directly to diluted bacterial cultures to assess inhibition activity in solution. Plates are incubated at 37° C. for 4-8 hours prior to assay. All conditions are performed in replicates of 6-8 in order to control for well-to-well variation. After the incubation, cultures are removed by aspiration or turning the plate over and shaking out the liquid. The plate is then washed with PBS to remove unattached cells and media. After washing, 125 μl of 0.1% solution crystal violet is added to each well, and the plate is incubated for 15 minutes at room temperature. Plates are rinsed 3-4 times with water or PBS using a plate washer. After the final wash, microtiter plates are inverted and left to dry for a few hours or overnight. To quantify biofilms, 125 1d 30% acetic acid is added to each dried well in order to solubilize the crystal violet. Plates are incubated at room temperature for 15 minutes, and the 125 ul of crystal violet is transferred to a new flat bottom microtiter dish. Well absorbance is then measured at 550 nm in a Varioskan plate reader using 30% acetic acid as a blank solution. Percent biofilm inhibition is estimated by subtracting the ratio of [fusion protein treated absorbance average/untreated absorbance average] from 1 and multiplying by 100.

A commercially available biofilm formation kit is also available from Dojindo (Donjindo Laboratories, Kumamoto, Japan) that uses pegged microtiter plates for biofilm formation, simplifying washing steps.

### Example 5: Construction of DNase1L3-Fc and

### DNase1L3-Fc-PON1 Fusion Proteins

Constructs were designed and synthesized and cassettes with the correct sequences are combined to generate two synthetic fusion genes encoding unique DNase1L3 fusion molecules. Each construct includes a variant of wild-type human DNase1L3 (nucleotide and encoded amino acid sequences as shown in SEQ ID NOs:33 and 34). Each DNase1L3 sequence is modified to remove amino acid residues 291-305 of the wild-type sequence, corresponding to the C-terminal nuclear localization signal (NLS2). Each DNase1L3 variant also contains amino acid substitutions, relative to the wild-type human sequence (SEQ ID NO:34), at positions R80, R95, and N96 (either all three positions changed to alanine or all three changed to serine) to inactivate a nuclear localization signal (NLS1) located in the N-terminal half of the molecule. The nucleotide and encoded amino acid sequences corresponding to the mature DNase1L3 variant containing the R80A/R95A/N96A mutations are shown in residues 61-870 of SEQ ID NO:35 and residues 21-290 of SEQ ID NO:36, respectively; the nucleotide and encoded amino acid sequences corresponding to the mature DNase1L3 variant containing the R80S/R95S/N96S mutations are shown in residues 61-870 of SEQ ID NO:37 and residues 21-290 of SEQ ID NO:38, respectively. The signal peptide sequence utilized was the human VK3 leader peptide (nucleotide and amino acid sequences as shown in SEQ ID NO:27 and SEQ ID NO:28). The DNase1 cassette was fused to the N-terminus of a human IgG1 Fe variant (SSS hinge, P238S, P331S) using a (Gly4Ser)4 peptide linker ((g4s)4; nucleotide and amino acid sequences as shown in SEQ ID NO:7 and SEQ ID NO:8). The Fe variant nucleotide and amino acid sequences are shown in SEQ ID NO:13 and SEQ ID NO:14. The completed DNase1L3-Fc constructs generated are as follows:
[hVK3LP]-[hDNase1L3 R80A-R95A-N96A NLS2delta]-(g4s)4-[SSShinge-P238S-P331S Fc] (nucleotide and amino acid sequences as shown in SEQ ID NO:35 and SEQ ID NO:36); and
[hVK3LP]-[hDNase1L3 R80S-R95S-N96S NLS2delta]-(g4s)4-[SSShinge-P238S-P331S Fc] (nucleotide and amino acid sequences as shown in SEQ ID NO:37 and SEQ ID NO:38) (also referred to hereinbelow as DNase1L3SSS-Fc).

Bispecific constructs further comprising a functional PON1 enzyme are also generated by fusing the carboxyl end of each variant DNase1L3-Fc sequence above to a peptide linker containing an N-linked glycosylation site (NGS; nucleotide and amino acid sequences as shown in SEQ ID NO:31 and SEQ ID NO:32) followed by a variant of human PON1 (PON1 Q192K; nucleotide and amino acid sequences as shown in SEQ ID NO:23 and SEQ ID NO:24) in which the first 15 amino acids of the uncleaved leader peptide are deleted. The completed DNase1L3-Fc-PON1 constructs are as follows:
hVK3LP-[hDNase1L3 R80A-R95A-N96A NLS2delta]-(g4s)4-[SSShinge-P238S-P331S Fc]-NGS-[PON1 Q192K] (nucleotide and amino acid sequences as shown in SEQ ID NO:67 and SEQ ID NO:68); and
hVK3LP-[hDNase1L3 R80S-R95S-N96S NLS2delta]-(g4s)4-[SSShinge-P238S-P331S Fc]-NGS-[PON1 Q192K] (nucleotide and amino acid sequences as shown in SEQ ID NO:69 and SEQ ID NO:70) (also referred to hereinbelow as DNase1L3SSS-Fc-PON1).

### Example 6: Transient Expression of DNase1L3-Fc and DNase1L3-Fc-PON1 Fusion Proteins

DNase1L3SSS-Fc and DNase1L3SSS-Fc-PON1 fusion genes as described in Example 5 were constructed and then inserted into a multiple cloning site of the mammalian expression vector pDG, a pcDNA3 plasmid derivative containing a CMV promoter to drive expression of the fusion gene. Plasmid DNA was prepared using QIAGEN (Germantown, MD) mini or maxiprep plasmid DNA kits. Purified plasmid DNA was transfected into HEK293 cells plated at approximately 50-75% confluence, using Polyfect (QIAGEN, Germantown, MD) transfection reagent according to the manufacturer's instructions. Culture media was changed to DMEM Fluorobrite™ (Life Technologies, Carlsbad, CA) serum free media on the day after transfections, and transfected cells incubated for an additional 48 hours prior to harvest of culture supernatants. Culture supernatants were filtered through 0.2 m PES syringe filters prior to analysis. Protein A-agarose slurry was incubated on a roller at 4° C., overnight (50 l/sample, Repligen, Waltham MA) with 0.5 ml culture supernatant to immunoprecipitate fusion proteins. Protein A agarose was washed several times in PBS prior to resuspension in 50 ul LDS sample loading buffer for SDS-PAGE and Western blotting. Samples were then heated at 72° C. for 10 minutes, vortexed, then the protein A slurry centrifuged, and an aliquot (15-20 ul) of the sample buffer loaded onto 4-12% BIS-TRIS NUPAGE gels (Invitrogen, Carlsbad, CA). After electrophoresis and staining or Western blotting, the DNase1L3 fusion proteins could be detected on denaturing gels at the approximate predicted MWs of >60 and 99 kDa.

### Example 7: Pharmacokinetics of DNase1-Fc Fusion Protein in Wild-type Mice

Pharmacokinetics of the DNase™-(g4s)4-Fc fusion protein was assessed in BALB/c wild type mice. Mice were injected intravenously (IV) in the tail vein with 100 μg DNase™-(g4s)4-Fc fusion protein in 100 1d sterile 0.9% NaCl, 0.75 mM sodium bicarbonate, 1 mM CaCl₂), pH 7.0. A total of thirty mice were used to perform the PK study. Animals were grouped into five animals per time point. A total of six time points were analyzed, including 1 hour, 6 hours, 24 hours, 72 hours, 7 days, and 10 days. Peripheral blood was collected at sacrifice with heparin. Concentration of DNase™-(g4s)4-Fc in the plasma was measured using an IgG sandwich ELISA. ELISA assays were performed on 96 well NUNC immulon II MaxiSorp plates (ThermoFisher Scientific, Waltham, MA). Wells were coated with 100 l/well goat-anti-human IgG (at 2 μg/ml, affini-pure F(ab′)2 goat anti-human Fe specific, Catalog #: 109-006-190, Jackson Immunoresearch, West Grove, PA) for 12 hours at 4° C. in 0.1 M carbonate buffer (pH 9.0). Plates were incubated overnight at 4° C., prior to blocking in 200 l/well PBS/2.0% BSA, overnight at 4° C. Plates were washed in PBS, 0.05% Tween-20, 0.005% Kathon (wash buffer), and serial dilutions of plasma in Dulbecco's PBS were added to each well in 100 1d volume/well. Plates were incubated with plasma dilutions at 4° C., overnight. Plates were washed four times in wash buffer, then incubated with horseradish peroxidase conjugated goat anti-human IgG Fe specific, at a dilution of 1:10,000 (Jackson Immunoresearch, West Grove, PA). Standard curve was generated using serial dilutions of purified DNase™-(g4s)4-Fe.

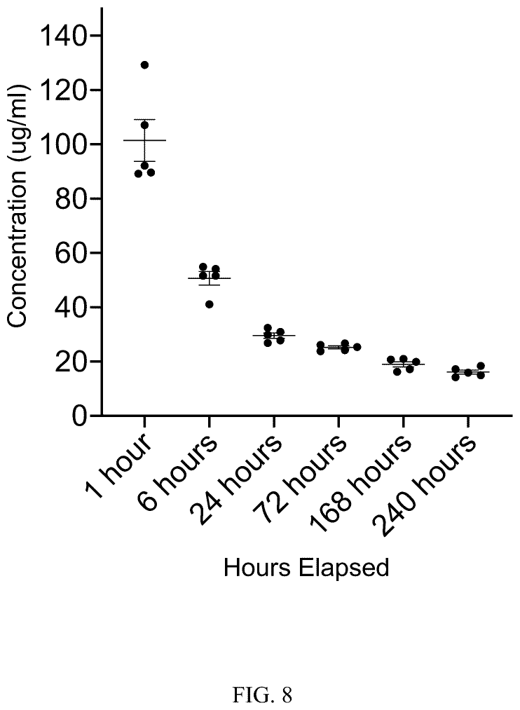

The results of the pharmacokinetic study are shown in FIG. 8. Each point represents the calculated serum concentration of DNase™-(g4s)4-Fc (in micrograms/ml, g/ml) in individual mice, and the average concentration for each time group is indicated on the Y-axis by the horizontal bar. Error bars indicate the standard error from the mean. The X-axis shows the time point after injection of fusion protein.

Nuclease activity of DNase™-(g4s)4-Fc fusion protein present in the mouse plasma from the pharmacokinetic study was also assessed using a modified SYTOX™ Green fluorescence assay. Diluted plasma samples from each mouse were added to a 96 well black microtiter plate coated with specific goat-anti-human IgG (at 2 μg/ml, affini-pure F(ab′)2 goat anti-human Fe specific, Catalog #: 109-006-190, Jackson Immunoresearch, West Grove, PA) for 12 hours at 4° C. Early time points were diluted 1:10, while later time points were diluted 1:2. Plates were washed three times with 1X-Dulbecco's PBS, 0.05% Tween 20, 0.004% Kathon prior to addition of 50 ul DNase reaction buffer (10 mM Tris, pH 7.5, 5 mM MgCl₂, 1 mM CaCl₂)). Substrate solution was prepared using SYTOX Green dye (5 mM solution in DMSO, Catalog #S7020) obtained from Molecular Probes/Invitrogen (ThermoFisher Scientific, Waltham, MA). Salmon sperm DNA was also obtained from ThermoFisher Scientific (UltraPure, sheared, phenol purified, Catalog #15632011). Enzyme activity assays were performed at a fixed substrate concentration with serial dilutions of enzyme. For substrate preparation, salmon sperm DNA was mixed with SYTOX Green stain at a ratio of 14 μM sytox green to 400 μM (140 ug/ml) salmon sperm DNA in 1X DNase reaction buffer (10 mM Tris, pH 7.5, 5 mM MgCl₂, 1 mM CaCl₂)), and the mixture was incubated at 37° C. for 1-2 hours to label and equilibrate. The stock labeled DNA (50 1d per well) was then added to wells of the plate containing immobilized enzyme from the mouse plasma (7 μM sytox green/100 m salmon sperm DNA) to give a final volume of 100 1d per well.

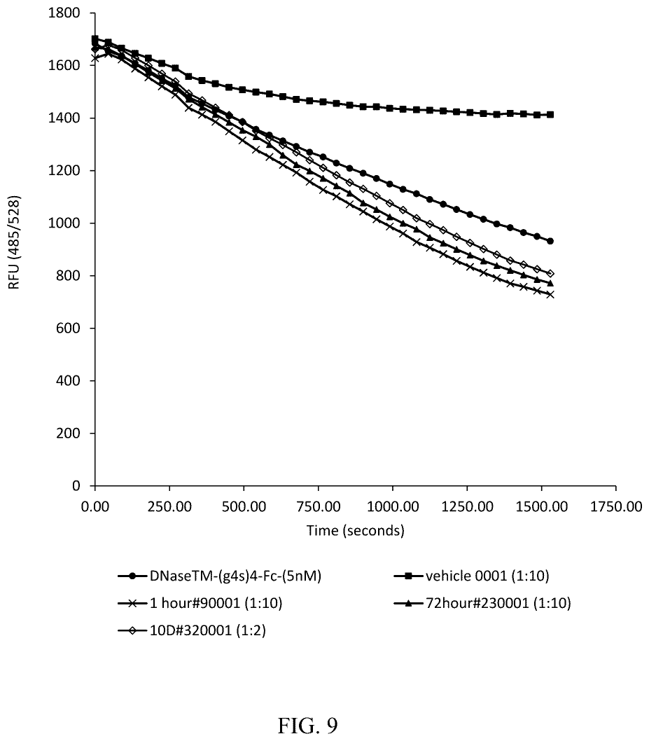

Plates were immediately transferred to the plate reader, and fluorescence was measured using 485 nm excitation and 528 nm emission wavelengths. Sample fluorescence was monitored using a kinetic assay on a VarioSkan LUX fluorescent plate reader (Thermo Scientific, Waltham, MA) with readings every 45 seconds for a total of 30 minutes. RFU (Relative Fluorescence Units) were plotted as a function of time for each well. The fluorescence signal in each well decreased more rapidly with increasing nuclease activity. All mice were assayed and showed similar DNase™-(g4s)4-Fc activity. Representative mice from early and late time points are shown in the FIG. 9.

### Example 8: Therapeutic Treatment of 4T1 Breast Tumor Model with DNase1-Fc Fusion Protein

Therapeutic activity of DNase™-(g4s)4-Fc in a 4T1 breast tumor model was evaluated. The 4T1 mammary carcinoma is a transplantable tumor cell line that is highly tumorigenic and invasive and, unlike most tumor models, can spontaneously metastasize from the primary tumor in the mammary gland to multiple distant sites including lymph nodes, blood, liver, lung, brain, and bone (Pulaski and Ostrand-Rosenberg, Curr. Protoc. Immunol. Chapter 20:Unit 20.2, 2001). The ability of 4T1 metastatic disease to develop spontaneously from the primary tumor and the progressive spread of 4T1 metastases to the draining lymph nodes and other organs, which is very similar to that of human mammary cancer, are among characteristics that make the 4T1 tumor a particularly suitable experimental animal model for human mammary cancer (see id.).

4T1 tumor cells were grown in vitro and 1×10e5 cells in 100 μl were injected subcutaneously into the flank of syngeneic BALB/c mice. Mice were divided into 4 groups of 10 mice each. Tumors were allowed to establish (50-80 mm³) and treatment with test articles began on Day 6. Group 1 was treated with vehicle IV on day 6, and IP on day 13. Group 2 received the DNase™-(g4s)4-Fc (100 μg in 100 μl) IV on day 6, and IP on day 13. Group 3 received the DNase™-(g4s)4-Fc (100 μg in 100 μl) IV on day 6 and IP on day 13, but in addition these mice received anti-PD-1 and anti-CTLA4 checkpoint inhibitors, given IP only (200 μg in 100 μl), on days 6, 10, 13, and 17. Group 4 received the checkpoint inhibitors alone given IP only (200 μg in 100 μl), on days 6, 10, 13, and 17. Body weight and tumor volume was measured three times per week until tumor size reached >2000 mm³, animals had ulcerated tumors, or clinical observations of prostration, paralysis, seizures or hemorrhages, at which time animals were euthanized.

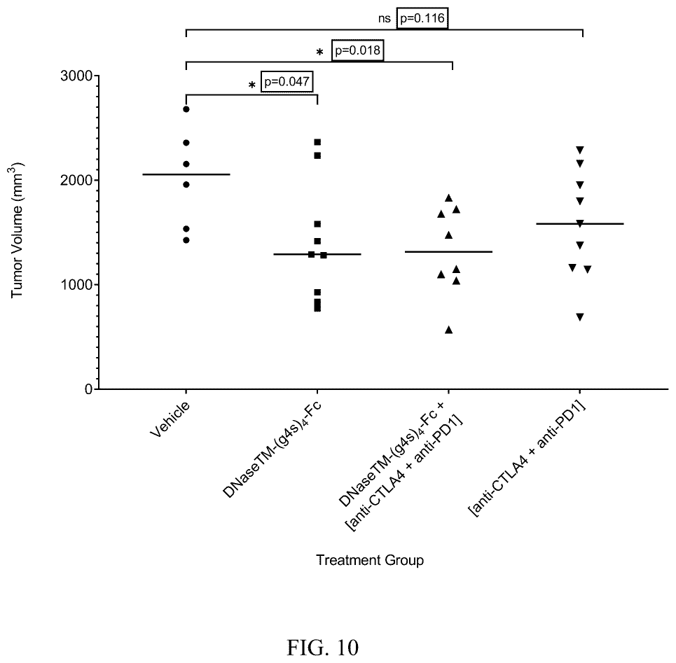

FIG. 10 shows the effect of DNase™-(g4s)4-Fc on 4T1 tumor growth on day 18. Significant differences in the different treatment groups are indicated on the graph. The average tumor volume is indicated by a horizontal line for each treatment group shown on the graph. Error bars indicate SEM. The data was analyzed by a two-tailed student's t test using GraphPad Prism 9.0 software. The DNase™-(g4s)4-Fc treatment group showed a significant difference from the vehicle group. The combination of DNase™-(g4s)4-Fc plus checkpoint inhibitors (anti-CTLA4⁺anti-PD1) was also significantly different from the vehicle group. Checkpoint inhibitors (anti-CTLA4⁺anti-PD1) alone was not significantly different.

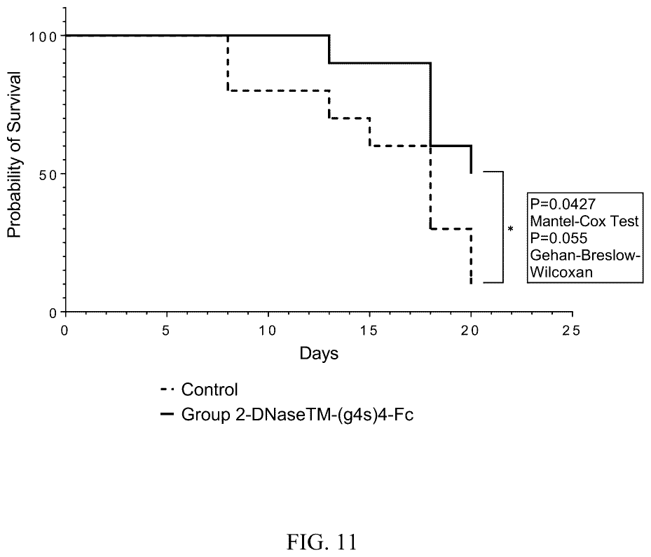

FIG. 11 shows survival data from the same 4T1 mouse mammary tumor model experiment, comparing the survival between the vehicle group and the DNase™-(g4s)4-Fc through day 20. Survival data was analyzed using GraphPad Prism 9.0 software; using the Mantel Cox analysis, the P value=0.0427, while the Gehan-Breslow-Wilcoxan test gave a P value of 0.055.

### Example 9: Treatment of Lung Fibrosis by Administration of DNase-Fc and DNase-Fc-PON Fusion Proteins

Effects of different DNase fusion proteins on lung fibrosis are assessed using in vivo mouse models of disease. One such model involves in vivo assessment of the efficacy of the purified fusion proteins for treatment of pulmonary fibrosis in a bleomycin induced lung fibrosis mouse model. Bleomycin (BLM)-induced pulmonary fibrosis is the most well-established disease model for IPF and is widely used to investigate the efficacy and mechanism of therapeutic candidates. In the model, alveolar injury and interstitial inflammation/fibrosis are induced by intratracheal BLM administration. The aim of this study is to examine the effect of DNase fusion test compounds on lung inflammation and fibrosis in BLM-induced pulmonary fibrosis model and compare responses to a control preparation of nintedanib. At day 0, mice are induced to develop pulmonary fibrosis by a single intratracheal administration of bleomycin hydrochloride in saline at a dose of 3.0 mg/kg, in a volume of 50 i per animal using Microsprayer. Mice are divided into groups of 12 mice based on the body weight changes on the day before the start of treatments with the test compounds on day 7. Mice are intranasally/intravenously/intraperitoneally administered test compounds (depending on the test group). Compounds are administered daily from Day 7 to 20, animals sacrificed on Day 21 and samples analyzed for fibrotic markers.

At study termination, whole blood samples without anticoagulant are obtained via abdominal vena cava under a mixture of medetomidine, midazolam and butorphanol anesthesia. The supernatants from separated blood are collected and stored at −80° C. until further processing. Lungs are harvested from the mice and two lobes left unfixed, one lobe is frozen and another harvested for biochemical assays such as measurements of hydroxyproline or inflammatory markers. Remaining lung lobes are fixed in 10% neutral buffered formalin, paraffin emedded for Masson's trichrome staining and histology. For quantitative analysis of lung fibrosis area, bright field images of Masson's Trichrome-stained sections are randomly captured using a digital camera at 100-fold magnification, and the subpleural regions in 20 fields/mouse are evaluated according to the criteria for grading lung fibrosis (Ashcroft, T., et al., J Clin Pathol, 1988; 41:467-70), to generate an Ashcroft score. All sections are blindly analyzed by an experimenter. Statistics are performed on each treatment group, calculating an average Aschroft score, and comparing the scores in the different treatment groups to the Vehicle group.

Alternative methods of fusion protein delivery may be utilized. As an example, rather than intranasal spray delivery which is confined to the upper airways, nebulization of the fusion proteins may improve delivery of the fusion molecules deeper into the lungs, thereby more effectively targeting all areas of the lung. Fusion proteins can be nebulized with or without excipients and tested for protein recovery, aggregation, and enzyme activity. A vibrating mesh (VM) nebulizer (PARI eFlow. Omron NE-U100, Lake Forest, IL) can be used since other proteins have been successfully nebulized using this apparatus and because VM nebulizers are small and portable. Nebulized proteins (500 μl) at 1 mg/ml in saline plus 1 mM CaCl₂)(Pulmozyme® buffer) are collected on a sterile 60 mm low protein binding tissue culture dish. Recovered fusion proteins are analyzed by SDS gels, size exclusion HPLC (SEC), and for protein concentration and enzyme activity before and after nebulization to evaluate the effects of the excipients and the effects of nebulization on the protein.

### Example 10: Transfection, Expression, and Purification of DNase-Fc Fusion Proteins with Intermediate Sensitivity to Inhibition by Actin

Stable production of the DNase1-Fc variant forms with intermediate actin resistance (e.g., a DNase1-Fc variant as shown in SEQ ID NO:86, SEQ ID NO:88, or SEQ ID NO:90) is achieved by electroporation of the plasmid, pDG, containing the DNase1 fusion molecule cDNAs under the control of the CMV promoter, into Chinese Hamster Ovary (CHO) DG44 cells. Plasmid DNA (200 ug) is prepared using QIAGEN HISPEED maxiprep kits. Purified plasmid is linearized at a unique AscI site (New England Biolabs, Ipswich, MA Catalog #R0558), purified by phenol extraction (Sigma-Aldrich, St Louis, MO), ethanol precipitated, washed, and resuspended in tissue culture media, Excell 302 (Catalog #14324, SAFC/Sigma Aldrich, St Louis, MO). Salmon sperm DNA (Sigma-Aldrich, St. Louis, MO) (200 μg) is added as carrier DNA just prior to phenol extraction and ethanol precipitation. Plasmid and carrier DNA are coprecipitated, and the 400 μg is used to transfect 2×10⁷ CHO DG44 cells by electroporation.

For electroporation, cells are grown to logarithmic phase in Excell 302 media (Catalog #13424C, SAFC Biosciences/Sigma-Aldrich, St. Louis, MO) as described in the previous examples. Electroporations are performed at 280 volts, 950 microFarads, using a BioRad (Hercules, CA) GenePulser electroporation unit with capacitance extender. Electroporation is performed in 0.4 cm gap sterile, disposable cuvettes. Electroporated cells are incubated for 5 minutes after electroporation prior to transfer of the treated cells to Excell 302 complete media without methotrexate and with hypoxanthine-thymidine (HT) addition, in T75 flasks. Transfected cells are incubated overnight at 37° C., 5% CO2 in the complete media to permit recovery, prior to selective plating in 96-well flat-bottom plates (Costar) at varying serial dilutions ranging from 250 cells/well (2500 cells/ml) to 2000 cells/well (20,000 cells/ml). Culture media for cell cloning is Excell 302 complete containing 50 nM methotrexate. Transfection plates are fed at five-day intervals with 80 μl fresh media. After the first couple of feedings, 100 μl media is removed and replaced with fresh media. Plates are monitored and individual wells with clones fed until clonal outgrowth becomes close to confluent, after which clones are expanded into 24-well dishes containing 1 ml media/well. Aliquots of the culture supernatants from the original 96-well plate are harvested to a second 96-well plate prior to transfer and expansion of the cells in the 24-well plates. This second plate is frozen until performing dilutions for ELISA analysis to estimate IgG concentrations.

Screening Culture Supernatants for Production Levels of Recombinant Fusion Proteins: Once clonal outgrowth of initial transfectants reaches approximately 50% confluence, serial dilutions of culture supernatants from master wells are thawed and the dilutions screened for expression of DNase1 fusion protein by use of an -IgG sandwich ELISA. Briefly, NUNC Maxisorp plates are coated overnight at 4° C. with 2 microgram/ml F(ab′2) goat anti-human IgG (Jackson Immunoresearch, West Grove, PA; Catalog #109-006-098) in PBS. Plates are blocked in PBS/3% BSA, and serial dilutions of culture supernatants incubated at room temperature for 2-3 hours or overnight at 4° C. Plates are washed three times in PBS/0.05% Tween 20, and incubated with horseradish-peroxidase-conjugated F(ab′2) goat anti-human IgG (Jackson Immunoresearch, West Grove, PA, Catalog #109-036-098) at 1:10,000 in PBS/0.5% BSA, for 1-2 hours at room temperature. Plates are washed five times in PBS/0.05% Tween 20, and binding detected with SureBlue Reserve™ TMB substrate (KPL Labs/SeraCare, Gaithersburg, MD; catalog #53-00-02). Reactions are stopped by addition of equal volume of iN HCl, and absorbance per well on each plate is read at 450 nM on a VarioSkan LUX plate reader (ThermoFisher Scientific, Waltham, MA). Concentrations are estimated by comparing the OD450 of the dilutions of culture supernatants to a standard curve generated using serial dilutions of a known standard, a protein-A-purified human -IgG fusion protein with the identical -Ig tail to clones described in this disclosure, typically THER4 (apoAl-(g4s)4-SSShinge-P238S-P331S Fc; see International PCT Publication No. WO 2017/044424) or one of the DNase-Fc mutants already described. Data is collected and analyzed using platereader software and Microsoft Office Excel or GraphPad Prism 8.4.3 (San Diego, CA). The best expressing DNase-lnk-Fc clones express in the range of 25-100 μg/ml, while the best expressing DNase-lnk-Fc-PON1-K clones expressed at approximately 10-50 μg/ml.

Culture supernatants are collected from spent CHO cell cultures expressing the fusion proteins, filtered through 0.2 μm PES express filters (Nalgene, Rochester, NY) and subjected to affinity chromatography using slow rotation of culture supernatants with Protein A-agarose (IPA 300 crosslinked agarose) slurry in 50 ml sterile, conical centrifuge tubes at 4° C. (Repligen, Waltham, MA). Fusion protein bound to protein A agarose is recovered by centrifugation, and culture supernatants removed, replaced, and the incubation process repeated until the desired volume of supernatant was processed. The final protein A agarose slurry is then loaded into sterile, acid-washed econocolumns (BioRad, Hercules, CA) to wash the resin. Columns are then washed with several column volumes of column wash buffer (Gentle Ag-Ab binding buffer, Pierce/ThermoFisher, Waltham, MA) to remove any residual culture supernatant, prior to elution. Bound protein is then eluted from the resin using gentle Ag/Ab elution buffer (Pierce/ThermoFisher, Waltham, MA). Fractions (0.8-1.0 ml) are collected and protein concentration of aliquots (2 μl) from each fraction are determined at 280 nM using a Nanodrop (Wilmington DE) microsample spectrophotometer, with blank determination using elution buffer alone. Fractions containing fusion protein are pooled, and buffer exchange performed by dialysis using Spectrum Laboratories G2 (Ranch Dominguez, CA, Catalog #G235057, Fisher Scientific catalog #08-607-007) float-a-lyzer units (MWCO 20 kDa) against [0.9% sodium chloride, 5 mM sodium bicarbonate, 1 mM HEPES buffer, 1 mM calcium chloride, pH 7.5]. Dialysis is performed in sterile, 2.2-liter Corning roller bottles at 4° C. overnight. After dialysis, protein was filtered using 0.2 μM filter units, and aliquots tested for endotoxin contamination using Pyrotell LAL gel clot system single test vials (STV) (Catalog #G2006, Associates of Cape Cod, East Falmouth, MA).

### Example 11: Evaluation of DNase-Fc Fusion Proteins with Intermediate Sensitivity to Inhibition by Actin for DNase Activity and Actin Sensitivity

Enzyme activity assays are performed in the presence or absence of actin to assess whether the DNase fusion proteins are less sensitive to actin inhibition. Actin inhibition is defined as 50% inhibition when twice the amount of DNase is required to see equivalent nuclease activity compared to the activity observed in the absence of actin. A 90% inhibition requires ten times the amount of enzyme to see nuclease activity equivalent to that observed in the absence of actin. These assays are performed either by titrating the substrate concentration with a fixed enzyme concentration, or by titrating the enzyme concentration with a fixed substrate concentration. For substrate titration experiments, salmon sperm DNA is mixed with SYTOX Green stain at a ratio of 14 μM sytox green to 400 μM (140 μg/ml) salmon sperm DNA in 1X DNase reaction buffer (10 mM Tris, pH 7.5, 5 mM MgCl₂, 1 mM CaCl₂)), and the mixture incubated at 37° C. for approximately 2 hours to label and equilibrate.

Each fusion protein or recombinant DNase1 enzyme is prepared at four times the desired final concentration (18 nM initial, or 4.5 nM final concentration) to be assessed in 1X DNase reaction buffer. Each 4X sample is then mixed 1:1 with solutions containing rabbit skeletal muscle actin protein (Cytoskeleton, Denver, CO., Catalog #AKL99-A) at 4X=160 μg/ml, or a final concentration in the nuclease reaction of 40 μg/ml for the highest concentration of actin; four-fold titrated amounts of actin down to a final concentration of 2.5 ng/ml (10 μg/ml, 2.5 μg/ml, 0.625 μg/ml, 0.156 μg/ml, 40 ng/ml, 10 ng/ml, 2.5 ng/ml) or reaction buffer containing no actin. Samples containing 9 nM fusion protein with 80 μg/ml actin or the titrated amounts of actin are incubated at 37° C. for 45 minutes prior to addition of the labeled SYTOX green substrate. Sytox green substrate is prepared by adding 160 μg/ml salmon sperm DNA to 14 μM Sytox green dye in 1X DNase reaction buffer and equilibrating at 37° C. for 60-120 minutes. Fifty microliters of each actin-treated fusion protein or recombinant enzyme are added to each well of the assay plate containing 50 μl of the labeled substrate. Plates are immediately transferred to the plate reader, and fluorescence measured using 485 nm excitation and 528 nm emission wavelengths. Sample fluorescence is monitored using a kinetic assay on a VarioSkan LUX fluorescent plate reader (Thermo Scientific, Waltham, MA) with readings every 45 seconds for a total of 40 minutes. RFU (Relative Fluorescence Units) are plotted as a function of time for each well. The fluorescence signal in each well decreases more rapidly with increasing nuclease activity. The data is analyzed by plotting the fraction of the relative RFU under actin inhibition over the relative RFU observed in the absence of actin. This fraction is then plotted as a function of actin concentration. The data can then be further analyzed by nonlinear regression analysis using computer software, solving for Ki, defined as the concentration of inhibitor required to reach 50% inhibition. See Ulmer et al. (Proc Natl Acad Sci USA 93:8225-8229, 1996).

$$Fractional activity = 1 - [ Eo ] + [ Io ] + Ki - √ ( [ Eo ] + [ Io ] + Ki 2 \hat{} - 4 * [ Eo ] * [ Io ] / ( 2 * [ Eo ] )$$

where [E₀] is the DNase I concentration and [I₀] is the total G-actin concentration.

Actin Binding by DNase Variants: DNase1 sequence variants with intermediate actin sensitivity are assessed for actin binding by an antigen binding ELISA. MaxiSorp plates (Nunc) are coated with 100 μl human Gc globulin (Millipore Sigma-Aldrich) at 10 μg/ml in 25 mM Hepes (pH 7.2), 4 mM MgCl₂, and 4 mM CaCl₂) at 4° C. for 16-24 h. After discarding the Gc globulin, excess reactive sites are blocked with 200 1d buffer A (25 mM Hepes, pH7.5/4 mM CaCl₂)/4 mM MgCl₂/0.1% BSA/0.5 mM ATP/0.01% thimerosal/0.05% Tween 20). Buffer A is used as dilution buffer; incubations are at room temperature for 1-2 hours. The wash buffer is PBS containing 0.05% Tween 20. Rabbit skeletal muscle G-actin (stock concentration 10 mg/ml; obtained from Cytoskeleton, Denver, CO) is diluted in buffer A to 50 μg/ml, and 100 μl added to plates, incubated for 1 hour at room temperature, washed, and 100 1d of DNase variant cell culture harvest at various dilutions or purified DNase variant fusion protein is added, serially diluted across the plate. After incubation and washing, 100 μl of anti-human DNase I rabbit polyclonal antibody-horseradish peroxidase conjugate (19 ng/ml) is added, or anti-human IgG1 Fe goat polyclonal antibody-horseradish peroxidase conjugate is added (1:7500, Jackson Immunoresearch, West Grove, PA) to detect binding of the fusion proteins to actin. Following incubation and washing, TMB single step substrate (SeraCare/LGC Clinical Diagnostics, Gaithersburg, MD) 100 l/well is added for color development. Reactions are stopped using iN HCl, and wells read at 450 nm using a VarioSkan LUX plate reader (ThermoFisher Scientific, Waltham, MA). Actin binding is compared to the wild type DNase-(g4s)4-Fc fusion protein to determine percent binding inhibition exhibited by the sequence variants.

### Example 12: Assessing DNase-Fc Fusion Proteins for Therapeutic Efficacy in an Acute DSS Colitis Mouse Model of IBD

Adult male C57BL/6 mice are randomized into experimental groups (10 mice/group) based on the bodyweight and allowed to acclimatize for one week prior to initiation of the study. On day 0, the drinking water is replaced by a solution of 5% dextran sulfate sodium (DSS) in water. Animals are given unrestricted access to the water replacement. Acute colitis is induced by administering DSS (dextran sulfate sodium) in the water from day 0 until the end of the experiment on day 7. Each treatment group contains 10 animals, and DNase-Fc fusion protein is administered once before DSS administration/experiment initiation, and once at day 4, IP (intraperitoneal) at 100 g/mouse. Animals are monitored daily for clinical signs of colitis, including bodyweight loss, loose stools and/or diarrhea, and the presence of occult or gross blood in the stool. Animals are assigned scores from 0 (normal) to 4 (severe symptoms) based on these criteria. At day 7, animals are culled, the colon dissected out and colon length measured. One sample of colon per animal is transferred in tissue fixative and processed for histopathology and scoring of mucosal thickness, mucosal ulceration, lamina propria mononuclear cell infiltration, lamina propria granulocyte infiltration and crypt abscesses/dilation/distortion. In addition to colon, blood, spleen, and lymph nodes are collected at study termination and stored frozen for later analysis of cytokines.

### Example 13: Assessing DNase-Fc Fusion Proteins for Therapeutic Efficacy in a Rheumatoid Arthritis Model

Collagen antibody-induced arthritis relies on the injection of a cocktail of monoclonal antibodies directed against type II collagen (C-II), followed by a single injection of LPS. In animals, a significant part of the inflammatory attack on the joints is mediated by pathogenic antibodies directed against C-II. Collagen antibody-induced arthritis is dependent on IL-1 and TNF-α, but is independent of the effects of IL-6. In this 21-day study, which bypasses the development of anti-collagen antibodies, arthritis is induced using a cocktail of monoclonal antibodies directed against type II collagen. Modeled after human rheumatoid arthritis, a key advantage of this model is its ability to induce the disease in many strains of mice that are resistant to the traditional collagen-induced arthritis methods.

Another advantage of this model is that compound assessment can be completed in a relatively short period of time. In collagen antibody-induced arthritis, the disease appears within 7-8 days and studies can be completed within 18 days. An additional 7 days can be added to assess the effects of agents on the resolution phase of the disease. For evaluation of DNase-Fc fusion proteins containing human DNase1 and Fe sequences and variants thereof as described herein, a shorter therapeutic window is necessary to assess efficacy prior to antibody responses in the animals to the fusion protein. After induction of disease, animals (10/group) will be treated 2X/week, IV for the first dose, and IP thereafter, with 100 μg/mouse fusion protein for each dose administered. Disease activity is assessed by measuring inflammation swelling in the affected joints (paw volume or thickness) over time. Treatments can be assessed in either prophylactic or therapeutic testing paradigms. Clinical scoring is accomplished by awarding a score of 1 for each swollen digit, a score of 5 for a swollen footpad and a score of 5 for a swollen wrist or ankle. These are added together to give a maximal score of 60 for each animal.

### Example 14: Assessing DNase-Fc Fusion Proteins for Therapeutic Efficacy in an Acute Respiratory Disease Model of Pseudomonas aeruizinosa-induced Pneumonia

The Pseudomonas aeruginosa-induced pneumonia model is performed over the course of a week. Mice for the experiment are approximately six weeks old, 20-25 g. Mice are treated with an inoculum of bacterial culture at day 0. The inoculum size depends on the mouse or rat strain, but for BALB/c would be approximately 5×10e6 CFU in 20 μl inoculum. Mice are anesthetized and the bacteria administered by intranasal route. Bioluminescent strains of bacteria can be used in order to perform in life analysis via bioluminescent imaging (IVIS) on inoculated mice. Mice are treated with the DNase1 sequence variants or with vehicle alone either just prior to bacterial infection or as a 100 ug/mouse injection IV 1 hour pre inoculum, or at 1 and 4 hours post inoculum. Mice are monitored for clinical score, change in body weight and temperature, bacterial load, CFU in lung homogenates, cellular infiltrate and cytokines in BAL or lung homogenates, gross pathology scores, and histopathology. Survival is also monitored over the course of 100 hours, when most untreated inoculated mice have died due to lung failure.

### Example 15: Assessing DNase-Fc Fusion Proteins for Therapeutic Efficacy in an Acute Asthma Model

DNase-Fc fusion proteins may also be assessed in steroid sensitive mouse or rat models using ovalbumin induced lung inflammation or dust mite induced lung inflammation similar to human asthma. The ovalbumin or house dust mite (HDM)-induced acute asthma model is used to assess the in vivo efficacy of anti-asthma drugs. These models feature many similarities to human allergic asthma, including the presence of eosinophilic lung inflammation and the release of inflammatory mediators and cytokines primarily associated with Th2-type inflammation. Allergen is initially administered IP in the presence of adjuvant, then may be administered a second time at days 7 to 14 by a second IP dose. After the sensitization period (usually 14-21 days), the animal is challenged with the allergen via the airway, usually over a period of several days. Allergen may be inhaled as a nebulised formulation (aerosol) or administered by intratracheal (i.t.) or intranasal (i.n.) instillation of an aqueous formulation.

Total and differential cell counts of inflammatory cells in the lung are performed on the bronchoalveolar lavage (BAL) fluid at various time points to observe the time course of inflammation and evaluate the effects of compounds. Inflammatory mediators, histopathologic evaluation and assessments of airway hyperresponsiveness can also be performed on animals sacrificed at various time points. The model is robust and reproducible and sensitive to steroids (both oral and inhaled). Rats are treated with the allergen, and reactive airways monitored prior to administration of vehicle, positive control (steroid), or DNase-Fc fusion protein(s). Mice are grouped into treatment groups containing 10 mice per group. Animals receive fusion protein by IV injection, 100 μg/mouse, every three days after the sensitization period, until completion of the experiment. Mice are sacrificed at days 4, 7, and 10 and lung tissue, blood, spleen, and lymph nodes harvested at termination and frozen until further analysis. During the course of the experiment, mice are monitored for clinical score, change in body weight and temperature, and after sacrifice, cellular infiltrate and cytokines in BAL or lung homogenates, gross pathology scores, and histopathology of treated lung tissue.

### Exemplary Embodiments

Embodiment 1. A fusion polypeptide comprising, from an amino-terminal position to a carboxyl-terminal position, D-L1-X, wherein: D is a biologically active DNase having at least 95% identity with the amino acid sequence shown in
(i) residues 21-280 of SEQ ID NO:4,
(ii) residues 21-280 of SEQ ID NO:52,
(iii) residues 21-280 of SEQ ID NO:74,
(iv) residues 21-280 of SEQ ID NO:76,
(v) residues 21-280 of SEQ ID NO:78, or
(vi) residues 21-290 of SEQ ID NO:34, L1 is a first polypeptide linker, wherein L1 comprises at least 26 amino acid residues, and X is an immunoglobulin Fc region.

Embodiment 2. The fusion polypeptide of Embodiment 1, wherein the DNase has at least 95% identity with the amino acid sequence shown in residues 21-280 of SEQ ID NO:4 or residues 21-280 of SEQ ID NO:52, and wherein the DNase contains at least one amino acid substitution at a position corresponding to an amino acid of human DNase1 (SEQ ID NO:30) selected from the group consisting of N74, G105, and A114, wherein the amino acid substitution at the position corresponding to N74 of human DNase1 (N74 substitution), if present, increases DNA binding of the DNase relative to human DNase1, the amino acid substitution at the position corresponding to G105 of human DNase1 (G105 substitution), if present, increases DNA binding of the DNase relative to human DNase1, and the amino acid substitution at the position corresponding to A114 of human DNase1 (A114 substitution), if present, decreases G-actin-induced inhibition of endonuclease activity of the DNase relative to human DNase1.

Embodiment 3. The fusion polypeptide of Embodiment 2, wherein the fusion polypeptide contains both the N74 and G105 substitutions.

Embodiment 4. The fusion polypeptide of Embodiment 3, wherein the amino acid at the position corresponding to N74 of human DNase1 is lysine, and/or the amino acid at the position corresponding to G105 of human DNase1 is arginine.

Embodiment 5. The fusion polypeptide of Embodiment 3 or 4, wherein the DNase does not contain any amino acid substitution, relative to human DNase1, that decreases G-actin-induced inhibition of endonuclease activity of the DNase relative to human DNase1.

Embodiment 6. The fusion polypeptide of Embodiment 2, wherein the fusion polypeptide contains both the G105 and A114 substitutions.

Embodiment 7. The fusion polypeptide of Embodiment 6, wherein the amino acid at the position corresponding to G105 of human DNase1 is arginine, and/or the amino acid at the position corresponding to A114 of human DNase1 is phenylalanine.

Embodiment 8. The fusion polypeptide of Embodiment 2, wherein the fusion polypeptide contains each of the N74, G105, and A114 substitutions.

Embodiment 9. The fusion polypeptide of Embodiment 8, wherein the DNase does not contain any other amino acid substitution, relative to human DNase1, that increases DNA binding of the DNase relative to human DNase1.

Embodiment 10. The fusion polypeptide of Embodiment 8 or 9, wherein the DNase does not contain any other amino acid substitution, relative to human DNase1, that decreases G-actin-induced inhibition of endonuclease activity of the DNase relative to human DNase1.

Embodiment 11. The fusion polypeptide of Embodiment 2 or 8, wherein the amino acid at the position corresponding to N74 of human DNase1 is lysine, the amino acid at the position corresponding to G105 of human DNase1 is arginine, and/or the amino acid at the position corresponding to A114 of human DNase1 is phenylalanine.

Embodiment 12. The fusion polypeptide of Embodiment 1, wherein the DNase has the amino acid sequence shown in (i) residues 21-280 of SEQ ID NO:4, (ii) residues 21-280 of SEQ ID NO:52, or (iii) residues 21-280 of SEQ ID NO:6.

Embodiment 13. The fusion polypeptide of Embodiment 1, wherein the DNase has at least 95% identity with the amino acid sequence shown in residues 21-280 of SEQ ID NO:74, residues 21-280 of SEQ ID NO:76, or residues 21-280 of SEQ ID NO:78, and wherein the DNase contains at least one amino acid substitution at a position corresponding to an amino acid of human DNase1 (SEQ ID NO:30) selected from the group consisting of D53, Y65, and E69, and optionally contains at least one amino acid substitution at a position corresponding to an amino acid of human DNase1 selected from the group consisting of N74 and G105, wherein the amino acid substitution at the position corresponding to D53 of human DNase1 (D53 substitution), if present, decreases G-actin-induced inhibition of endonuclease activity of the DNase relative to human DNase1, the amino acid substitution at the position corresponding to Y65 of human DNase1 (Y65 substitution), if present, decreases G-actin-induced inhibition of endonuclease activity of the DNase relative to human DNase1, the amino acid substitution at the position corresponding to E69 of human DNase1 (E69 substitution), if present, decreases G-actin-induced inhibition of endonuclease activity of the DNase relative to human DNase1, the amino acid substitution at the position corresponding to N74 of human DNase1 (N74 substitution), if present, increases DNA binding of the DNase relative to human DNase1, and the amino acid substitution at the position corresponding to G105 of human DNase1 (G105 substitution), if present, increases DNA binding of the DNase relative to human DNase1.

Embodiment 14. The fusion polypeptide of Embodiment 13, wherein the fusion polypeptide contains (i) the D53 substitution, (ii) the Y65 substitution, (iii) the E69 substitution, (iv) each of the D53, N74, and G105 substitutions, (v) each of the Y65, N74, and G105 substitutions, (vi) each of the E69, N74, and G105 substitutions, (vii) each of the D53, Y65, N74, and G105 substitutions, (viii) each of the D53, E69, N74, and G105 substitutions, (ix) each of the Y65, E69, N74, and G105 substitutions, or (x) each of the D53, Y65, E69, N74, and G105 substitutions.

Embodiment 15. The fusion polypeptide of Embodiment 14, wherein the amino acid at the position corresponding to D53 of human DNase1 is arginine, the amino acid at the position corresponding to Y65 of human DNase1 is alanine, the amino acid at the position corresponding to E69 of human DNase1 is lysine, the amino acid at the position corresponding to N74 of human DNase1 is lysine, and/or the amino acid at the position corresponding to G105 of human DNase1 is arginine.

Embodiment 16. The fusion polypeptide of Embodiment 14 or 15, wherein the enhanced DNase1 contains each of the D53, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

Embodiment 17. The fusion polypeptide of Embodiment 14 or 15, wherein the enhanced DNase1 contains each of the Y65, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

Embodiment 18. The fusion polypeptide of Embodiment 14 or 15, wherein the enhanced DNase1 contains each of the E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

Embodiment 19. The fusion polypeptide of Embodiment 14 or 15, wherein the enhanced DNase1 contains each of the D53, Y65, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

Embodiment 20. The fusion polypeptide of Embodiment 14 or 15, wherein the enhanced DNase1 contains each of the D53, E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

Embodiment 21. The fusion polypeptide of Embodiment 14 or 15, wherein the enhanced DNase1 contains each of the Y65, E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

Embodiment 22. The fusion polypeptide of Embodiment 14 or 15, wherein the enhanced DNase1 contains each of the D53, Y65, E69, N74, and G105 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1.

Embodiment 23. The fusion of Embodiment 1, wherein the DNase has the amino acid sequence shown in
(i) residues 21-280 of SEQ ID NO:74,
(ii) residues 21-280 of SEQ ID NO:76,
(iii) residues 21-280 of SEQ ID NO:78,
(iv) residues 21-280 of SEQ ID NO:80,
(v) residues 21-280 of SEQ ID NO:82, or
(vi) residues 21-280 of SEQ ID NO:84.

Embodiment 24. The fusion polypeptide of Embodiment 1, wherein the DNase has at least 95% identity with the amino acid sequence shown in residues 21-290 of SEQ ID NO:34, and wherein each of the amino acids at positions corresponding to R80, R95, and N96 of SEQ ID NO:34 is alanine or serine.

Embodiment 25. The fusion polypeptide of Embodiment 24, wherein the DNase has the amino acid sequence shown in (i) residues 21-290 of SEQ ID NO:36 or (ii) residues 21-290 of SEQ ID NO:38.

Embodiment 26. The fusion polypeptide of any one of Embodiments 1 to 25, wherein L1 consists of from 26 to 60 amino acid residues.

Embodiment 27. The fusion polypeptide of Embodiment 26, wherein L1 consists of from 26 to 36 amino acid residues.

Embodiment 28. The fusion polypeptide of any one of Embodiments 1 to 27, wherein L1 comprises four or more tandem repeats of the amino acid sequence of SEQ ID NO:29.

Embodiment 29. The fusion polypeptide of Embodiment 26, wherein L1 has an amino acid sequence selected from the group consisting of SEQ ID NO:8 and SEQ ID NO:10.

Embodiment 30. The fusion polypeptide of any one of Embodiments 1 to 29, wherein the Fc region is a human Fc region.

Embodiment 31. The fusion polypeptide of Embodiment 30, wherein the human Fc region is an Fe variant comprising one or more amino acid substitutions relative to the wild-type human sequence.

Embodiment 32. The fusion polypeptide of Embodiment 30 or 31, wherein the Fc region is a human γ1 Fc region or a human γ4 Fc region.

Embodiment 33. The fusion polypeptide of Embodiment 31, wherein the Fc region is a human γ1 Fe variant in which Eu residue C220 is replaced by serine.

Embodiment 34. The fusion polypeptide of Embodiment 33, wherein Eu residues C226 and C229 are each replaced by serine.

Embodiment 35. The fusion polypeptide of Embodiment 34, wherein Eu residue P238 is replaced by serine.

Embodiment 36. The fusion polypeptide of Embodiment 32, wherein the Fc region is a human γ1 Fe variant in which Eu residue P331 is replaced by serine.

Embodiment 37. The fusion polypeptide of any one of Embodiments 33 to 35, wherein EU residue P331 is replaced by serine.

Embodiment 38. The fusion polypeptide of any one of Embodiments 1 to 29, wherein the Fc region has the amino acid sequence shown in (i) residues 1-232 or 1-231 of SEQ ID NO:12, (ii) residues 1-232 or 1-231 of SEQ ID NO:14, or (iii) residues 1-232 or 1-231 of SEQ ID NO:16.

Embodiment 39. The fusion polypeptide of Embodiment 1, wherein the fusion polypeptide comprises an amino acid sequence having at least 95% identity with the amino acid sequence shown in (i) residues 21-538 or 21-537 of SEQ ID NO:18, (ii) residues 21-548 or 21-547 of SEQ ID NO:20, (iii) residues 21-538 or 21-537 of SEQ ID NO:55, (iv) residues 21-548 or 21-547 of SEQ ID NO:56, (v) residues 21-538 or 21-537 of SEQ ID NO:86, (vi) residues 21-538 or 21-537 of SEQ ID NO:88, (vii) residues 21-538 or 21-537 of SEQ ID NO:90, (viii) residues 21-538 or 21-537 of SEQ ID NO:92, (ix) residues 21-538 or 21-537 of SEQ ID NO:94, (x) residues 21-538 or 21-537 of SEQ ID NO:96, (xi) residues 21-548 or 21-547 of SEQ ID NO:36, or (xii) residues 21-548 or 21-547 of SEQ ID NO:38.

Embodiment 40. The fusion polypeptide of any one of Embodiments 2 to 11, wherein the fusion polypeptide comprises an amino acid sequence having at least 95% identity with the amino acid sequence shown in (i) residues 21-538 or 21-537 of SEQ ID NO:18, (ii) residues 21-548 or 21-547 of SEQ ID NO:20, (iii) residues 21-538 or 21-537 of SEQ ID NO:55, or (iv) residues 21-548 or 21-547 of SEQ ID NO:56.

Embodiment 41. The fusion polypeptide of Embodiment 1, wherein the fusion polypeptide comprises the amino acid sequence shown in SEQ ID NO:49, SEQ ID NO:50, SEQ ID NO:53, or SEQ ID NO:54.

Embodiment 42. The fusion polypeptide of Embodiment 1, wherein the fusion polypeptide comprises the amino acid sequence shown in (i) residues 21-538 or 21-537 of SEQ ID NO:18 or (ii) residues 21-538 or 21-537 of SEQ ID NO:20.

Embodiments 43. The fusion polypeptide of Embodiment 1, wherein the fusion polypeptide comprises the amino acid sequence shown in (i) residues 21-538 or 21-537 of SEQ ID NO:55 or (ii) residues 21-548 or 21-547 of SEQ ID NO:56.

Embodiment 44. The fusion polypeptide of any one of Embodiments 13 to 22, wherein the fusion polypeptide comprises an amino acid sequence having at least 95% identity with the amino acid sequence shown in (i) residues 21-538 or 21-537 of SEQ ID NO:86, (ii) residues 21-538 or 21-537 of SEQ ID NO:88, (iii) residues 21-538 or 21-537 of SEQ ID NO:90, (iv) residues 21-538 or 21-537 of SEQ ID NO:92, (v) residues 21-538 or 21-537 of SEQ ID NO:94, or (vi) residues 21-538 or 21-537 of SEQ ID NO:96.

Embodiment 45. The fusion polypeptide of Embodiment 1, wherein the fusion polypeptide comprises the amino acid sequence shown in SEQ ID NO:97, SEQ ID NO:98, SEQ ID NO:99, SEQ ID NO:100, SEQ ID NO:101, SEQ ID NO:102, SEQ ID NO:103, or SEQ ID NO:104.

Embodiment 46. The fusion polypeptide of Embodiment 1, wherein the fusion polypeptide comprises the amino acid sequence shown in (i) residues 21-538 or 21-537 of SEQ ID NO:86, (ii) residues 21-538 or 21-537 of SEQ ID NO:88, (iii) residues 21-538 or 21-537 of SEQ ID NO:90, (iv) residues 21-538 or 21-537 of SEQ ID NO:92, (v) residues 21-538 or 21-537 of SEQ ID NO:94, or (vi) residues 21-538 or 21-537 of SEQ ID NO:96.

Embodiment 47. The fusion polypeptide of Embodiment 1, wherein the fusion polypeptide comprises the amino acid sequence shown in (i) residues 21-548 or 21-547 of SEQ ID NO:36 or (ii) residues 21-548 or 21-547 of SEQ ID NO:38.

Embodiment 48. A fusion polypeptide comprising, from an amino-terminal position to a carboxyl-terminal position, D-L1-X, wherein: D is a biologically active DNase having the amino acid sequence shown in
(i) residues 21-280 of SEQ ID NO:4,
(ii) residues 21-280 of SEQ ID NO:6,
(iii) residues 21-280 of SEQ ID NO:52,
(iv) residues 21-280 of SEQ ID NO:74,
(v) residues 21-280 of SEQ ID NO:76,
(vi) residues 21-280 of SEQ ID NO:78,
(vii) residues 21-280 of SEQ ID NO:80,
(viii) residues 21-280 of SEQ ID NO:82,
(ix) residues 21-280 of SEQ ID NO:84, or
(x) residues 21-290 of SEQ ID NO:34.

L1 is a first polypeptide linker, wherein L1 comprises at least 16 amino acid residues, and X is an immunoglobulin Fc region.

Embodiment 49. The fusion polypeptide of Embodiment 48, wherein L1 consists of from 16 to 60 amino acid residues.

Embodiment 50. The fusion polypeptide of Embodiment 49, wherein L1 consists of from 26 to 36 amino acid residues.

Embodiment 51. The fusion polypeptide of any one of Embodiments 48 to 50, wherein L1 comprises two or more tandem repeats of the amino acid sequence of SEQ ID NO:29.

Embodiment 52. The fusion polypeptide of Embodiment 48, wherein L1 has an amino acid sequence selected from the group consisting of SEQ ID NO:8 and SEQ ID NO:10.

Embodiment 53. The fusion polypeptide of any one of Embodiments 48 to 52, wherein the Fc region is a human Fc region.

Embodiment 54. The fusion polypeptide of Embodiment 53, wherein the human Fc region is an Fe variant comprising one or more amino acid substitutions relative to the wild-type human sequence.

Embodiment 55. The fusion polypeptide of Embodiment 53 or 54, wherein the Fc region is a human γ1 Fc region or a human γ4 Fc region.

Embodiment 56. The fusion polypeptide of Embodiment 54, wherein the Fc region is a human γ1 Fe variant in which Eu residue C220 is replaced by serine.

Embodiment 57. The fusion polypeptide of Embodiment 56, wherein Eu residues C226 and C229 are each replaced by serine.

Embodiment 58. The fusion polypeptide of Embodiment 57, wherein Eu residue P238 is replaced by serine.

Embodiment 59. The fusion polypeptide of Embodiment 55, wherein the Fc region is a human γ1 Fe variant in which Eu residue P331 is replaced by serine.

Embodiment 60. The fusion polypeptide of any one of Embodiments 56 to 58, wherein EU residue P331 is replaced by serine.

Embodiment 61. The fusion polypeptide of any one of Embodiments 48 to 52, wherein the Fc region has the amino acid sequence shown in (i) residues 1-232 or 1-231 of SEQ ID NO:12, (ii) residues 1-232 or 1-231 of SEQ ID NO:14, or (iii) residues 1-232 or 1-231 of SEQ ID NO:16.

Embodiment 62. A dimeric protein comprising a first fusion polypeptide and a second fusion polypeptide, wherein each of the first and second fusion polypeptides is a fusion polypeptide as defined in any one of Embodiments 1 to 61.

Embodiment 63. A polynucleotide encoding the fusion polypeptide of any one of Embodiments 1 to 61.

Embodiment 64. An expression cassette comprising a DNA segment encoding the fusion polypeptide of any one of Embodiments 1 to 61, wherein the DNA segment is operably linked to a promoter.

Embodiment 65. A cultured cell into which has been introduced the expression cassette of Embodiment 64, wherein the cell expresses the DNA segment.

Embodiment 66. A method of making a fusion polypeptide, the method comprising: culturing a cell into which has been introduced the expression cassette of Embodiment 64, wherein the cell expresses the DNA segment and the encoded fusion polypeptide is produced; and recovering the fusion polypeptide.

Embodiment 67. A method of making a dimeric protein, the method comprising: culturing a cell into which has been introduced the expression cassette of Embodiment 64, wherein the cell expresses the DNA segment and the encoded fusion polypeptide is produced as a dimeric protein; and recovering the dimeric protein.

Embodiment 68. An expression cassette comprising a DNA segment encoding the fusion polypeptide of any one of Embodiments 2 to 23, 40 to 46, and 48 to 61, wherein the DNA segment is operably linked to a promoter.

Embodiment 69. A stable cell line comprising, within its genomic DNA, the expression cassette of Embodiment 68, wherein the stable cell line constitutively expresses the DNA segment.

Embodiment 70. The stable cell line of Embodiment 69, wherein the stable cell line is a Chinese hamster ovary (CHO) cell line.

Embodiment 71. A method of making a fusion polypeptide, the method comprising: culturing a stable cell line comprising, within its genomic DNA, the expression cassette of Embodiment 68, wherein the stable cell line constitutively expresses the DNA segment and the encoded fusion polypeptide is produced; and recovering the fusion polypeptide.

Embodiment 72. A method of making a dimeric protein, the method comprising: culturing a stable cell line comprising, within its genomic DNA, the expression cassette of Embodiment 68, wherein the stable cell line constitutively expresses the DNA segment and the encoded fusion polypeptide is produced as a dimeric protein; and recovering the dimeric protein.

Embodiment 73. The method of Embodiment 71 or 72, wherein the stable cell line is a Chinese hamster ovary (CHO) cell line.

Embodiment 74. A vector comprising the expression cassette of Embodiment 64 or 68.

Embodiment 75. A composition comprising: a fusion polypeptide of any one of Embodiments 1 to 61; and a pharmaceutically acceptable carrier.

Embodiment 76. A composition comprising: a dimeric protein of Embodiment 62; and a pharmaceutically acceptable carrier.

Embodiment 77. The composition of Embodiment 75 or 76, wherein the composition is formulated for delivery to the lung by nebulization.

Embodiment 78. A method for treating a disease or disorder characterized by NETosis, the method comprising: administering to a subject having the disease or disorder characterized by NETosis an effective amount of a fusion polypeptide of any one of Embodiments 1 to 61 or a dimeric protein of Embodiment 62.

Embodiment 79. The method of Embodiment 78, wherein the disease or disorder is an inflammatory lung disease.

Embodiment 80. The method of Embodiment 79, wherein the inflammatory lung disease is selected from the group consisting of chronic obstructive pulmonary disease (COPD), asthma, cystic fibrosis (CF), bronchiectasis, hypoxia, acute respiratory distress syndrome (ARDS), and interstitial lung disease.

Embodiment 81. The method of Embodiment 80, wherein the interstitial lung disease is selected from the group consisting of idiopathic pulmonary fibrosis (IPF) and sarcoidosis.

Embodiment 82. The method of Embodiment 80, wherein the acute respiratory distress syndrome (ARDS) is associated with COVID-19.

Embodiment 83. The method of Embodiment 78, wherein the disease or disorder is an inflammatory skin disease.

Embodiment 84. The method of Embodiment 83, wherein the inflammatory skin disease is selected from the group consisting of psoriasis and atopic dermatitis.

Embodiment 85. The method of Embodiment 78, wherein the disease or disorder is an autoimmune disease.

Embodiment 86. The method of Embodiment 85, wherein the autoimmune disease is selected from the group consisting of systemic lupus erythematosus (SLE), rheumatoid arthritis (RA), psoriasis, type 1 diabetes mellitus, antiphospholipid syndrome, vasculitis, and systemic sclerosis.

Embodiment 87. The method of Embodiment 86, wherein the autoimmune disease is systemic lupus erythematosus (SLE) with lupus nephritis.

Embodiment 88. The method of Embodiment 78, wherein the disease or disorder is an autoinflammatory disease.

Embodiment 89. The method of Embodiment 88, wherein the autoinflammatory disease is selected from the group consisting of an inflammatory bowel disease (IBD) and gout.

Embodiment 90. The method of Embodiment 78, wherein the disease or disorder is a neurological disease or disorder.

Embodiment 91. The method of Embodiment 90, wherein the neurological disease or disorder is selected from the group consisting of a chronic neurodegenerative disease, a central nervous system (CNS) infection, and ischemic stroke.

Embodiment 92. The method of Embodiment 91, wherein the chronic neurodegenerative disease is selected from the group consisting of Alzheimer's disease and multiple sclerosis.

Embodiment 93. The method of Embodiment 91, wherein the CNS infection is selected from the group consisting of meningitis and cerebral malaria.

Embodiment 94. The method of Embodiment 78, wherein the disease or disorder is a metabolic disease.

Embodiment 95. The method of Embodiment 94, wherein the metabolic disease is selected from the group consisting of type 2 diabetes and obesity.

Embodiment 96. The method of Embodiment 78, wherein the disease or disorder is a cardiovascular disease.

Embodiment 97. The method of Embodiment 96, wherein the cardiovascular disease is characterized by atherosclerosis.

Embodiment 98. The method of Embodiment 97, wherein the cardiovascular disease is selected from the group consisting of coronary heart disease and ischemic stroke.

Embodiment 99. The method of Embodiment 98, wherein the coronary heart disease is characterized by acute coronary syndrome.

Embodiment 100. The method of Embodiment 78, wherein the disease or disorder is thrombosis.

Embodiment 101. The method of Embodiment 78, wherein the disease or disorder is sepsis.

Embodiment 102. The method of Embodiment 78, wherein the disease or disorder is ischemia reperfusion.

Embodiment 103. The method of Embodiment 78, wherein the disease or disorder is a chronic liver disease.

Embodiment 104. The method of Embodiment 103, wherein the chronic liver disease is nonalcoholic steatohepatitis (NASH).

Embodiment 105. The method of Embodiment 78, wherein the disease or disorder is a fibrotic disease.

Embodiment 106. The method of Embodiment 105, wherein the fibrotic disease is selected from the group consisting of systemic sclerosis, systemic lupus erythematosus (SLE), an inflammatory lung disease, a chronic liver disease, and a chronic kidney disease.

Embodiment 107. The method of Embodiment 106, wherein the chronic kidney disease is selected from the group consisting of lupus nephritis, IgA nephropathy, or membranous glomerulonephritis.

Embodiment 108. The method of Embodiment 78, wherein the disease or disorder is a cancer.

Embodiment 109. The method of Embodiment 108, wherein the cancer treatment is a combination therapy.

Embodiment 110. The method of Embodiment 109, wherein the combination therapy comprises an immunomodulatory therapy.

Embodiment 111. The method of Embodiment 110, wherein the immunomodulatory therapy comprises an immune checkpoint inhibitor.

Embodiment 112. The method of Embodiment 111, wherein the immunomodulatory therapy comprises an anti-PD-1/PD-Li therapy, an anti-CTLA-4 therapy, an anti-LAG-3 therapy, or a combination thereof.

Embodiment 113. The method of Embodiment 110, wherein the immunomodulatory therapy comprises a CAR T-cell therapy.

Embodiment 114. The method of Embodiment 109, wherein the combination therapy comprises a targeted therapy.

Embodiment 115. The method of Embodiment 114, wherein the targeted therapy comprises an antibody targeted therapy.

Embodiment 116. The method of Embodiment 115, wherein the targeted therapy comprises an antibody targeting an antigen selected from the group consisting of CD30, CD20, VEGF, BCMA, CD19, CD52, VEGFR2, ganglioside GD2, CD38, SLAMF7, HER2, EGFR, PDGFR, CD33, Nectin-4, CD79b, and Tissue Factor.

Embodiment 117. The method of Embodiment 114, wherein the targeted therapy comprises a small molecule targeted therapy.

Embodiment 118. The method of Embodiment 117, wherein the targeted therapy comprises a small molecule kinase inhibitor.

Embodiment 119. The method of Embodiment 118, wherein the kinase inhibitor is selected from the group consisting of alectinib, brigatinib, avapritinib, erdafitinib, bosutinib, encorafenib, zanubrutinib, vandetanib, cabozantinib, cobimetinib, mobocertinib, trametinib, binimetinib, neratinib, sorafenib, pemigatinib, erlotinib, sunitinib, regorafenib, dasatinib, asciminib, dabrafenib, upadacitinib, entrectinib, ripretinib, selpercatinib, ivosidenib, infigratinib, tucatinib, pexidartinib, iapatinib, crizotinib, gilteritinib, dacomitinib, larotrectinib, gefitinib, fedratinib, ruxolitinib, selumetinib, lenvatinib, lorlatinib, ibrutinib, afatinib, pralsetinib, enasidenib, ceritinib, niraparib, and vemurafenib.

Embodiment 120. The method of Embodiment 109, wherein the combination therapy comprises a chemotherapy.

Embodiment 121. The method of Embodiment 120, wherein the chemotherapy comprises a chemotherapy agent selected from the group consisting of methotrexate, adriamycin, 5-flurouracil, paclitaxel, busulfan, bleomycin, chlorambucil, idarubucin, hydroxyurea, gemcitabine, thalidomide, etoposide, arsenic trioxide, vinblastine, daunorubicin, vincristine, doxorubicin, procarbazine, tamoxifen, fludarabine, prednisolone, and a kinase inhibitor.

Embodiments 122. A method for reducing NETosis in a subject, the method comprising: administering to a subject having NETosis an effective amount of a fusion polypeptide of any one of Embodiments 1 to 61 or a dimeric protein of Embodiment 62.

Embodiment 123. A method for protecting a subject from aging, the method comprising: administering to the subject an effective amount of a fusion polypeptide of any one of Embodiments 1 to 61 or a dimeric protein of Embodiment 62.

### Exemplary Sequences

Exemplary nucleotide and amino acid sequences in accordance with the present disclosure are shown below in Table 5a. Variant amino acids of SEQ ID NOs:49, 50, 53, 54, and 97-104 are shown in Table 5b.

TABLE 5a
Nucleotide and Amino Acid Sequences

| SEQ | | |
|---|---|---|
| ID NO | Description | Sequence |
| 1 | Human wild- | atgaggggcatgaagctgctgggggcgctgctggcactggcggccctactgc |
| | type DNase1 | agggggccgtgtccctgaagatcgcagccttcaacatccagacatttgggga |
| | nucleotide | gaccaagatgtccaatgccaccctcgtcagctacattgtgcagatcctgagc |
| | | cgctatgacatcgccctggtccaggaggtcagagacagccacctgactgccg |
| | | tggggaagctgctggacaacctcaatcaggatgcaccagacacctatcacta |
| | | cgtggtcagtgagccactgggacggaacagctataaggagcgctacctgttc |
| | | gtgtacaggcctgaccaggtgtctgcggtggacagctactactacgatgatg |
| | | gctgcgagccctgcgggaacgacaccttcaaccgagagccagccattgtcag |
| | | gttcttctcccggttcacagaggtcagggagtttgccattgttcccctgcat |
| | | gcggccccgggggacgcagtagccgagatcgacgctctctatgacgtctacc |
| | | tggatgtccaagagaaatggggcttggaggacgtcatgttgatgggcgactt |
| | | caatgcgggctgcagctatgtgagaccctcccagtggtcatccatccgcctg |
| | | tggacaagccccaccttccagtggctgatccccgacagcgctgacaccacag |
| | | ctacacccacgcactgtgcctatgacaggatcgtggttgcagggatgctgct |
| | | ccgaggcgccgttgttcccgactcggctcttccctttaacttccaggctgcc |
| | | tatggcctgagtgaccaactggcccaagccatcagtgaccactatccagtgg |
| | | aggtgatgctgaagtga |
| 2 | Human wild- | MRGMKLLGALLALAALLQGAVSLKIAAFNIQTFGETKMSNATLVSYIVQILS |
| | type DNase1 | RYDIALVQEVRDSHLTAVGKLLDNLNQDAPDTYHYVVSEPLGRNSYKERYLF |
| | amino acid | VYRPDQVSAVDSYYYDDGCEPCGNDTFNREPAIVRFFSRFTEVREFAIVPLH |
| | | AAPGDAVAEIDALYDVYLDVQEKWGLEDVMLMGDFNAGCSYVRPSQWSSIRL |
| | | WTSPTFQWLIPDSADTTATPTHCAYDRIVVAGMLLRGAVVPDSALPFNFQAA |
| | | YGLSDQLAQAISDHYPVEVMLK |
| 3 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | N74K-G105R- | gatgtccaatgccaccctcgtcagctacattgtgcagatcctgagccgctat |
| | A114F] | gacatcgccctggtccaggaggtcagagacagccacctgactgccgtgggga |
| | nucleotide | agctgctggacaacctcaatcaggatgcaccagacacctatcactacgtggt |
| | | cagtgagccactgggacggaagagctataaggagcgctacctgttcgtgtac |
| | | aggcctgaccaggtgtctgcggtggacagctactactacgatgatggctgcg |
| | | agccctgcaggaacgacaccttcaaccgagagccattcattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 4 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R- | RPDQVSAVDSYYYDDGCEPCRNDTFNREPFIVRFFSRFTEVREFAIVPLHAA |
| | A114F] | PGDAVAEIDALYDVYLDVQEKWGLEDVMLMGDFNAGCSYVRPSQWSSIRLWT |
| | amino acid | SPTFQWLIPDSADTTATPTHCAYDRIVVAGMLLRGAVVPDSALPFNFQAAYG |
| | | LSDQLAQAISDHYPVEVMLK |
| 5 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | G105R-A114F] | gatgtccaatgccaccctcgtcagctacattgtgcagatcctgagccgctat |
| | nucleotide | gacatcgccctggtccaggaggtcagagacagccacctgactgccgtgggga |
| | | agctgctggacaacctcaatcaggatgcaccagacacctatcactacgtggt |
| | | cagtgagccactgggacggaacagctataaggagcgctacctgttcgtgtac |
| | | aggcctgaccaggtgtctgcggtggacagctactactacgatgatggctgcg |
| | | agccctgcaggaacgacaccttcaaccgagagccattcattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 6 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRNSYKERYLFVY |
| | G105R-A114F] | RPDQVSAVDSYYYDDGCEPCRNDTFNREPFIVRFFSRFTEVREFAIVPLHAA |
| | amino acid | PGDAVAEIDALYDVYLDVQEKWGLEDVMLMGDFNAGCSYVRPSQWSSIRLWT |
| | | SPTFQWLIPDSADTTATPTHCAYDRIVVAGMLLRGAVVPDSALPFNFQAAYG |
| | | LSDQLAQAISDHYPVEVMLK |
| 7 | (gly4ser)4 | agatctctccggaggaggtggctcaggtggtggaggatctggaggaggtggg |
| | linker | agtggtggaggtggttctaccggtctcgag |
| | nucleotide | |
| 8 | (gly4ser)4 | DLSGGGGSGGGGSGGGGSGGGGSTGL |
| | linker | |
| | amino acid | |
| 9 | (gly4ser)6 | agatctctccggaggaggtggctcaggtggtggaggatctggaggaggtggg |
| | linker | agtggtggaggtggttctggaggaggtggtagtggaggtggaggttctaccg |
| | nucleotide | gtctcgag |
| 10 | (gly4ser)6 | DLSGGGGSGGGGSGGGGSGGGGSGGGGSGGGGSTGL |
| | linker | |
| | amino acid | |
| 11 | Human IgG1 | gagcccaaatcttctgacaaaactcacacatgtccaccgtgcccagcacctg |
| | variant Fc | aactcctggggggaccgtcagtcttcctcttccccccaaaacccaaggacac |
| | nucleotide | cctcatgatctcccggacccctgaggtcacatgcgtggtggtggacgtgagc |
| | (SCC hinge, | cacgaagaccctgaggtcaagttcaactggtacgtggacggcgtggaggtgc |
| | wild-type CH2 | ataatgccaagacaaagccgcgggaggagcagtacaacagcacgtaccgtgt |
| | and CH3) | ggtcagcgtcctcaccgtcctgcaccaggactggctgaatggcaaggagtac |
| | | aagtgcaaggtctccaacaaagccctcccagcccccatcgagaaaaccatct |
| | | ccaaagccaaagggcagccccgagaaccacaggtgtacaccctgcccccatc |
| | | ccgggatgagctgaccaagaaccaggtcagcctgacctgcctggtcaaaggc |
| | | ttctatcccagcgacatcgccgtggagtgggagagcaatgggcagccggaga |
| | | acaactacaagaccacgcctcccgtgctggactccgacggctccttcttcct |
| | | ctacagcaagctcaccgtggacaagagcaggtggcagcaggggaacgtcttc |
| | | tcatgctccgtgatgcatgaggctctgcacaaccactacacgcagaagagcc |
| | | tctctctgtctccgggtaaa |
| 12 | Human IgG1 | EPKSSDKTHTCPPCPAPELLGGPSVFLFPPKPKDTLMISRTPEVTCVVVDVS |
| | variant Fc | HEDPEVKFNWYVDGVEVHNAKTKPREEQYNSTYRVVSVLTVLHQDWLNGKEY |
| | amino acid | KCKVSNKALPAPIEKTISKAKGQPREPQVYTLPPSRDELTKNQVSLTCLVKG |
| | (SCC hinge, | FYPSDIAVEWESNGQPENNYKTTPPVLDSDGSFFLYSKLTVDKSRWQQGNVF |
| | wild-type CH2 | SCSVMHEALHNHYTQKSLSLSPGK |
| | and CH3) | |
| 13 | Human IgG1 | gagcccaaatcttctgacaaaactcacacatctccaccgtccccagcacctg |
| | variant Fc | aactcctgggaggatcgtcagtcttcctcttccccccaaaacccaaggacac |
| | nucleotide | cctcatgatctcccggacccctgaggtcacatgcgtggtggtggacgtgagc |
| | (SSS hinge, | cacgaagaccctgaggtcaagttcaactggtacgtggacggcgtggaggtgc |
| | P238S, P331S) | ataatgccaagacaaagccgcgggaggagcagtacaacagcacgtaccgtgt |
| | | ggtcagcgtcctcaccgtcctgcaccaggactggctgaatggcaaggagtac |
| | | aagtgcaaggtctccaacaaagccctcccagcctccatcgagaaaaccatct |
| | | ccaaagccaaagggcagccccgagaaccacaggtgtacaccctgcccccatc |
| | | ccgggatgagctgaccaagaaccaggtcagcctgacctgcctggtcaaaggc |
| | | ttctatcccagcgacatcgccgtggagtgggagagcaatgggcagccggaga |
| | | acaactacaagaccacgcctcccgtgctggactccgacggctccttcttcct |
| | | ctacagcaagctcaccgtggacaagagcaggtggcagcaggggaacgtcttc |
| | | tcatgctccgtgatgcatgaggctctgcacaaccactacacgcagaagagcc |
| | | tctctctctctccgggtaaa |
| 14 | Human IgG1 | EPKSSDKTHTSPPSPAPELLGGSSVFLFPPKPKDTLMISRTPEVTCVVVDVS |
| | variant Fc | HEDPEVKFNWYVDGVEVHNAKTKPREEQYNSTYRVVSVLTVLHQDWLNGKEY |
| | amino acid | KCKVSNKALPASIEKTISKAKGQPREPQVYTLPPSRDELTKNQVSLTCLVKG |
| | (SSS hinge, | FYPSDIAVEWESNGQPENNYKTTPPVLDSDGSFFLYSKLTVDKSRWQQGNVF |
| | P238S, P331S) | SCSVMHEALHNHYTQKSLSLSPGK |
| 15 | Human IgG1 | gagcccaaatcttctgacaaaactcacacatctccaccgtccccagcacctg |
| | variant Fc | aactcctggggggatcgtcagtcttcctcttccccccaaaacccaaggacac |
| | nucleotide | cctcatgatctcccggacccctgaggtcacatgcgtggtggtggacgtgagc |
| | (SSS hinge, | cacgaagaccctgaggtcaagttcaactggtacgtggacggcgtggaggtgc |
| | P238S) | ataatgccaagacaaagccgcgggaggagcagtacaacagcacgtaccgtgt |
| | | ggtcagcgtcctcaccgtcctgcaccaggactggctgaatggcaaggagtac |
| | | aagtgcaaggtctccaacaaagccctcccagcccccatcgagaaaaccatct |
| | | ccaaagccaaagggcagccccgagaaccacaggtgtacaccctgcccccatc |
| | | ccgggatgagctgaccaagaaccaggtcagcctgacctgcctggtcaaaggc |
| | | ttctatcccagcgacatcgccgtggagtgggagagcaatgggcagccggaga |
| | | acaactacaagaccacgcctcccgtgctggactccgacggctccttcttcct |
| | | ctacagcaagctcaccgtggacaagagcaggtggcagcaggggaacgtcttc |
| | | tcatgctccgtgatgcatgaggctctgcacaaccactacacgcagaagagcc |
| | | tctctctgtctccgggtaaa |
| 16 | Human IgG1 | EPKSSDKTHTSPPSPAPELLGGSSVFLFPPKPKDTLMISRTPEVTCVVVDVS |
| | variant Fc | HEDPEVKFNWYVDGVEVHNAKTKPREEQYNSTYRVVSVLTVLHQDWLNGKEY |
| | amino acid | KCKVSNKALPAPIEKTISKAKGQPREPQVYTLPPSRDELTKNQVSLTCLVKG |
| | (SSS hinge, | FYPSDIAVEWESNGQPENNYKTTPPVLDSDGSFFLYSKLTVDKSRWQQGNVF |
| | P238S) | SCSVMHEALHNHYTQKSLSLSPGK |
| 17 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | N74K-G105R- | gatgtccaatgccaccctcgtcagctacattgtgcagatcctgagccgctat |
| | A114F]-(g4s)4- | gacatcgccctggtccaggaggtcagagacagccacctgactgccgtgggga |
| | [SSShinge- | agctgctggacaacctcaatcaggatgcaccagacacctatcactacgtggt |
| | P238S-P331S | cagtgagccactgggacggaagagctataaggagcgctacctgttcgtgtac |
| | Fc] | aggcctgaccaggtgtctgcggtggacagctactactacgatgatggctgcg |
| | nucleotide | agccctgcaggaacgacaccttcaaccgagagccattcattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 18 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N96K-G127R- | RPDQVSAVDSYYYDDGCEPCRNDTFNREPFIVRFFSRFTEVREFAIVPLHAA |
| | A136F]-(g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc] | KTHTSPPSPAPELLGGSSVFLFPPKPKDTLMISRTPEVTCVVVDVSHEDPEV |
| | amino acid | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 19 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | N74K-G105R- | gatgtccaatgccaccctcgtcagctacattgtgcagatcctgagccgctat |
| | A114F]-(g4s)6- | gacatcgccctggtccaggaggtcagagacagccacctgactgccgtgggga |
| | [SSShinge- | agctgctggacaacctcaatcaggatgcaccagacacctatcactacgtggt |
| | P238S-P331S | cagtgagccactgggacggaagagctataaggagcgctacctgttcgtgtac |
| | Fc] | aggcctgaccaggtgtctgcggtggacagctactactacgatgatggctgcg |
| | nucleotide | agccctgcaggaacgacaccttcaaccgagagccattcattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctggaggaggtggtagtggaggtggaggt |
| | | tctaccggtctcgagcccaaatcttctgacaaaactcacacatctccaccgt |
| | | ccccagcacctgaactcctgggaggatcgtcagtcttcctcttccccccaaa |
| | | acccaaggacaccctcatgatctcccggacccctgaggtcacatgcgtggtg |
| | | gtggacgtgagccacgaagaccctgaggtcaagttcaactggtacgtggacg |
| | | gcgtggaggtgcataatgccaagacaaagccgcgggaggagcagtacaacag |
| | | cacgtaccgtgtggtcagcgtcctcaccgtcctgcaccaggactggctgaat |
| | | ggcaaggagtacaagtgcaaggtctccaacaaagccctcccagcctccatcg |
| | | agaaaaccatctccaaagccaaagggcagccccgagaaccacaggtgtacac |
| | | cctgcccccatcccgggatgagctgaccaagaaccaggtcagcctgacctgc |
| | | ctggtcaaaggcttctatcccagcgacatcgccgtggagtgggagagcaatg |
| | | ggcagccggagaacaactacaagaccacgcctcccgtgctggactccgacgg |
| | | ctccttcttcctctacagcaagctcaccgtggacaagagcaggtggcagcag |
| | | gggaacgtcttctcatgctccgtgatgcatgaggctctgcacaaccactaca |
| | | cgcagaagagcctctctctctctccgggtaaa |
| 20 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R- | RPDQVSAVDSYYYDDGCEPCRNDTFNREPFIVRFFSRFTEVREFAIVPLHAA |
| | A136F]-(g4s)6- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSGGGGSGGGG |
| | Fc] | STGLEPKSSDKTHTSPPSPAPELLGGSSVFLFPPKPKDTLMISRTPEVTCVV |
| | amino acid | VDVSHEDPEVKFNWYVDGVEVHNAKTKPREEQYNSTYRVVSVLTVLHQDWLN |
| | | GKEYKCKVSNKALPASIEKTISKAKGQPREPQVYTLPPSRDELTKNQVSLTC |
| | | LVKGFYPSDIAVEWESNGQPENNYKTTPPVLDSDGSFFLYSKLTVDKSRWQQ |
| | | GNVFSCSVMHEALHNHYTQKSLSLSPGK |
| 21 | Human PON1 | atggcgaagctgattgcgctcaccctcttggggatgggactggcactcttca |
| | (paraoxonase/ | ggaaccaccagtcttcttaccaaacacgacttaatgctctccgagaggtaca |
| | arylesterase) | acccgtagaacttcctaactgtaatttagttaaaggaatcgaaactggctct |
| | nucleotide | gaagacttggagatactgcctaatggactggctttcattagctctggattaa |
| | | agtatcctggaataaagagcttcaaccccaacagtcctggaaaaatacttct |
| | | gatggacctgaatgaagaagatccaacagtgttggaattggggatcactgga |
| | | agtaaatttgatgtatcttcatttaaccctcatgggattagcacattcacag |
| | | atgaagataatgccatgtacctcctggtggtgaaccatccagatgccaagtc |
| | | cacagtggagttgtttaaatttcaagaagaagaaaaatcgcttttgcatcta |
| | | aaaaccatcagacataaacttctgcctaatttgaatgatattgttgctgtgg |
| | | gacctgagcacttttatggcacaaatgatcactattttcttgacccctactt |
| | | acaatcctgggagatgtatttgggtttagcgtggtcgtatgttgtctactat |
| | | agtccaagtgaagttcgagtggtggcagaaggatttgattttgctaatggaa |
| | | tcaacatttcacccgatggcaagtatgtctatatagctgagttgctggctca |
| | | taagattcatgtgtatgaaaagcatgctaattggactttaactccattgaag |
| | | tcccttgactttaataccctcgtggataacatatctgtggatcctgagacag |
| | | gagacctttgggttggatgccatcccaatggcatgaaaatcttcttctatga |
| | | ctcagagaatcctcctgcatcagaggtgcttcgaatccagaacattctaaca |
| | | gaagaacctaaagtgacacaggtttatgcagaaaatggcacagtgttgcaag |
| | | gcagtacagttgcctctgtgtacaaagggaaactgctgattggcacagtgtt |
| | | tcacaaagctctttactgtgagctctaacagaccgatttgcacccatgccat |
| | | agaaact |
| 22 | Human PON1 | MAKLIALTLLGMGLALFRNHQSSYQTRLNALREVQPVELPNCNLVKGIETGS |
| | amino acid | EDLEILPNGLAFISSGLKYPGIKSFNPNSPGKILLMDLNEEDPTVLELGITG |
| | | SKFDVSSFNPHGISTFTDEDNAMYLLVVNHPDAKSTVELFKFQEEEKSLLHL |
| | | KTIRHKLLPNLNDIVAVGPEHFYGTNDHYFLDPYLQSWEMYLGLAWSYVVYY |
| | | SPSEVRVVAEGFDFANGINISPDGKYVYIAELLAHKIHVYEKHANWTLTPLK |
| | | SLDFNTLVDNISVDPETGDLWVGCHPNGMKIFFYDSENPPASEVLRIQNILT |
| | | EEPKVTQVYAENGTVLQGSTVASVYKGKLLIGTVFHKALYCEL |
| 23 | Human PON1 | atggcgaagctgattgcgctcaccctcttggggatgggactggcactcttca |
| | Q192K | ggaaccaccagtcttcttaccaaacacgacttaatgctctccgagaggtaca |
| | nucleotide | acccgtagaacttcctaactgtaatttagttaaaggaatcgaaactggctct |
| | | gaagacttggagatactgcctaatggactggctttcattagctctggattaa |
| | | agtatcctggaataaagagcttcaaccccaacagtcctggaaaaatacttct |
| | | gatggacctgaatgaagaagatccaacagtgttggaattggggatcactgga |
| | | agtaaatttgatgtatcttcatttaaccctcatgggattagcacattcacag |
| | | atgaagataatgccatgtacctcctggtggtgaaccatccagatgccaagtc |
| | | cacagtggagttgtttaaatttcaagaagaagaaaaatcgcttttgcatcta |
| | | aaaaccatcagacataaacttctgcctaatttgaatgatattgttgctgtgg |
| | | gacctgagcacttttatggcacaaatgatcactattttcttgacccctactt |
| | | aaaatcctgggagatgtatttgggtttagcgtggtcgtatgttgtctactat |
| | | agtccaagtgaagttcgagtggtggcagaaggatttgattttgctaatggaa |
| | | tcaacatttcacccgatggcaagtatgtctatatagctgagttgctggctca |
| | | taagattcatgtgtatgaaaagcatgctaattggactttaactccattgaag |
| | | tcccttgactttaataccctcgtggataacatatctgtggatcctgagacag |
| | | gagacctttgggttggatgccatcccaatggcatgaaaatcttcttctatga |
| | | ctcagagaatcctcctgcatcagaggtgcttcgaatccagaacattctaaca |
| | | gaagaacctaaagtgacacaggtttatgcagaaaatggcacagtgttgcaag |
| | | gcagtacagttgcctctgtgtacaaagggaaactgctgattggcacagtgtt |
| | | tcacaaagctctttactgtgagctctaa |
| 24 | Human PON1 | MAKLIALTLLGMGLALFRNHQSSYQTRLNALREVQPVELPNCNLVKGIETGS |
| | Q192K | EDLEILPNGLAFISSGLKYPGIKSFNPNSPGKILLMDLNEEDPTVLELGITG |
| | amino acid | SKFDVSSFNPHGISTFTDEDNAMYLLVVNHPDAKSTVELFKFQEEEKSLLHL |
| | | KTIRHKLLPNLNDIVAVGPEHFYGTNDHYFLDPYLKSWEMYLGLAWSYVVYY |
| | | SPSEVRVVAEGFDFANGINISPDGKYVYIAELLAHKIHVYEKHANWTLTPLK |
| | | SLDFNTLVDNISVDPETGDLWVGCHPNGMKIFFYDSENPPASEVLRIQNILT |
| | | EEPKVTQVYAENGTVLQGSTVASVYKGKLLIGTVFHKALYCEL |
| 25 | hVK3LP- | aagcttgccgccatggaaaccccagcgcagcttctcttcctcctgctactct |
| | [hDNase1 | ggctcccagataccaccggtctgaagatcgcagccttcaacatccagacatt |
| | N74K-G105R- | tggggagaccaagatgtccaatgccaccctcgtcagctacattgtgcagatc |
| | A114F]-(g4s)4- | ctgagccgctatgacatcgccctggtccaggaggtcagagacagccacctga |
| | [SSShinge- | ctgccgtggggaagctgctggacaacctcaatcaggatgcaccagacaccta |
| | P238S-P331S | tcactacgtggtcagtgagccactgggacggaagagctataaggagcgctac |
| | Fc]-NGS- | ctgttcgtgtacaggcctgaccaggtgtctgcggtggacagctactactacg |
| | [PON1 Q192K] | atgatggctgcgagccctgcaggaacgacaccttcaaccgagagccattcat |
| | nucleotide | tgtcaggttcttctcccggttcacagaggtcagggagtttgccattgttccc |
| | | ctgcatgcggccccgggggacgcagtagccgagatcgacgctctctatgacg |
| | | tctacctggatgtccaagagaaatggggcttggaggacgtcatgttgatggg |
| | | cgacttcaatgcgggctgcagctatgtgagaccctcccagtggtcatccatc |
| | | cgcctgtggacaagccccaccttccagtggctgatccccgacagcgctgaca |
| | | ccacagctacacccacgcactgtgcctatgacaggatcgtggttgcagggat |
| | | gctgctccgaggcgccgttgttcccgactcggctcttccctttaacttccag |
| | | gctgcctatggcctgagtgaccaactggcccaagccatcagtgaccactatc |
| | | cagtggaggtgatgctgaaagatctctccggaggaggtggctcaggtggtgg |
| | | aggatctggaggaggtgggagtggtggaggtggttctaccggtctcgagccc |
| | | aaatcttctgacaaaactcacacatctccaccgtccccagcacctgaactcc |
| | | tgggaggatcgtcagtcttcctcttccccccaaaacccaaggacaccctcat |
| | | gatctcccggacccctgaggtcacatgcgtggtggtggacgtgagccacgaa |
| | | gaccctgaggtcaagttcaactggtacgtggacggcgtggaggtgcataatg |
| | | ccaagacaaagccgcgggaggagcagtacaacagcacgtaccgtgtggtcag |
| | | cgtcctcaccgtcctgcaccaggactggctgaatggcaaggagtacaagtgc |
| | | aaggtctccaacaaagccctcccagcctccatcgagaaaaccatctccaaag |
| | | ccaaagggcagccccgagaaccacaggtgtacaccctgcccccatcccggga |
| | | tgagctgaccaagaaccaggtcagcctgacctgcctggtcaaaggcttctat |
| | | cccagcgacatcgccgtggagtgggagagcaatgggcagccggagaacaact |
| | | acaagaccacgcctcccgtgctggactccgacggctccttcttcctctacag |
| | | caagctcaccgtggacaagagcaggtggcagcaggggaacgtcttctcatgc |
| | | tccgtgatgcatgaggctctgcacaaccactacacgcagaagagcctctctc |
| | | tctctccgggtaaagtcgacggagctagcagccccgtgaacgtgagcagccc |
| | | cagcgtgcaggatatcctcttcaggaaccaccagtcttcttaccaaacacga |
| | | cttaatgctctccgagaggtacaacccgtagaacttcctaactgtaatttag |
| | | ttaaaggaatcgaaactggctctgaagacttggagatactgcctaatggact |
| | | ggctttcattagctctggattaaagtatcctggaataaagagcttcaacccc |
| | | aacagtcctggaaaaatacttctgatggacctgaatgaagaagatccaacag |
| | | tgttggaattggggatcactggaagtaaatttgatgtatcttcatttaaccc |
| | | tcatgggattagcacattcacagatgaagataatgccatgtacctcctggtg |
| | | gtgaaccatccagatgccaagtccacagtggagttgtttaaatttcaagaag |
| | | aagaaaaatcgcttttgcatctaaaaaccatcagacataaacttctgcctaa |
| | | tttgaatgatattgttgctgtgggacctgagcacttttatggcacaaatgat |
| | | cactattttcttgacccctacttaaaatcctgggagatgtatttgggtttag |
| | | cgtggtcgtatgttgtctactatagtccaagtgaagttcgagtggtggcaga |
| | | aggatttgattttgctaatggaatcaacatttcacccgatggcaagtatgtc |
| | | tatatagctgagttgctggctcataagattcatgtgtatgaaaagcatgcta |
| | | attggactttaactccattgaagtcccttgactttaataccctcgtggataa |
| | | catatctgtggatcctgagacaggagacctttgggttggatgccatcccaat |
| | | ggcatgaaaatcttcttctatgactcagagaatcctcctgcatcagaggtgc |
| | | ttcgaatccagaacattctaacagaagaacctaaagtgacacaggtttatgc |
| | | agaaaatggcacagtgttgcaaggcagtacagttgcctctgtgtacaaaggg |
| | | aaactgctgattggcacagtgtttcacaaagctctttactgtgagctctaat |
| | | aatctagaa |
| 26 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYWSEPLGRKSYKERYLFVY |
| | N74K-G105R- | RPDQVSAVDSYYYDDGCEPCRNDTFNREPFIVRFFSRFTEVREFAIVPLHAA |
| | A114F]-(g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIWAGMLLRGAWPDSALPFNFQAAYG |
| | P238S-P331S | LSDQLAQAISDHYPVEVMLKDLSGGGGSGGGGSGGGGSGGGGSTGLEPKSSD |
| | Fc]-NGS- | KTHTSPPSPAPELLGGSSVFLFPPKPKDTLMISRTPEVTCVVVDVSHEDPEV |
| | [PON1 Q192K] | KFNWYVDGVEVHNAKTKPREEQYNSTYRWSVLTVLHQDWLNGKEYKCKVSN |
| | amino acid | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGKVDGASSPVNVSSPSVQDILFRNHQSSYQTRLNAL |
| | | REVQPVELPNCNLVKGIETGSEDLEILPNGLAFISSGLKYPGIKSFNPNSPG |
| | | KILLMDLNEEDPTVLELGITGSKFDVSSFNPHGISTFTDEDNAMYLLVVNHP |
| | | DAKSTVELFKFQEEEKSLLHLKTIRHKLLPNLNDIVAVGPEHFYGTNDHYFL |
| | | DPYLKSWEMYLGLAWSYWYYSPSEVRWAEGFDFANGINISPDGKYVYIAE |
| | | LLAHKIHVYEKHANWTLTPLKSLDFNTLVDNISVDPETGDLWVGCHPNGMKI |
| | | FFYDSENPPASEVLRIQNILTEEPKVTQVYAENGTVLQGSTVASVYKGKLLI |
| | | GTVFHKALYCEL |
| 27 | Human VK3 | aagcttgccgccatggaaaccccagcgcagcttctcttcctcctgctactct |
| | leader peptide | ggctcccagataccaccggt |
| | nucleotide | |
| 28 | Human VK3 | METPAQLLFLLLLWLPDTTG |
| | leader peptide | |
| | amino acid | |
| 29 | (gly4ser) | GGGGS |
| | tandem repeat | |
| | sequence | |
| | amino acid | |
| 30 | Human wild- | LKIAAFNIQTFGETKMSNATLVSYIVQILSRYDIALVQEVRDSHLTAVGKLL |
| | type DNase1 | DNLNQDAPDTYHYVVSEPLGRNSYKERYLFVYRPDQVSAVDSYYYDDGCEPC |
| | (mature) | GNDTFNREPAIVRFFSRFTEVREFAIVPLHAAPGDAVAEIDALYDVYLDVQE |
| | amino acid | KWGLEDVMLMGDFNAGCSYVRPSQWSSIRLWTSPTFQWLIPDSADTTATPTH |
| | | CAYDRIVVAGMLLRGAVVPDSALPFNFQAAYGLSDQLAQAISDHYPVEVMLK |
| 31 | Peptide linker | gtcgacggagctagcagccccgtgaacgtgagcagccccagcgtgcaggata |
| | containing N- | tc |
| | linked | |
| | glycosylation | |
| | site (NGS | |
| | linker) | |
| | nucleotide | |
| 32 | NGS linker | VDGASSPVNVSSPSVQDI |
| | amino acid | |
| 33 | Human wild- | atgtcacgggagctggccccactgctgcttctcctcctctccatccacagcg |
| | type DNase1L3 | ccctggccatgaggatctgctccttcaacgtcaggtcctttggggaaagcaa |
| | nucleotide | gcaggaagacaagaatgccatggatgtcattgtgaaggtcatcaaacgctgt |
| | | gacatcatactcgtgatggaaatcaaggacagcaacaacaggatctgcccca |
| | | tactgatggagaagctgaacagaaattcaaggagaggcataacgtacaacta |
| | | tgtgattagctctcggcttggaagaaacacatataaagaacaatatgccttt |
| | | ctctacaaggaaaagctggtgtctgtgaagaggagttatcactaccatgact |
| | | atcaggatggagacgcagatgtgttttccagggagccctttgtggtctggtt |
| | | ccaatctccccacactgctgtcaaagacttcgtgattatccccctgcacacc |
| | | accccagagacatccgttaaggagatcgatgagttggttgaggtctacacgg |
| | | acgtgaaacaccgctggaaggcggagaatttcattttcatgggtgacttcaa |
| | | tgccggctgcagctacgtccccaagaaggcctggaagaacatccgcttgagg |
| | | actgaccccaggtttgtttggctgatcggggaccaagaggacaccacggtga |
| | | agaagagcaccaactgtgcatatgacaggattgtgcttagaggacaagaaat |
| | | cgtcagttctgttgttcccaagtcaaacagtgtttttgacttccagaaagct |
| | | tacaagctgactgaagaggaggccctggatgtcagcgaccactttccagttg |
| | | aatttaaactacagtcttcaagggccttcaccaacagcaaaaaatctgtcac |
| | | tctaaggaagaaaacaaagaggaaacgctcctacccaactttcttgtacaaa |
| | | gttggcattataagaaagcattgcttatcaatttgttgcaacgaac |
| 34 | Human wild- | MSRELAPLLLLLLSIHSALAMRICSFNVRSFGESKQEDKNAMDVIVKVIKRC |
| | type DNase1L3 | DIILVMEIKDSNNRICPILMEKLNRNSRRGITYNYVISSRLGRNTYKEQYAF |
| | amino acid | LYKEKLVSVKRSYHYHDYQDGDADVFSREPFVVWFQSPHTAVKDFVIIPLHT |
| | | TPETSVKEIDELVEVYTDVKHRWKAENFIFMGDFNAGCSYVPKKAWKNIRLR |
| | | TDPRFVWLIGDQEDTTVKKSTNCAYDRIVLRGQEIVSSVVPKSNSVFDFQKA |
| | | YKLTEEEALDVSDHFPVEFKLQSSRAFTNSKKSVTLRKKTKRKRS |
| 35 | hVK3LP- | atggaaaccccagcgcagcttctcttcctcctgctactctggctcccagata |
| | [hDNase1L3 | ccaccggtatgaggatctgctccttcaacgtcaggtcctttggggaaagcaa |
| | NLS1mutAAA, | gcaggaagacaagaatgccatggatgtcattgtgaaggtcatcaaacgctgt |
| | NLS2delta]- | gacatcatactcgtgatggaaatcaaggacagcaacaacaggatctgcccca |
| | (g4s)4- | tactgatggagaagctgaacagaaattcagcaagaggcataacgtacaacta |
| | [SSShinge- | tgtgattagctctcggcttggagcagctacatataaagaacaatatgccttt |
| | P238S-P331S | ctctacaaggaaaagctggtgtctgtgaagaggagttatcactaccatgact |
| | Fc] | atcaggatggagacgcagatgtgttttccagggagccctttgtggtctggtt |
| | nucleotide | ccaatctccccacactgctgtcaaagacttcgtgattatccccctgcacacc |
| | | accccagagacatccgttaaggagatcgatgagttggttgaggtctacacgg |
| | | acgtgaaacaccgctggaaggcggagaatttcattttcatgggtgacttcaa |
| | | tgccggctgcagctacgtccccaagaaggcctggaagaacatccgcttgagg |
| | | actgaccccaggtttgtttggctgatcggggaccaagaggacaccacggtga |
| | | agaagagcaccaactgtgcatatgacaggattgtgcttagaggacaagaaat |
| | | cgtcagttctgttgttcccaagtcaaacagtgtttttgacttccagaaagct |
| | | tacaagctgactgaagaggaggccctggatgtcagcgaccactttccagttg |
| | | aatttaaactacagtcttcaagggccttcaccaactcagatctctccggagg |
| | | aggtggctcaggtggtggaggatctggaggaggtgggagtggtggaggtggt |
| | | tctaccggtctcgagcccaaatcttctgacaaaactcacacatctccaccgt |
| | | ccccagcacctgaactcctgggaggatcgtcagtcttcctcttccccccaaa |
| | | acccaaggacaccctcatgatctcccggacccctgaggtcacatgcgtggtg |
| | | gtggacgtgagccacgaagaccctgaggtcaagttcaactggtacgtggacg |
| | | gcgtggaggtgcataatgccaagacaaagccgcgggaggagcagtacaacag |
| | | cacgtaccgtgtggtcagcgtcctcaccgtcctgcaccaggactggctgaat |
| | | ggcaaggagtacaagtgcaaggtctccaacaaagccctcccagcctccatcg |
| | | agaaaaccatctccaaagccaaagggcagccccgagaaccacaggtgtacac |
| | | cctgcccccatcccgggatgagctgaccaagaaccaggtcagcctgacctgc |
| | | ctggtcaaaggcttctatcccagcgacatcgccgtggagtgggagagcaatg |
| | | ggcagccggagaacaactacaagaccacgcctcccgtgctggactccgacgg |
| | | ctccttcttcctctacagcaagctcaccgtggacaagagcaggtggcagcag |
| | | gggaacgtcttctcatgctccgtgatgcatgaggctctgcacaaccactaca |
| | | cgcagaagagcctctctctctctccgggtaaataa |
| 36 | hVK3LP- | METPAQLLFLLLLWLPDTTGMRICSFNVRSFGESKQEDKNAMDVIVKVIKRC |
| | [hDNase1L3 | DIILVMEIKDSNNRICPILMEKLNRNSARGITYNYVISSRLGAATYKEQYAF |
| | NLS1mutAAA, | LYKEKLVSVKRSYHYHDYQDGDADVFSREPFVVWFQSPHTAVKDFVIIPLHT |
| | NLS2delta]- | TPETSVKE1DELVEVYTDVKHRWKAENFIFMGDFNAGCSYVPKKAWKNIRLR |
| | (g4s)4- | TDPRFVWLIGDQEDTTVKKSTNCAYDRIVLRGQEIVSSVVPKSNSVFDFQKA |
| | [SSShinge- | YKLTEEEALDVSDHFPVEFKLQSSRAFTNSDLSGGGGSGGGGSGGGGSGGGG |
| | P238S-P331S | STGLEPKSSDKTHTSPPSPAPELLGGSSVFLFPPKPKDTLMISRTPEVTCVV |
| | Fc] | VDVSHEDPEVKFNWYVDGVEVHNAKTKPREEQYNSTYRVVSVLTVLHQDWLN |
| | amino acid | GKEYKCKVSNKALPASIEKTISKAKGQPREPQVYTLPPSRDELTKNQVSLTC |
| | | LVKGFYPSDIAVEWESNGQPENNYKTTPPVLDSDGSFFLYSKLTVDKSRWQQ |
| | | GNVFSCSVMHEALHNHYTQKSLSLSPGK |
| 37 | hVK3LP- | atggaaaccccagcgcagcttctcttcctcctgctactctggctcccagata |
| | [hDNase1L3 | ccaccggtatgaggatctgctccttcaacgtcaggtcctttggggaaagcaa |
| | NLS1mutSSS, | gcaggaagacaagaatgccatggatgtcattgtgaaggtcatcaaacgctgt |
| | NLS2delta]- | gacatcatactcgtgatggaaatcaaggacagcaacaacaggatctgcccca |
| | (g4s)4- | tactgatggagaagctgaacagaaattcatcaagaggcataacgtacaacta |
| | [SSShinge- | tgtgattagctctcggcttggaagttctacatataaagaacaatatgccttt |
| | P238S-P331S | ctctacaaggaaaagctggtgtctgtgaagaggagttatcactaccatgact |
| | Fc] | atcaggatggagacgcagatgtgttttccagggagccctttgtggtctggtt |
| | nucleotide | ccaatctccccacactgctgtcaaagacttcgtgattatccccctgcacacc |
| | | accccagagacatccgttaaggagatcgatgagttggttgaggtctacacgg |
| | | acgtgaaacaccgctggaaggcggagaatttcattttcatgggtgacttcaa |
| | | tgccggctgcagctacgtccccaagaaggcctggaagaacatccgcttgagg |
| | | actgaccccaggtttgtttggctgatcggggaccaagaggacaccacggtga |
| | | agaagagcaccaactgtgcatatgacaggattgtgcttagaggacaagaaat |
| | | cgtcagttctgttgttcccaagtcaaacagtgtttttgacttccagaaagct |
| | | tacaagctgactgaagaggaggccctggatgtcagcgaccactttccagttg |
| | | aatttaaactacagtcttcaagggccttcaccaactcagatctctccggagg |
| | | aggtggctcaggtggtggaggatctggaggaggtgggagtggtggaggtggt |
| | | tctaccggtctcgagcccaaatcttctgacaaaactcacacatctccaccgt |
| | | ccccagcacctgaactcctgggaggatcgtcagtcttcctcttccccccaaa |
| | | acccaaggacaccctcatgatctcccggacccctgaggtcacatgcgtggtg |
| | | gtggacgtgagccacgaagaccctgaggtcaagttcaactggtacgtggacg |
| | | gcgtggaggtgcataatgccaagacaaagccgcgggaggagcagtacaacag |
| | | cacgtaccgtgtggtcagcgtcctcaccgtcctgcaccaggactggctgaat |
| | | ggcaaggagtacaagtgcaaggtctccaacaaagccctcccagcctccatcg |
| | | agaaaaccatctccaaagccaaagggcagccccgagaaccacaggtgtacac |
| | | cctgcccccatcccgggatgagctgaccaagaaccaggtcagcctgacctgc |
| | | ctggtcaaaggcttctatcccagcgacatcgccgtggagtgggagagcaatg |
| | | ggcagccggagaacaactacaagaccacgcctcccgtgctggactccgacgg |
| | | ctccttcttcctctacagcaagctcaccgtggacaagagcaggtggcagcag |
| | | gggaacgtcttctcatgctccgtgatgcatgaggctctgcacaaccactaca |
| | | cgcagaagagcctctctctctctccgggtaaataa |
| 38 | hVK3LP- | METPAQLLFLLLLWLPDTTGMRICSFNVRSFGESKQEDKNAMDVIVKVIKRC |
| | [hDNase1L3 | DIILVMEIKDSNNRICPILMEKLNRNSSRGITYNYVISSRLGSSTYKEQYAF |
| | NLS1mutSSS, | LYKEKLVSVKRSYHYHDYQDGDADVFSREPFVVWFQSPHTAVKDFVIIPLHT |
| | NLS2delta]- | TPETSVKE1DELVEVYTDVKHRWKAENFIFMGDFNAGCSYVPKKAWKNIRLR |
| | (g4s)4- | TDPRFVWLIGDQEDTTVKKSTNCAYDRIVLRGQEIVSSVVPKSNSVFDFQKA |
| | [SSShinge- | YKLTEEEALDVSDHFPVEFKLQSSRAFTNSDLSGGGGSGGGGSGGGGSGGGG |
| | P238S-P331S | STGLEPKSSDKTHTSPPSPAPELLGGSSVFLFPPKPKDTLMISRTPEVTCVV |
| | Fc] | VDVSHEDPEVKFNWYVDGVEVHNAKTKPREEQYNSTYRVVSVLTVLHQDWLN |
| | amino acid | GKEYKCKVSNKALPASIEKTISKAKGQPREPQVYTLPPSRDELTKNQVSLTC |
| | | LVKGFYPSDIAVEWESNGQPENNYKTTPPVLDSDGSFFLYSKLTVDKSRWQQ |
| | | GNVFSCSVMHEALHNHYTQKSLSLSPGK |
| 39 | Human PON1 | atggctaaactgacagcgctcacactcttggggctgggattggcactcttcg |
| | variant G3C9 | atggacagaagtcttctttccaaacacgatttaatgttcaccgtgaagtaac |
| | nucleotide | tccagtggaacttcctaactgtaatttagttaaaggggttgacaatggttct |
| | | gaagacttggaaatactgcccaatggactggctttcatcagctccggattaa |
| | | agtatcctggaataatgagctttgaccctgataagtctggaaagatacttct |
| | | aatggacctgaatgaggaagacccagtagtgttggaactgggcattactgga |
| | | aatacattggatatatcttcatttaaccctcatgggattagcacattcacag |
| | | atgaagataacactgtgtacctactggtggtaaaccatccagactcctcgtc |
| | | caccgtggaggtgtttaaatttcaagaagaagaaaaatcacttttgcatctg |
| | | aaaaccatcagacacaagcttctgcctagtgtgaatgacattgtcgctgtgg |
| | | gacctgaacacttttatgccacaaatgatcactattttgctgacccttactt |
| | | aaaatcctgggaaatgcatttgggattagcgtggtcatttgttacttattat |
| | | agtcccaatgatgttcgagtagtggcagaaggatttgattttgctaacggaa |
| | | tcaacatctcaccagacggcaagtatgtctatatagctgagttgctggctca |
| | | taagatccatgtgtatgaaaagcacgctaattggactttaactccattgaag |
| | | tccctcgactttgacacccttgtggataacatctctgtggatcctgtgacag |
| | | gggacctctgggtgggatgccatcccaacggaatgcgaatcttctactatga |
| | | cccaaagaatcctcccggctcagaggtgcttcgaatccaggacattttatcc |
| | | gaagagcccaaagtgacagtggtttatgcagaaaatggcactgtgttacagg |
| | | gcagcacggtggccgctgtgtacaaagggaaactgctgattggcacagtgtt |
| | | tcacaaagctctttactgtgagctg |
| 40 | Human PON1 | MAKLTALTLLGLGLALFDGQKSSFQTRFNVHREVTPVELPNCNLVKGVDNGS |
| | variant G3C9 | EDLEILPNGLAFISSGLKYPGIMSFDPDKSGKILLMDLNEEDPVVLELGITG |
| | amino acid | NTLDISSFNPHGISTFTDEDNTVYLLVVNHPDSSSTVEVFKFQEEEKSLLHL |
| | | KTIRHKLLPSVNDIVAVGPEHFYATNDHYFADPYLKSWEMHLGLAWSFVTYY |
| | | SPNDVRVVAEGFDFANGINISPDGKYVYIAELLAHKIHVYEKHANWTLTPLK |
| | | SLDFDTLVDNISVDPVTGDLWVGCHPNGMRIFYYDPKNPPGSEVLRIQDILS |
| | | EEPKVTVVYAENGTVLQGSTVAAVYKGKLLIGTVFHKALYCEL |
| 41 | PON1 variant | atggctaaactgacagcgctcacactcttggggctgggattggcactcttcg |
| | M-IIGI | atggacagaagtcttctttccaaacacgatttaatgttcaccgtgaagtaac |
| | nucleotide | tccagtggaacttcctaactgtaatttagttaaaggggttgacaatggttct |
| | | gaagacattgaaatactgcccaatggactggctttcatcagctccggagtta |
| | | agtatcctggaataatgagctttgaccctgataagtctggaaagatacttct |
| | | aatggacctgaatgaggaagacccagtagtgttggaactgggcattactgga |
| | | aatacattggatatatcttcatttaaccctgctgggattagcacattcacag |
| | | atgaagataacactgtgtacctactggtggtaaaccgaccagactcctcgtc |
| | | caccgtggaggtgtttaaatttcaagaagaagaaaaatcacttttgcatctg |
| | | aaaaccatcagacacaagcttctgcctagtgtgaatgacattgtcgctgtgg |
| | | gacctgaacacttttatgccacaaatgatcactattttgctgacccttactt |
| | | aaaatcctgggaaatgcatttgggattagcgtggtcatttgttacttattat |
| | | agtcccaatgatgttcgagtagtggcagaaggatttgatatggctaacggaa |
| | | tcaacatctcaccagacggcaagtatgtctatatagctgagttgctggctca |
| | | taagatccatgtgtatgaaaagcacgctaattggactttaactccattgaag |
| | | tccctcgactttgacacccttgtggataacatctctgtggatcctgtgacag |
| | | gggacctctgggtgggatgccatcccaacggaatgcgattattctactatga |
| | | cccaaagaatcctcccggctcagaggtgcttcgaatccaggacattttatcc |
| | | gaagagcccaaagtgacagtggtttatgcagaaaatggcactgtgttacagg |
| | | gcagcagtgtggccgctgtgtacaaagggaaactgctgattggcacagtgtt |
| | | tcacaaagctctttactgtgagctg |
| 42 | PON1 variant | MAKLTALTLLGLGLALFDGQKSSFQTRFNVHREVTPVELPNCNLVKGVDNGS |
| | M-IIGI | EDIEILPNGLAFISSGVKYPGIMSFDPDKSGKILLMDLNEEDPVVLELGITG |
| | amino acid | NTLDISSFNPAGISTFTDEDNTVYLLVVNRPDSSSTVEVFKFQEEEKSLLHL |
| | | KTIRHKLLPSVNDIVAVGPEHFYATNDHYFADPYLKSWEMHLGLAWSFVTYY |
| | | SPNDVRVVAEGFDMANGINISPDGKYVYIAELLAHKIHVYEKHANWTLTPLK |
| | | SLDFDTLVDNISVDPVTGDLWVGCHPNGMRLFYYDPKNPPGSEVLRIQDILS |
| | | EEPKVTVVYAENGTVLQGSSVAAVYKGKLLIGTVFHKALYCEL |
| 43 | hVK3LP- | aagcttgccgccatggaaaccccagcgcagcttctcttcctcctgctactct |
| | [hDNase1 | ggctcccagataccaccggtctgaagatcgcagccttcaacatccagacatt |
| | N74K-G105R- | tggggagaccaagatgtccaatgccaccctcgtcagctacattgtgcagatc |
| | A114F]-(g4s)4- | ctgagccgctatgacatcgccctggtccaggaggtcagagacagccacctga |
| | [SSShinge- | ctgccgtggggaagctgctggacaacctcaatcaggatgcaccagacaccta |
| | P238S-P331S | tcactacgtggtcagtgagccactgggacggaagagctataaggagcgctac |
| | Fc]-NGS- | ctgttcgtgtacaggcctgaccaggtgtctgcggtggacagctactactacg |
| | [G3C9 PON1] | atgatggctgcgagccctgcaggaacgacaccttcaaccgagagccattcat |
| | nucleotide | tgtcaggttcttctcccggttcacagaggtcagggagtttgccattgttccc |
| | | ctgcatgcggccccgggggacgcagtagccgagatcgacgctctctatgacg |
| | | tctacctggatgtccaagagaaatggggcttggaggacgtcatgttgatggg |
| | | cgacttcaatgcgggctgcagctatgtgagaccctcccagtggtcatccatc |
| | | cgcctgtggacaagccccaccttccagtggctgatccccgacagcgctgaca |
| | | ccacagctacacccacgcactgtgcctatgacaggatcgtggttgcagggat |
| | | gctgctccgaggcgccgttgttcccgactcggctcttccctttaacttccag |
| | | gctgcctatggcctgagtgaccaactggcccaagccatcagtgaccactatc |
| | | cagtggaggtgatgctgaaagatctctccggaggaggtggctcaggtggtgg |
| | | aggatctggaggaggtgggagtggtggaggtggttctaccggtctcgagccc |
| | | aaatcttctgacaaaactcacacatctccaccgtccccagcacctgaactcc |
| | | tgggaggatcgtcagtcttcctcttccccccaaaacccaaggacaccctcat |
| | | gatctcccggacccctgaggtcacatgcgtggtggtggacgtgagccacgaa |
| | | gaccctgaggtcaagttcaactggtacgtggacggcgtggaggtgcataatg |
| | | ccaagacaaagccgcgggaggagcagtacaacagcacgtaccgtgtggtcag |
| | | cgtcctcaccgtcctgcaccaggactggctgaatggcaaggagtacaagtgc |
| | | aaggtctccaacaaagccctcccagcctccatcgagaaaaccatctccaaag |
| | | ccaaagggcagccccgagaaccacaggtgtacaccctgcccccatcccggga |
| | | tgagctgaccaagaaccaggtcagcctgacctgcctggtcaaaggcttctat |
| | | cccagcgacatcgccgtggagtgggagagcaatgggcagccggagaacaact |
| | | acaagaccacgcctcccgtgctggactccgacggctccttcttcctctacag |
| | | caagctcaccgtggacaagagcaggtggcagcaggggaacgtcttctcatgc |
| | | tccgtgatgcatgaggctctgcacaaccactacacgcagaagagcctctctc |
| | | tctctccgggtaaagtcgacggagctagcagccccgtgaacgtgagcagccc |
| | | cagcgtgcaggatatcctcttcgatggacagaagtcttctttccaaacacga |
| | | tttaatgttcaccgtgaagtaactccagtggaacttcctaactgtaatttag |
| | | ttaaaggggttgacaatggttctgaagacttggaaatactgcccaatggact |
| | | ggctttcatcagctccggattaaagtatcctggaataatgagctttgaccct |
| | | gataagtctggaaagatacttctaatggacctgaatgaggaagacccagtag |
| | | tgttggaactgggcattactggaaatacattggatatatcttcatttaaccc |
| | | tcatgggattagcacattcacagatgaagataacactgtgtacctactggtg |
| | | gtaaaccatccagactcctcgtccaccgtggaggtgtttaaatttcaagaag |
| | | aagaaaaatcacttttgcatctgaaaaccatcagacacaagcttctgcctag |
| | | tgtgaatgacattgtcgctgtgggacctgaacacttttatgccacaaatgat |
| | | cactattttgctgacccttacttaaaatcctgggaaatgcatttgggattag |
| | | cgtggtcatttgttacttattatagtcccaatgatgttcgagtagtggcaga |
| | | aggatttgattttgctaacggaatcaacatctcaccagacggcaagtatgtc |
| | | tatatagctgagttgctggctcataagatccatgtgtatgaaaagcacgcta |
| | | attggactttaactccattgaagtccctcgactttgacacccttgtggataa |
| | | catctctgtggatcctgtgacaggggacctctgggtgggatgccatcccaac |
| | | ggaatgcgaatcttctactatgacccaaagaatcctcccggctcagaggtgc |
| | | ttcgaatccaggacattttatccgaagagcccaaagtgacagtggtttatgc |
| | | agaaaatggcactgtgttacagggcagcacggtggccgctgtgtacaaaggg |
| | | aaactgctgattggcacagtgtttcacaaagctctttactgtgagctgtaat |
| | | aatctagaa |
| 44 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R- | RPDQVSAVDSYYYDDGCEPCRNDTFNREPFIVRFFSRFTEVREFAIVPLHAA |
| | A114F]-(g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc]-NGS- | KTHTSPPSPAPELLGGSSVFLFPPKPKDTLMISRTPEVTCVVVDVSHEDPEV |
| | [G3C9 PON1] | KFNWYVDGVEVHNAKTKPREEQYNSTYRVVSVLTVLHQDWLNGKEYKCKVSN |
| | amino acid | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGKVDGASSPVNVSSPSVQDILFDGQKSSFQTRFNVH |
| | | REVTPVELPNCNLVKGVDNGSEDLEILPNGLAFISSGLKYPGIMSFDPDKSG |
| | | KILLMDLNEEDPVVLELGITGNTLDISSFNPHGISTFTDEDNTVYLLVVNHP |
| | | DSSSTVEVFKFQEEEKSLLHLKTIRHKLLPSVNDIVAVGPEHFYATNDHYFA |
| | | DPYLKSWEMHLGLAWSFVTYYSPNDVRVVAEGFDFANGINISPDGKYVYIAE |
| | | LLAHKIHVYEKHANWTLTPLKSLDFDTLVDNISVDPVTGDLWVGCHPNGMRI |
| | | FYYDPKNPPGSEVLRIQDILSEEPKVTVVYAENGTVLQGSTVAAVYKGKLLI |
| | | GTVFHKALYCEL |
| 45 | hVK3LP- | atggaaaccccagcgcagcttctcttcctcctgctactctggctcccagata |
| | hDNase1 | ccaccggtctgaagatcgcagccttcaacatccagacatttggggagaccaa |
| | N74K-G105R- | gatgtccaatgccaccctcgtcagctacattgtgcagatcctgagccgctat |
| | A114F]-(g4s)4- | gacatcgccctggtccaggaggtcagagacagccacctgactgccgtgggga |
| | [SSShinge- | agctgctggacaacctcaatcaggatgcaccagacacctatcactacgtggt |
| | P238S-P331S | cagtgagccactgggacggaagagctataaggagcgctacctgttcgtgtac |
| | Fc]-NGS-[M- | aggcctgaccaggtgtctgcggtggacagctactactacgatgatggctgcg |
| | IIGI PON1] | agccctgcaggaacgacaccttcaaccgagagccattcattgtcaggttctt |
| | nucleotide | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aagtcgacggagctagcagccccgtgaacgtgagcagccccagcgtgcagga |
| | | tatcctcttcgatggacagaagtcttctttccaaacacgatttaatgttcac |
| | | cgtgaagtaactccagtggaacttcctaactgtaatttagttaaaggggttg |
| | | acaatggttctgaagacattgaaatactgcccaatggactggctttcatcag |
| | | ctccggagttaagtatcctggaataatgagctttgaccctgataagtctgga |
| | | aagatacttctaatggacctgaatgaggaagacccagtagtgttggaactgg |
| | | gcattactggaaatacattggatatatcttcatttaaccctgctgggattag |
| | | cacattcacagatgaagataacactgtgtacctactggtggtaaaccgacca |
| | | gactcctcgtccaccgtggaggtgtttaaatttcaagaagaagaaaaatcac |
| | | ttttgcatctgaaaaccatcagacacaagcttctgcctagtgtgaatgacat |
| | | tgtcgctgtgggacctgaacacttttatgccacaaatgatcactattttgct |
| | | gacccttacttaaaatcctgggaaatgcatttgggattagcgtggtcatttg |
| | | ttacttattatagtcccaatgatgttcgagtagtggcagaaggatttgatat |
| | | ggctaacggaatcaacatctcaccagacggcaagtatgtctatatagctgag |
| | | ttgctggctcataagatccatgtgtatgaaaagcacgctaattggactttaa |
| | | ctccattgaagtccctcgactttgacacccttgtggataacatctctgtgga |
| | | tcctgtgacaggggacctctgggtgggatgccatcccaacggaatgcgatta |
| | | ttctactatgacccaaagaatcctcccggctcagaggtgcttcgaatccagg |
| | | acattttatccgaagagcccaaagtgacagtggtttatgcagaaaatggcac |
| | | tgtgttacagggcagcagtgtggccgctgtgtacaaagggaaactgctgatt |
| | | ggcacagtgtttcacaaagctctttactgtgagctg |
| 46 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R- | RPDQVSAVDSYYYDDGCEPCRNDTFNREPFIVRFFSRFTEVREFAIVPLHAA |
| | A114F]-(g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc]-NGS-[M- | KTHTSPPSPAPELLGGSSVFLFPPKPKDTLMISRTPEVTCVVVDVSHEDPEV |
| | IIGI PON1] | KFNWYVDGVEVHNAKTKPREEQYNSTYRVVSVLTVLHQDWLNGKEYKCKVSN |
| | amino acid | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGKVDGASSPVNVSSPSVQDILFDGQKSSFQTRFNVH |
| | | REVTPVELPNCNLVKGVDNGSEDIEILPNGLAFISSGVKYPGIMSFDPDKSG |
| | | KILLMDLNEEDPVVLELGITGNTLDISSFNPAGISTFTDEDNTVYLLVVNRP |
| | | DSSSTVEVFKFQEEEKSLLHLKTIRHKLLPSVNDIVAVGPEHFYATNDHYFA |
| | | DPYLKSWEMHLGLAWSFVTYYSPNDVRVVAEGFDMANGINISPDGKYVYIAE |
| | | LLAHKIHVYEKHANWTLTPLKSLDFDTLVDNISVDPVTGDLWVGCHPNGMRL |
| | | FYYDPKNPPGSEVLRIQDILSEEPKVTVVYAENGTVLQGSSVAAVYKGKLLI |
| | | GTVFHKALYCEL |
| 47 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | N74K-G105R- | gatgtccaatgccaccctcgtcagctacattgtgcagatcctgagccgctat |
| | A114F]-(g4s)6- | gacatcgccctggtccaggaggtcagagacagccacctgactgccgtgggga |
| | [SSShinge- | agctgctggacaacctcaatcaggatgcaccagacacctatcactacgtggt |
| | P238S-P331S | cagtgagccactgggacggaagagctataaggagcgctacctgttcgtgtac |
| | Fc]-NGS- | aggcctgaccaggtgtctgcggtggacagctactactacgatgatggctgcg |
| | [PON1 Q192K] | agccctgcaggaacgacaccttcaaccgagagccattcattgtcaggttctt |
| | nucleotide | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctggaggaggtggtagtggaggtggaggt |
| | | tctaccggtctcgagcccaaatcttctgacaaaactcacacatctccaccgt |
| | | ccccagcacctgaactcctgggaggatcgtcagtcttcctcttccccccaaa |
| | | acccaaggacaccctcatgatctcccggacccctgaggtcacatgcgtggtg |
| | | gtggacgtgagccacgaagaccctgaggtcaagttcaactggtacgtggacg |
| | | gcgtggaggtgcataatgccaagacaaagccgcgggaggagcagtacaacag |
| | | cacgtaccgtgtggtcagcgtcctcaccgtcctgcaccaggactggctgaat |
| | | ggcaaggagtacaagtgcaaggtctccaacaaagccctcccagcctccatcg |
| | | agaaaaccatctccaaagccaaagggcagccccgagaaccacaggtgtacac |
| | | cctgcccccatcccgggatgagctgaccaagaaccaggtcagcctgacctgc |
| | | ctggtcaaaggcttctatcccagcgacatcgccgtggagtgggagagcaatg |
| | | ggcagccggagaacaactacaagaccacgcctcccgtgctggactccgacgg |
| | | ctccttcttcctctacagcaagctcaccgtggacaagagcaggtggcagcag |
| | | gggaacgtcttctcatgctccgtgatgcatgaggctctgcacaaccactaca |
| | | cgcagaagagcctctctctctctccgggtaaagtcgacggagctagcagccc |
| | | cgtgaacgtgagcagccccagcgtgcaggatatcctcttcaggaaccaccag |
| | | tcttcttaccaaacacgacttaatgctctccgagaggtacaacccgtagaac |
| | | ttcctaactgtaatttagttaaaggaatcgaaactggctctgaagacttgga |
| | | gatactgcctaatggactggctttcattagctctggattaaagtatcctgga |
| | | ataaagagcttcaaccccaacagtcctggaaaaatacttctgatggacctga |
| | | atgaagaagatccaacagtgttggaattggggatcactggaagtaaatttga |
| | | tgtatcttcatttaaccctcatgggattagcacattcacagatgaagataat |
| | | gccatgtacctcctggtggtgaaccatccagatgccaagtccacagtggagt |
| | | tgtttaaatttcaagaagaagaaaaatcgcttttgcatctaaaaaccatcag |
| | | acataaacttctgcctaatttgaatgatattgttgctgtgggacctgagcac |
| | | ttttatggcacaaatgatcactattttcttgacccctacttaaaatcctggg |
| | | agatgtatttgggtttagcgtggtcgtatgttgtctactatagtccaagtga |
| | | agttcgagtggtggcagaaggatttgattttgctaatggaatcaacatttca |
| | | cccgatggcaagtatgtctatatagctgagttgctggctcataagattcatg |
| | | tgtatgaaaagcatgctaattggactttaactccattgaagtcccttgactt |
| | | taataccctcgtggataacatatctgtggatcctgagacaggagacctttgg |
| | | gttggatgccatcccaatggcatgaaaatcttcttctatgactcagagaatc |
| | | ctcctgcatcagaggtgcttcgaatccagaacattctaacagaagaacctaa |
| | | agtgacacaggtttatgcagaaaatggcacagtgttgcaaggcagtacagtt |
| | | gcctctgtgtacaaagggaaactgctgattggcacagtgtttcacaaagctc |
| | | tttactgtgagctctaa |
| 48 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R- | RPDQVSAVDSYYYDDGCEPCRNDTFNREPFIVRFFSRFTEVREFAIVPLHAA |
| | A114F]-(g4s)6- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSGGGGSGGGG |
| | Fc]-NGS- | STGLEPKSSDKTHTSPPSPAPELLGGSSVFLFPPKPKDTLMISRTPEVTCVV |
| | [PON1 Q192K] | VDVSHEDPEVKFNWYVDGVEVHNAKTKPREEQYNSTYRVVSVLTVLHQDWLN |
| | amino acid | GKEYKCKVSNKALPASIEKTISKAKGQPREPQVYTLPPSRDELTKNQVSLTC |
| | | LVKGFYPSDIAVEWESNGQPENNYKTTPPVLDSDGSFFLYSKLTVDKSRWQQ |
| | | GNVFSCSVMHEALHNHYTQKSLSLSPGKVDGASSPVNVSSPSVQDILFRNHQ |
| | | SSYQTRLNALREVQPVELPNCNLVKGIETGSEDLEILPNGLAFISSGLKYPG |
| | | IKSFNPNSPGKILLMDLNEEDPTVLELGITGSKFDVSSFNPHGISTFTDEDN |
| | | AMYLLVVNHPDAKSTVELFKFQEEEKSLLHLKTIRHKLLPNLNDIVAVGPEH |
| | | FYGTNDHYFLDPYLKSWEMYLGLAWSYVVYYSPSEVRVVAEGFDFANGINIS |
| | | PDGKYVYIAELLAHKIHVYEKHANWTLTPLKSLDFNTLVDNISVDPETGDLW |
| | | VGCHPNGMKIFFYDSENPPASEVLRIQNILTEEPKVTQVYAENGTVLQGSTV |
| | | ASVYKGKLLIGTVFHKALYCEL |
| 49 | [hDNase1 | LKIAAFNIQTFGETKMSNATLVSYIVQILSRYDIALVQEVRDSHLTAVGKLL |
| | N74X-G105X- | DNLNQDAPDTYHYVVSEPLGRXSYKERYLFVYRPDQVSAVDSYYYDDGCEPC |
| | A114X]-[gs | XNDTFNREPXIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 50 | [hDNase1 | LKIAAFNIQTFGETKMSNATLVSYIVQILSRYDIALVQEVRDSHLTAVGKLL |
| | N74K-G105R- | DNLNQDAPDTYHYVVSEPLGRKSYKERYLFVYRPDQVSAVDSYYYDDGCEPC |
| | A114F]-[gs | RNDTFNREPFIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 51 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | N74K-G105R] | gatgtccaatgccaccctcgtcagctacattgtgcagatcctgagccgctat |
| | nucleotide | gacatcgccctggtccaggaggtcagagacagccacctgactgccgtgggga |
| | | agctgctggacaacctcaatcaggatgcaccagacacctatcactacgtggt |
| | | cagtgagccactgggacggaagagctataaggagcgctacctgttcgtgtac |
| | | aggcctgaccaggtgtctgcggtggacagctactactacgatgatggctgcg |
| | | agccctgcaggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 52 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R] | RPDQVSAVDSYYYDDGCEPCRNDTFNREPAIVRFFSRFTEVREFAIVPLHAA |
| | amino acid | PGDAVAEIDALYDVYLDVQEKWGLEDVMLMGDFNAGCSYVRPSQWSSIRLWT |
| | | SPTFQWLIPDSADTTATPTHCAYDRIVVAGMLLRGAVVPDSALPFNFQAAYG |
| | | LSDQLAQAISDHYPVEVMLK |
| 53 | [hDNase1 | LKIAAFNIQTFGETKMSNATLVSYIVQILSRYDIALVQEVRDSHLTAVGKLL |
| | N74X-G105X]- | DNLNQDAPDTYHYVVSEPLGRXSYKERYLFVYRPDQVSAVDSYYYDDGCEPC |
| | [gs linker]- | XNDTFNREPAIVRFFSRFTEVREFAIVPLHAAPGDAVAEIDALYDVYLDVQE |
| | [SSShinge Fc] | KWGLEDVMLMGDFNAGCSYVRPSQWSSIRLWTSPTFQWLIPDSADTTATPTH |
| | amino acid | CAYDRIVVAGMLLRGAVVPDSALPFNFQAAYGLSDQLAQAISDHYPVEVMLK |
| | | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 54 | [hDNase1 | LKIAAFNIQTFGETKMSNATLVSYIVQILSRYDIALVQEVRDSHLTAVGKLL |
| | N74K-G105R]- | DNLNQDAPDTYHYVVSEPLGRKSYKERYLFVYRPDQVSAVDSYYYDDGCEPC |
| | [gs linker]- | RNDTFNREPAIVRFFSRFTEVREFAIVPLHAAPGDAVAEIDALYDVYLDVQE |
| | [SSShinge Fc] | KWGLEDVMLMGDFNAGCSYVRPSQWSSIRLWTSPTFQWLIPDSADTTATPTH |
| | amino acid | CAYDRIVVAGMLLRGAVVPDSALPFNFQAAYGLSDQLAQAISDHYPVEVMLK |
| | | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 55 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R]- | RPDQVSAVDSYYYDDGCEPCRNDTFNREPAIVRFFSRFTEVREFAIVPLHAA |
| | (g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc] | KTHTSPPSPAPELLGGSSVFLFPPKPKDTLMISRTPEVTCVVVDVSHEDPEV |
| | amino acid | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 56 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R]- | RPDQVSAVDSYYYDDGCEPCRNDTFNREPAIVRFFSRFTEVREFAIVPLHAA |
| | (g4s)6- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSGGGGSGGGG |
| | Fc] | STGLEPKSSDKTHTSPPSPAPELLGGSSVFLFPPKPKDTLMISRTPEVTCVV |
| | amino acid | VDVSHEDPEVKFNWYVDGVEVHNAKTKPREEQYNSTYRVVSVLTVLHQDWLN |
| | | GKEYKCKVSNKALPASIEKTISKAKGQPREPQVYTLPPSRDELTKNQVSLTC |
| | | LVKGFYPSDIAVEWESNGQPENNYKTTPPVLDSDGSFFLYSKLTVDKSRWQQ |
| | | GNVFSCSVMHEALHNHYTQKSLSLSPGK |
| 57 | hVK3LP- | aagcttgccgccatggaaaccccagcgcagcttctcttcctcctgctactct |
| | [hDNase1 | ggctcccagataccaccggtctgaagatcgcagccttcaacatccagacatt |
| | N74K-G105R]- | tggggagaccaagatgtccaatgccaccctcgtcagctacattgtgcagatc |
| | (g4s)4- | ctgagccgctatgacatcgccctggtccaggaggtcagagacagccacctga |
| | [SSShinge- | ctgccgtggggaagctgctggacaacctcaatcaggatgcaccagacaccta |
| | P238S-P331S | tcactacgtggtcagtgagccactgggacggaagagctataaggagcgctac |
| | Fc]-NGS- | ctgttcgtgtacaggcctgaccaggtgtctgcggtggacagctactactacg |
| | [PON1 Q192K] | atgatggctgcgagccctgcaggaacgacaccttcaaccgagagccagccat |
| | nucleotide | tgtcaggttcttctcccggttcacagaggtcagggagtttgccattgttccc |
| | | ctgcatgcggccccgggggacgcagtagccgagatcgacgctctctatgacg |
| | | tctacctggatgtccaagagaaatggggcttggaggacgtcatgttgatggg |
| | | cgacttcaatgcgggctgcagctatgtgagaccctcccagtggtcatccatc |
| | | cgcctgtggacaagccccaccttccagtggctgatccccgacagcgctgaca |
| | | ccacagctacacccacgcactgtgcctatgacaggatcgtggttgcagggat |
| | | gctgctccgaggcgccgttgttcccgactcggctcttccctttaacttccag |
| | | gctgcctatggcctgagtgaccaactggcccaagccatcagtgaccactatc |
| | | cagtggaggtgatgctgaaagatctctccggaggaggtggctcaggtggtgg |
| | | aggatctggaggaggtgggagtggtggaggtggttctaccggtctcgagccc |
| | | aaatcttctgacaaaactcacacatctccaccgtccccagcacctgaactcc |
| | | tgggaggatcgtcagtcttcctcttccccccaaaacccaaggacaccctcat |
| | | gatctcccggacccctgaggtcacatgcgtggtggtggacgtgagccacgaa |
| | | gaccctgaggtcaagttcaactggtacgtggacggcgtggaggtgcataatg |
| | | ccaagacaaagccgcgggaggagcagtacaacagcacgtaccgtgtggtcag |
| | | cgtcctcaccgtcctgcaccaggactggctgaatggcaaggagtacaagtgc |
| | | aaggtctccaacaaagccctcccagcctccatcgagaaaaccatctccaaag |
| | | ccaaagggcagccccgagaaccacaggtgtacaccctgcccccatcccggga |
| | | tgagctgaccaagaaccaggtcagcctgacctgcctggtcaaaggcttctat |
| | | cccagcgacatcgccgtggagtgggagagcaatgggcagccggagaacaact |
| | | acaagaccacgcctcccgtgctggactccgacggctccttcttcctctacag |
| | | caagctcaccgtggacaagagcaggtggcagcaggggaacgtcttctcatgc |
| | | tccgtgatgcatgaggctctgcacaaccactacacgcagaagagcctctctc |
| | | tctctccgggtaaagtcgacggagctagcagccccgtgaacgtgagcagccc |
| | | cagcgtgcaggatatcctcttcaggaaccaccagtcttcttaccaaacacga |
| | | cttaatgctctccgagaggtacaacccgtagaacttcctaactgtaatttag |
| | | ttaaaggaatcgaaactggctctgaagacttggagatactgcctaatggact |
| | | ggctttcattagctctggattaaagtatcctggaataaagagcttcaacccc |
| | | aacagtcctggaaaaatacttctgatggacctgaatgaagaagatccaacag |
| | | tgttggaattggggatcactggaagtaaatttgatgtatcttcatttaaccc |
| | | tcatgggattagcacattcacagatgaagataatgccatgtacctcctggtg |
| | | gtgaaccatccagatgccaagtccacagtggagttgtttaaatttcaagaag |
| | | aagaaaaatcgcttttgcatctaaaaaccatcagacataaacttctgcctaa |
| | | tttgaatgatattgttgctgtgggacctgagcacttttatggcacaaatgat |
| | | cactattttcttgacccctacttaaaatcctgggagatgtatttgggtttag |
| | | cgtggtcgtatgttgtctactatagtccaagtgaagttcgagtggtggcaga |
| | | aggatttgattttgctaatggaatcaacatttcacccgatggcaagtatgtc |
| | | tatatagctgagttgctggctcataagattcatgtgtatgaaaagcatgcta |
| | | attggactttaactccattgaagtcccttgactttaataccctcgtggataa |
| | | catatctgtggatcctgagacaggagacctttgggttggatgccatcccaat |
| | | ggcatgaaaatcttcttctatgactcagagaatcctcctgcatcagaggtgc |
| | | ttcgaatccagaacattctaacagaagaacctaaagtgacacaggtttatgc |
| | | agaaaatggcacagtgttgcaaggcagtacagttgcctctgtgtacaaaggg |
| | | aaactgctgattggcacagtgtttcacaaagctctttactgtgagctctaat |
| | | aatctagaa |
| 58 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R]- | RPDQVSAVDSYYYDDGCEPCRNDTFNREPAIVRFFSRFTEVREFAIVPLHAA |
| | (g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc]-NGS- | KTHTSPPSPAPELLGGSSVFLFPPKPKDTLMISRTPEVTCVVVDVSHEDPEV |
| | [PON1 Q192K] | KFNWYVDGVEVHNAKTKPREEQYNSTYRVVSVLTVLHQDWLNGKEYKCKVSN |
| | amino acid | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGKVDGASSPVNVSSPSVQDILFRNHQSSYQTRLNAL |
| | | REVQPVELPNCNLVKGIETGSEDLEILPNGLAFISSGLKYPGIKSFNPNSPG |
| | | KILLMDLNEEDPTVLELGITGSKFDVSSFNPHGISTFTDEDNAMYLLVVNHP |
| | | DAKSTVELFKFQEEEKSLLHLKTIRHKLLPNLNDIVAVGPEHFYGTNDHYFL |
| | | DPYLKSWEMYLGLAWSYVVYYSPSEVRVVAEGFDFANGINISPDGKYVYIAE |
| | | LLAHKIHVYEKHANWTLTPLKSLDFNTLVDNISVDPETGDLWVGCHPNGMKI |
| | | FFYDSENPPASEVLRIQNILTEEPKVTQVYAENGTVLQGSTVASVYKGKLLI |
| | | GTVFHKALYCEL |
| 59 | hVK3LP- | aagcttgccgccatggaaaccccagcgcagcttctcttcctcctgctactct |
| | [hDNase1 | ggctcccagataccaccggtctgaagatcgcagccttcaacatccagacatt |
| | N74K-G105R]- | tggggagaccaagatgtccaatgccaccctcgtcagctacattgtgcagatc |
| | (g4s)6- | ctgagccgctatgacatcgccctggtccaggaggtcagagacagccacctga |
| | [SSShinge- | ctgccgtggggaagctgctggacaacctcaatcaggatgcaccagacaccta |
| | P238S-P331S | tcactacgtggtcagtgagccactgggacggaagagctataaggagcgctac |
| | Fc]-NGS- | ctgttcgtgtacaggcctgaccaggtgtctgcggtggacagctactactacg |
| | [PON1 Q192K] | atgatggctgcgagccctgcaggaacgacaccttcaaccgagagccagccat |
| | nucleotide | tgtcaggttcttctcccggttcacagaggtcagggagtttgccattgttccc |
| | | ctgcatgcggccccgggggacgcagtagccgagatcgacgctctctatgacg |
| | | tctacctggatgtccaagagaaatggggcttggaggacgtcatgttgatggg |
| | | cgacttcaatgcgggctgcagctatgtgagaccctcccagtggtcatccatc |
| | | cgcctgtggacaagccccaccttccagtggctgatccccgacagcgctgaca |
| | | ccacagctacacccacgcactgtgcctatgacaggatcgtggttgcagggat |
| | | gctgctccgaggcgccgttgttcccgactcggctcttccctttaacttccag |
| | | gctgcctatggcctgagtgaccaactggcccaagccatcagtgaccactatc |
| | | cagtggaggtgatgctgaaagatctctccggaggaggtggctcaggtggtgg |
| | | aggatctggaggaggtgggagtggtggaggtggttctggaggaggtggtagt |
| | | ggaggtggaggttctaccggtctcgagcccaaatcttctgacaaaactcaca |
| | | catctccaccgtccccagcacctgaactcctgggaggatcgtcagtcttcct |
| | | cttccccccaaaacccaaggacaccctcatgatctcccggacccctgaggtc |
| | | acatgcgtggtggtggacgtgagccacgaagaccctgaggtcaagttcaact |
| | | ggtacgtggacggcgtggaggtgcataatgccaagacaaagccgcgggagga |
| | | gcagtacaacagcacgtaccgtgtggtcagcgtcctcaccgtcctgcaccag |
| | | gactggctgaatggcaaggagtacaagtgcaaggtctccaacaaagccctcc |
| | | cagcctccatcgagaaaaccatctccaaagccaaagggcagccccgagaacc |
| | | acaggtgtacaccctgcccccatcccgggatgagctgaccaagaaccaggtc |
| | | agcctgacctgcctggtcaaaggcttctatcccagcgacatcgccgtggagt |
| | | gggagagcaatgggcagccggagaacaactacaagaccacgcctcccgtgct |
| | | ggactccgacggctccttcttcctctacagcaagctcaccgtggacaagagc |
| | | aggtggcagcaggggaacgtcttctcatgctccgtgatgcatgaggctctgc |
| | | acaaccactacacgcagaagagcctctctctctctccgggtaaagtcgacgg |
| | | agctagcagccccgtgaacgtgagcagccccagcgtgcaggatatcctcttc |
| | | aggaaccaccagtcttcttaccaaacacgacttaatgctctccgagaggtac |
| | | aacccgtagaacttcctaactgtaatttagttaaaggaatcgaaactggctc |
| | | tgaagacttggagatactgcctaatggactggctttcattagctctggatta |
| | | aagtatcctggaataaagagcttcaaccccaacagtcctggaaaaatacttc |
| | | tgatggacctgaatgaagaagatccaacagtgttggaattggggatcactgg |
| | | aagtaaatttgatgtatcttcatttaaccctcatgggattagcacattcaca |
| | | gatgaagataatgccatgtacctcctggtggtgaaccatccagatgccaagt |
| | | ccacagtggagttgtttaaatttcaagaagaagaaaaatcgcttttgcatct |
| | | aaaaaccatcagacataaacttctgcctaatttgaatgatattgttgctgtg |
| | | ggacctgagcacttttatggcacaaatgatcactattttcttgacccctact |
| | | taaaatcctgggagatgtatttgggtttagcgtggtcgtatgttgtctacta |
| | | tagtccaagtgaagttcgagtggtggcagaaggatttgattttgctaatgga |
| | | atcaacatttcacccgatggcaagtatgtctatatagctgagttgctggctc |
| | | ataagattcatgtgtatgaaaagcatgctaattggactttaactccattgaa |
| | | gtcccttgactttaataccctcgtggataacatatctgtggatcctgagaca |
| | | ggagacctttgggttggatgccatcccaatggcatgaaaatcttcttctatg |
| | | actcagagaatcctcctgcatcagaggtgcttcgaatccagaacattctaac |
| | | agaagaacctaaagtgacacaggtttatgcagaaaatggcacagtgttgcaa |
| | | ggcagtacagttgcctctgtgtacaaagggaaactgctgattggcacagtgt |
| | | ttcacaaagctctttactgtgagctctaataatctagaa |
| 60 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSEPLGRKSYKERYLFVY |
| | N74K-G105R]- | RPDQVSAVDSYYYDDGCEPCRNDTFNREPAIVRFFSRFTEVREFAIVPLHAA |
| | (g4s)6- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSGGGGSGGGG |
| | Fc]-NGS- | STGLEPKSSDKTHTSPPSPAPELLGGSSVFLFPPKPKDTLMISRTPEVTCVV |
| | [PON1 Q192K] | VDVSHEDPEVKFNWYVDGVEVHNAKTKPREEQYNSTYRVVSVLTVLHQDWLN |
| | amino acid | GKEYKCKVSNKALPASIEKTISKAKGQPREPQVYTLPPSRDELTKNQVSLTC |
| | | LVKGFYPSDIAVEWESNGQPENNYKTTPPVLDSDGSFFLYSKLTVDKSRWQQ |
| | | GNVFSCSVMHEALHNHYTQKSLSLSPGKVDGASSPVNVSSPSVQDILFRNHQ |
| | | SSYQTRLNALREVQPVELPNCNLVKGIETGSEDLEILPNGLAFISSGLKYPG |
| | | IKSFNPNSPGKILLMDLNEEDPTVLELGITGSKFDVSSFNPHGISTFTDEDN |
| | | AMYLLVVNHPDAKSTVELFKFQEEEKSLLHLKTIRHKLLPNLNDIVAVGPEH |
| | | FYGTNDHYFLDPYLKSWEMYLGLAWSYVVYYSPSEVRVVAEGFDFANGINIS |
| | | PDGKYVYIAELLAHKIHVYEKHANWTLTPLKSLDFNTLVDNISVDPETGDLW |
| | | VGCHPNGMKIFFYDSENPPASEVLRIQNILTEEPKVTQVYAENGTVLQGSTV |
| | | ASVYKGKLLIGTVFHKALYCEL |
| 61 | hVK3LP-[KDL- | METPAQLLFLLLLWLPDTTGKDLEPKSSDKTHTCPPCPAPELLGGSSVFLFP |
| | SCChinge- | PKPKDTLMISRTPEVTCVVVDVSHEDPEVKFNWYVDGVEVHNAKTKPREEQY |
| | P238S-P331S | NSTYRVVSVLTVLHQDWLNGKEYKCKVSNKALPASIEKTISKAKGQPREPQV |
| | Fc]-NGS- | YTLPPSRDELTKNQVSLTCLVKGFYPSDIAVEWESNGQPENNYKTTPPVLDS |
| | [PON1 Q192K] | DGSFFLYSKLTVDKSRWQQGNVFSCSVMHEALHNHYTQKSLSLSPGKVDGAS |
| | amino acid | SPVNVSSPSVQDILFDGQKSSFQTRFNVHREVTPVELPNCNLVKGVDNGSED |
| | | LEILPNGLAFISSGLKYPGIMSFDPDKSGKILLMDLNEEDPVVLELGITGNT |
| | | LDISSFNPHGISTFTDEDNTVYLLVVNHPDSSSTVEVFKFQEEEKSLLHLKT |
| | | IRHKLLPSVNDIVAVGPEHFYATNDHYFADPYLKSWEMHLGLAWSFVTYYSP |
| | | NDVRVVAEGFDFANGINISPDGKYVYIAELLAHKIHVYEKHANWTLTPLKSL |
| | | DFDTLVDNISVDPVTGDLWVGCHPNGMRIFYYDPKNPPGSEVLRIQDILSEE |
| | | PKVTVVYAENGTVLQGSTVAAVYKGKLLIGTVFHKALYCEL |
| 62 | hVK3LP-[KDL- | METPAQLLFLLLLWLPDTTGKDLEPKSSDKTHTCPPCPAPELLGGSSVFLFP |
| | SCChinge- | PKPKDTLMISRTPEVTCVVVDVSHEDPEVKFNWYVDGVEVHNAKTKPREEQY |
| | P238S-P331S | NSTYRVVSVLTVLHQDWLNGKEYKCKVSNKALPASIEKTISKAKGQPREPQV |
| | Fc]-NGS- | YTLPPSRDELTKNQVSLTCLVKGFYPSDIAVEWESNGQPENNYKTTPPVLDS |
| | [G3C9 PON1] | DGSFFLYSKLTVDKSRWQQGNVFSCSVMHEALHNHYTQKSLSLSPGKVDGAS |
| | amino acid | SPVNVSSPSVQDILFDGQKSSFQTRFNVHREVTPVELPNCNLVKGVDNGSED |
| | | IEILPNGLAFISSGVKYPGIMSFDPDKSGKILLMDLNEEDPVVLELGITGNT |
| | | LDISSFNPAGISTFTDEDNTVYLLVVNRPDSSSTVEVFKFQEEEKSLLHLKT |
| | | IRHKLLPSVNDIVAVGPEHFYATNDHYFADPYLKSWEMHLGLAWSFVTYYSP |
| | | NDVRVVAEGFDMANGINISPDGKYVYIAELLAHKIHVYEKHANWTLTPLKSL |
| | | DFDTLVDNISVDPVTGDLWVGCHPNGMRLFYYDPKNPPGSEVLRIQDILSEE |
| | | PKVTWYAENGTVLQGSSVAAVYKGKLL1GTVFHKALYCEL |
| 63 | hVK3LP-[KDL- | METPAQLLFLLLLWLPDTTGKDLEPKSSDKTHTCPPCPAPELLGGSSVFLFP |
| | SCChinge- | PKPKDTLMISRTPEVTCVVVDVSHEDPEVKFNWYVDGVEVHNAKTKPREEQY |
| | P238S-P331S | NSTYRVVSVLTVLHQDWLNGKEYKCKVSNKALPASIEKTISKAKGQPREPQV |
| | Fc]-NGS-[M- | YTLPPSRDELTKNQVSLTCLVKGFYPSDIAVEWESNGQPENNYKTTPPVLDS |
| | IIGI PON1] | DGSFFLYSKLTVDKSRWQQGNVFSCSVMHEALHNHYTQKSLSLSPGKVDGAS |
| | amino acid | SPVNVSSPSVQDILFDGQKSSFQTRFNVHREVTPVELPNCNLVKGVDNGSED |
| | | IEILPNGLAFISSGVKYPGIMSFDPDKSGKILLMDLNEEDPVVLELGITGNT |
| | | LDISSFNPAGISTFTDEDNTVYLLVVNRPDSSSTVEVFKFQEEEKSLLHLKT |
| | | IRHKLLPSVNDIVAVGPEHFYATNDHYFADPYLKSWEMHLGLAWSFVTYYSP |
| | | NDVRVVAEGFDMANGINISPDGKYVYIAELLAHKIHVYEKHANWTLTPLKSL |
| | | DFDTLVDNISVDPVTGDLWVGCHPNGMRLFYYDPKNPPGSEVLRIQDILSEE |
| | | PKVTVVYAENGTVLQGSSVAAVYKGKLLIGTVFHKALYCEL |
| 64 | hVK3LP- | METPAQLLFLLLLWLPDTTGEPKSSDKTHTCPPCPAPELLGGSSVFLFPPKP |
| | [SCChinge- | KDTLMISRTPEVTCVVVDVSHEDPEVKFNWYVDGVEVHNAKTKPREEQYNST |
| | P238S-P331S | YRVVSVLTVLHQDWLNGKEYKCKVSNKALPASIEKTISKAKGQPREPQVYTL |
| | Fc]-NGS- | PPSRDELTKNQVSLTCLVKGFYPSDIAVEWESNGQPENNYKTTPPVLDSDGS |
| | [PON1 Q192K] | FFLYSKLTVDKSRWQQGNVFSCSVMHEALHNHYTQKSLSLSPGKVDGASSPV |
| | amino acid | NVSSPSVQDILFRNHQSSYQTRLNALREVQPVELPNCNLVKGIETGSEDLEI |
| | | LPNGLAFISSGLKYPGIKSFNPNSPGKILLMDLNEEDPTVLELGITGSKFDV |
| | | SSFNPHGISTFTDEDNAMYLLVVNHPDAKSTVELFKFQEEEKSLLHLKTIRH |
| | | KLLPNLNDIVAVGPEHFYGTNDHYFLDPYLKSWEMYLGLAWSYVVYYSPSEV |
| | | RVVAEGFDFANGINISPDGKYVYIAELLAHKIHVYEKHANWTLTPLKSLDFN |
| | | TLVDNISVDPETGDLWVGCHPNGMKIFFYDSENPPASEVLRIQNILTEEPKV |
| | | TQVYAENGTVLQGSTVASVYKGKLLIGTVFHKALYCEL |
| 65 | hVK3LP- | METPAQLLFLLLLWLPDTTGEPKSSDKTHTCPPCPAPELLGGSSVFLFPPKP |
| | [SCChinge- | KDTLMISRTPEVTCVVVDVSHEDPEVKFNWYVDGVEVHNAKTKPREEQYNST |
| | P238S-P331S | YRVVSVLTVLHQDWLNGKEYKCKVSNKALPASIEKTISKAKGQPREPQVYTL |
| | Fc]-NGS- | PPSRDELTKNQVSLTCLVKGFYPSDIAVEWESNGQPENNYKTTPPVLDSDGS |
| | [G3C9 PON1] | FFLYSKLTVDKSRWQQGNVFSCSVMHEALHNHYTQKSLSLSPGKVDGASSPV |
| | amino acid | NVSSPSVQDILFDGQKSSFQTRFNVHREVTPVELPNCNLVKGVDNGSEDLEI |
| | | LPNGLAFISSGLKYPGIMSFDPDKSGKILLMDLNEEDPVVLELGITGNTLDI |
| | | SSFNPHGISTFTDEDNTVYLLVVNHPDSSSTVEVFKFQEEEKSLLHLKTIRH |
| | | KLLPSVNDIVAVGPEHFYATNDHYFADPYLKSWEMHLGLAWSFVTYYSPNDV |
| | | RVVAEGFDFANGINISPDGKYVYIAELLAHKIHVYEKHANWTLTPLKSLDFD |
| | | TLVDNISVDPVTGDLWVGCHPNGMRIFYYDPKNPPGSEVLRIQDILSEEPKV |
| | | TWYAENGTVLQGSTVAAVYKGKLLIGTVFHKALYCEL |
| 66 | hVK3LP- | METPAQLLFLLLLWLPDTTGEPKSSDKTHTCPPCPAPELLGGSSVFLFPPKP |
| | [SCChinge- | KDTLMISRTPEVTCVVVDVSHEDPEVKFNWYVDGVEVHNAKTKPREEQYNST |
| | P238S-P331S | YRVVSVLTVLHQDWLNGKEYKCKVSNKALPASIEKTISKAKGQPREPQVYTL |
| | Fc]-NGS-[M- | PPSRDELTKNQVSLTCLVKGFYPSDIAVEWESNGQPENNYKTTPPVLDSDGS |
| | IIGI PON1] | FFLYSKLTVDKSRWQQGNVFSCSVMHEALHNHYTQKSLSLSPGKVDGASSPV |
| | amino acid | NVSSPSVQDILFDGQKSSFQTRFNVHREVTPVELPNCNLVKGVDNGSEDIEI |
| | | LPNGLAFISSGVKYPGIMSFDPDKSGKILLMDLNEEDPVVLELGITGNTLDI |
| | | SSFNPAGISTFTDEDNTVYLLVVNRPDSSSTVEVFKFQEEEKSLLHLKTIRH |
| | | KLLPSVNDIVAVGPEHFYATNDHYFADPYLKSWEMHLGLAWSFVTYYSPNDV |
| | | RVVAEGFDMANGINISPDGKYVYIAELLAHKIHVYEKHANWTLTPLKSLDFD |
| | | TLVDNISVDPVTGDLWVGCHPNGMRLFYYDPKNPPGSEVLRIQDILSEEPKV |
| | | TVVYAENGTVLQGSSVAAVYKGKLLIGTVFHKALYCEL |
| 67 | hVK3LP- | atggaaaccccagcgcagcttctcttcctcctgctactctggctcccagata |
| | [hDNase1L3 | ccaccggtatgaggatctgctccttcaacgtcaggtcctttggggaaagcaa |
| | R80A-R95A- | gcaggaagacaagaatgccatggatgtcattgtgaaggtcatcaaacgctgt |
| | N96A | gacatcatactcgtgatggaaatcaaggacagcaacaacaggatctgcccca |
| | NLS2delta]- | tactgatggagaagctgaacagaaattcagcaagaggcataacgtacaacta |
| | (g4s)4- | tgtgattagctctcggcttggagcagctacatataaagaacaatatgccttt |
| | [SSShinge- | ctctacaaggaaaagctggtgtctgtgaagaggagttatcactaccatgact |
| | P238S-P331S | atcaggatggagacgcagatgtgttttccagggagccctttgtggtctggtt |
| | Fc]-NGS- | ccaatctccccacactgctgtcaaagacttcgtgattatccccctgcacacc |
| | [PON1 Q192K] | accccagagacatccgttaaggagatcgatgagttggttgaggtctacacgg |
| | nucleotide | acgtgaaacaccgctggaaggcggagaatttcattttcatgggtgacttcaa |
| | | tgccggctgcagctacgtccccaagaaggcctggaagaacatccgcttgagg |
| | | actgaccccaggtttgtttggctgatcggggaccaagaggacaccacggtga |
| | | agaagagcaccaactgtgcatatgacaggattgtgcttagaggacaagaaat |
| | | cgtcagttctgttgttcccaagtcaaacagtgtttttgacttccagaaagct |
| | | tacaagctgactgaagaggaggccctggatgtcagcgaccactttccagttg |
| | | aatttaaactacagtcttcaagggccttcaccaactcagatctctccggagg |
| | | aggtggctcaggtggtggaggatctggaggaggtgggagtggtggaggtggt |
| | | tctaccggtctcgagcccaaatcttctgacaaaactcacacatctccaccgt |
| | | ccccagcacctgaactcctgggaggatcgtcagtcttcctcttccccccaaa |
| | | acccaaggacaccctcatgatctcccggacccctgaggtcacatgcgtggtg |
| | | gtggacgtgagccacgaagaccctgaggtcaagttcaactggtacgtggacg |
| | | gcgtggaggtgcataatgccaagacaaagccgcgggaggagcagtacaacag |
| | | cacgtaccgtgtggtcagcgtcctcaccgtcctgcaccaggactggctgaat |
| | | ggcaaggagtacaagtgcaaggtctccaacaaagccctcccagcctccatcg |
| | | agaaaaccatctccaaagccaaagggcagccccgagaaccacaggtgtacac |
| | | cctgcccccatcccgggatgagctgaccaagaaccaggtcagcctgacctgc |
| | | ctggtcaaaggcttctatcccagcgacatcgccgtggagtgggagagcaatg |
| | | ggcagccggagaacaactacaagaccacgcctcccgtgctggactccgacgg |
| | | ctccttcttcctctacagcaagctcaccgtggacaagagcaggtggcagcag |
| | | gggaacgtcttctcatgctccgtgatgcatgaggctctgcacaaccactaca |
| | | cgcagaagagcctctctctctctccgggtaaagtcgacggagctagcagccc |
| | | cgtgaacgtgagcagccccagcgtgcaggatatcctcttcaggaaccaccag |
| | | tcttcttaccaaacacgacttaatgctctccgagaggtacaacccgtagaac |
| | | ttcctaactgtaatttagttaaaggaatcgaaactggctctgaagacttgga |
| | | gatactgcctaatggactggctttcattagctctggattaaagtatcctgga |
| | | ataaagagcttcaaccccaacagtcctggaaaaatacttctgatggacctga |
| | | atgaagaagatccaacagtgttggaattggggatcactggaagtaaatttga |
| | | tgtatcttcatttaaccctcatgggattagcacattcacagatgaagataat |
| | | gccatgtacctcctggtggtgaaccatccagatgccaagtccacagtggagt |
| | | tgtttaaatttcaagaagaagaaaaatcgcttttgcatctaaaaaccatcag |
| | | acataaacttctgcctaatttgaatgatattgttgctgtgggacctgagcac |
| | | ttttatggcacaaatgatcactattttcttgacccctacttaaaatcctggg |
| | | agatgtatttgggtttagcgtggtcgtatgttgtctactatagtccaagtga |
| | | agttcgagtggtggcagaaggatttgattttgctaatggaatcaacatttca |
| | | cccgatggcaagtatgtctatatagctgagttgctggctcataagattcatg |
| | | tgtatgaaaagcatgctaattggactttaactccattgaagtcccttgactt |
| | | taataccctcgtggataacatatctgtggatcctgagacaggagacctttgg |
| | | gttggatgccatcccaatggcatgaaaatcttcttctatgactcagagaatc |
| | | ctcctgcatcagaggtgcttcgaatccagaacattctaacagaagaacctaa |
| | | agtgacacaggtttatgcagaaaatggcacagtgttgcaaggcagtacagtt |
| | | gcctctgtgtacaaagggaaactgctgattggcacagtgtttcacaaagctc |
| | | tttactgtgagctctaa |
| 68 | hVK3LP- | METPAQLLFLLLLWLPDTTGMRICSFNVRSFGESKQEDKNAMDVIVKVIKRC |
| | [hDNase1L3 | DIILVMEIKDSNNRICPILMEKLNRNSARGITYNYVISSRLGAATYKEQYAF |
| | R80A-R95A- | LYKEKLVSVKRSYHYHDYQDGDADVFSREPFVVWFQSPHTAVKDFVIIPLHT |
| | N96A | TPETSVKE1DELVEVYTDVKHRWKAENFIFMGDFNAGCSYVPKKAWKNIRLR |
| | NLS2delta]- | TDPRFVWLIGDQEDTTVKKSTNCAYDRIVLRGQEIVSSVVPKSNSVFDFQKA |
| | (g4s)4- | YKLTEEEALDVSDHFPVEFKLQSSRAFTNSDLSGGGGSGGGGSGGGGSGGGG |
| | [SSShinge- | STGLEPKSSDKTHTSPPSPAPELLGGSSVFLFPPKPKDTLMISRTPEVTCVV |
| | P238S-P331S | VDVSHEDPEVKFNWYVDGVEVHNAKTKPREEQYNSTYRVVSVLTVLHQDWLN |
| | Fc]-NGS- | GKEYKCKVSNKALPASIEKTISKAKGQPREPQVYTLPPSRDELTKNQVSLTC |
| | [PONI Q192K] | LVKGFYPSDIAVEWESNGQPENNYKTTPPVLDSDGSFFLYSKLTVDKSRWQQ |
| | amino acid | GNVFSCSVMHEALHNHYTQKSLSLSPGKVDGASSPVNVSSPSVQDILFRNHQ |
| | | SSYQTRLNALREVQPVELPNCNLVKGIETGSEDLEILPNGLAFISSGLKYPG |
| | | IKSFNPNSPGKILLMDLNEEDPTVLELGITGSKFDVSSFNPHGISTFTDEDN |
| | | AMYLLVVNHPDAKSTVELFKFQEEEKSLLHLKTIRHKLLPNLNDIVAVGPEH |
| | | FYGTNDHYFLDPYLKSWEMYLGLAWSYVVYYSPSEVRVVAEGFDFANGINIS |
| | | PDGKYVYIAELLAHKIHVYEKHANWTLTPLKSLDFNTLVDNISVDPETGDLW |
| | | VGCHPNGMKIFFYDSENPPASEVLRIQNILTEEPKVTQVYAENGTVLQGSTV |
| | | ASVYKGKLLIGTVFHKALYCEL |
| 69 | hVK3LP- | atggaaaccccagcgcagcttctcttcctcctgctactctggctcccagata |
| | [hDNase1L3 | ccaccggtatgaggatctgctccttcaacgtcaggtcctttggggaaagcaa |
| | R80S-R95S- | gcaggaagacaagaatgccatggatgtcattgtgaaggtcatcaaacgctgt |
| | N96S | gacatcatactcgtgatggaaatcaaggacagcaacaacaggatctgcccca |
| | NLS2delta]- | tactgatggagaagctgaacagaaattcatcaagaggcataacgtacaacta |
| | (g4s)4- | tgtgattagctctcggcttggaagttctacatataaagaacaatatgccttt |
| | [SSShinge- | ctctacaaggaaaagctggtgtctgtgaagaggagttatcactaccatgact |
| | P238S-P331S | atcaggatggagacgcagatgtgttttccagggagccctttgtggtctggtt |
| | Fc]-NGS- | ccaatctccccacactgctgtcaaagacttcgtgattatccccctgcacacc |
| | [PON1 Q192K] | accccagagacatccgttaaggagatcgatgagttggttgaggtctacacgg |
| | nucleotide | acgtgaaacaccgctggaaggcggagaatttcattttcatgggtgacttcaa |
| | | tgccggctgcagctacgtccccaagaaggcctggaagaacatccgcttgagg |
| | | actgaccccaggtttgtttggctgatcggggaccaagaggacaccacggtga |
| | | agaagagcaccaactgtgcatatgacaggattgtgcttagaggacaagaaat |
| | | cgtcagttctgttgttcccaagtcaaacagtgtttttgacttccagaaagct |
| | | tacaagctgactgaagaggaggccctggatgtcagcgaccactttccagttg |
| | | aatttaaactacagtcttcaagggccttcaccaactcagatctctccggagg |
| | | aggtggctcaggtggtggaggatctggaggaggtgggagtggtggaggtggt |
| | | tctaccggtctcgagcccaaatcttctgacaaaactcacacatctccaccgt |
| | | ccccagcacctgaactcctgggaggatcgtcagtcttcctcttccccccaaa |
| | | acccaaggacaccctcatgatctcccggacccctgaggtcacatgcgtggtg |
| | | gtggacgtgagccacgaagaccctgaggtcaagttcaactggtacgtggacg |
| | | gcgtggaggtgcataatgccaagacaaagccgcgggaggagcagtacaacag |
| | | cacgtaccgtgtggtcagcgtcctcaccgtcctgcaccaggactggctgaat |
| | | ggcaaggagtacaagtgcaaggtctccaacaaagccctcccagcctccatcg |
| | | agaaaaccatctccaaagccaaagggcagccccgagaaccacaggtgtacac |
| | | cctgcccccatcccgggatgagctgaccaagaaccaggtcagcctgacctgc |
| | | ctggtcaaaggcttctatcccagcgacatcgccgtggagtgggagagcaatg |
| | | ggcagccggagaacaactacaagaccacgcctcccgtgctggactccgacgg |
| | | ctccttcttcctctacagcaagctcaccgtggacaagagcaggtggcagcag |
| | | gggaacgtcttctcatgctccgtgatgcatgaggctctgcacaaccactaca |
| | | cgcagaagagcctctctctctctccgggtaaagtcgacggagctagcagccc |
| | | cgtgaacgtgagcagccccagcgtgcaggatatcctcttcaggaaccaccag |
| | | tcttcttaccaaacacgacttaatgctctccgagaggtacaacccgtagaac |
| | | ttcctaactgtaatttagttaaaggaatcgaaactggctctgaagacttgga |
| | | gatactgcctaatggactggctttcattagctctggattaaagtatcctgga |
| | | ataaagagcttcaaccccaacagtcctggaaaaatacttctgatggacctga |
| | | atgaagaagatccaacagtgttggaattggggatcactggaagtaaatttga |
| | | tgtatcttcatttaaccctcatgggattagcacattcacagatgaagataat |
| | | gccatgtacctcctggtggtgaaccatccagatgccaagtccacagtggagt |
| | | tgtttaaatttcaagaagaagaaaaatcgcttttgcatctaaaaaccatcag |
| | | acataaacttctgcctaatttgaatgatattgttgctgtgggacctgagcac |
| | | ttttatggcacaaatgatcactattttcttgacccctacttaaaatcctggg |
| | | agatgtatttgggtttagcgtggtcgtatgttgtctactatagtccaagtga |
| | | agttcgagtggtggcagaaggatttgattttgctaatggaatcaacatttca |
| | | cccgatggcaagtatgtctatatagctgagttgctggctcataagattcatg |
| | | tgtatgaaaagcatgctaattggactttaactccattgaagtcccttgactt |
| | | taataccctcgtggataacatatctgtggatcctgagacaggagacctttgg |
| | | gttggatgccatcccaatggcatgaaaatcttcttctatgactcagagaatc |
| | | ctcctgcatcagaggtgcttcgaatccagaacattctaacagaagaacctaa |
| | | agtgacacaggtttatgcagaaaatggcacagtgttgcaaggcagtacagtt |
| | | gcctctgtgtacaaagggaaactgctgattggcacagtgtttcacaaagctc |
| | | tttactgtgagctctaa |
| 70 | hVK3LP- | METPAQLLFLLLLWLPDTTGMRICSFNVRSFGESKQEDKNAMDVIVKVIKRC |
| | [hDNase1L3 | DIILVMEIKDSNNRICPILMEKLNRNSSRGITYNYVISSRLGSSTYKEQYAF |
| | R80S-R95S- | LYKEKLVSVKRSYHYHDYQDGDADVFSREPFVVWFQSPHTAVKDFVIIPLHT |
| | N96S | TPETSVKE1DELVEVYTDVKHRWKAENFIFMGDFNAGCSYVPKKAWKNIRLR |
| | NLS2delta]- | TDPRFVWLIGDQEDTTVKKSTNCAYDRIVLRGQEIVSSVVPKSNSVFDFQKA |
| | (g4s)4- | YKLTEEEALDVSDHFPVEFKLQSSRAFTNSDLSGGGGSGGGGSGGGGSGGGG |
| | [SSShinge- | STGLEPKSSDKTHTSPPSPAPELLGGSSVFLFPPKPKDTLMISRTPEVTCVV |
| | P238S-P331S | VDVSHEDPEVKFNWYVDGVEVHNAKTKPREEQYNSTYRVVSVLTVLHQDWLN |
| | Fc]-NGS- | GKEYKCKVSNKALPASIEKTISKAKGQPREPQVYTLPPSRDELTKNQVSLTC |
| | [PON1 Q192K] | LVKGFYPSDIAVEWESNGQPENNYKTTPPVLDSDGSFFLYSKLTVDKSRWQQ |
| | amino acid | GNVFSCSVMHEALHNHYTQKSLSLSPGKVDGASSPVNVSSPSVQDILFRNHQ |
| | | SSYQTRLNALREVQPVELPNCNLVKGIETGSEDLEILPNGLAFISSGLKYPG |
| | | IKSFNPNSPGKILLMDLNEEDPTVLELGITGSKFDVSSFNPHGISTFTDEDN |
| | | AMYLLVVNHPDAKSTVELFKFQEEEKSLLHLKTIRHKLLPNLNDIVAVGPEH |
| | | FYGTNDHYFLDPYLKSWEMYLGLAWSYVVYYSPSEVRVVAEGFDFANGINIS |
| | | PDGKYVYIAELLAHKIHVYEKHANWTLTPLKSLDFNTLVDNISVDPETGDLW |
| | | VGCHPNGMKIFFYDSENPPASEVLRIQNILTEEPKVTQVYAENGTVLQGSTV |
| | | ASVYKGKLLIGTVFHKALYCEL |
| 71 | hVK3LP- | atggaaaccccagcgcagcttctcttcctcctgctactctggctcccagata |
| | [hDNase1 WT]- | ccaccggtctgaagatcgcagccttcaacatccagacatttggggagaccaa |
| | (g4s)4- | gatgtccaatgccaccctcgtcagctacattgtgcagatcctgagccgctat |
| | [SSShinge- | gacatcgccctggtccaggaggtcagagacagccacctgactgccgtgggga |
| | P238S-P331S | agctgctggacaacctcaatcaggatgcaccagacacctatcactacgtggt |
| | Fc] | cagtgagccactgggacggaacagctataaggagcgctacctgttcgtgtac |
| | nucleotide | aggcctgaccaggtgtctgcggtggacagctactactacgatgatggctgcg |
| | | agccctgcgggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 72 | hVK3LP- | METPAQLLFLLLLWLPDTTGLKIAAFNIQTFGETKMSNATLVSYIVQILSRY |
| | [hDNase1 WT]- | DIALVQEVRDSHLTAVGKLLDNLNQDAPDTYHYVVSEPLGRNSYKERYLFVY |
| | (g4s)4- | RPDQVSAVDSYYYDDGCEPCGNDTFNREPAIVRFFSRFTEVREFAIVPLHAA |
| | [SSShinge- | PGDAVAEIDALYDVYLDVQEKWGLEDVMLMGDFNAGCSYVRPSQWSSIRLWT |
| | P238S-P331S | SPTFQWLIPDSADTTATPTHCAYDRIVVAGMLLRGAVVPDSALPFNFQAAYG |
| | Fc] | LSDQLAQAISDHYPVEVMLKDLSGGGGSGGGGSGGGGSGGGGSTGLEPKSSD |
| | amino acid | KTHTSPPSPAPELLGGSSVFLFPPKPKDTLMISRTPEVTCVVVDVSHEDPEV |
| | | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 73 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | D53R-N74K- | gatgtccaatgccaccctcgtcagctacattgtgcagatcctgagccgctat |
| | G105R] | gacatcgccctggtccaggaggtcagagacagccacctgactgccgtgggga |
| | nucleotide | agctgctgaggaacctcaatcaggatgcaccagacacctatcactacgtggt |
| | | cagtgagccactgggacggaagagctataaggagcgctacctgttcgtgtac |
| | | aggcctgaccaggtgtctgcggtggacagctactactacgatgatggctgcg |
| | | agccctgcaggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 74 | hVK3LP- | METPAQLLFLLLLWLPDTTGLKIAAFNIQTFGETKMSNATLVSYIVQILSRY |
| | [hDNase1 | DIALVQEVRDSHLTAVGKLLRNLNQDAPDTYHYVVSEPLGRKSYKERYLFVY |
| | D53R-N74K- | RPDQVSAVDSYYYDDGCEPCRNDTFNREPAIVRFFSRFTEVREFAIVPLHAA |
| | G105R] | PGDAVAEIDALYDVYLDVQEKWGLEDVMLMGDFNAGCSYVRPSQWSSIRLWT |
| | amino acid | SPTFQWLIPDSADTTATPTHCAYDRIVVAGMLLRGAVVPDSALPFNFQAAYG |
| | | LSDQLAQAISDHYPVEVMLK |
| 75 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | Y65A-N74K- | gatgtccaatgccaccctcgtcagctacattgtgcagatcctgagccgctat |
| | G105R] | gacatcgccctggtccaggaggtcagagacagccacctgactgccgtgggga |
| | nucleotide | agctgctggacaacctcaatcaggatgcaccagacacctatcacgccgtggt |
| | | cagtgagccactgggacggaagagctataaggagcgctacctgttcgtgtac |
| | | aggcctgaccaggtgtctgcggtggacagctactactacgatgatggctgcg |
| | | agccctgcaggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 76 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHAVVSEPLGRKSYKERYLFVY |
| | Y65A-N74K- | RPDQVSAVDSYYYDDGCEPCRNDTFNREPAIVRFFSRFTEVREFAIVPLHAA |
| | G105R] | PGDAVAEIDALYDVYLDVQEKWGLEDVMLMGDFNAGCSYVRPSQWSSIRLWT |
| | amino acid | SPTFQWLIPDSADTTATPTHCAYDRIVVAGMLLRGAVVPDSALPFNFQAAYG |
| | | LSDQLAQAISDHYPVEVMLK |
| 77 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | E69K-N74K- | gatgtccaatgccaccctcgtcagctacattgtgcagatcctgagccgctat |
| | G105R] | gacatcgccctggtccaggaggtcagagacagccacctgactgccgtgggga |
| | nucleotide | agctgctggacaacctcaatcaggatgcaccagacacctatcactacgtggt |
| | | cagtaagccactgggacggaagagctataaggagcgctacctgttcgtgtac |
| | | aggcctgaccaggtgtctgcggtggacagctactactacgatgatggctgcg |
| | | agccctgcaggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 78 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSKPLGRKSYKERYLFVY |
| | E69K-N74K- | RPDQVSAVDSYYYDDGCEPCRNDTFNREPAIVRFFSRFTEVREFAIVPLHAA |
| | G105R] | PGDAVAEIDALYDVYLDVQEKWGLEDVMLMGDFNAGCSYVRPSQWSSIRLWT |
| | amino acid | SPTFQWLIPDSADTTATPTHCAYDRIVVAGMLLRGAVVPDSALPFNFQAAYG |
| | | LSDQLAQAISDHYPVEVMLK |
| 79 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | D53R] | gatgtccaatgccaccctcgtcagctacattgtgcagatcctgagccgctat |
| | nucleotide | gacatcgccctggtccaggaggtcagagacagccacctgactgccgtgggga |
| | | agctgctgaggaacctcaatcaggatgcaccagacacctatcactacgtggt |
| | | cagtgagccactgggacggaacagctataaggagcgctacctgttcgtgtac |
| | | aggcctgaccaggtgtctgcggtggacagctactactacgatgatggctgcg |
| | | agccctgcgggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 80 | hVK3LP- | METPAQLLFLLLLWLPDTTGLKIAAFNIQTFGETKMSNATLVSYIVQILSRY |
| | [hDNase1 | DIALVQEVRDSHLTAVGKLLRNLNQDAPDTYHYVVSEPLGRNSYKERYLFVY |
| | D53R] | RPDQVSAVDSYYYDDGCEPCGNDTFNREPAIVRFFSRFTEVREFAIVPLHAA |
| | amino acid | PGDAVAEIDALYDVYLDVQEKWGLEDVMLMGDFNAGCSYVRPSQWSSIRLWT |
| | | SPTFQWLIPDSADTTATPTHCAYDRIVVAGMLLRGAVVPDSALPFNFQAAYG |
| | | LSDQLAQAISDHYPVEVMLK |
| 81 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | Y65A] | gatgtccaatgccaccctcgtcagctacattgtgcagatcctgagccgctat |
| | nucleotide | gacatcgccctggtccaggaggtcagagacagccacctgactgccgtgggga |
| | | agctgctggacaacctcaatcaggatgcaccagacacctatcacgccgtggt |
| | | cagtgagccactgggacggaacagctataaggagcgctacctgttcgtgtac |
| | | aggcctgaccaggtgtctgcggtggacagctactactacgatgatggctgcg |
| | | agccctgcgggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 82 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHAVVSEPLGRNSYKERYLFVY |
| | Y65A] | RPDQVSAVDSYYYDDGCEPCGNDTFNREPAIVRFFSRFTEVREFAIVPLHAA |
| | amino acid | PGDAVAEIDALYDVYLDVQEKWGLEDVMLMGDFNAGCSYVRPSQWSSIRLWT |
| | | SPTFQWLIPDSADTTATPTHCAYDRIVVAGMLLRGAVVPDSALPFNFQAAYG |
| | | LSDQLAQAISDHYPVEVMLK |
| 83 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | E69K] | gatgtccaatgccaccctcgtcagctacattgtgcagatcctgagccgctat |
| | nucleotide | gacatcgccctggtccaggaggtcagagacagccacctgactgccgtgggga |
| | | agctgctggacaacctcaatcaggatgcaccagacacctatcactacgtggt |
| | | cagtaagccactgggacggaacagctataaggagcgctacctgttcgtgtac |
| | | aggcctgaccaggtgtctgcggtggacagctactactacgatgatggctgcg |
| | | agccctgcgggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaa |
| 84 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSKPLGRNSYKERYLFVY |
| | E69K] | RPDQVSAVDSYYYDDGCEPCGNDTFNREPAIVRFFSRFTEVREFAIVPLHAA |
| | amino acid | PGDAVAEIDALYDVYLDVQEKWGLEDVMLMGDFNAGCSYVRPSQWSSIRLWT |
| | | SPTFQWLIPDSADTTATPTHCAYDRIVVAGMLLRGAVVPDSALPFNFQAAYG |
| | | LSDQLAQAISDHYPVEVMLK |
| 85 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | D53R-N74K- | gatgtccaatgccaccctcgtcagctacattgtgcagatcctgagccgctat |
| | G105R]-(g4s)4- | gacatcgccctggtccaggaggtcagagacagccacctgactgccgtgggga |
| | [SSShinge- | agctgctgaggaacctcaatcaggatgcaccagacacctatcactacgtggt |
| | P238S-P331S | cagtgagccactgggacggaagagctataaggagcgctacctgttcgtgtac |
| | Fc] | aggcctgaccaggtgtctgcggtggacagctactactacgatgatggctgcg |
| | nucleotide | agccctgcaggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 86 | hVK3LP- | METPAQLLFLLLLWLPDTTGLKIAAFNIQTFGETKMSNATLVSYIVQILSRY |
| | [hDNase1 | DIALVQEVRDSHLTAVGKLLRNLNQDAPDTYHYVVSEPLGRKSYKERYLFVY |
| | D53R-N74K- | RPDQVSAVDSYYYDDGCEPCRNDTFNREPAIVRFFSRFTEVREFAIVPLHAA |
| | G105R]-(g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc] | KTHTSPPSPAPELLGGSSVFLFPPKPKDTLMISRTPEVTCVVVDVSHEDPEV |
| | amino acid | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 87 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | Y65A-N74K- | gatgtccaatgccaccctcgtcagctacattgtgcagatcctgagccgctat |
| | G105R]-(g4s)4- | gacatcgccctggtccaggaggtcagagacagccacctgactgccgtgggga |
| | [SSShinge- | agctgctggacaacctcaatcaggatgcaccagacacctatcacgccgtggt |
| | P238S-P331S | cagtgagccactgggacggaagagctataaggagcgctacctgttcgtgtac |
| | Fc] | aggcctgaccaggtgtctgcggtggacagctactactacgatgatggctgcg |
| | nucleotide | agccctgcaggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 88 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHAVVSEPLGRKSYKERYLFVY |
| | Y65A-N74K- | RPDQVSAVDSYYYDDGCEPCRNDTFNREPAIVRFFSRFTEVREFAIVPLHAA |
| | G105R]-(g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc] | KTHTSPPSPAPELLGGSSVFLFPPKPKDTLMISRTPEVTCVVVDVSHEDPEV |
| | amino acid | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 89 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | E69K-N74K- | gatgtccaatgccaccctcgtcagctacattgtgcagatcctgagccgctat |
| | G105R]-(g4s)4- | gacatcgccctggtccaggaggtcagagacagccacctgactgccgtgggga |
| | [SSShinge- | agctgctggacaacctcaatcaggatgcaccagacacctatcactacgtggt |
| | P238S-P331S | cagtaagccactgggacggaagagctataaggagcgctacctgttcgtgtac |
| | Fc] | aggcctgaccaggtgtctgcggtggacagctactactacgatgatggctgcg |
| | nucleotide | agccctgcaggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 90 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSKPLGRKSYKERYLFVY |
| | E69K-N74K- | RPDQVSAVDSYYYDDGCEPCRNDTFNREPAIVRFFSRFTEVREFAIVPLHAA |
| | G105R]-(g4s)4- | PGDAVAEIDALYDVYLDVQEKWGLEDVMLMGDFNAGCSYVRPSQWSSIRLWT |
| | [SSShinge- | SPTFQWLIPDSADTTATPTHCAYDRIVVAGMLLRGAVVPDSALPFNFQAAYG |
| | P238S-P331S | LSDQLAQAISDHYPVEVMLKDLSGGGGSGGGGSGGGGSGGGGSTGLEPKSSD |
| | Fc] | KTHTSPPSPAPELLGGSSVFLFPPKPKDTLMISRTPEVTCVVVDVSHEDPEV |
| | amino acid | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 91 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | D53R]-(g4s)4- | gatgtccaatgccaccctcgtcagctacattgtgcagatcctgagccgctat |
| | [SSShinge- | gacatcgccctggtccaggaggtcagagacagccacctgactgccgtgggga |
| | P238S-P331S | agctgctgaggaacctcaatcaggatgcaccagacacctatcactacgtggt |
| | Fc] | cagtgagccactgggacggaacagctataaggagcgctacctgttcgtgtac |
| | nucleotide | aggcctgaccaggtgtctgcggtggacagctactactacgatgatggctgcg |
| | | agccctgcgggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 92 | hVK3LP- | METPAQLLFLLLLWLPDTTGLKIAAFNIQTFGETKMSNATLVSYIVQILSRY |
| | [hDNase1 | DIALVQEVRDSHLTAVGKLLRNLNQDAPDTYHYVVSEPLGRNSYKERYLFVY |
| | D53R]-(g4s)4- | RPDQVSAVDSYYYDDGCEPCGNDTFNREPAIVRFFSRFTEVREFAIVPLHAA |
| | [SSShinge- | PGDAVAEIDALYDVYLDVQEKWGLEDVMLMGDFNAGCSYVRPSQWSSIRLWT |
| | P238S-P331S | SPTFQWLIPDSADTTATPTHCAYDRIVVAGMLLRGAVVPDSALPFNFQAAYG |
| | Fc] | LSDQLAQAISDHYPVEVMLKDLSGGGGSGGGGSGGGGSGGGGSTGLEPKSSD |
| | amino acid | KTHTSPPSPAPELLGGSSVFLFPPKPKDTLMISRTPEVTCVVVDVSHEDPEV |
| | | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 93 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | Y65A]-(g4s)4- | gatgtccaatgccaccctcgtcagctacattgtgcagatcctgagccgctat |
| | [SSShinge- | gacatcgccctggtccaggaggtcagagacagccacctgactgccgtgggga |
| | P238S-P331S | agctgctggacaacctcaatcaggatgcaccagacacctatcacgccgtggt |
| | Fc] | cagtgagccactgggacggaacagctataaggagcgctacctgttcgtgtac |
| | nucleotide | aggcctgaccaggtgtctgcggtggacagctactactacgatgatggctgcg |
| | | agccctgcgggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 94 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHAVVSEPLGRNSYKERYLFVY |
| | Y65A]-(g4s)4- | RPDQVSAVDSYYYDDGCEPCGNDTFNREPAIVRFFSRFTEVREFAIVPLHAA |
| | [SSShinge- | PGDAVAEIDALYDVYLDVQEKWGLEDVMLMGDFNAGCSYVRPSQWSSIRLWT |
| | P238S-P331S | SPTFQWLIPDSADTTATPTHCAYDRIVVAGMLLRGAVVPDSALPFNFQAAYG |
| | Fc] | LSDQLAQAISDHYPVEVMLKDLSGGGGSGGGGSGGGGSGGGGSTGLEPKSSD |
| | amino acid | KTHTSPPSPAPELLGGSSVFLFPPKPKDTLMISRTPEVTCVVVDVSHEDPEV |
| | | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 95 | hVK3LP- | atggaaaccccagcgcagcttctcttcctcctgctactctggctcccagata |
| | [hDNase1 | ccaccggtctgaagatcgcagccttcaacatccagacatttggggagaccaa |
| | E69K]-(g4s)4- | gatgtccaatgccaccctcgtcagctacattgtgcagatcctgagccgctat |
| | [SSShinge- | gacatcgccctggtccaggaggtcagagacagccacctgactgccgtgggga |
| | P238S-P331S | agctgctggacaacctcaatcaggatgcaccagacacctatcactacgtggt |
| | Fc] | cagtaagccactgggacggaacagctataaggagcgctacctgttcgtgtac |
| | nucleotide | aggcctgaccaggtgtctgcggtggacagctactactacgatgatggctgcg |
| | | agccctgcgggaacgacaccttcaaccgagagccagccattgtcaggttctt |
| | | ctcccggttcacagaggtcagggagtttgccattgttcccctgcatgcggcc |
| | | ccgggggacgcagtagccgagatcgacgctctctatgacgtctacctggatg |
| | | tccaagagaaatggggcttggaggacgtcatgttgatgggcgacttcaatgc |
| | | gggctgcagctatgtgagaccctcccagtggtcatccatccgcctgtggaca |
| | | agccccaccttccagtggctgatccccgacagcgctgacaccacagctacac |
| | | ccacgcactgtgcctatgacaggatcgtggttgcagggatgctgctccgagg |
| | | cgccgttgttcccgactcggctcttccctttaacttccaggctgcctatggc |
| | | ctgagtgaccaactggcccaagccatcagtgaccactatccagtggaggtga |
| | | tgctgaaagatctctccggaggaggtggctcaggtggtggaggatctggagg |
| | | aggtgggagtggtggaggtggttctaccggtctcgagcccaaatcttctgac |
| | | aaaactcacacatctccaccgtccccagcacctgaactcctgggaggatcgt |
| | | cagtcttcctcttccccccaaaacccaaggacaccctcatgatctcccggac |
| | | ccctgaggtcacatgcgtggtggtggacgtgagccacgaagaccctgaggtc |
| | | aagttcaactggtacgtggacggcgtggaggtgcataatgccaagacaaagc |
| | | cgcgggaggagcagtacaacagcacgtaccgtgtggtcagcgtcctcaccgt |
| | | cctgcaccaggactggctgaatggcaaggagtacaagtgcaaggtctccaac |
| | | aaagccctcccagcctccatcgagaaaaccatctccaaagccaaagggcagc |
| | | cccgagaaccacaggtgtacaccctgcccccatcccgggatgagctgaccaa |
| | | gaaccaggtcagcctgacctgcctggtcaaaggcttctatcccagcgacatc |
| | | gccgtggagtgggagagcaatgggcagccggagaacaactacaagaccacgc |
| | | ctcccgtgctggactccgacggctccttcttcctctacagcaagctcaccgt |
| | | ggacaagagcaggtggcagcaggggaacgtcttctcatgctccgtgatgcat |
| | | gaggctctgcacaaccactacacgcagaagagcctctctctctctccgggta |
| | | aa |
| 96 | hVK3LP- | METPAQLLFLLLLWLPDTTGLKIAAFNIQTFGETKMSNATLVSYIVQILSRY |
| | [hDNase1 | DIALVQEVRDSHLTAVGKLLDNLNQDAPDTYHYVVSKPLGRNSYKERYLFVY |
| | E69K]-(g4s)4- | RPDQVSAVDSYYYDDGCEPCGNDTFNREPAIVRFFSRFTEVREFAIVPLHAA |
| | [SSShinge- | PGDAVAEIDALYDVYLDVQEKWGLEDVMLMGDFNAGCSYVRPSQWSSIRLWT |
| | P238S-P331S | SPTFQWLIPDSADTTATPTHCAYDRIVVAGMLLRGAVVPDSALPFNFQAAYG |
| | Fc] | LSDQLAQAISDHYPVEVMLKDLSGGGGSGGGGSGGGGSGGGGSTGLEPKSSD |
| | amino acid | KTHTSPPSPAPELLGGSSVFLFPPKPKDTLMISRTPEVTCVVVDVSHEDPEV |
| | | KFNWYVDGVEVHNAKTKPREEQYNSTYRVVSVLTVLHQDWLNGKEYKCKVSN |
| | | KALPASIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMH |
| | | EALHNHYTQKSLSLSPGK |
| 97 | [hDNase1 | LKIAAFNIQTFGETKMSNATLVSYIVQILSRYDIALVQEVRDSHLTAVGKLL |
| | D53X-Y65X- | XNLNQDAPDTYHXVVSXPLGRXSYKERYLFVYRPDQVSAVDSYYYDDGCEPC |
| | E69X-N74X- | XNDTFNREPAIVRFFSRFTEVREFAIVPLHAAPGDAVAEIDALYDVYLDVQE |
| | G105X]-[gs | KWGLEDVMLMGDFNAGCSYVRPSQWSSIRLWTSPTFQWLIPDSADTTATPTH |
| | linker]- | CAYDRIVVAGMLLRGAVVPDSALPFNFQAAYGLSDQLAQAISDHYPVEVMLK |
| | [SSShinge Fc] | XXXXXXXXXXXXXXXXXXXXXXXXXXXXXXXXXXXXEPKSSDKTHTSPPSPA |
| | amino acid | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 98 | [hDNase1 | LKIAAFNIQTFGETKMSNATLVSYIVQILSRYDIALVQEVRDSHLTAVGKLL |
| | D53X-N74X- | XNLNQDAPDTYHYVVSEPLGRXSYKERYLFVYRPDQVSAVDSYYYDDGCEPC |
| | G105X]-[gs | XNDTFNREPAIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 99 | [hDNase1 | LKIAAFNIQTFGETKMSNATLVSYIVQILSRYDIALVQEVRDSHLTAVGKLL |
| | Y65X-N74X- | DNLNQDAPDTYHXVVSEPLGRXSYKERYLFVYRPDQVSAVDSYYYDDGCEPC |
| | G105X]-[gs | XNDTFNREPAIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 100 | [hDNase1 | LKIAAFNIQTFGETKMSNATLVSYIVQILSRYDIALVQEVRDSHLTAVGKLL |
| | E69X-N74X- | DNLNQDAPDTYHYVVSXPLGRXSYKERYLFVYRPDQVSAVDSYYYDDGCEPC |
| | G105X]-[gs | XNDTFNREPAIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 101 | [hDNase1 | LKIAAFNIQTFGETKMSNATLVSYIVQILSRYDIALVQEVRDSHLTAVGKLL |
| | D53X-Y65X- | XNLNQDAPDTYHXVVSEPLGRXSYKERYLFVYRPDQVSAVDSYYYDDGCEPC |
| | N74X-G105X]- | XNDTFNREPAIVRFFSRFTEVREFAIVPLHAAPGDAVAEIDALYDVYLDVQE |
| | [gs linker] - | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 102 | [hDNase1 | LKIAAFNIQTFGETKMSNATLVSYIVQILSRYDIALVQEVRDSHLTAVGKLL |
| | D53R-N74K- | RNLNQDAPDTYHYVVSEPLGRKSYKERYLFVYRPDQVSAVDSYYYDDGCEPC |
| | G105R]-[gs | RNDTFNREPAIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 103 | [hDNase1 | LKIAAFNIQTFGETKMSNATLVSYIVQILSRYDIALVQEVRDSHLTAVGKLL |
| | Y65A-N74K- | DNLNQDAPDTYHAVVSEPLGRKSYKERYLFVYRPDQVSAVDSYYYDDGCEPC |
| | G105R]-[gs | RNDTFNREPAIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |
| 104 | [hDNase1 | LKIAAFNIQTFGETKMSNATLVSYIVQILSRYDIALVQEVRDSHLTAVGKLL |
| | E69K-N74K- | DNLNQDAPDTYHYVVSKPLGRKSYKERYLFVYRPDQVSAVDSYYYDDGCEPC |
| | G105R]-[gs | RNDTFNREPAIVRFFSRFTEVREFAIVPLHAAPGDAVAEIDALYDVYLDVQE |
| | linker]- | KWGLEDVMLMGDFNAGCSYVRPSQWSSIRLWTSPTFQWLIPDSADTTATPTH |
| | [SSShinge Fc] | CAYDRIVVAGMLLRGAVVPDSALPFNFQAAYGLSDQLAQAISDHYPVEVMLK |
| | amino acid | XXXXXXXXXXXXXXXXXXXXXXXXXXXXXXXXXXXXEPKSSDKTHTSPPSPA |
| | | PEXXGGXSVFLFPPKPKDTLXIXRXPEVTCVVVXVSHEDPEVKFNWYVDGVE |
| | | VHNAKTKPREEQYXSTYRVVSVLTVLHQDWLNGKEYKCKVSNKALXAXIEKT |
| | | ISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVXHEALHXHYTQK |
| | | SLSLSPGX |

TABLE 5b
Variant Amino Acids of SEQ ID NOs: 49, 50, 53, 54, and 97-104

| Relevant SEQ ID | AA | Eu | |
|---|---|---|---|
| NO(s) | Position(s) | Numbering | Definition |
| 97 | 53 | NA | X is D, A, M,R, or Y |
| 98, 101 | 53 | NA | X is A, M,R, or Y |
| 97 | 65 | NA | X is Y, A, or R |
| 99, 101 | 65 | NA | X is A or R |
| 97 | 69 | NA | X is E, K, or R |
| 100 | 69 | NA | X is K or R |
| 49, 53, 97-101 | 74 | NA | X is K, R, or H |
| 49, 53, 97-101 | 105 | NA | X is K, or, or H |
| 49 | 114 | NA | X is F, E, M, R, or Y |
| 49, 50, 53, 54, 97-104 | 261-263, | NA | X is any naturally |
| | 294-296 | | occurring non-cysteine |
| | | | amino acid |
| 49, 50, 53, 54, 97-104 | 264-283 | NA | X is G or S |
| 49, 50, 53, 54, 97-104 | 284-293 | NA | X is G or S or is absent |
| 49, 50, 53, 54, 97-104 | 315, 316 | 234, 235 | X is L or A |
| 49, 50, 53, 54, 97-104 | 319 | 238 | X is P or S |
| 49, 50, 53, 54, 97-104 | 333 | 252 | X is M or Y |
| 49, 50, 53, 54, 97-104 | 335 | 254 | X is S or T |
| 49, 50, 53, 54, 97-104 | 337 | 256 | X is T or E |
| 49, 50, 53, 54, 97-104 | 346 | 265 | X is D or A |
| 49, 50, 53, 54, 97-104 | 378 | 297 | X is N, A, Q, or G |
| 49, 50, 53, 54, 97-104 | 410 | 329 | X is P, A, G, or S |
| 49, 50, 53, 54, 97-104 | 412 | 331 | X is P, S, or A |
| 49, 50, 53, 54, 97-104 | 509 | 428 | X is M or L |
| 49, 50, 53, 54, 97-104 | 515 | 434 | X is N or S |
| 49, 50, 53, 54, 97-104 | 528 | 447 | X is K or is absent |

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

What is claimed is:

1. A polynucleotide encoding a DNase-Fc fusion polypeptide, wherein the fusion polypeptide comprises the amino acid sequence shown in
(i) residues 21-538 or 21-537 of SEQ ID NO:18; or
(ii) residues 21-548 or 21-547 of SEQ ID NO:20.

2. An expression cassette comprising a DNA segment encoding a DNase-Fc fusion polypeptide, wherein the fusion polypeptide comprises the amino acid sequence shown in
(i) residues 21-538 or 21-537 of SEQ ID NO:18; or
(ii) residues 21-548 or 21-547 of SEQ ID NO:20; and
wherein the DNA segment is operably linked to a promoter.

3. A cultured cell into which has been introduced the expression cassette of claim 2, wherein the cell expresses the DNA segment.

4. The cultured cell of claim 3, wherein the cultured cell is a stable cell line comprising the expression cassette within its genomic DNA, and wherein the stable cell line constitutively expresses the DNA segment.

5. A method of making a fusion polypeptide, the method comprising:
culturing a cell into which has been introduced the expression cassette of claim 2, wherein the cell expresses the DNA segment and the encoded fusion polypeptide is produced; and
recovering the fusion polypeptide.

6. The method of claim 5, wherein the encoded fusion polypeptide is produced in the cell and recovered as a dimeric protein.
